(19) United States
(12) Patent Application Publication
(10) Pub. No.: US 2024/0091259 A1
(43) Pub. Date: Mar. 21, 2024

(54) GENERATION OF ANTI-TUMOR T CELLS

(71) Applicant: Dana-Farber Cancer Institute, Inc., Boston, MA (US)

(72) Inventors: Catherine J. Wu, Brookline, MA (US); Giacomo Oliveira, Boston, MA (US)

(21) Appl. No.: 18/224,865

(22) Filed: Jul. 21, 2023

Related U.S. Application Data
(60) Provisional application No. 63/391,141, filed on Jul. 21, 2022.

(51) Int. Cl. A61K 35/17; C12Q 1/6881

(52) U.S. Cl. CPC A61K 35/17; C12Q 1/6881

(57) ABSTRACT

Disclosed are methods for identifying expanded, exhausted, and tumor-specific T-cell clonotypes for adoptive cell transfer, and methods of cancer treatment and modified T cells with anti-tumor T cell receptors (TCRs).

### CROSS-REFERENCE TO RELATED APPLICATIONS

This application claims the benefit of U.S. Provisional Application Ser. No. 63/391,141, filed on Jul. 21, 2022, which is incorporated herein by reference in its entirety.

### STATEMENT REGARDING FEDERALLY SPONSORED RESEARCH

This invention was made with government support under grant number R01 CA155010 awarded by the National Institutes of Health and W81XWH-18-1-0367 awarded by the Assistant Secretary of Defense for Health Affairs, endorsed by the Department of Defense. The government has certain rights in the invention.

### SEQUENCE LISTING

This application contains a Sequence Listing electronically submitted to the United States Patent and Trademark Office via Patent Center as an XML file entitled “0680002976US02” having a size of 343 kilobytes and created on Jul. 21, 2023. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### BACKGROUND

Tumor-infiltrating lymphocytes (TILs) are known to have heterogeneous phenotypic cellular states. However, the correlation among phenotypic state and T cell receptor (TCR) sequences and antitumor reactivity is unknown.

Despite the dramatic successes achieved with cellular therapy for B cell malignancies, translation of the same successes in solid tumors (i.e., tumours) has been elusive. The limited results achieved thus far have been attributed to the intrinsic tumor diversity and lack of conserved tumor antigens that could be targeted by gene-modified lymphocytes. Adoptive transfer of polyclonal tumor-infiltrating T cells (TILs) has been long-appreciated as a promising approach to controlling solid tumors. However, a major challenge to the consistent robustness of this strategy relies on the variable degree to which TIL products are comprised of T cells with tumor-specificity. Hence, the identification of tumor-reactive T cells and the definition of their properties are high-priority goals. In addition, growing evidence has pointed to the highly dysfunctional states of tumor-infiltrating T cells. Therefore, strategies to effectively re-activate the functionality of these cells to effectuate consistent tumor killing are needed.

### SUMMARY

An aspect of the present disclosure is directed to a method of identifying T cell receptors (TCR) sequences expressed in exhausted T cells of a subject (i.e., patient) with cancer. The method comprises: collecting T cells from a tumor biopsy obtained from the subject (e.g., a cancer patient); assigning the T cells into a plurality of clonotype families on the basis of TCR sequences determined by single cell T cell receptor sequencing (scTCRseq); identifying an expanded clonotype family from among the plurality of clonotype families, wherein the T cells in the thus-identified expanded clonotype family expresses one or more exhaustion markers comprising a) one or more of PDCD1, HAVCR2, CTLA4, ENTPD1, LAG3, and TOXRNA transcripts determined using high throughput single cell transcriptome sequencing (scRNA seq), and/or b) one or more of PD1, Tim-3, CTLA4, CD39, and LAG3 surface proteins (wherein a and b are also referred to herein as “exhaustion markers”); and sequencing a TCR sequence from a T cell in the expanded TCR clonotype family. This method may further comprise generating a cDNA encoding said TCR sequence.

Another aspect of the present disclosure is directed to a non-exhausted T cell, which may be autologous or allogeneic, and which is modified with an exogenous nucleic acid comprising a sequence encoding a TCR expressed on an exhausted T cell in a subject with a cancer, wherein the exhausted T cell expresses one or more exhaustion markers comprising a) one or more of PDCD1, HAVCR2, CTLA4, ENTPD1, LAG3, and TOXRNA transcripts and/or b) one or more of PD1, Tim-3, CTLA4, CD39 and LAG3 surface proteins.

Another aspect of the present disclosure is directed to a method of treating cancer in a subject. The method entails administering to the subject non-expressed T cells modified with an exogenous nucleic acid comprising a sequence encoding a TCR expressed on an exhausted T cell isolated from the subject or from a subject (different from the subject receiving the treatment) who has a malignant tumor, wherein the exhausted T cell expresses one or more exhaustion markers comprising a) one or more of PDCD1, HAVCR2, CTLA4, ENTPD1, LAG3, and TOX RNA transcripts and/or b) one or more of PD1, Tim-3, CTLA4, CD39, and LAG3 surface proteins.

In some embodiments of the disclosure, the exhausted T cells express one or more of PDCD1, HAVCR2, and LAG3 RNA transcripts, and/or one or more of PD1, Tim-3, LAG3, and CD39 surface proteins. In some embodiments of the disclosure, the exhausted T cells co-express PD1 and CD39 surface proteins. In some embodiments of the disclosure, the exhausted T cells contain PDCD1 and ENTPD1 RNA transcripts.

In some embodiments of the disclosure, the T cell modified with the exogenous nucleic acid comprising a sequence encoding a TCR expressed on an exhausted T cell in a subject with cancer is an allogeneic T cell with at least a partial HLA-match with the subject.

In some embodiments of the disclosure, the T cells modified with the exogenous nucleic acid comprising a sequence encoding a TCR expressed on an exhausted T cell in a subject with cancer are autologous non-exhausted T cells isolated from the subject. In some embodiments, the exhausted T cell is a CD8+ T cell. In some embodiments, the autologous T cells are obtained from the peripheral blood of the subject. In some embodiments, the autologous T cells are memory T cells.

The methods of the present disclosure apply to a wide variety of cancers, particularly those having solid tumors. In some embodiments of the disclosure, the subject has a carcinoma. In some embodiments, the subject has breast cancer. In some embodiments, the subject has lung cancer. In some embodiments, the subject has a gastrointestinal cancer. In some embodiments, the subject has colorectal cancer. In some embodiments, the subject has melanoma. In some embodiments, the subject has lymphoma. In some embodiments, the subject has a sarcoma, In some embodiments, the subject has renal cell carcinoma.

### BRIEF DESCRIPTION OF THE DRAWINGS

FIG. 1A is a schematic of sample collection, processing, and single-cell (sc) sequencing analysis.

FIG. 1B provides UMAPs illustrating the distinct pattern of cell states of intratumoral CD8+ TCR clonotype families in patients with melanoma.

FIG. 1C is a bar plot showing the top 100 TCR clonotype families from four patients.

FIG. 2A is a schematic representation of the workflow for in vitro TCR reconstruction and specificity screening.

FIG. 2B includes heatmaps showing the reactivity of dominant TCRs originating from cells in exhausted (T_{Ex}, top) and/or non-exhausted memory (T_{NExM}, bottom) clusters infiltrating 4 melanoma specimens.

FIG. 2C is a box plot showing tumor-specific (left) and EBV-specific (right) TCR clonotypes.

FIG. 2D is a bar plot showing TCRs from T_{Ex} or T_{NExM} clusters that perfectly matched with known TCR sequences.

FIG. 2E is a UMAP of scRNA-seq data from CD8+ TILs.

FIG. 2F is a bar plot showing the CD8+ phenotypes of TCRs.

FIG. 3A includes four pie plots showing a summary of the de-orphanized antigen specificity of intratumoral TCRs with confirmed antitumor reactivity.

FIG. 3B is a series of UMAP plots showing the antigenic specificity and recognition avidity of tumor-specific TCRs.

FIG. 4A is a series of heatmaps depicting the mean cluster expression of a panel of T-cell related genes.

FIG. 4B shows violin plots quantifying relative transcriptional expression of genes (columns) with high differential expression among CD8+ TIL clusters (rows).

FIG. 4C shows UMAPs depicting the single-cell expression of representative T cell markers among CD8+ TILs.

FIG. 5 is a series of dot plots showing the antitumor reactivity of in vitro reconstructed TCRs.

FIG. 6A and FIG. 6B are dot plots showing antigen specificity screening of 94 TCRs sequenced from clonally expanded CD8+ T cells.

FIG. 6C is a table showing antigen specificity screening of 94 TCRs sequenced from clonally expanded CD8+ T cells.

FIG. 6D is a UMAP showing single-cell phenotype of TILs with antiviral or anti-MAA TCRs.

FIG. 6E is a UMAP showing single-cell phenotype of TILs with antiviral or anti-MAA TCRs. Pie charts shown in FIG. 6E summarize the assignment of single cells harboring antiviral (top) or anti-MAA (bottom) TCRs to one of the previously reported 6 clusters.

FIG. 6F is a heatmap showing single-cell phenotype of TILs with antiviral or anti-MAA TCRs.

FIG. 6G is a heatmap showing deregulated genes in exhausted clusters (T_{Ex}), enriched in tumor-reactive T cells, from the discovery cohort.

FIG. 6H shows dot plots depicting expression of representative RNA-transcripts (top) or surface proteins (bottom) in each TCR clonotype family with antiviral (black) or antitumor (grey) specificity.

FIG. 7A and FIG. 7B are dot plots showing antigen specificity of tumor-reactive TCRs.

FIG. 7C includes four pie charts showing distribution of antigen specificities of antitumor TCRs per patient successfully de-orphanized after screening.

FIG. 8 is a heatmap showing genes differentially expressed between CD8+ TILs with identified MAA, NeoAg-specific, or virus-specific TCRs.

FIG. 9 is a series of line plots showing normalized antitumor TCR reactivity and avidity.

FIG. 10A is a schematic of sample collection, processing, and single-cell sequencing analysis and identification of antitumor TCRs in clear cell renal cell carcinoma (ccRCC) samples collected from treatment-naïve patients.

FIG. 10B shows UMAPs of scRNA-seq data from CD8+ clear cell renal cell carcinoma (ccRCC) samples TILs.

FIG. 10C shows UMAPs of CD8+ TILs colored based on enrichment of gene-signatures of exhaustion and memory T cells (left) or associated with CD8+ TILs with validated antiviral (top) or antitumor (bottom) reactivity.

FIG. 10D is a bar chart showing the frequencies of T cell metaclusters, as detected by scRNA-seq in normal kidney tissues and tumor biopsies

FIG. 11A shows a series of heatmaps showing the reactivity of dominant TCRs sequenced among T_{Ex} (top) or T_{NExM} (bottom) clusters in 5 ccRCC patients A-E.

FIG. 11B is a bar chart showing the number of TCRs tested for each patient (columns) and classified as tumor specific (black).

FIG. 11C is a bar chart showing the proportion of TCRs classified as tumor-specific among TEx-TCRs or TNExM-TCRs in 5 patients with ccRCC.

FIG. 12A is a series of line charts showing reactivity and avidity of ccRCC-TCRs with de-orphanized antigen specificity.

FIG. 12B shows the phenotypes of antigen specific TCR clonotypes in ccRCC. The UMAPs on the left show the phenotypic distribution of T cells bearing antitumor TCRs specific for TAAs-, NeoAgs- or virus-specific TCR clonotypes. The pie charts on the right show the frequency of T cells within each metacluster.

FIG. 12C is a heatmap showing the phenotypes of antigen specific TCR clonotypes infiltrating ccRCC tumors.

### DETAILED DESCRIPTION

### Definitions

Unless defined otherwise, all technical and scientific terms used herein have the same meaning as is commonly understood by one of skill in the art to which the subject matter herein pertains. As used in the specification and the appended claims, unless specified to the contrary, the following terms have the meaning indicated to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising” is synonymous with “including,” “containing,” or “characterized by.” The transitional phrase “consisting essentially of” limits the scope of a claim to the specified materials or steps “and those that do not materially affect the basic and novel characteristic(s)” of the claimed disclosure (e.g., the claimed methods).

The present disclosure is based, at least in part, on several discoveries. As disclosed in the working examples that describe experiments conducted in the context of melanoma and renal cell carcinoma, Applicant has discovered the following. First, TCRs derived from PD-1+ CD39+ exhausted cells possess high anti-melanoma potential against personal and shared tumor antigens. Conversely, non-exhausted PD1-CD39-bystander cells with a memory phenotype were composed predominantly of TCRs with anti-viral specificity, and rarely antitumor TCRs. Therefore, the exhausted intratumoral compartment is highly enriched in polyclonal tumor-reactive T cells.

Second, these dysfunctional phenotypes were observed among TCR clonotypes displaying a broad range of avidities whether for public melanoma antigens or personal neoantigens. Therefore, recognition of tumor antigens, but not antigen class per se, determines the intratumoral phenotype of anti-melanoma T cells.

Third, interaction with tumor antigens led to selection of TCRs with avidities inversely related to the expression level of cognate targets in melanoma cells and proportional to the binding affinity of peptide-HLA class I complexes.

Fourth, the TCR clonotypes from intratumoral exhausted lymphocytes persisted in peripheral blood at higher levels in patients with poor response to immune checkpoint blockade compared to those achieving durable disease regression, consistent with chronic stimulation mediated by the presence of residual tumor antigen.

Similar findings have been confirmed for TCRs detected in renal cell carcinoma: antitumor TCRs that are able to recognize and kill renal cell carcinoma cells are enriched among T cells with an exhausted phenotype, identified from expression of exhaustion markers. These include T cell specificities that are able to recognize personal neoantigens or shared tumor antigens. Conversely, antiviral bystander specificities are mainly observed within memory non-exhausted T cells.

Not intending to be bound by any particular theory of operation, Applicant believes that arming T lymphocytes with TCR specificities of polyclonal T cells enriched in tumor-reactivity will promote effective tumor regression. Accordingly, the present disclosure provides a personal cancer treatment. By arming T lymphocytes with TCR specificities of polyclonal T cells enriched in tumor-reactivity (the TCRs having been identified in exhausted T cells), the present methods may promote effective solid tumor regression in solid cancers including gastrointestinal carcinomas, sarcomas, and melanoma.

As described herein, state of the art single-cell technologies (single-cell RNAseq, single-cell TCRseq) on tumor biopsies collected from cancer patients may be utilized to identify TCR clonotypes that are expanded within the tumor and their phenotype. This step, in turn, facilitates selection of highly exhausted TCR clonotypes. In some embodiments, TCRs from TCR clonotypes with high co-expression of PD-1 and CD39 surface proteins are highly cytotoxic against the tumor and may comprise a broad range of strong antitumor specificities including recognition of diverse tumor antigens.

As also described herein, non-exhausted autologous T cells or allogeneic T cells may be modified or engineered in accordance with known techniques, to express these TCRs of interest. Such modified T cells may possess strong antitumor potential and provide potent and durable anti-cancer therapy. They may also be used to create T cell banks and provide the basis for personalized anti-cancer therapy. The present methods entail collecting T cells from a specimen (e.g., a tumor biopsy) from a subject having or suspected of having a cancer (e.g., melanoma or renal cell carcinoma).

### T Cell Populations

The present disclosure is directed, at least in part, to the identification of tumor reactive T cells in a patient suffering from cancer characterized by the presence of a solid tumor (also referred to herein as a “solid cancer”). The working examples herein demonstrate the properties (i.e. phenotypes, antigen specificities and dynamics) of antitumor T cell clones, as identified through their TCRs within the tumor microenvironment. It has now been discovered that the majority of tumor reactive T cells had exhausted phenotypes. This has been discovered by performing single-cell profiling of CD8+ T cells from melanoma and renal cell carcinoma samples, combined with reconstruction and specificity testing of hundreds of cloned TCRs.

T cells may be collected from a tumor biopsy (also “specimen”) obtained from the subject, in accordance with standard techniques. The T cells are analyzed and assigned into clonotype families, which are defined on the basis of single cell TCR sequencing. Members of a clonotype family all have identical sequences of TCRα and TCRβ chains, which are typically assessed through single-cell TCR sequencing. The combination of TCRα and TCRβ sequences define the T cell clonotype. Clonotyping is a process to identify the unique nucleotide sequences, typically limited to the CDR3 region, of a TCR chain. Clonotyping may be performed by PCR amplification of the cDNA using V-region-specific primers and either constant region (C) specific or J-region-specific primer pairs, followed by nucleotide sequencing of the amplicon as known in the art or by single cell TCR sequencing.

The TCR clonotype families may be compared in order to identify expanded clonotypes, especially TCR clonotype families that dominate over others. Expanded and dominant TCR clonotype families may further be classified as having an exhausted or non-exhausted phenotype.

As used herein, the terms “exhausted”, “exhaustion”, “unresponsiveness” and “exhausted phenotype” are used interchangeably and refer to a state of a cell where the cell is impaired in its usual functions or activities in response to normal input signals. Such functions or activities include proliferation, cell division, entrance into the cell cycle, migration, phagocytosis, cytokine production, cytotoxicity, or any combination thereof. Normal input signals include stimulation via a receptor (e.g., the TCR or a co-stimulatory receptor, for example, CD3 or CD28). The term “exhausted T cell” refers to a T cell that does not respond with effector function when stimulated with antigen and/or stimulatory cytokines sufficient to elicit an effector response in non-exhausted T cells and encompasses T cell tolerance, which is a normal state required to avoid self-reactivity. This state of dysfunction is due to the expression of receptors (e.g., PD-1 and CD39) that provide inhibitory signals to the T cells, limiting their ability to respond to the stimulation provided by an antigen on a tumor cell.

In some embodiments, a cell that is exhausted is a CD8+ cytotoxic T lymphocyte (CTL). CD8+ T cells normally proliferate, lyse target cells (cytotoxicity), and/or produce cytokines such as IL-2, TNFα, IFNγ, or a combination therein in response to TCR and/or co-stimulatory receptor stimulation. Non-exhausted CD8+ T cells proliferate and produce cell killing enzymes (e.g., cytotoxins perforin, granzymes, and granulysin) upon receiving an input signal (e.g., TCR stimulation). However, exhausted CD8+ T cells do not respond adequately to TCR stimulation, and they display poor effector function, sustained expression of inhibitory receptors, and a transcriptional state distinct from that of functional effector or memory T cells. Exhaustion of T cells thus prevents optimal control of infection and tumors. Exhausted T cells, particularly CD8+ T cells, may produce reduced amounts of IFNγ, TNFα, and immunostimulatory cytokines (e.g., IL-2) as compared to functional T cells. Thus, an exhausted CD8+ T cell fails to do one or more of proliferate, lyse target cells, and produce cytokines in response to normal input signals.

In some embodiments, the exhausted T cell is a CD8+ T cell (i.e., a T cell that expresses the CD8⁺ cell surface marker). In some embodiments, the exhausted T cell is a memory T cell (T_{M}). In some embodiments, the exhausted T cell is an effector memory T cell (T_{EM}). In some embodiments, the exhausted T cell is an NK-like T cell (T_{NK-like}). In some embodiments, the exhausted T cell is a γδ-like T cell (T_{γδ-like}). In some embodiments, the exhausted T cell is an activated T cell (T_{Act}). In some embodiments, the exhausted T cell is an apoptotic T cell (T_{Ap}). In some embodiments, the exhausted T cell is a regulatory-like T cell (T_{reg-like}). In some embodiments, the exhausted T cell is a proliferating T cell (Tₚᵣₒₗ). In some embodiments, the exhausted T cell is a progenitor exhausted T cell (T_{PE}). In some embodiments, the exhausted T cell is a terminal exhausted T cell (T_{TE}).

In some embodiments, the exhausted T cell is a CD4+ helper T lymphocyte (T_{H}). Such T_{H} cells normally proliferate and/or produce cytokines such as IL-2, IFNγ, TNFα, IL-4, IL-5, IL-17, IL-10, or a combination thereof, in response to TCR and/or co-stimulatory receptor stimulation. The cytokines produced by T_{H} cells act, in part, to activate and/or otherwise modulate, i.e., “provide help,” to other immune cells such as B cells and CD8+ cells. Thus, an exhausted T_{H} cell or CD4+ T cell shows disfunction as impaired proliferation and/or cytokine production upon TCR stimulation.

Persistent antigenic stimulation induces the exhaustion dysfunctional state in CD8+ and CD4+ T cells. Though T cell exhaustion limits the damage caused by an immune response, it also leads to attenuated effector function where CD8+ T cells fail to control tumor progression. T cell exhaustion is a dynamic process starting from T cell activation to progenitor exhaustion, and finally to terminal exhaustion, with each stage having distinct properties. See, Wherry et al., Nat. Immunol. 12:492-9 (2011).

The profiling methods of T cells isolated from specimens (either from a subject in need of treatment or from a subject having a malignant tumor) described herein can identify exhausted and non-exhausted T cell phenotypes. Once cells are profiled, known sorting methods may be employed to sort, select, and isolate a desired population of T cells based on phenotype and/or clonotype.

Further characterization of exhausted and non-exhausted T cells, in turn, enables selection of optimal cells or cell populations to use as TCR donor T cells or adoptive cell transfer recipient T cells. Thus, T cells collected from a subject's specimen may be analyzed and further characterized into distinct cell states, for example, tumor specific terminally exhausted T cells (T_{TE}), activated T cells (T_{Act}), proliferating T cells (Tₚᵣₒₗ), progenitor exhausted T cells (T_{PE}), and effector memory T cells (T_{EM}). In some embodiments, and as described in the working examples below, antitumor specificity of the individual TCRs affects the relative proportion of each phenotype per T cell clonotype family or population, since the transcriptional profiles for the majority of cells are typically skewed towards an exhausted T cell state. In some embodiments, one cell state (T_{TE}) is selected for TCR sequencing, cloning, and transfer into recipient T cells.

### Expression Profiling

The present disclosure provides methods for generating gene transcription or protein expression profiles (including selected gene sequences) of T cells from a collected specimen from a subject. The subject from which the specimen is collected may be a subject with a cancer and in need of treatment therefore, or a subject with a malignant tumor who is different from the subject receiving the treatment. The profiles define the collected T cells, typically in relation to cellular transcriptome and TCR clonality. In some embodiments, the profiling includes high-throughput single cell transcriptome sequencing (scRNAseq), single cell TCR sequencing (scTCRseq), and cellular indexing of transcriptomes and epitopes by sequencing (CITEseq). Profiling results in the quantification or qualification discovery T cell receptors expressed by T cells with specific cellular markers (referred to as “exhaustion markers” herein).

The terms “express” and “expression” as used herein refer to transcription, translation, or both transcription and translation of a nucleic acid sequence within a cell. The level of expression of a nucleic acid or protein may thus indicate either the amount of nucleic acid (e.g., mRNA) that is present in the cell, or the amount of the desired polypeptide encoded by a selected sequence.

The profiling methods and techniques described herein allow for the use of the nucleic acid and protein as described herein to identify, analyze, and select specific cells, clonotype families, or cell clusters. It is common in the art to refer to a cell as “positive” or “negative” for a particular marker; however, the actual expression levels are preferably quantitatively determined. The number of molecules detected (e.g., on the cell surface) may vary by several logs, yet still be characterized as “positive.” Likewise, it is understood in the art that a cell which is negative for staining, i.e., the level of marker binding a specific reagent is not detectably different from a control, such as an isotype matched control, may express small amounts of the marker, and may be referred to as relatively “dim” or having “low” expression.

Characterization, or grouping, of the level of expression of a marker permits subtle distinctions between cell populations. The expression level of a marker in cells can be monitored by flow cytometry, where lasers detect the quantitative levels of fluorochrome (which is proportional to the amount of cell surface marker bound by specific reagents, e.g., antibodies). Flow cytometry, or FACS, can also be used to separate cell populations based on the intensity of binding to a specific reagent, as well as other parameters such as cell size and light scatter. Although the absolute amount of reagent binding may differ with a particular fluorochrome and reagent preparation, the data can be normalized to a control.

The terms “low,” “relatively low,” and “dim” as used herein to modify positivity or expression levels, which refers to cells having a level of marker staining above the brightness of a control, such as an isotype matched control, but not as intense as the most brightly stained cells normally found in a population. Dim cells may have unique properties that differ from the negative and brightly stained positive cells of a sample. An alternative control may utilize a substrate having a defined density of marker on its surface, for example a fabricated bead or cell line, which provides a positive control for intensity.

The terms “high,” “relatively high,” and “bright” as used herein to modify positivity or expression levels, which refers to cells having a level of marker staining above the brightness of other positive populations of cells, and higher than any cells having a “relatively low” expression and are typically the most brightly stained cells normally found in a population. Bright cells may have unique properties that differ from the positive and dimly stained cells of a sample.

The term “isotype control” as used herein and as is known in the art indicates an antibody that lacks specificity to a target of interest, but matches the class and type of an antibody used in the same assay or test. Isotype controls are used as negative controls to help differentiate non-specific background signal or “staining” from specific antibody signal. Depending upon the isotype of the antibody used for detection and the target cell types involved, background signal may be a significant issue in various experiments.

Applicable methods of nucleic acid measurement and quantification include Northern blot hybridization, ribonuclease RNA protection, in situ hybridization to cellular RNA, microarray analysis, RT-PCR (reverse-transcription polymerase chain reaction) and scRNAseq. Applicable methods of protein measurement and quantification include ELISA, Western blotting, radioimmunoassays, immunoprecipitation, assaying for the biological activity of the protein, immunostaining of the protein (including, e.g., immunohistochemistry and immunocytochemistry), flow cytometry, fluorescence activated cell sorting (FACS) analysis, and homogeneous time-resolved fluorescence (HTRF) assays.

scRNAseq provides information relating to the multi-tiered complexity of different cells within the same tissue or specimen type. scRNAseq is a genomic, single cell approach for the detection and quantitative analysis of messenger RNA molecules in a biological specimen and is useful for studying cellular responses. scRNAseq may be combined with additional methods for the detection and quantitation of RNA, including other single-cell RNA sequencing methods. The scRNAseq method includes isolating single cells, typically in a single cell suspension. The single cell suspension is lysed, then, mRNAs are purified and primed with a poly(T) primer for reverse transcription. Unreactive primers are removed. Poly(A) tails are added to the first strand cDNA at the 3′ end and annealed to poly(T) primers for second-strand cDNA generation. Finally, the cDNAs are PCR-amplified, sheared, and prepared into sequencing libraries. The methods of scRNAseq utilized herein enable single-cell-resolution transcriptomic analysis, phenotypic profiling, and clustering of T cells into distinct cell states.

High-throughput scTCRseq technologies allow for the identification of TCR sequences (e.g., paired α- and β-chain information), analysis of their antigen specificities using experimental and computational tools, assigning T cells into clonotype families, and the pairing of TCRs with transcriptional and epigenetic phenotypes in single cells. Furthermore, these methods allow for the rapid cloning and expression of the identified TCRs to functionally test antigen specificity. Cloned TCRs may be tested in vitro, or ex vivo, or once validated, administered in vivo.

CITEseq is a method for performing RNA sequencing that also gains quantitative and qualitative information on surface proteins of the sequence cells with available antibodies on a single cell level. CITEseq provides an additional information for a cell by combining both proteomics and transcriptomics data. For phenotyping, this method has been shown to be as accurate as flow cytometry. CITEseq is currently one of the main methods, along with RNA expression and protein sequencing assay (REAPseq), to evaluate both gene expression and protein levels simultaneously in different species.

CITEseq has been used to characterize tumor heterogeneity in cancers, aid in tumor classification, identify rare subpopulations of cells in different contexts, immune cell characterization, and host-pathogen interactions. CITEseq enables these applications by utilizing single-cell output of both protein and transcript data, which also leads to novel information on protein-RNA correlation. One important aspect of CITEseq profiling, is that it enables the detection of surface proteins at the single-cell level.

### Exhaustion Markers

The collected T cells are profiled and selected for an exhaustion phenotype, on the basis of specific markers, referred to herein as exhaustion markers. The exhaustion markers include one or more RNA transcripts of the PDCD1, HAVCR2, CTLA4, ENTPD1, LAG3, and TOX genes, and/or (i.e., alternatively, or in addition) one or more of the PD1, Tim-3, CTLA4, CD39, and LAG3 surface proteins. Transcript levels of these surface proteins may also be used as an exhaustion marker.

In some embodiments, the exhaustion marker is a combination of PDCD1, HAVCR2, CTLA4, ENTPD1, LAG3, and TOX gene transcripts (i.e., RNA transcripts). In some embodiments, the exhaustion marker is a combination of PD1, Tim-3, CTLA4, CD39, and LAG3 surface proteins. These enable the selection of exhausted T cells having a progenitor exhausted (T_{PE}) phenotype. In some embodiments, the exhaustion markers include the CD39 and PD1 surface proteins. In some embodiments, the exhaustion markers include the ENTPD1 and PDCD1 RNA transcripts.

An exemplary thymocyte selection associated high mobility group box (TOX) nucleic acid sequence is provided at NCBI Accession No. NM 014729, version NM 014729.3, incorporated herein by reference.

An exemplary TOX polypeptide sequence is provided at NCBI Accession No. NP_055544, version NP_055544.1, incorporated herein by reference.

An exemplary programmed cell death 1 (PDCD1) nucleic acid sequence is provided at NCBI Accession No. NM_005018, version NM_005018.3, incorporated herein by reference.

An exemplary PDCD1 polypeptide (PD1) sequence is provided at NCBI Accession No. NP_005009.2, version NP_005009.2, incorporated herein by reference.

An exemplary hepatitis A virus cellular receptor 2 (HAVCR2) nucleic acid sequence is provided at NCBI Accession No. NM_032782, version NM_032782.5, incorporated herein by reference.

An exemplary HAVCR2 polypeptide sequence is provided at NCBI Accession No. NP_116171.3, version NP_116171.3, incorporated herein by reference.

An exemplary cytotoxic T-lymphocyte associated protein 4 (CTLA4) nucleic acid sequence is provided at NCBI Accession No. NM_001037631, version NM_001037631.3, incorporated herein by reference.

An exemplary CTLA4 polypeptide sequence is provided at NCBI Accession No. NP_001032720, version NP 001032720.1, incorporated herein by reference.

An exemplary ectonucleoside triphosphate diphosphohydrolase 1 (ENTPD1) also known as CD39, nucleic acid sequence is provided at NCBI Accession No. NM 001098175, version NM_001098175.2, incorporated herein by reference.

An exemplary ENTPD1 polypeptide sequence is provided at NCBI Accession No. NP_001091645, version NP_001091645.1, incorporated herein by reference.

An exemplary Tim-3, also known as hepatitis A virus cellular receptor 2 (HAVCR2), nucleic acid sequence is provided at NCBI Accession No. NM 032782, version NM_032782.5, incorporated herein by reference.

An exemplary Tim-3 polypeptide sequence is provided at NCBI Accession No. NP_116171, version NP_116171.3, incorporated herein by reference.

An exemplary lymphocyte activating 3 (LAG3) nucleic acid sequence is provided at NCBI Accession No. NM 002286, version NM_002286.6, incorporated herein by reference.

An exemplary LAG3 polypeptide sequence is provided at NCBI Accession No. NP_002277, version NP_002277.4, incorporated herein by reference.

### Adoptive Cell Transfer

Adoptive cell transfer (ACT) is a therapy in which the active ingredient is, wholly or in part, a living cell. Adoptive immunotherapy is an ACT that involves the removal of immune cells from a subject, ex vivo processing (e.g., genetic modification, purification, activation, and/or expansion), and subsequent infusion of the either the original cells or other genetically modified autologous cells back into the same subject. ACT has been used in, for example, lymphocytes generally, LAK cells, TILs, cytotoxic CD8+ T-cells, CD4+ T cells, and tumor draining lymph node cells. See, U.S. Pat. Nos. 4,690,915, 5,126,132, 5,443,983, 5,766,920, 5,846,827, 6,040,180, 6,194,201, 6,251,385, and 6,255,073.

ACT often involves two populations of cells, donor cells that provide the TCR genes and recipient cells that are genetically modified with the donor cell's TCR genes. Previous approaches in ACT studies used unselected TIL T cells, either as one or both of the donor and recipient cells in an ACT treatment. The use of TCRs from donor tumor specific (TS) T cells for ACT, especially profiled TS T cells, has potential to improve patient outcomes. However, as shown in the working examples below, it has now been surprisingly discovered that most of the TS T cells had exhausted phenotypes and that the majority of these cells would be poor candidates as recipient ACT cells. However, the exhausted TS T cells present ideal TCR donor cells for cloning and transfer into allogenic or autologous non-exhausted T cells, preferably memory stem cell-like recipient T cells. Therefore, TCR gene-modification of allogenic or a subject's own non-exhausted T cells with TCRs from exhausted, TS T cells and adoptive transfer of those recipient T cells would enable instantaneous generation of a defined T cell immunity with a desired and profiled phenotype.

This approach allows the introduction of TCRs with specificities that, while present in the subject's T cell pool, are not present in the desired phenotype. Previous in vitro studies have shown that TCR-gene modified (TGM) recipient T cells containing TCRs with high affinity for their peptide/MHC complex, produce high avidity T cells. See, Heemskerk et al., Blood 102:3530-40 (2002); Heemskerk et al., J. Exp. Med. 199:885-94 (2004). TGM T cells have been used in adoptive transfer clinical trials. See, Johnson et al., Blood 114:535-46 (2009); Morgan et at, Science 314:126-9 (2006).

Previous adoptive transfer attempts have included melanoma studies, where T cells TCRs to melanoma antigens MART-1 (Melanoma Antigen Recognized By T Cells 1; also known as MLANA) or gp100 (as known as Premelanosome Protein, PMEL) were isolated, cloned, and transfected into autologous recipient peripheral blood lymphocytes (PBLs). While these TGM PBLs bound to target tetramers, clinical trials only resulted in cancer regression in 19-30% of patients. And normal melanocytes in the skin, eye, and ear were destroyed by the TGM PBLs. The most likely occurrence for these toxicities resulted from tumor-associated antigens being expressed on normal tissues.

Collecting, profiling, and selecting the T cells presents ideal TCR candidates for gene transfer and subsequent adoptive transfer. Furthermore, use of selection markers ensures proper T cell selection for TCR cloning (e.g., TCRA and TCRB) of exhausted, TS donor T cells as well as for TGM recipient T cells. In some embodiments, one TCR from an identified exhausted T cell clonotype family (i.e., an expanded clonotype family that expresses one or more of PDCD1, HAVCR2, CTLA4, ENTPD1, LAG3, and TOXRNA transcripts and/or one or more of PD1, Tim-3, CTLA4, CD39, and LAG3 surface proteins) is cloned into recipient T cells. In some embodiments, TCRs from multiple identified exhausted T cell clonotype families are cloned into recipient T cells. In some embodiments multiple TCRs (from 1 to 10 TCRs, preferably 1-3 TCRs) or 1 TCR is cloned from an identified exhausted T cell clonotype family or families into recipient T cells. In some embodiments, expression of multiple TCRs into a population of non-exhausted T cells is performed to achieve the expression of a single TCR per recipient T cell.

ACT is typically restricted by human leukocyte antigen (HLA)/MHC matching in that recipient T cells typically have to have at least a partial HLA/MHC match with the subject. In contrast, both autologous and non-autologous (e.g., allogeneic, or syngenic) T cells can be used in the ACT therapy methods disclosed herein. The term “autologous” as used herein refers to any material (e.g., T cells) derived from the same subject to whom it is later re-introduced. The term “allogeneic” as used herein refers to any material derived from a different subject of the same species as the subject to whom the material is later introduced. Two or more individual subjects are allogeneic when the genes at one or more loci are not identical.

In some embodiments, the recipient T cells are isolated from the same subject in need thereof, producing autologous cells having a complete HLA/MHC match. In some embodiments, peripheral blood T lymphocytes are isolated from the subject through leukapheresis. Methods for isolating, producing, and stimulating autologous or allogeneic T cells isolated from a subject are known in the art. Stimulation and expansion ex vivo, to increases cell number and cytotoxicity functionality in the recipient T cells, may be accomplished by adding cytokines and co-factors to the cell culture, e.g., IL-2, GM-CSF, CD3, and CD28. Validation of recipient T cell activation may be performed in vitro by co-culturing a population or recipient T cells with antigen presenting cells pulsed with antigens, and subsequent measurement of surface expression of CD69 or IL-2 secretion. See, U.S. Pat. Nos. 7,399,633, 7,575,925, 10,072,062, 10,370,452, and 10,829,735 and U.S. Patent Publication Nos. 2019/0000880 and 2021/0407639.

In subjects at risk for developing a cancer or suspected of having a cancer, a TCR from a previously identified exhausted T cell clonotype family (i.e., an expanded clonotype family that expresses one or more of PDCM, HAVCR2, CTLA4, ENTPD1, LAG3, and TOXRNA transcripts and/or one or more of PD1, Tim-3, CTLA4, CD39, and LAG3 surface proteins) is stored in a suitable fashion as known in the art for cloning into autologous or allogeneic non-exhausted T cells. For example, TCRs may be stored as nucleic acids in a centralized TCR bank or produced synthetically from a TCR database, using known methods, after identification through the methods described herein.

Typically, ex vivo expansion is performed in tissue culture flasks and gas-permeable bags. First the recipient cells are co-cultured with irradiated autologous or allogeneic peripheral blood mononuclear cells (PBMCs) as feeder cells in T-175 flasks in media with IL-2 (e.g., 3000 IU/mL) and anti-CD3 (e.g., 30 nanograms per milliliter (ng/mL)) for 7 days. The recipient cells are then transferred to gas-permeable bags and are cultured for an additional 7 days. Optimal density of cells cultured in bags is about 0.5-2×10⁶ cells/mL, the final volume of the culture typically ranges from 30 liter (L) to 60 L. Finally, the recipient cells are concentrated, washed, and resuspended in an acceptable formulation, typically in a volume that can be administered over a period of several hours. See, Jin et al., J. Immunother. 35:283-292 (2012).

Further profiling and selection of memory markers in the recipient T cells may be performed. Profiling and selection results in recipient cells that are more persistent and as a result more effective, in adoptive immunotherapy.

Any suitable method of transgenic (i.e., modified) recipient T cell generation may be used. In one embodiment, TCR genes are cloned into a plasmid library. In some cases, a single plasmid vector is used for both TCRA and TCRβ genes; in other embodiments, two plasmid vectors are used to contain each gene individually. In other embodiments, polynucleotides encoding TCRA and TCRβ from donor cells are synthesized in vitro and transferred (e.g., transfected or electroporated) into recipient cells.

Another embodiment relies on viral vectors to deliver and randomly integrate the therapeutic constructs.

In other embodiments, non-viral CRISPR-Cas9 genome targeting is used. This approach makes use of three components: a Cas protein or polynucleotide encoding a Cas protein (e.g., Cas9), a guide RNA (gRNA), and a Homology Directed Repair Template (HDRT) polynucleotide. The Cas9 and gRNA are pre-assembled into a ribonucleoprotein (RNP) and delivered with the cognate HDRT into cells ex vivo by a suitable method (e.g., electroporation). The RNP component generates a targeted double-stranded break (DSB) at a genomic locus complementary to the gRNA sequence. The HDRT facilitates precise integration of the therapeutic construct at that desired location. The HDRT comprises two regions of homology, a left homology arm and a right homology arm, each arm is partially or fully homologous to a target sequence of DNA. Between the arms is a sequence encoding the therapeutic construct (e.g., the cloned TS TCR). The target sequences of the left and right homology arms span the DSB introduced by the Cas protein. Improvements in cellular handling, electroporation conditions, RNP assembly, and HDRT modifications have made this approach well suited to generate high efficiency T cell knock-ins of chimeric antigen receptors (CAR) and TCRs.

A treatment-effective amount of recipient T cells in ACT is typically at least 10⁸, at least 10⁹, typically greater than 10⁹, at least 10¹⁰ cells, generally more than 10¹⁰, or more than 10¹¹ cells. The number of cells will depend, at least in part, upon the cancer to be treated. Desirably, the cells will match the clonotype of the recipient. For example, if the recipient T cells that are a specific clonotype, then the treatment effective amount will contain greater than 70%, generally greater than 80%, greater than 85%, or 90-95% of that specific clonotype. For treatments provided herein, the cells are generally provided in a volume of fluid of a liter or less, or 500 milliliters (mL) or less, or even 250 mL, or 100 mL or less. Hence the density of the recipient T cells is typically greater than 10⁶ cells/mL and generally is greater than 10⁷ cells/mL, generally 10⁸ cells/mL or greater. The clinically relevant number of T cells can be apportioned into multiple infusions that cumulatively equal or exceed 10⁹, 10¹⁰, or 10¹¹ cells.

Recipient T cells may be administered by a single infusion, or by multiple infusions over a range of time. However, since different individuals are expected to vary in responsiveness, the type and number of cells infused, the number of infusions, and the time range over which multiple infusions are given, are determined by the attending physician or veterinarian, and can be determined by routine examination.

The methods of the present disclosure may entail administration of recipient T cells of the disclosure or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5 or 6 weeks, and in other embodiments, administration entails a 28-day cycle which includes daily administration for 3 weeks (21 days).

### Cancer

In some aspects, the present disclosure is directed to treating cancer in a subject. The terms “cancer characterized by a solid tumor” and “malignant neoplasm” are used interchangeably herein.

The term “subject” (or “patient”) as used herein includes all members of the animal kingdom prone (or disposed) to or suffering from the indicated cancer. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. Therefore, a subject “having a cancer” or “in need of” treatment according to the present disclosure broadly embraces subjects who have been positively diagnosed, including subjects having active disease who may have been previously treated with one or more rounds of therapy, and subjects who are not currently being treated (e.g., in remission) but who might still be at risk of relapse, and subjects who have not been positively diagnosed but who are predisposed to cancer (e.g., on account of the basis of prior medical history and/or family medical history, or who otherwise present with one or more risk factors such that a medical professional might reasonably suspect that the subject was predisposed to cancer).

Solid tumors have intrinsic tumor diversity and often lack common, conserved tumor antigens that can targeted with broadly applicable, genetically modified immune cells. In some embodiments, the cancer is a carcinoma. Carcinomas may include adenocarcinoma (breast cancer), adrenocortical carcinoma, basal cell carcinoma, ductal carcinoma in situ, invasive ductal carcinoma, squamous cell carcinoma, and renal cell carcinoma. Exemplary carcinomas well suited for the inventive methods disclosed herein include breast, lung, renal cell carcinoma and gastrointestinal (e.g., colorectal) cancers. See, Caushi et al., Nature 596:126-132 (2021); Hanada et al., Cancer Cell 40:479-493 (2022); Liu et al., Cancer Cell 40:424-437 (2022); Lowery et al., Science 375:877-884 (2022).

### Melanoma

In some embodiments, the cancer is melanoma. Melanoma is a cancer that usually starts in a certain type of skin cell, i.e., melanocytes. Melanocytes make a brown pigment called melanin, which gives the skin its tan or brown color. Melanin protects the deeper layers of the skin from some of the harmful effects of the sun. For most people, when skin is exposed to the sun, melanocytes make more melanin, causing the skin to tan or darken. Melanoma is also called malignant melanoma and cutaneous melanoma. Melanomas are most common on the skin, but may occur rarely in the mouth, intestines, or eye. Melanoma is the fifth most common cancer in men and the sixth most common cancer in women (Rastrelli et al., In Vivo 28:1005-11 (2014)).

### Breast Cancer

In some embodiments, the cancer is breast cancer. Breast cancer is a group of cancers in which cells in the breast grow out of control. The term “breast cancer” includes all forms of cancers affecting breast cells, including breast cancer, a precancer or precancerous condition of the breast, and metastatic lesions in tissue and organs in the body other than the breast. Breast cancer can begin in different parts of the breast. A breast is made up of three main parts: lobules, ducts, and connective tissue. The lobules are the glands that produce milk. The ducts are tubes that early milk to the nipple. The connective tissue (which consists of fibrous and fatty tissue) surrounds and connects the breast tissue. Most breast cancers begin in the ducts or lobules.

Exemplary breast cancers may include hyperplasia, metaplasia, and dysplasia of the breast. The two most common types of breast cancer are invasive ductal carcinoma and invasive lobular carcinoma. In invasive ductal carcinoma, cancer cells originate in the ducts and then spread, or metastasize, outside the ducts into other parts of the breast tissue. Invasive cancer cells can also spread, or metastasize, to other parts of the body. In invasive lobular carcinoma, cancer cells originate in the lobules and then spread from the lobules to the breast tissues that are close by. These invasive cancer cells can also spread to other parts of the body. Less common forms of breast cancer include Paget's disease, medullary, mucinous, and inflammatory breast cancer. Breast cancer can spread outside the breast, typically through blood vessels and lymph vessels.

### Lung Cancer

In some embodiments, the cancer is lung cancer. Lung cancer is cancer that forms in tissues of the lung, usually in the cells lining air passages. Lung cancer is the third most common cancer type and is the main cause of cancer-related death in the United States.

Lung cancers usually are grouped into two main types, small cell lung cancer and non-small cell lung cancer (including adenocarcinoma and squamous cell carcinoma). Non-small cell lung cancer is more common than small cell lung cancer.

### Gastrointestinal Cancer

Gastrointestinal cancers are cancers that develop along the gastrointestinal (digestive) tract. The gastrointestinal tract starts at the esophagus and ends at the anus. Gastrointestinal cancers include anal cancer, bile duct cancer, colon cancer, esophageal cancer, gallbladder cancer, gastrointestinal stromal tumors, liver cancer, pancreatic cancer, colorectal cancer, small intestine cancer, and gastric (stomach) cancer. Colorectal cancers are the most common gastrointestinal cancers in the United States.

### Colorectal Cancer

Colorectal cancer is a type of gastrointestinal cancer that starts in the colon or the rectum. These cancers are also called colon cancer or rectal cancer, depending on where they start. The colon is the large intestine or large bowel. The rectum is the passageway that connects the colon to the anus. Colon cancer and rectal cancer are often grouped together because they have many features in common. Sometimes abnormal growths, called polyps, form in the colon or rectum. Over time, some polyps may turn into cancer. Screening tests can find polyps so they can be removed before turning into cancer. Screening aids in the detection colorectal cancer at early stages, when treatment is most successful at treating the cancer. The most common type of colorectal cancer is adenocarcinoma. Adenocarcinomas of the colon and rectum make up 95% of all colorectal cancer cases in the United States. In the gastrointestinal tract, rectal and colon adenocarcinomas develop in the cells of the lining inside the large intestine. These adenocarcinomas typically start as a polyp.

### Sarcomas

Sarcoma is the general term for a broad group of cancers that originate in the bones and in the soft (i.e., connective) tissues, for example, soft tissue sarcomas. Soft tissue sarcomas forms in the tissues that connect, support, and surround other body structures. These tissues include muscle, fat, blood vessels, nerves, tendons, and joint linings.

There are over 70 types of sarcomas. The three most common types of sarcomas are undifferentiated pleomorphic sarcoma (previously called malignant fibrous histiocytoma), liposarcoma, and leiomyosarcoma. Treatment varies depending on sarcoma type and location, as well as additional factors. Certain types of sarcomas occur more often in certain parts of the body. For example, leiomyosarcomas are the most common type of sarcoma found in the abdomen, while liposarcomas and undifferentiated pleomorphic sarcomas are most common in legs. Due to their similar microscopic appearances, many sarcomas are classified as sarcomas of uncertain type.

### Renal Cell Carcinoma

some embodiments, the cancer is renal cell carcinoma. Renal cell carcinoma (RCC) is a kidney cancer that originates in the lining of the proximal convoluted tubule, a part of the very small tubes in the kidney that transport primary urine. RCC is the most common type of kidney cancer in adults, responsible for approximately 90-95% of cases. Initial treatment is most commonly either partial or complete removal of the affected kidney(s). When RCC metastasizes, it most commonly spreads to the lymph nodes, lungs, liver, adrenal glands, brain or bones.

### Combination Therapy

The non-exhausted, modified T cells of the present disclosure may be used as part of a combination therapy wherein the subject is treated in combination with the non-exhausted modified T cells and one or more other active agents. The term “in combination” in the context of combination therapy means that the cells and active agent(s) are co-administered, which includes substantially contemporaneous administration, by the same or separate dosage forms, or sequentially, e.g., as part of the same treatment regimen or by way of successive treatment regimens. Thus, if given sequentially, at the onset of administration of the second therapy, the first therapy of the two therapies is, in some cases, still detectable at effective concentrations at the site of treatment. The sequence and time interval may be determined such that the therapies can act together (e.g., in some cases, synergistically to provide an increased benefit relative to the additive benefit of each administered independently). For example, the cells and active agents may be administered at the same time or sequentially in any order at different points in time; however, if not administered at the same time, they may be administered sufficiently close in time so as to provide the desired therapeutic effect, which may in some instances be synergistic.

The dosage of the additional active agent(s) may be the same or even lower than known or recommended doses. See, Hardman et al., eds., Goodman & Gilman's The Pharmacological Basis of Therapeutics, 10th ed., McGraw-Hill, New York, 2001; Physician's Desk Reference 60th ed., 2006. Active agents, such as anti-cancer agents, that may be used in combination with the modified T cells are known in the art. See, e.g., U.S. Pat. No. 9,101,622 (Section 5.2 thereof). An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other active agents would be provided in an amount effective to kill or inhibit proliferation of cancerous cells. This process may involve contacting the cancer cells with modified T cells and the agent(s) at the same time. This may be achieved by contacting the cancer cells with a single composition or pharmacological formulation that includes both the agent(s) and modified cells, or by contacting the cancer cells with two distinct compositions or formulations, at the same time, wherein one composition includes recipient cells and the other includes the other active agent(s).

In some embodiments, the cells of the present disclosure are used in conjunction with chemotherapeutic, radiotherapeutic, immunotherapeutic intervention, targeted therapy, pro-apoptotic therapy, or cell cycle regulation therapy.

In some embodiments, the administration of the cells of the present disclosure may precede or follow the additional active agent (e.g., anti-cancer agent) treatment by intervals ranging from minutes to weeks. In embodiments where the additional active agent(s) and cells of the present disclosure are applied separately to the subject, one would generally ensure that a significant period of time did not expire between the times of each delivery, such that the agent agent(s) and cells would still be able to exert an advantageously combined effect on the subject's cancer. In such instances, it is contemplated that one may administer the subject with both modalities within about 12-24 hours (h) of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In some embodiments, the modified cells of the present disclosure and the additional active agent(s) may be administered within the same patient visit; in other embodiments, the modified cells and the active agent(s) are administered during different patient visits.

In some embodiments, the modified T cells of the disclosure and the additional active agent(s) (e.g., anti-cancer agent(s)) are cyclically administered. Cycling therapy involves the administration of one anti-cancer therapeutic for a period of time, followed by the administration of a second anti-cancer therapeutic for a period of time and repeating this sequential administration, i.e., the cycle, in order to reduce the development of resistance to one or both of the anti-cancer therapeutics, to avoid or reduce the side effects of one or both of the anti-cancer therapeutics, and/or to improve the efficacy of the therapies. In one example, cycling therapy involves the administration of a first anti-cancer therapeutic for a period of time, followed by the administration of a second anti-cancer therapeutic for a period of time, optionally, followed by the administration of a third anti-cancer therapeutic for a period of time and so forth, and repeating this sequential administration, i.e., the cycle. It is expected that the treatment cycles would be repeated as necessary. It also is contemplated that various standard therapies, as well as surgical intervention, may be applied in combination with the cells of the present disclosure.

Representative types of additional anti-cancer therapies are described below. Melanoma therapeutics that are suitable for the combination with the methods described herein include encorafenib (Braftovi®), cobimetinib fumarate (Cotellic®), dacarbazine, talimogene haherparepvec (Imlygic®), recombinant Interferon Alfa-2b (Intron A®), pembrolizumab (Keytruda®), tebentafusp-tebn (Kimmtrak®), trametinib dimethyl sulfoxide (Mekinist®), binimetinib (Mektovi®), nivolumab (Opdivo®), nivolumab and relatlimab-rmbw (Opdualag®), peginterferon Alfa-2b (PEG-Intron®, Sylatron®), aldesleukin (Proleukin®), dabrafenib mesylate (Tafinlar®), ipilimumab (Yervoy®), and vemurafenib (Zelboraf®).

Breast cancer prevention and therapeutics that are suitable for the combination with the methods described herein may also include raloxifene and tamoxifen citrate (Soltamox®), abemaciclib (Verzenio®), paclitaxel (Abraxane®), ado-trastuzumab emtansine (Kadcyla®), everolimus (Afinitor®, Zortress®, Afinitor Disperz®), alpelisib (Piqray®), anastrozole (Arimidex®), pamidronate disodium (Aredia®), exemestane (Aromasin®), cyclophosphamide, doxorubicin hydrochloride, epirubicin hydrochloride (Ellence®), fam-trastuzumab deruxtecan-nxki (Enhertu®), fluorouracil (5-FU; Adrucil®), toremifene (Fareston®), letrozole (Femara®), gemcitabine (Gemzar®, Infugem®), eribulin mesylate (Halaven®), trastuzumab and hyaluronidase-oysk (Herceptin Hylecta®), trastuzumab (Herceptin®), palbociclib (Ibrance®), ixabepilone (Ixempra®), pembrolizumab (Keytruda®), ribociclib (KisqaHO), olaparib (Lynparza®), margetuximab-cmkb (Margenza®), neratinib maleate (Nerlynx®), pertuzumab (Perjeta®), pertuzumab trastuzumab and hyaluronidase-zzxf (Phesgo®), talazoparib tosylate (Talzenna®), docetaxel (Taxotere®), atezolizumab (Tecentriq®), thiotepa (Tepadina®), methotrexate sodium (Trexall®), sacituzumab govitecan-hziy (Trodelvy®), tucatinib (Tukysa®), lapatinib ditosylate (Tykerb®), vinblastine sulfate, capecitabine (Xeloda®), and goserelin acetate (Zoladex®).

Lung cancer therapeutics that are suitable for the combination with the methods described herein include paclitaxel albumin-stabilized nanoparticle formulation (Abraxane®), everolimus (Afinitor®, Zortress®, Afinitor Disperz®), alectinib (Alecensa®), pemetrexed disodium (Alimta®), brigatinib (Alunbrig®), bevacizumab (Alymsys®, MvasiO, Avastin®, Zirabev®), amivantamab-vmjw (Rybrevant®), Ramucirumab (Cyramza®), doxorubicin hydrochloride, mobocertinib succinate (Exkivity®), pralsetinib (Gavreto®), afatinib dimaleate (Gilotrif®), gemcitabine (Gemzar®, Infugem®), durvalumab (Imfinzi), gefitinib (Iressa®), pembrolizumab (Keytruda®), cemiplimab-rwlc (Libtayo®), lorlatinib (Lorbrena®), sotorasib (Lumakras®), trametinib dimethyl sulfoxide (Mekinist®), nivolumab (Opdivo®), necitumumab (Portrazza®), selpercatinib (Retevmo®), Entrectinib (Rozlytrek®), capmatinib (Tabrecta®), dabrafenib mesylate (Tafinlar®), osimertinib mesylate (Tagrisso®), erlotinib (Tarceva®), docetaxel (Taxotere®), atezolizumab (Tecentriq®), tepotinib Hydrochloride (Tepmetko®), methotrexate (Trexall®), dacomitinib (Vizimpro®), vinorelbine tartrate, crizotinib (Xalkori®), ipilimumab (Yervoy®), and ceritinib (Zykadia®).

Colorectal cancer therapeutics, as well as renal cell carcinoma therapeutics, that are suitable for the combination with the methods described herein, include, for example, bevacizumab-maly (Alymsys®), bevacizumab (Avastin®, Mvasi®, Zirabev®), irinotecan (Camptosar®), Ramucirumab (Cyramza®), oxaliplatin (Eloxatin®), cetuximab (Erbitux®), 5-FU (Adrucil®), ipilimumab (Yervoy®), pembrolizumab (Keytruda®), leucovorin, trifluridine and tipiracil hydrochloride (Lonsurf®), nivolumab (Opdivo®), regorafenib (Stivarga®), panitumumab (Vectibix®), capecitabine (Xeloda®), and ziv-aflibercept (Zaltrap®).

### Immunotherapy

Immunotherapy, including immune checkpoint inhibitors may be employed to treat a diagnosed cancer. The immune system reacts to foreign antigens that are associated with exogenous or endogenous signals (so called danger signals), which triggers a proliferation of antigen-specific CD8+ T cells and/or CD4+ helper cells. The mammalian immune system is highly regulated, including central and peripheral tolerance. Central tolerance prevents the immune system reacting to self-molecules and peripheral tolerance prevents over-reactivity of the immune system to various environmental entities (e.g., allergens and gut microbes). Immune checkpoint pathways exist to modulate the responses of immune cells. Stimulatory immune checkpoint pathways activate cell activity, while suppressive immune checkpoint pathways block cell activity. T cells express suppressive immune checkpoint receptors, that after binding of an immune checkpoint ligand, transmits inhibitory signals that reduces the proliferation of these T cells and can also induce apoptosis. Upregulation of immune checkpoint ligands are one means cancers use to evade the host immune system.

Immune checkpoint inhibitors block these inhibitory signaling pathways (dysfunctional in the tumor microenvironment), inducing cancer-cell killing by CD8+ T cells, and enabling the subject's immune system to control a cancer. Immune checkpoint inhibitors have revolutionized the management of many cancers. Immune checkpoint inhibitors may be used to treat a subject at risk for developing cancer or diagnosed with cancer as disclosed herein.

Immune checkpoint molecules include, for example, PD1, CTLA4, KIR, TIGIT, TIM-3, LAG-3, BTLA, VISTA, CD47, and NKG2A. Programmed death-ligand 1 (PDL1) also known as cluster of differentiation 274 (CD274) or B7 homolog 1 (B7-H1) is a protein that is encoded by the CD274 gene in humans.

PDL1 is a 40 kDa type 1 transmembrane protein that plays a major role in suppressing the immune system. Many PD-L1 inhibitors are in development as immuno-oncology therapies and are showing good results in clinical trials. Clinically available examples include durvalumab (Imfinzi®), atezolizumab (Tecentriq®), and avelumab (Bavencio®). Clinically available examples of PD1 inhibitors include nivolumab (Opdivo®), pembrolizumab (Keytruda®), and cemiplimab (Libtayo®).

CTLA4, also known as CD152 (cluster of differentiation 152), is a protein receptor that, functioning as an immune checkpoint, downregulates immune responses. CTLA4 is constitutively expressed in regulatory T cells (Tregs), but only upregulated in conventional T cells after activation. CTLA4 acts as an “off” switch when bound to CD80 or CD86 on the surface of antigen-presenting cells. Recent reports suggest that blocking CTLA4 (using antagonistic antibodies against CTLA such as ipilimumab (Yervoy®)) results in therapeutic benefit. CTLA4 blockade inhibits immune system tolerance to tumors and provides a useful immunotherapy strategy for patients with cancer. See, Grosso J. and Jure-Kunkel M., Cancer Immun., 13:5 (2013). Examples of checkpoint inhibitors include pembrolizumab (Keytruda), ipilimumab (Yervoy), nivolumab (Opdivo) and atezolizumab (Tecentriq).

Additional immunotherapies include the immune modulating antibodies anti-PD1 or anti-PDL1, the cell-cycle inhibitors such as palbociclib, ribociclib or abemaciclib. Melanoma therapies may also be used in combination with the cells of the present disclosure, including the B-Raf inhibitors Vemurafenib (Zelboraf®), dabrafenib (Tafinlar®), encorafenib (Braftovi®), Mirdametinib, and Sorafenib, the MEK inhibitors trametinib, cobimetinib, and binimetinib. Additional investigational MAPK inhibitors may be used as well, including selumetinib, bosutinib, Cobimetinib, AZD8330, U0126-EtOH, PD184352, PD98059, Pimasertib, TAK-733, BI-847325, and GDC-0623. Additional inhibitors that may be useful in the practice of the present disclosure are known in the art. See, e.g., U.S. Patent Publications 2012/0321637, 2014/0194442, and 2020/0155520.

### Chemotherapy

Anti-cancer therapies also include a variety of combination therapies with both chemical and radiation-based treatments. Combination chemotherapies include, for example, Abraxane®, altretamine, docetaxel, Herceptin®, methotrexate, Novantrone®, Zoladex®, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, Taxol®, gemcitabien, Navelbine®, farnesyl-protein tansferase inhibitors, transplatinum, 5-fluorouracil, vincristine, vinblastine and methotrexate, or any analog or derivative variant of the foregoing and also combinations thereof.

Additional chemotherapies involving mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy are applicable to the combination therapies contemplated herein. In specific embodiments, chemotherapy for the individual is employed before, during and/or after administration of the cells of the present disclosure.

### Radiotherapy

Anti-cancer therapies also include radiation-based, DNA-damaging treatments. Combination radiotherapies include what are commonly known as gamma-rays, X-rays, and/or the directed delivery of radioisotopes to tumor cells. Other forms of radiotherapies are also contemplated such as microwaves and UV-irradiation. It is most likely that all of these therapies cause a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may include external radiation therapy, hypofractionated radiation therapy, internal radiation therapy, or radiopharmaceutical therapy. External radiation therapy involves a radiation source outside the subject's body and sending the radiation toward the area of the cancer within the body. Conformal radiation is an external radiation therapy that uses computer-assisted 3-dimensional (3D) imaging of the tumor and shapes the radiation beams to fit the tumor; allowing a high dose of radiation to reach the tumor specifically, while causing less damage to surrounding healthy tissue.

Hypofractionated radiation therapy is radiation treatment in which a larger than usual total dose of radiation is given once a day over a shorter period of time (fewer days) compared to standard radiation therapy. Hypofractionated radiation therapy may have worse side effects than standard radiation therapy, depending on the schedules used.

Internal radiation therapy uses a radioactive substance sealed in needles, seeds, wires, or catheters that are placed directly into or near the cancer. In early-stage prostate cancer, the radioactive seeds are placed in the prostate using needles that are inserted through the skin between the scrotum and rectum. The placement of the radioactive seeds in the prostate is guided by computer-assisted images, typically from transrectal ultrasound or computed tomography (CT). The needles are removed after the radioactive seeds are placed at or in the tumor.

Radiopharmaceutical therapy uses a radioactive substance to treat cancer. Radiopharmaceutical therapy typically includes alpha emitter radiation therapy, which uses a radioactive substance to treat prostate cancer that has spread to the bone. A radioactive substance, e.g., radium-223, is injected into a vein and travels through the bloodstream. The radioactive substance collects in areas of bone with cancer and kills the cancer cells.

These and other aspects of the present disclosure will be further appreciated upon consideration of the following working examples, which are intended to illustrate certain embodiments of the disclosure but are not intended to limit its scope, as defined by the claims.

### EXAMPLES

The working examples that follow show that recognition of tumor antigens, but not antigen class per se, determine the intratumoral phenotype of anti-melanoma T cells. Interaction with tumor antigens led to selection of TCRs with avidities inversely related to the expression level of cognate targets in melanoma cells and proportional to the binding affinity of peptide-HLA class I complexes. Non-tumor reactive T cells were enriched for viral specificities and had non-exhausted memory phenotypes. In contrast, melanoma-reactive lymphocytes predominantly displayed an exhausted state that encompassed diverse levels of cellular differentiation, but only rarely an effector state with memory properties. These dysfunctional phenotypes were observed among TCR clonotypes displaying a broad range of avidities whether for public melanoma antigens or personal neoantigens. The TCR clonotypes from intratumoral exhausted lymphocytes persisted in peripheral blood at higher levels in patients with poor response to immune checkpoint blockade compared to those achieving durable disease regression, consistent with chronic stimulation mediated by the presence of residual tumor antigen. By revealing how quality and quantity of tumor antigens drive the features of T cell responses within the tumor microenvironment, insights were gained with respect to into the properties of the anti-cancer TCR repertoire.

### DESCRIPTION OF THE DRAWINGS

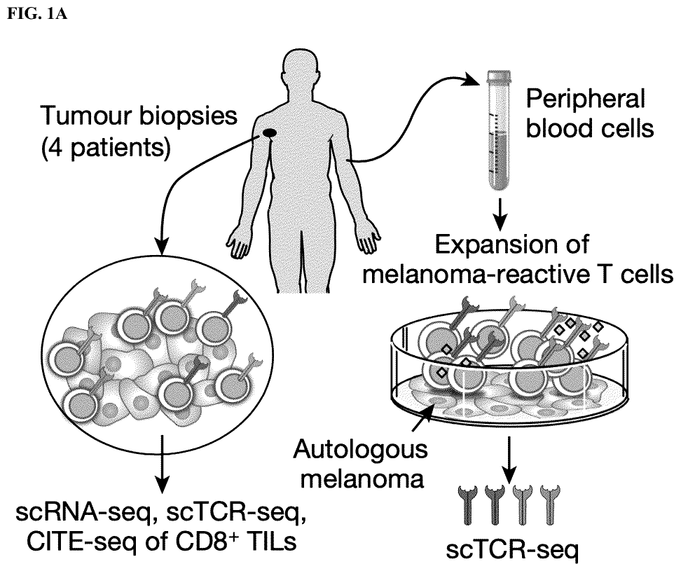

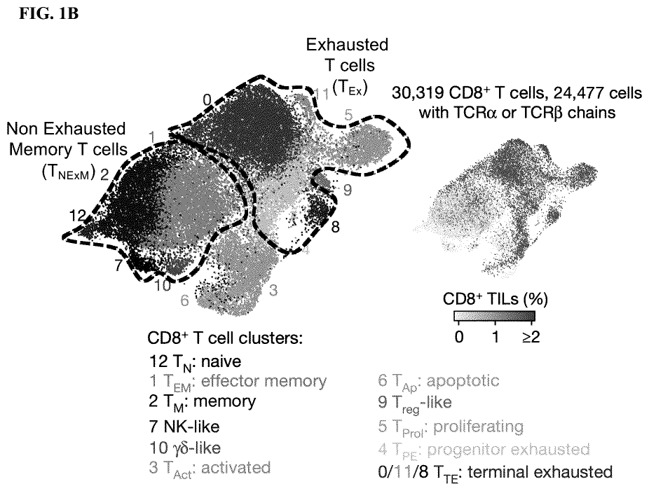

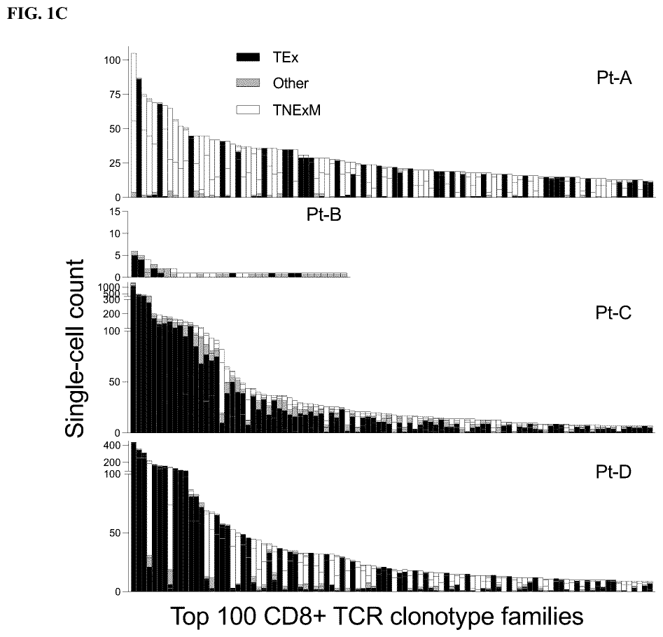

Generally, FIG. 1A-1C are a series of schematics, UMAPs, and bar plots illustrating the distinct pattern of cell states of intratumoral CD8+ TCR clonotype families in patients with melanoma.

FIG. 1A is a schematic of sample collection, processing, and single-cell (sc) sequencing analysis. FIG. 1A shows the process that allows isolation and selection of T cell receptors from patient's biopsies: after single cell profiling of T cells infiltrating the tumors, expanded T cell clones are identified based on their transcriptional profile (the expression state) and their TCR is selected. Therefore, assigning the T cells into a plurality of clonotype families on the basis of TCR sequences (through scTCR-seq) and definition of their state (through scRNA-seq) allows one to identify and select TCR clonotypes associated with specific expression states.

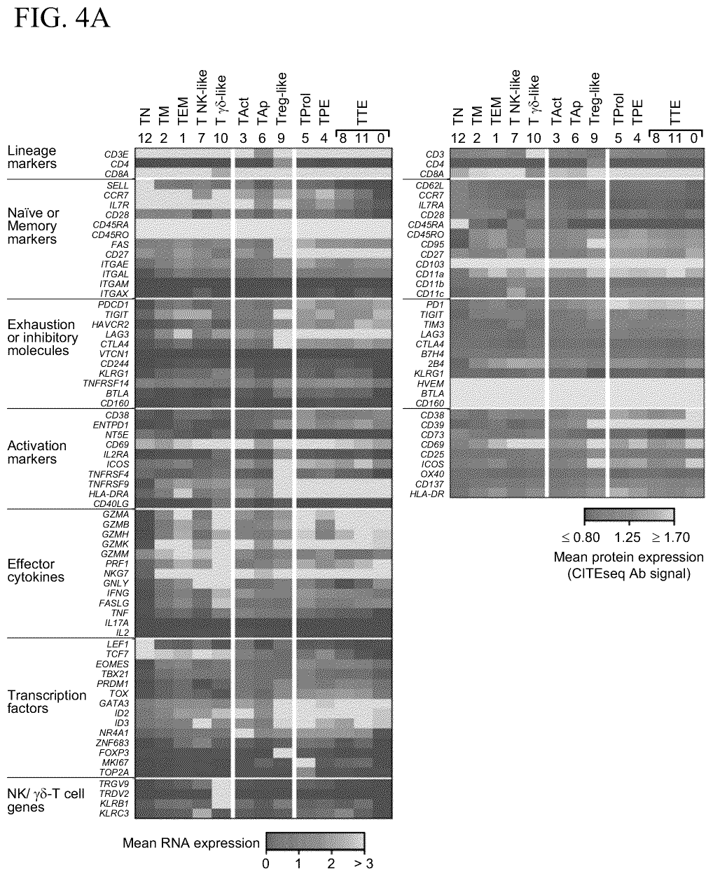

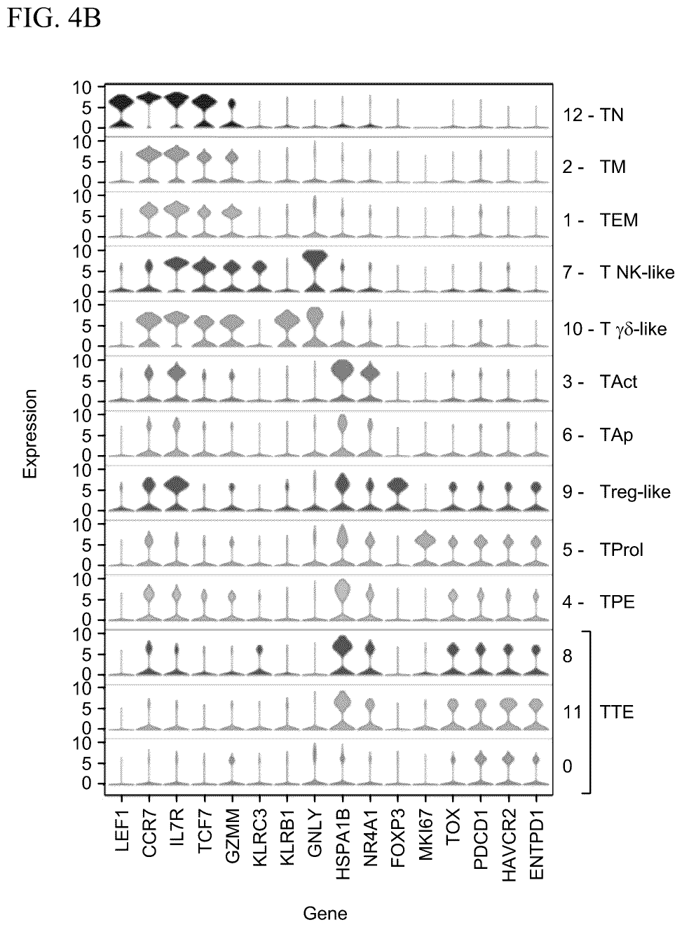

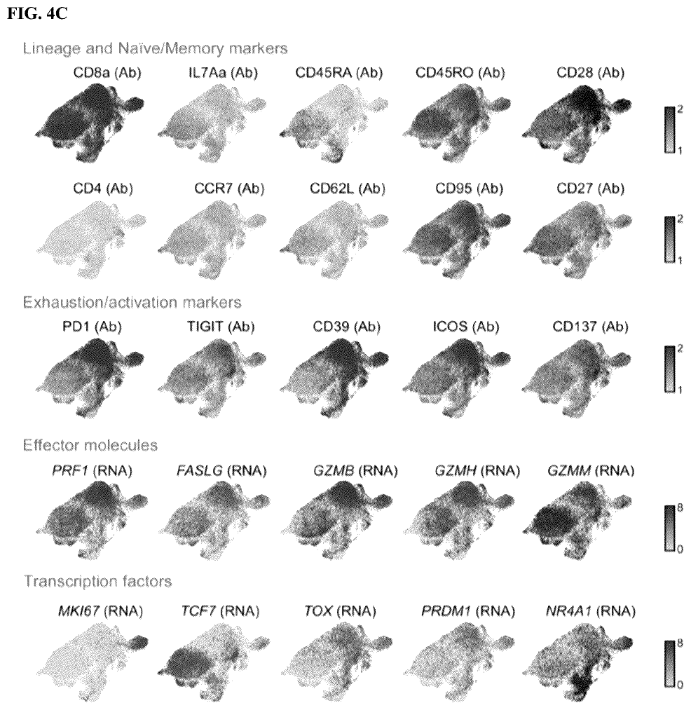

FIG. 1B is a UMAP of scRNA-seq data from CD8+ melanoma-infiltrating T cells. FIG. 1B depicts the cellular states of CD8+ T cells infiltrating tumor lesions, as defined through single-cell RNA-seq. The different states are named based on expression of different markers (see FIG. 4A, 4B, 4C). Based on the expression of memory or exhaustion markers, CD8+ TILs can be divided in two major compartments: exhausted (TEx) or non-exhausted memory (TNExM) T cells. Analysis of TCR representation demonstrates that expanded clones have a preferential exhausted phenotype (FIG. 1B-right). This figure demonstrates the detection of T cell clones that are exhausted and expanded within the tumors, allowing their selection for therapeutic purposes.

FIG. 1C is a bar plot showing the top 100 TCR clonotype families from four patients. FIG. 1C demonstrates that the TCR clonotype families expanded within the tumor microenvironment and identified based on TCR identity can be distinguished based on their cellular state in exhausted (TEx) or non-exhausted memory (TNExM). This allows the selection of those TCR clonotypes with an exhausted cellular state.

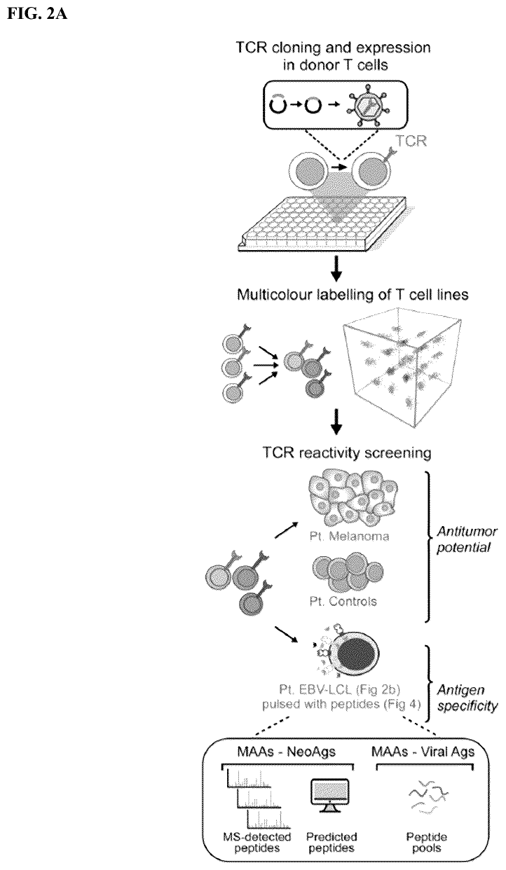

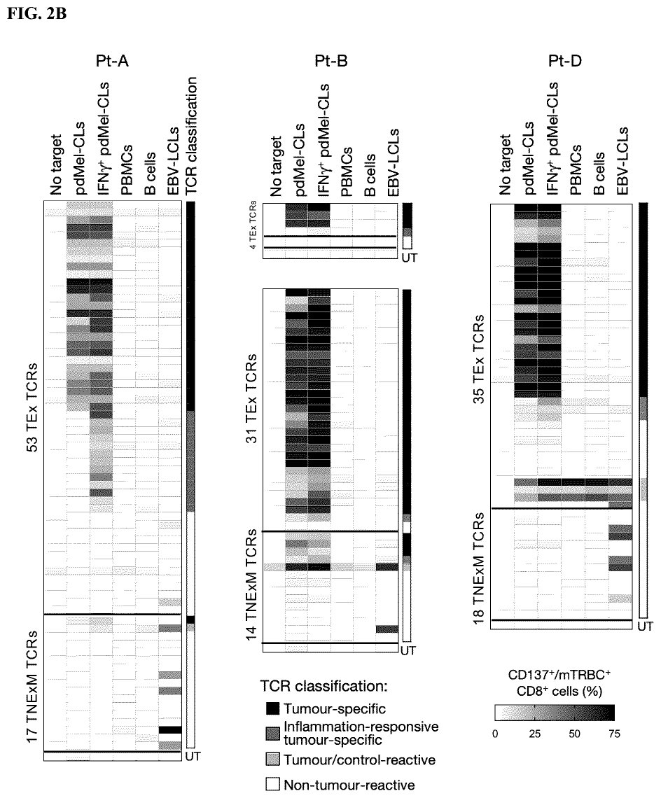

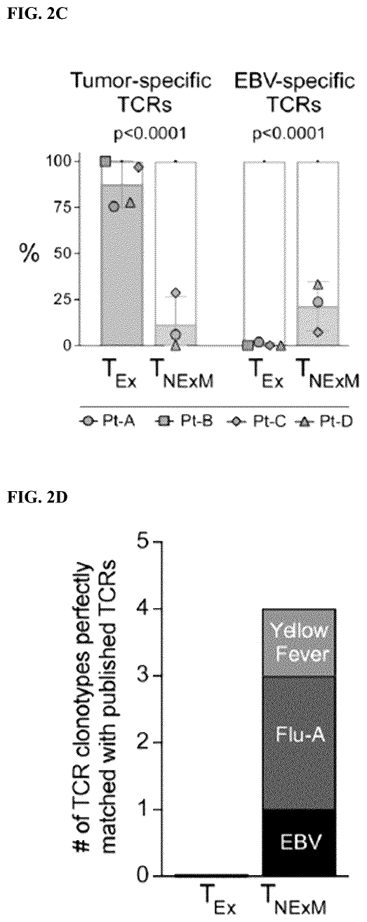

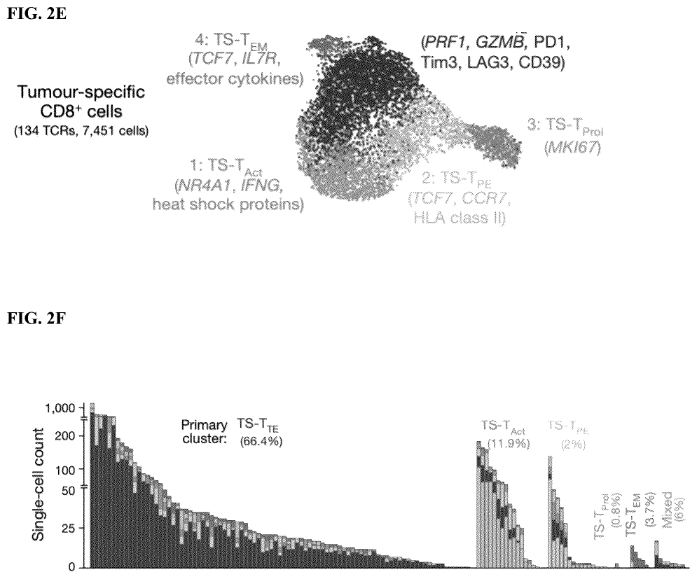

Generally, FIG. 2A-2F are a series of schematics, heatmaps, box, bar, and UMAP plots showing the tumor-specificity and cellular states of CD8+ TCR clonotype families.

FIG. 2A is a schematic representation of the workflow for in vitro TCR reconstruction and specificity screening. FIG. 2A shows the experimental process that was applied to TCR detection within the tumor microenvironment to demonstrate that antitumor TCRs can be isolated from clones with an exhausted phenotype. TCRs identified in tumor specimens were cloned and expressed in T cells from healthy donors, and screened for their reactivity against tumor or non-tumor cells and against tumor antigens. This process further demonstrates that it is possible to modify T cells with TCRs from expanded tumor infiltrating T cells to generate T cells with antitumor potential.

FIG. 2B includes heatmaps showing the reactivity of dominant TCRs originating from cells in exhausted (T_{Ex}, top) or non-exhausted memory (T_{NExM}, bottom) clusters infiltrating 4 melanoma specimens. Results depicted in FIG. 2B demonstrate that TCRs isolated from exhausted and expanded TILs are highly tumor reactive and therefore they can be used for therapeutic purposes. These experiments further demonstrate that it is possible to modify T cells with TCRs identified from exhausted and expanded TCR clonotype families to achieve an antitumor reactivity in vitro.

FIG. 2C is a box plot showing tumor-specific (left) and EBV-specific (right) TCR clonotypes. FIG. 2D is a bar plot showing TCRs from T_{Ex} or T_{NExM} clusters that perfectly matched with known TCR sequences. FIG. 2C-2D show the frequency of tumor specific or virus-specific TCRs that were isolated from exhausted (Tex) or non-exhausted memory (T_{NExM}) TILs harvested from 4 patients (Pt-A-D). The data provided demonstrates that only T_{Ex} TCRs are significantly enriched in antitumor specificities, and therefore they can be isolated for the manipulation of T cells to achieve antitumor effects.

FIG. 2E is a UMAP of scRNA-seq data from CD8+ TILs. FIG. 2F is a bar plot showing the CD8+ phenotypes of TCRs. FIG. 2E-2F show the cellular states identified from analysis of T cells with tumor-specific TCRs. They demonstrate that the vast majority of T cells with validated antitumor reactivity have a terminally exhausted phenotype (T_{TE}). Therefore, isolation of TCRs from exhausted cells grants the possibility to discover TCRs with antitumor reactivity that can be unexploited to treat cancer patients.

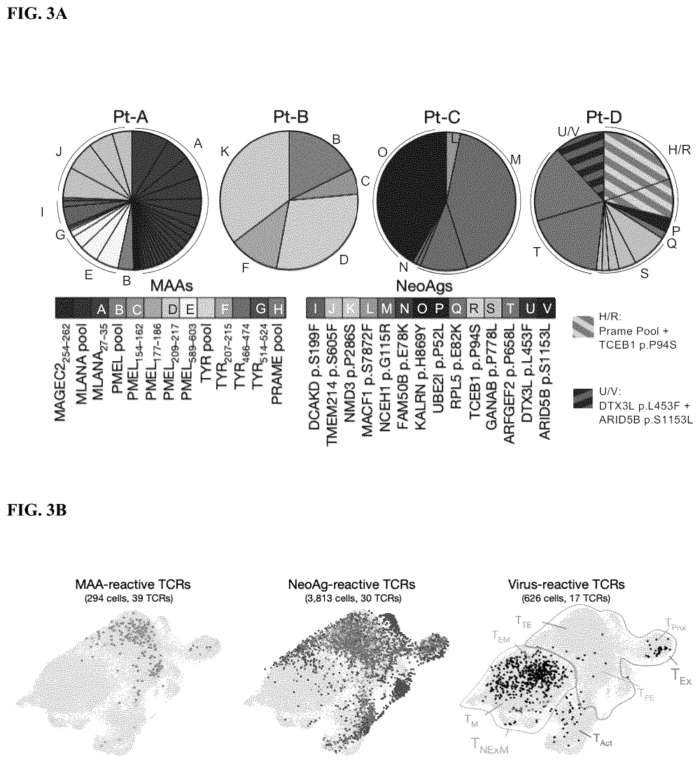

Generally, FIG. 3A-3B are a series of pie and UMAP plots showing the antigenic specificity and recognition avidity of tumor-specific TCRs.

FIG. 3A includes four pie plots showing a summary of the de-orphanized antigen specificity of intratumoral TCRs with confirmed antitumor reactivity. FIG. 3A documents the tumor antigens that are recognized by antitumor exhausted TCRs, as established in 4 patients with melanoma. These data demonstrate that isolation of TCRs from exhausted intratumoral expanded T cells allows one to find T cells specific for tumor specific antigens, such as melanoma associated antigens (MAAs) or neoantigens (NeoAgs).

FIG. 3B is a series of UMAPs showing the phenotypic distribution of T cells bearing antitumor TCRs specific for MAAs or NeoAgs or TCRs specific for viral peptides. FIG. 3B shows that TCR clonotype families expressing TCRs specific for tumor antigens localize within the portion of the UMAP that is specific for the exhausted T cells (TEx). Conversely, anti-viral specificities localize among memory T cells. Therefore, the selection of TCRs from exhausted TILs allows the isolation of TCRs with antitumor specificity.

Generally, FIG. 4A-4C is a series of heatmaps, violin plots, and UMAPs showing the single-cell profiling of CD8+ tumor infiltrating lymphocytes.

FIG. 4A is a series of heatmaps depicting the mean cluster expression of a panel of T-cell related genes. FIG. 4B shows violin plots quantifying relative transcriptional expression of genes (columns) with high differential expression among CD8+ TIL clusters (rows). FIG. 4C shows UMAPs depicting the single-cell expression of representative T cell markers among CD8+ TILs. These three figures show the markers that are characteristic of exhausted of memory T cells, as established from single-cell analysis of T cells from tumor biopsies. The results demonstrate that exhausted T cells can be isolated based on the expression on several markers, including PDCD1, HAVCR2, CTLA4, ENTPD1, LAG3, and TOX RNA transcripts (determined using scRNAseq) and one or more of PD1, Tim-3, CTLA4, CD39 proteins (determined by CITE-seq).

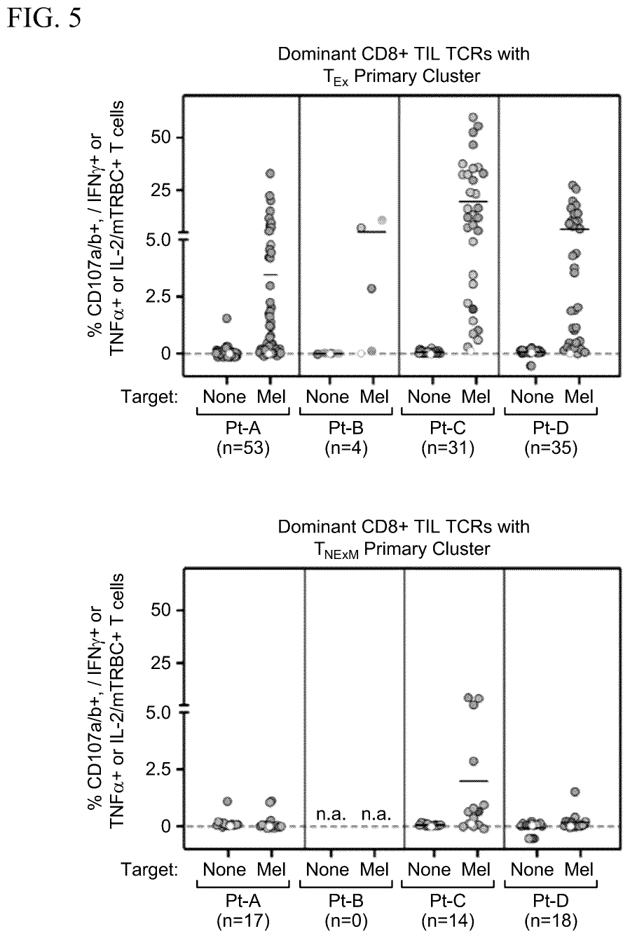

Generally, FIG. 5 is a series of dot plots showing the antitumor reactivity of in vitro reconstructed TCRs.

More specifically, FIG. 5 includes two dot plots showing cytotoxic potential provided by TCRs with exhausted (left) or non-exhausted (right) primary clusters isolated from all 4 studied patients. The data depicted in FIG. 5 show that TCRs isolated from TEx (left) are able to convey antitumor reactivity when expressed in T cells from healthy donor. Conversely, most of TCRs isolated from memory cells (right) are not able to determine antitumor cytotoxicity, as measured in vitro. These results document that TCRs isolated from exhausted TILs are highly enriched in antitumor specificities. Furthermore, it is possible to use such TCRs to modify and reprogram T cells. This process allows one to obtain T cells with high antitumor specificity that can be used to treat cancer cells, as demonstrated in vitro. Note, in FIG. 5, the shading of the dots indicates TCR clonotypes belonging to different subsets of TEx (left) or TNExM (right), as indicated in FIG. 1B.

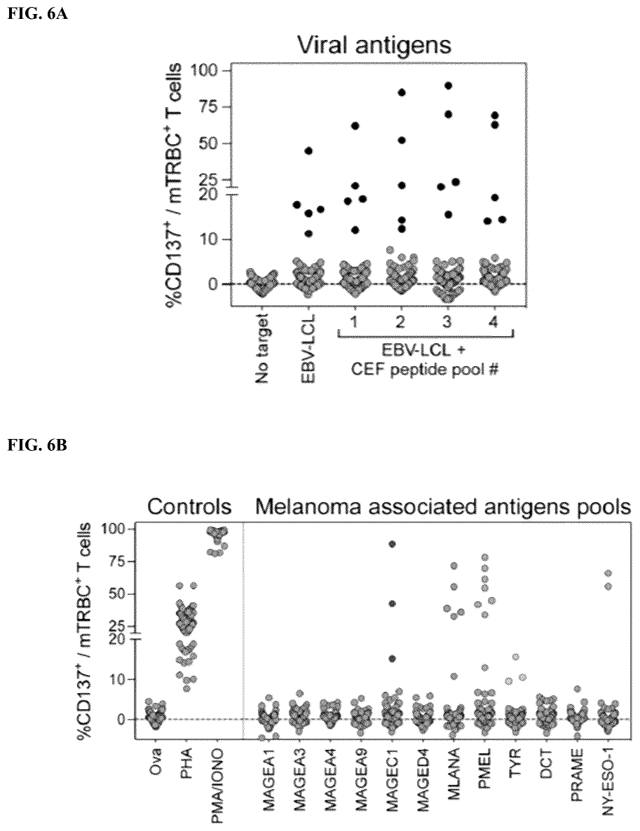

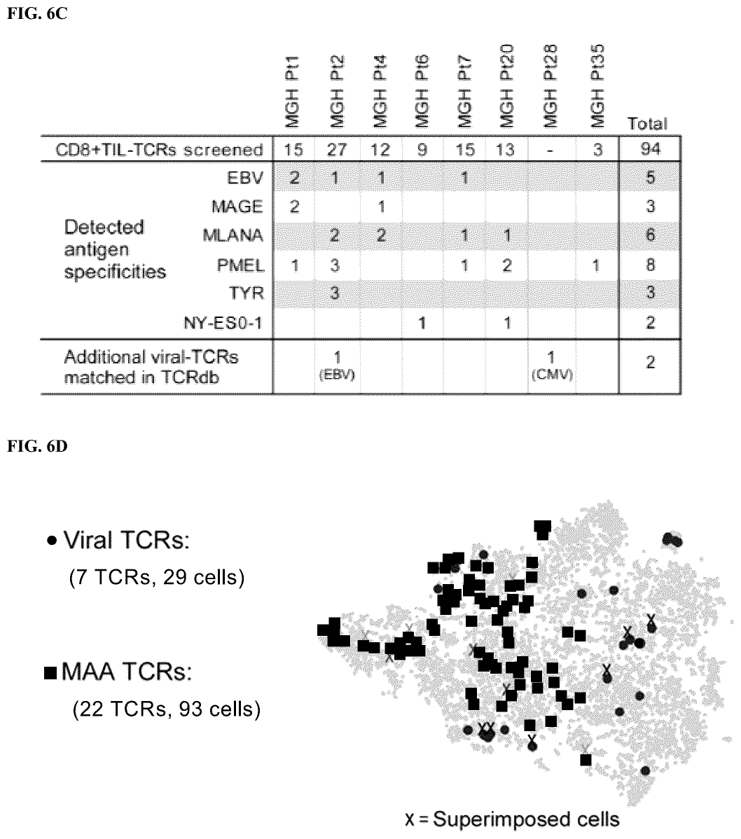

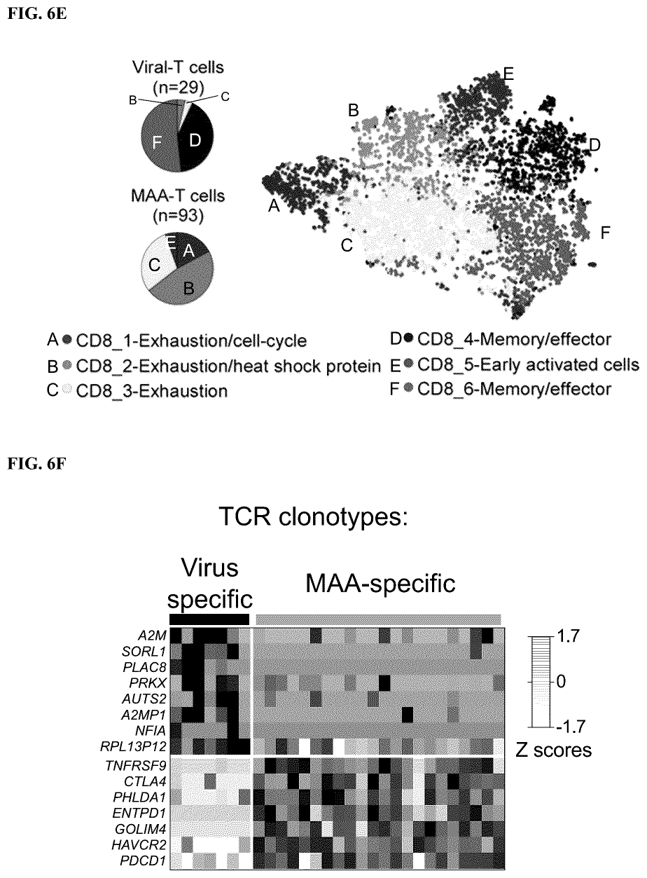

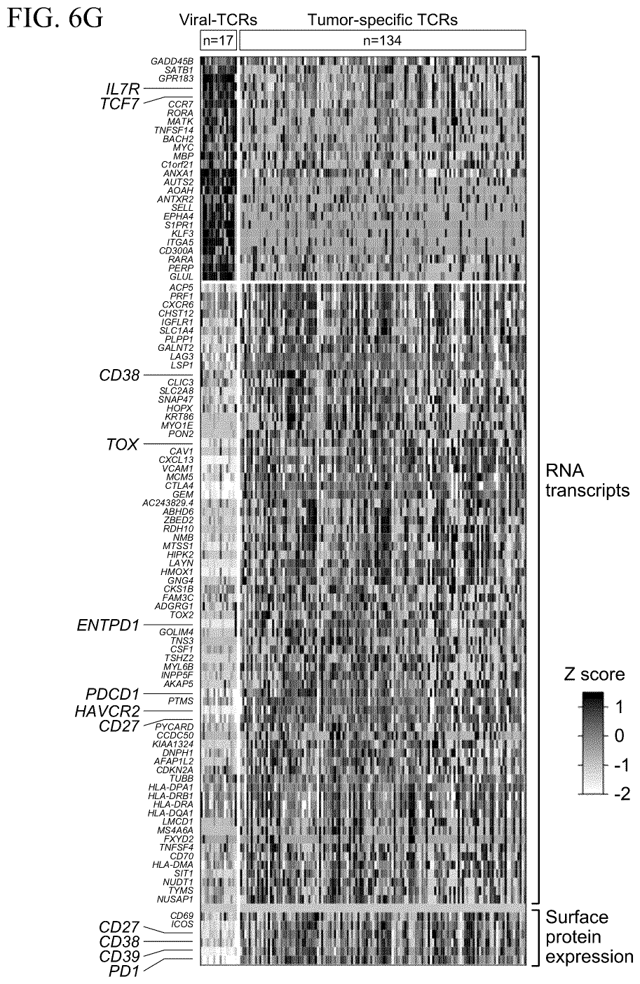

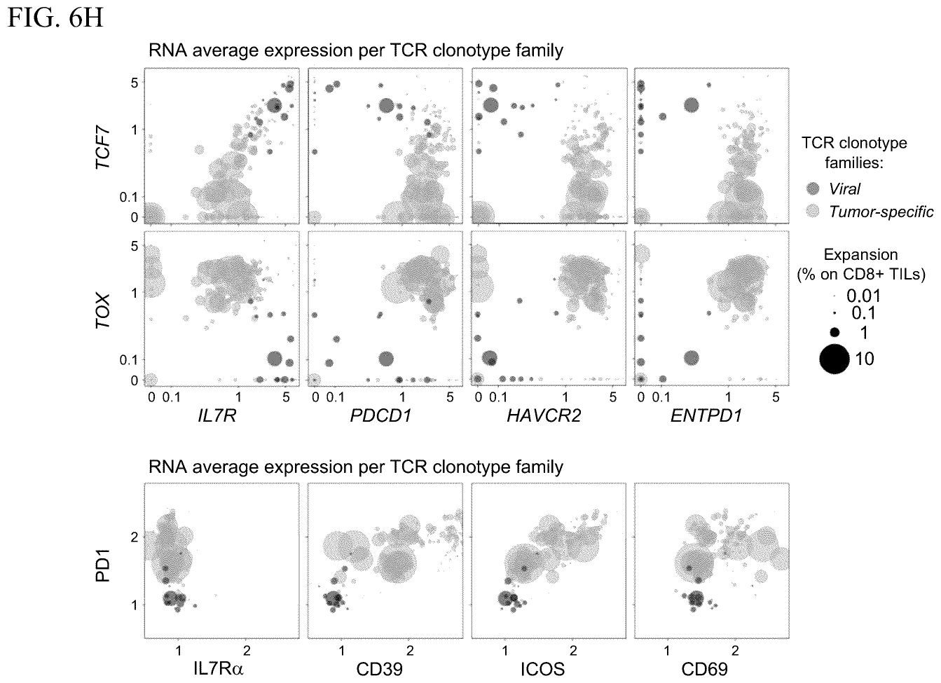

Generally, FIG. 6A-6H are a series of dot plots, tables, UMAPS, pie charts and heatmaps showing cell states of tumor-specific CD8+ TILs. Note, in FIGS. 6A and 6B, the shading of the dots indicates specificity for different viral or tumor antigens, as indicated on the x axis.

FIG. 6A-6C are dot plots and a table showing antigen specificity screening of 94 TCRs sequenced from clonally expanded CD8+ T cells. FIG. 6D-6F are two UMAPs and a heatmap showing single-cell phenotype of TILs with antiviral or anti-MAA TCRs. FIG. 6G-6H are a heatmap and a series of dot plots show the analysis of deregulated genes in exhausted clusters (T_{Ex}), enriched in tumor-reactive T cells, from the discovery cohort.

The data depicted in FIG. 6A-6C summarize the specificities of TCRs isolated from exhausted or memory T cells infiltrating tumor lesions of 8 patients. The data reported in FIG. 6D-6F demonstrate that among such TCRs, those specific for tumor antigens can be isolated from the exhausted T cells, which carry expression of exhaustion markers. Conversely, anti-viral T cells can be isolated from memory T cells with no expression of exhaustion markers. This validates the process of isolation of antitumor TCRs from exhausted T cells infiltrating tumor lesions. The data reported in FIG. 6G-6H report a comparison between the gene expression profiles of T cells with tumor-specific TCRs and with anti-viral TCRs. The results demonstrate that T cells with antitumor TCRs are characterized by high expression of exhaustion markers, both at the levels of RNA transcripts and surface proteins. Therefore, these data prove that antitumor TCRs can be isolated from T cell clones identified base on the expression of one or more of a) PDCD1, HAVCR2, CTLA4, ENTPD1, LAG3, and TOXRNA transcripts determined using scRNAseq, and/or b) one or more of PD1, Tim-3, CTLA4, CD39, and LAG3 surface proteins (exhaustion markers).

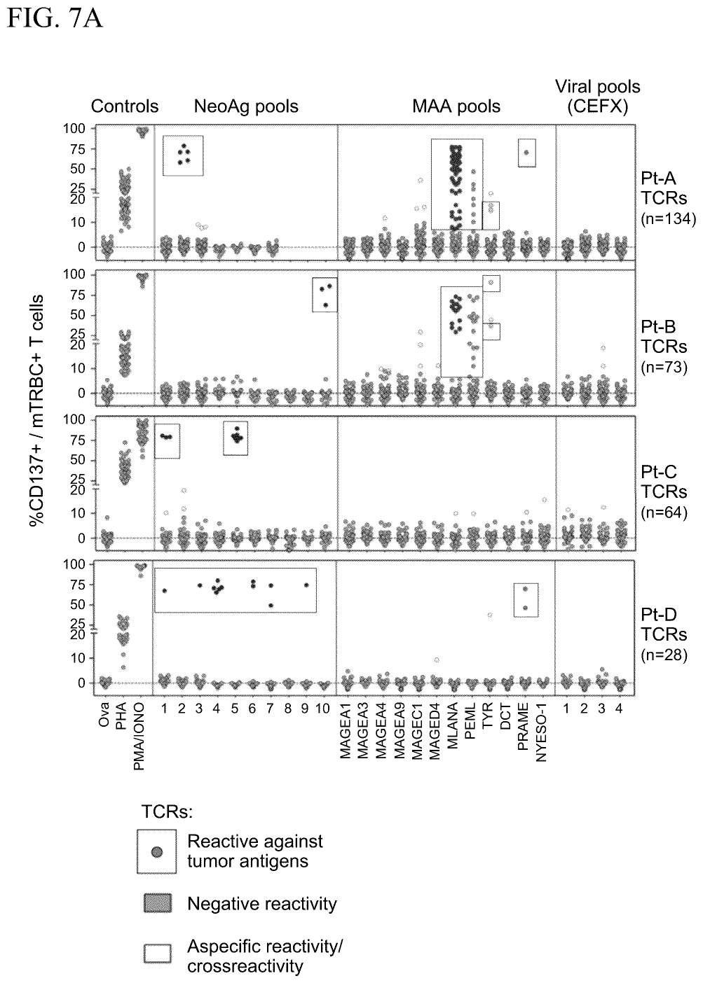

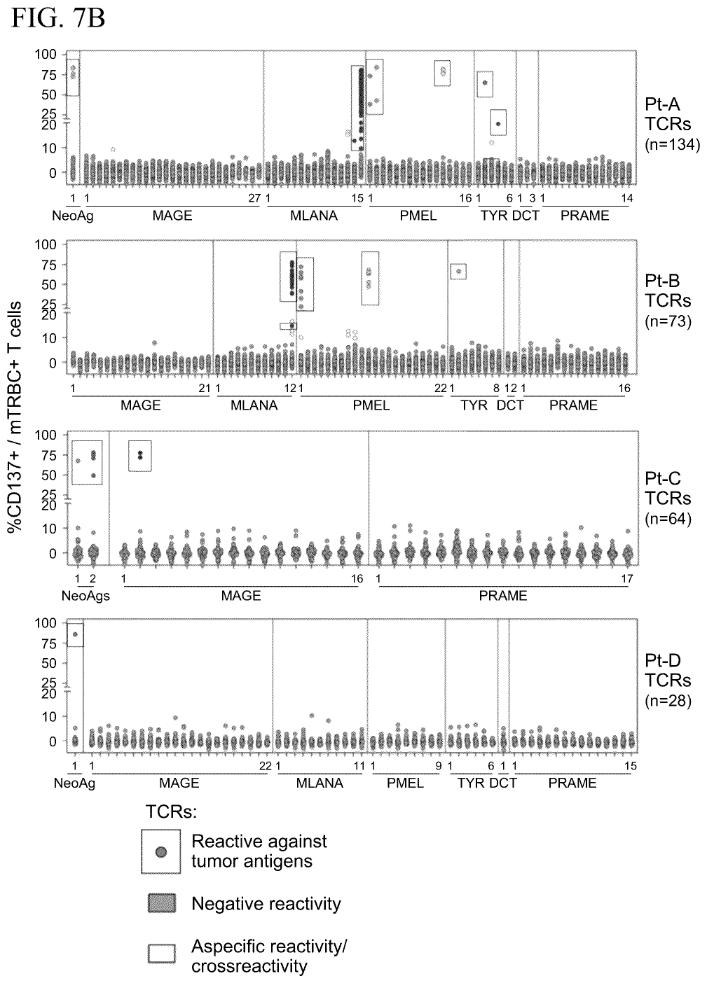

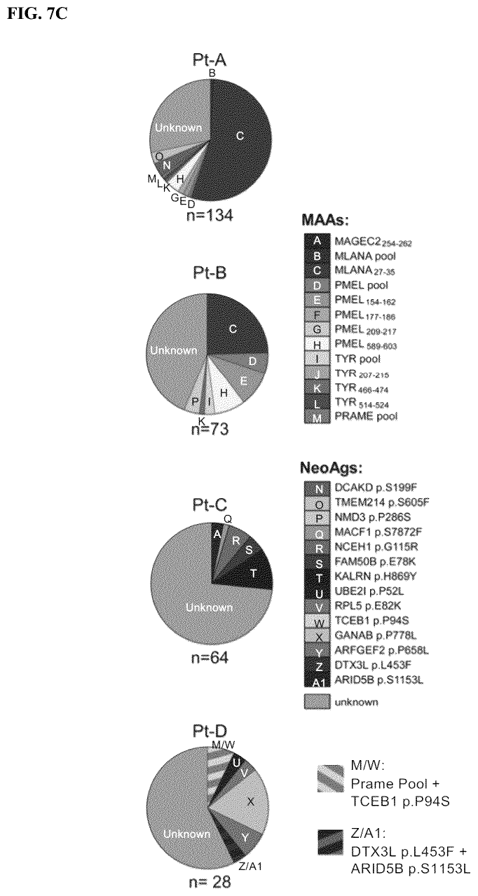

Generally, FIG. 7A-7C are a series of dot plots and pie charts showing antigen specificity of tumor-reactive TCRs.

FIG. 7A-7B are a series of dot plots showing antigen specificity screening of 299 antitumor TCRs. FIG. 7C includes four pie charts showing distribution of antigen specificities of antitumor TCRs per patient successfully de-orphanized after screening FIG. 7A-7C report the results of the specificity of antitumor TCRs isolated from exhausted T cells, demonstrating that they can recognize tumor antigens such as melanoma associated antigens (MAAs) or neoantigens (NeoAgs). The data prove also that gene-manipulation of T cells with TCRs identified among exhausted T cells is able to confer the ability to recognize tumor antigen. Therefore, such TCRs can be exploited to achieve an antitumor effect, as demonstrated here in vitro.

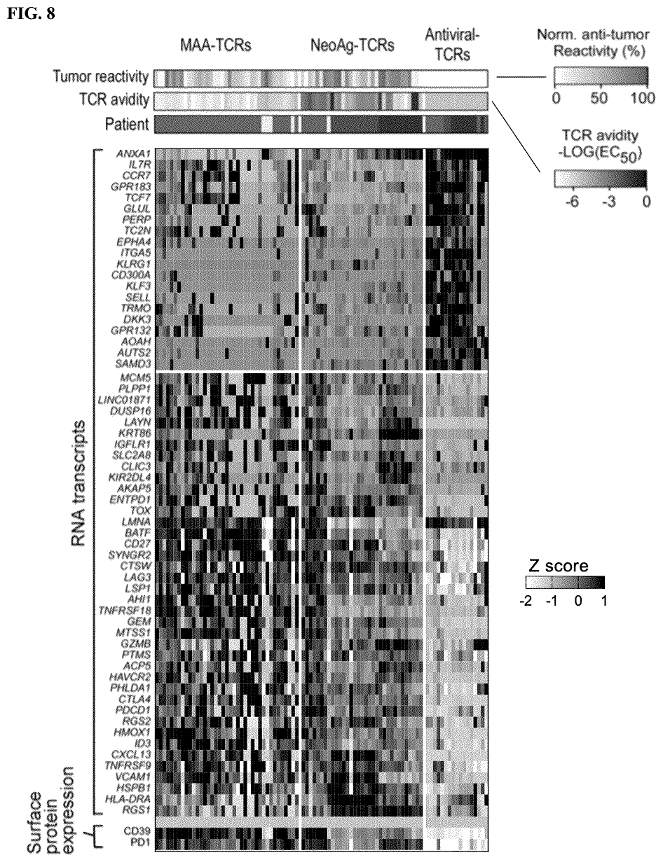

Generally, FIG. 8 is a heatmap showing genes differentially expressed between CD8+ TILs with identified MAA, NeoAg-specific, or virus-specific TCRs.

FIG. 8 demonstrates that antitumor TCRs, including those specific for melanoma associated antigens or neoantigens, are harbored by T cells with high expression of exhausted markers. These cells can be separated from T cells with no antitumor reactivity (anti-viral T cells) thanks to the expression of transcripts and surface proteins indicative of exhaustion (PDCD1, HAVCR2, CTLA4, ENTPD1, LAG3, and TOXRNA transcripts; PD1 and CD39 surface proteins).

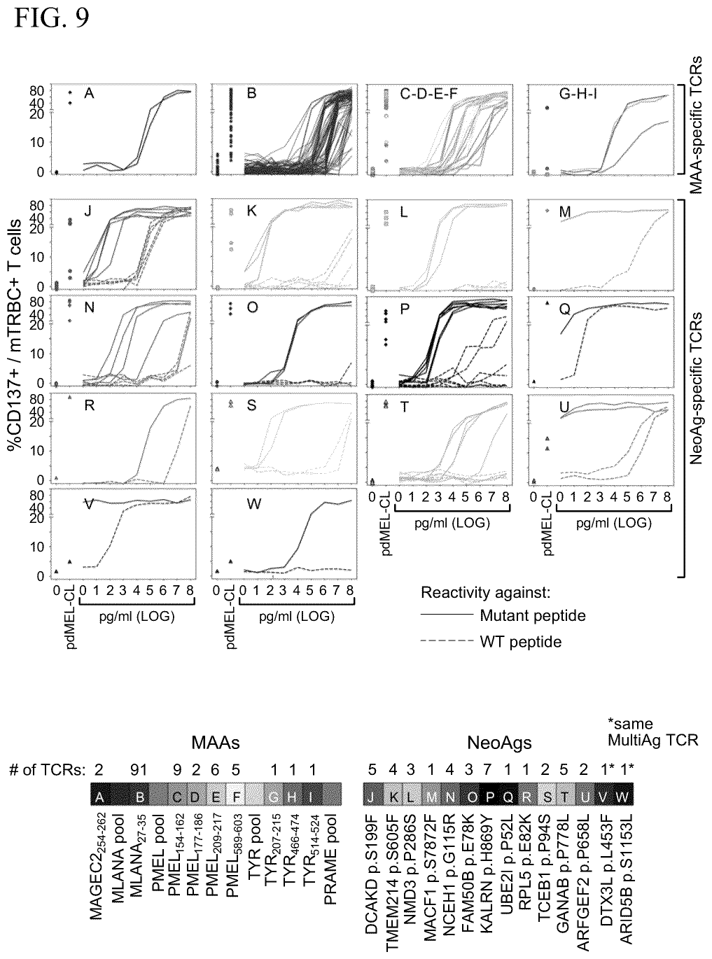

Generally, FIG. 9 is a series of line plots showing normalized antitumor TCR reactivity and avidity.

FIG. 9 reports the reactivity of T cells modified to express the TCRs isolated from exhausted T cells infiltrating tumor lesions. The reactivity of TCRs with de-orphanized cognate antigens is reported. These data shows that expression of such TCRs in non-exhausted T cells isolated from peripheral blood of healthy donors allow to generate T cells with high antitumor efficacy, as demonstrated in vitro.

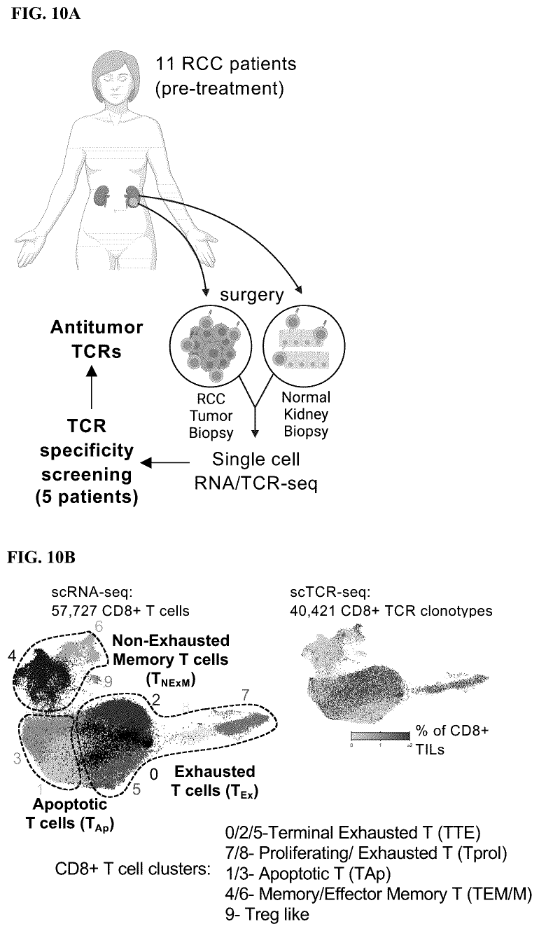

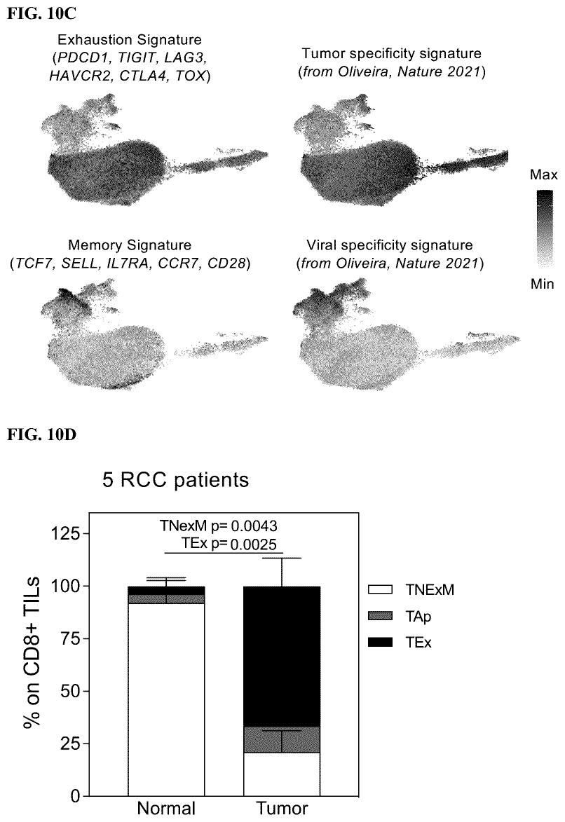

Generally, FIG. 10A-10D are a series of schematics, UMAP plots and bar charts illustrating the characterization of T cells infiltrating renal cell carcinoma specimens and the identification of antitumor TCRs in clear cell renal cell carcinoma (ccRCC) samples.

FIG. 10A is a schematic of sample collection, processing, and single-cell sequencing analysis and identification of antitumor TCRs in clear cell renal cell carcinoma (ccRCC) samples collected from treatment-naïve patients. FIG. 10B is a UMAP of scRNA-seq data from CD8+ renal cell carcinoma TILs. Clusters are denoted by numbers and labelled with inferred cell states. T cell subsets are further divided in metaclusters of non-exhausted memory (T_{NExM}), Exhausted (T_{Ex}) or Apoptotic (Ta p) T cells. The same UMAP (right) shows TILs marked on the basis of intrapatient TCR clone frequency defined through scTCR-seq. FIG. 10C are UMAPs of CD8+ TILs colored based on enrichment of gene-signatures of exhaustion and memory T cells (left) or associated with CD8+ TILs with validated antiviral (top) or antitumor (bottom) reactivity, as established in Oliveira et al., Nature 596, 119-125 (2021)). FIG. 10D is a bar chart showing the frequencies of T cell metaclusters, as detected by scRNA-seq in normal kidney tissues and tumor biopsies. Data are reported for 5 ccRCC patients selected for analysis of antitumor specificities. P values indicate significant comparisons between metaclusters in tumor and normal specimens, as calculated using a two-side t-test. In sum, these figures show that T cells infiltrating renal cell carcinomas are highly exhausted. That is, the data demonstrates that expanded tumor-infiltrating T cell clones express markers of exhaustion.

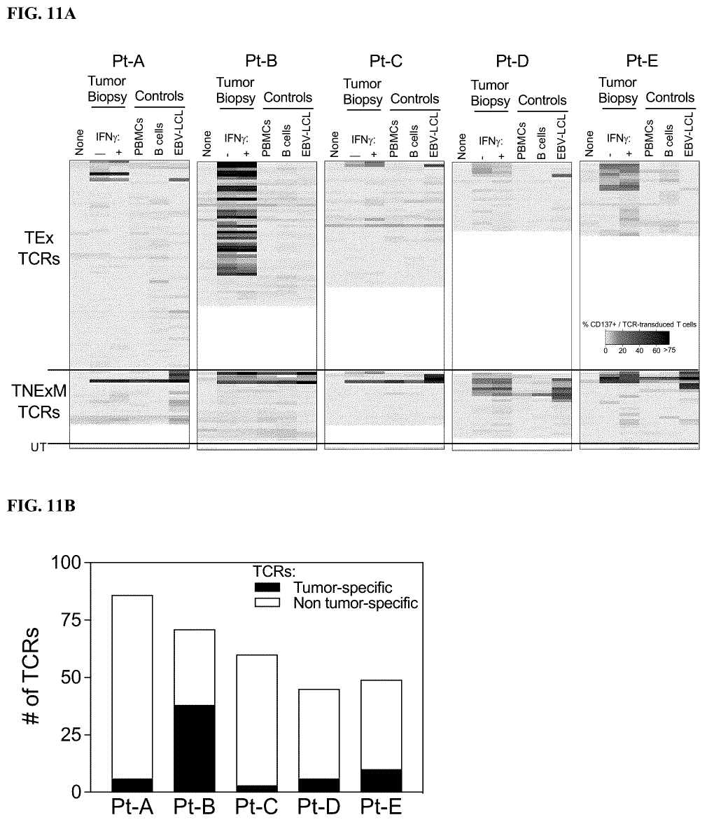

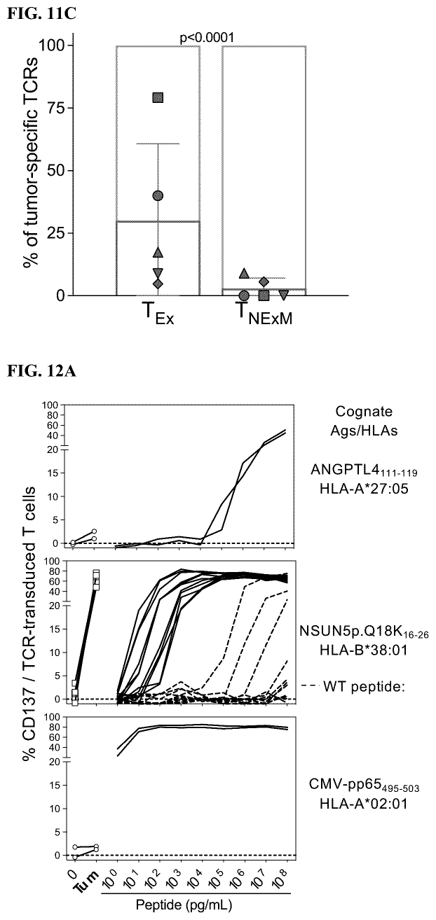

Generally, FIG. 11A-11C are a series of heatmaps and bar charts showing the reactivity of dominant TCRs sequenced among Ta or T_{NExM} clusters in 5 ccRCC patients.

FIG. 11A shows a series of heatmaps showing the reactivity of dominant TCRs sequenced among T_{Ex} (top) or T_{NExM} (bottom) clusters in 5 ccRCC patients A-E. CD137 upregulation was measured on TCR-transduced CD8+ T cells cultured alone (no target) or in the presence of autologous cells from tumor biopsy (cultured with or without interferon-γ (IFNγ) pre-treatment) or controls (peripheral blood mononuclear cells (PBMCs), B cells and EBV-LCLs). Background detected on CD8+ T cells transduced with an irrelevant TCR was subtracted. UT, untransduced cells. FIG. 11B is a bar chart showing the number of TCRs tested for each patient (columns) and classified as tumor specific (black). FIG. 11C shows the proportion of TCRs classified as tumor-specific among T_{Ex}-TCRs or T_{NExM}-TCRs in 5 patients with ccRCC, where each symbol identifies a different patient. Mean±s.d. are shown. P values were calculated using two-tailed Fisher's exact test on the total distribution of tested TCRs. In sum, these figures show that TCR clonotypes with antitumor potential are enriched among RCC-infiltrating T cells with an exhausted phenotype, and support the evidence that T cells can be reprogrammed to express TCRs isolated from exhausted T cells to achieve recognition of tumors.

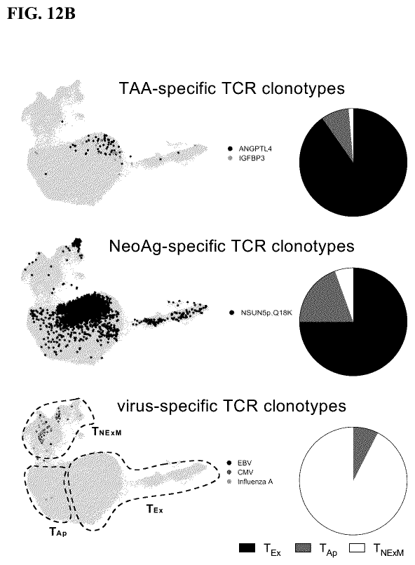

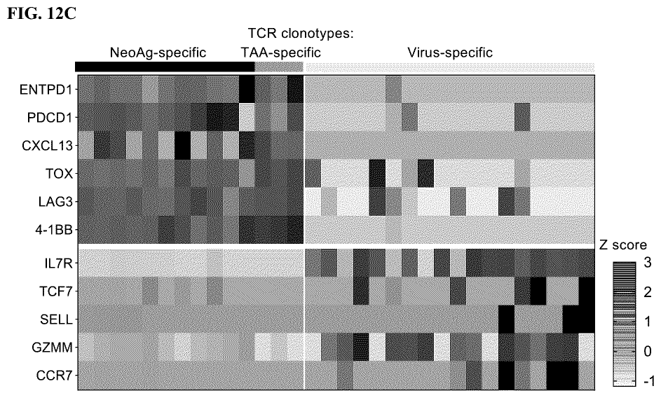

Generally, FIG. 12A-12C are a series of line charts, UMAPs, pie charts, and heatmaps showing the phenotypes of antigen specific TCR clonotypes infiltrating ccRCC tumors.

FIG. 12A is a series of line charts showing reactivity and avidity of ccRCC-TCRs with de-orphanized antigen specificity. TCR-dependent CD137 upregulation was measured on TCR-transduced (mTRBC+) CD8+ cells upon culture with patient-derived EBV-LCLs pulsed with increasing concentrations of the cognate antigen (tumor associated antigens TAAs in the top panel; NeoAgs in middle panel; viral Ags in bottom panel). Reactivity to DMSO-pulsed targets (0) and autologous tumor cultures (Tum) are reported on the left, to indicate the antitumor potential of each TCR specificity; for NeoAg-specific TCRs, the dashed lines report reactivity against wild-type peptides. The cognate antigens and HLA-restrictions of the TCRs is reported on the right. FIG. 12B shows the phenotypes of antigen specific TCR clonotypes in ccRCC. The UMAPs on the left show the phenotypic distribution of T cells bearing antitumor TCRs specific for TAAs-, NeoAgs- or virus-specific TCR clonotypes. The pie charts on the right show the frequency of T cells within each metacluster, as defined in FIG. 10B and reported on the UMAPs. FIG. 12C is a heatmap showing exhaustion (top) and memory (bottom) genes differentially expressed between CD8+ ccRCC TILs with identified TAA-specific, NeoAg-specific or virus-specific TCRs. The heatmap colors depict Z scores of average gene expression within a TCR clonotype (columns). Top tracks: annotations of antigen specificity. In sum, these figures show that intra-tumoral T cells with TCRs specific for tumor antigens (neoantigens or tumor associated) have an exhaustion phenotype, and therefore can be isolated from tumor specimens using markers of exhaustion.

### Example 1: Materials and Methods

Study subjects and patient samples. Single-cell sequencing and TCR screening analyses were conducted on four patients with high-risk melanoma enrolled between May 2014 and July 2016 to a single center, phase I clinical trial approved by the Dana-Farber/Harvard Cancer Center Institutional Review Board (IRB) (NCT01970358). This study was conducted in accordance with the Declaration of Helsinki. The details about eligibility criteria have been described previously (Ott et al., Nature 547:217-221, 2017), and all subjects received neoantigen-targeting peptide vaccines, as previously reported (Table 1) (Ott et al., Nature 547:217-221, 2017). Tumor samples were obtained immediately following surgery and processed as previously described. See, Ott et al., Nature 547:217-221 (2017). Heparinized blood and serum samples were obtained from subjects as known in the art. Peripheral blood mononuclear cells (PBMCs) were isolated by Ficoll/Hypaque density-gradient centrifugation (GE healthcare) and cryopreserved with 10% dimethylsulfoxide in FBS (Sigma-Aldrich, St. Louis, MO www.sigmaaldrich.com). Cells and serum from patients were stored in vapor-phase liquid nitrogen until the time of analysis. HLA class I and class II molecular typings were determined by PCR-rSSO (reverse sequence specific oligonucleotide probe), with ambiguities resolved by PCR-SSP (sequence specific primer) techniques (One Lambda Inc., West Hills CA, www.thermofisher.com/onelambda).

TABLE 1
Characteristics of discovery cohort

| Patient ID | Pt-A | Pt-B | Pt-C** | Pt-D |
|---|---|---|---|---|
| Patient #* | Pt-1 | Pt-3 | Pt-6 | Pt-12 |
| Age | 26 | 51 | 61 | 63 |
| Gender | M | F | M | F |
| Primary Site | Back | Left Calf | Chest | Right Forearm |
| Site of resected | Axillary LN | Skin - in transit | Lug | Axillary LN |
| disease | | | | |
| Stage | IIIC | IIIC | IVM1B | IIC |
| | (T3bN3M0) | (T3bN3cM0) | (T2aNoM1b) | (T2aN1bM0) |
| Previous | IFNα | — | IFNα | — |
| treatments | | | | |
| Treatments after | Neoantigen | Neoantigen | Neoantigen | Neoantigen |
| surgery | peptide | peptide | peptide | peptide |
| | vaccination | vaccination | vaccination | vaccination |
| Recurrence | Y (41/brain) | Y (28/Skin, left | Y (8/Skin, left | N |
| (months from | | calf) | back) | |
| surgery/site) | | | | |
| Treatments after | Radiations, | Surgery, | Anti-PD1 | — |
| recurrence | surgery, anti- | radiations | | |
| | PD1, targeted | | | |
| | (BRAF/MEK | | | |
| | inhibitors) | | | |
| HLA-A alleles | 02:01 | 02:02 | 66:01 | 02:01 |
| | 24:02 | 03:01 | 01:03 | 02:02 |
| HLA-B alleles | 44:02 | 47:01 | 08:01 | 13:02 |
| | 15:01 | 01:02 | 07:01 | 02:02 |
| HLA-C alleles | 07:02 | 06:02 | 07:01 | 06:02 |

M: Male, F: Female, LN: Lymph Node, Y: Yes, N: No
*as reported in Hu et al., Nat. Med. 27: 515-25 (2021)
**relapse sample corresponding to Pt-C-rel sample

Patient tumor samples were obtained immediately following surgery. A portion of the sample was removed for formalin fixation and paraffin embedding (FFPE). The remainder of the tissue was carefully minced manually, suspended in a solution of collagenase D (200 units/mL) and DNAse I (20 units/mL) (Roche Life Sciences, Penzberg, Germany, lifescience.roche.com), transferred to a sealable plastic bag and incubated with regular agitation in a Seward Stomacher Lab Blender for 30-60 min. After digestion, any remaining clumps were removed and the single cell suspension was recovered, washed, and immediately frozen in aliquots and stored in vapor-phase liquid nitrogen. In some cases, the frozen tumor cell suspensions were used for whole-exome (WES) and RNA-sequencing (RNA-Seq). In other cases, WES and RNA-Seq were performed on scrolls from the FFPE tissue.

The analysis of TCR dynamics was extended to an independent cohort of 14 metastatic melanoma patients treated with immune checkpoint blockade therapy (Massachusetts General Hospital, Boston, MA), as previously reported (Sade-Feldman et al., Cell 176:1-20 (2019)). The updated clinical data of such patients are summarized in Table 2-Table 4. All patients provided written informed consent for the collection of tissue and blood samples for research and genomic profiling, as approved by the Dana-Farber/Harvard Cancer Center Institutional Review Board (DF/HCC Protocol 11-181).

TABLE 2
Patient Characteristics

| | | | Site of resected disease for scSEQ | |
|---|---|---|---|---|
| Patient ID | Age | Gender | (1st/2nd/3rd) | Stage |
| MGH Pt1 | 49 | M | right chest/anterior neck/anterior neck | IV(M1c) |
| MGH Pt2 | 75 | M | small bowel/left anxilla | IV(M1d) |
| MGH Pt4 | 29 | M | left shoulder/left shoulder | IV(M1c) |
| MGH Pt6 | 66 | F | Left upper back/cecum | IV(M1c) |
| MGH Pt7 | 74 | M | left forehead/left forehead | IV(M1c) |
| MGH Pt12 | 68 | M | small bowel/left anterior shoulder | IV(M1d) |
| MGH Pt13 | 48 | M | NA/porta hepatis | IV(M1c) |
| MGH Pt20 | 75 | F | NA/jejunum | IV(M1c) |
| MGH Pt23 | 73 | M | left lower back | IV(M1d) |
| MGH Pt26 | 72 | M | axillary lymph node | IV(M1c) |
| MGH Pt27 | 62 | F | upper abdomen | IV(M1d) |
| MGH Pt28 | 67 | F | right groin/right groin/right groin | IV(M1b) |
| MGH Pt29 | 79 | M | left axillary lymph node | IV(M1c) |
| MGH Pt30 | 64 | M | left laparoscopic adrenalectomy | IV(M1c) |
| MGH Pt31 | 52 | M | right axilla | IIIB |
| MGH Pt35 | 70 | M | Right iliac lymph node | IV(M1c) |

TABLE 3
Patient Characteristics

| | | Status | Overall |
|---|---|---|---|
| | | (Alive = 0; | survival |
| Patient ID | Immune therapies | Dead = 1) | (days) |
| MGH Pt1 | ipilimumab, pembrolizumab | 0 | 2055 |
| MGH Pt2 | pembrolizumab | 1 | 354 |
| MGH Pt4 | ipilimumab, nivolumab | 0 | 1755 |
| MGH Pt6 | ipilimumab, pembrolizumab | 0 | 1871 |
| MGH Pt7 | ipilimumab, nivolumab | 1 | 1091 |
| MGH Pt12 | nivolumab, lirilumab, ipilimumab, | 1 | 761 |
| | pembrolizumab | | |
| MGH Pt13 | ipilimumab, nivolumab | 0 | 1756 |
| MGH Pt20 | pembrolizumab | 1 | 1447 |
| MGH Pt23 | ipilimumab, pembrolizumab, | 1 | 756 |
| | nivolumab, urelomab | | |
| MGH Pt26 | ipilimumab, nivolumab, | 0 | 1749 |
| | pembrolizumab | | |
| MGH Pt27 | pembrolizumab | 1 | 100 |
| MGH Pt28 | ipilimumab, nivolumab | 0 | 1365 |
| MGH Pt29 | pembrolizumab | 0 | 1697 |
| MGH Pt30 | pembrolizumab | 1 | 1767 |
| MGH Pt31 | pembrolizumab | 0 | 1375 |
| MGH Pt35 | Atezolizumab | 0 | 1370 |

TABLE 4
Patient Characteristics

| | | # of TCRs | # of PBMC | | |
|---|---|---|---|---|---|
| | | screened for | samples | T_{Ex} | T_{NExM} |
| | Clinical | antigen | analyzed by | primary | primary |
| Patient ID | classification | specificity | bulk TCRseq | clusters | cluster |
| MGH Pt1 | Alive with | 15 | 6 | 170 | 82 |
| | disease | | | | |
| | progression | | | | |
| MGH Pt2 | Deceased | 27 | 2 | 94 | 25 |
| MGH Pt4 | Alive without | 12 | 7 | 48 | 57 |
| | disease | | | | |
| | progression | | | | |
| MGH Pt6 | Alive without | 9 | — | — | — |
| | disease | | | | |
| | progression | | | | |
| MGH Pt7 | Deceased | 15 | 10 | 93 | 56 |
| MGH Pt12 | Deceased | — | 7 | 89 | 25 |
| MGH Pt13 | Alive with | — | 5 | 30 | 10 |
| | disease | | | | |
| | progression | | | | |
| MGH Pt20 | Deceased | 13 | 7 | 106 | 30 |
| MGH Pt23 | Deceased | — | 5 | 172 | 31 |
| MGH Pt26 | Alive without | — | 7 | 6 | 23 |
| | disease | | | | |
| | progression | | | | |
| MGH Pt27 | Deceased | — | 1 | 111 | 16 |
| MGH Pt28 | Alive with | — | 9 | 61 | 65 |
| | disease | | | | |
| | progression | | | | |
| MGH Pt29 | Alive without | — | 3 | 12 | 10 |
| | disease | | | | |
| | progression | | | | |
| MGH Pt30 | Deceased | — | 9 | 55 | 8 |
| MGH Pt31 | Alive without | — | 7 | 40 | 16 |
| | disease | | | | |
| | progression | | | | |
| MGH Pt35 | Alive without | 3 | — | 3 | — |
| | disease | | | | |
| | progression | | | | |

Melanoma cell lines were characterized with whole exome sequencing and RNA sequencing as previously described. See, Ott et al., Nature 547:217-21 (2017); Sarkizova et al., Nat. Biotechnol. 38:199-209 (2020). HLA class I expression and the HLA class I binding immunopeptidome of melanoma cell lines were detected using mass spectrometry-based proteomics. A detailed description is reported herein.

Melanoma cell line generation and characterization. Thawed cryopreserved tumor cells were washed and cultured in tissue culture plates containing OptiMEM GlutaMax media (Gibco, Thermofisher, Waltham, MA, www.thermofisher.com) supplemented with FBS (5%), sodium pyruvate (1 mM), penicillin and streptomycin (100 U/mL), gentamycin (50 μg/mL), insulin (5 μg/mL), and epidermal growth factor (5 ng/mL; Sigma-Aldrich). After one day, non-adherent cells, including immune cells, were removed by replacing the culture with fresh medium. Cell cultures were dissociated and passaged using versene (Gibco, Thermofisher). The expanding melanoma cell lines tested mycoplasma-free and were verified as melanoma cells by flow-cytometry using antibodies against human MCSP melanoma marker (PE, clone LHM-2, R&D Systems), human CD45 immune marker (PE-Cy7, clone 2D1, Biolegend, San Diego, CA www.biolegend.com) and human Fibroblast Antigen (FITC, clone REA165, Miltenyi Biotec, Bergisch Gladbach, Germany, www.miltenyibiotec.com) in the presence of Zombie Aqua viability die (Biolegend). For Pt-A, a pure melanoma cell line was obtained after 2 serial rounds of depletion of contaminant fibroblasts using Anti-Fibroblast Microbeads (Miltenyi Biotec). Control fibroblast cell lines were generated from 3 distinct patient biopsies harvested in the same study, whose cultures tested positive for the expression of the Fibroblast Antigen.

HLA class I expression and HLA class I binding immunopeptidome of melanoma cell lines. Upon expansion, patient-derived cell lines were cultured for 3 days with or without IFNγ (2000 U/mL, Peprotech) and harvested. Surface HLA class I expression was characterized through flow-cytometry using antibodies specific for pan-human HLA-A,B,C (PE conjugated, clone DX17, BD Biosciences, Franklin Lakes, NJ, www.bdbiosciences.com) and human HLA-A2 (FITC conjugated, clone BB7.2, Biolegend), coupled with staining using a viability dye (Zombie Aqua, Biolegend). Corresponding isotype antibodies were used as negative controls.

HLA—peptide complexes were immunoprecipitated from 0.1-0.2 gram (g) tissue or up to 50 million cells. Solid tumor samples were dissociated using a tissue homogenizer (Fisher Scientific 150) and HLA complexes were enriched as previously described (Abelin et al., Immunity 46:315-26 (2017)). Briefly, soluble lysates were immunoprecipitated with a pan-HLA class I antibody (clone W6/32, Santa Cruz). Two immunoprecipitates were combined, acid-eluted either on SepPak cartridges (Bassani-Sternberg et al., Nat. Commun. 7:1-16 (2016)), fractionated using high pH reverse phase fractionation and analyzed using high-resolution LC-MS/MS on a Fusion Lumos (Thermo Scientific) equipped with a FAIMS pro interface. Mass spectra were interpreted using the Spectrum Mill software package v7.1 pre-release (Agilent Technologies, Santa Clara, CA www.agilent.com). Tandem MS (MS/MS) spectra were excluded from searching if they did not have a precursor sequence MH+ in the range 600-4,000, had a precursor charge >5 or had a minimum of <5 detected peaks. The merging of similar spectra with the same precursor m/z acquired in the same chromatographic peak was disabled. Before searches, all MS/MS spectra were required to pass the spectral quality filter with a sequence tag length >0. MS/MS spectra were searched against a protein sequence database containing 98,298 entries, including all UCSC Genome Browser genes with hg19 annotation of the genome and its protein-coding transcripts (63,691 entries), common human virus sequences (30,181 entries) and recurrently mutated proteins observed in tumors from 26 tissues (4,167 entries), 259 common laboratory contaminants including proteins present in cell culture media and immunoprecipitation reagents as well as patient-specific neoantigen sequences (Sarkizova et al., Nat. Biotechnol. 38:199-209 (2020)). MS/MS search parameters included: no-enzyme specificity; fixed modification: cysteinylation of cysteine; variable modifications: carbamidomethylation of cysteine, oxidation of methionine and pyroglutamic acid at peptide N-terminal glutamine; precursor mass tolerance of ±10 ppm; product mass tolerance of ±10 ppm; and a minimum matched peak intensity of 30%. Peptide spectrum matches (PSMs) for individual spectra were automatically designated as confidently assigned using the Spectrum Mill autovalidation module to apply target-decoy-based FDR estimation at the PSM level of <1% FDR. Score threshold determination required that peptides had a minimum sequence length of 7, and PSMs had a minimum backbone cleavage score (BCS) of 5 (Sarkizova et al., Nat. Biotechnol. 38:199-209 (2020)). The BCS metric serves to decrease false positives associated with spectra having fragmentation in a limited portion of the peptide that yields multiple ion types. PSMs were consolidated to the peptide level to generate lists of confidently observed peptides for each allele using the Spectrum Mill Protein/Peptide summary module's Peptide-Distinct mode with filtering distinct peptides set to case sensitive.

The list of LC-MS/MS-identified peptides was filtered to remove potential contaminating peptides as follows, namely those: (1) observed in negative controls runs (blank beads and blank immunoprecipitates); (2) originating from species reported as common laboratory contaminants; (3) for which both the preceding and C-terminal amino acids were tryptic residues (R or K).

Sequencing of melanoma cell lines and parental tumors. Whole-Exome Sequencing (WES): Details of tumor WES have been previously reported (Ott et al, Nature 547:217-221, 2017). Library construction from surgical melanoma specimens, from matched germline and cell-line DNA or from unrelated fibroblasts was performed as previously described. See, Ott et al., Nature 547:217-21 (2017); Fisher et al., Genome Biol. 12:1-15 (2011). Briefly, cell suspensions were used for WES, and whole-exome capture was performed using the Illumina Nextera Rapid Capture Exome v1.2 bait set. Resulting libraries were then qPCR quantified, pooled, and sequenced with 76 base paired-end reads using HiSeq 2500 sequencers (Illumina, San Diego, CA, www.illumina.com). Data were analyzed using the Broad Picard Pipeline which includes de-multiplexing, duplicate marking, and data aggregation.

RNA sequencing (RNA-seq). RNA sequencing was performed as previously described See, Ott et al., Nature 547:217-21 (2017). Briefly, for sequencing library construction, RNA was extracted from frozen cell suspensions using a Qiagen RNeasy RNA extraction kit. RNA-seq libraries were prepared using Illumina TruSeq Stranded mRNA Library Prep Kit. Flowcell cluster amplification and sequencing were performed according to the manufacturer's protocols using the HiSeq 2500. Each run was a 101 bp paired-end with an eight-base index barcode read. Data were analyzed using the Broad Picard Pipeline which includes de-multiplexing and data aggregation.

DNA quality control. Standard Broad Institute (BI) protocols as previously described (Chapman et al., Nature 471:467-72 (2011); Berger et al., Nature 470:214-20 (2011)) were used for DNA quality control. The identities of all tumor and normal DNA samples were confirmed by mass spectrometric fingerprint genotyping of 95 common SNPs by Fluidigm Genotyping (Fluidigm, South San Francisco, CA, www.standardbio.com). Sample contamination from foreign DNA was assessed using ContEst (Cibulskis et al., Bioinformatics 27:2601-2 (2011)).

RNA quality control. All RNA was quantified using the Quant-It RiboGreen RNA reagent, an ultrasensitive fluorescent nucleic acid stain used for quantitating RNA in solution, and a dual standard curve. The experimental details are described in Hu et al., Nat. Med. 27:515-25 (2021).

Somatic mutation calling. Analyses of whole-exome sequencing data of parental tumors, patient-derived melanoma cell lines and matched PBMCs (as source of normal germline DNA) were used to identify somatic alterations in the tumor and cell line samples using the hg19 human genome reference. Aligned BAM files were first generated using the bwa aligner (version 0.5.9). GATK Calculate Contamination was used to assess potential contamination from foreign individuals in each sample (5% threshold). Mutations and small insertions/deletions in the exome were identified using the Mutect2 tool (v2.7.0). Filters specifically designed to identify and remove orientation bias and alignment error related artifacts were also implemented (github.com/gatk-workflows/gatk4-somatic-snvs-indels/Mutect2). Finally, manual review of a subset of alterations was performed using the integrated genome viewer. The final list of somatic events was annotated using Funcotator.

Transcriptomic analysis. RNA-seq data were aligned using the STAR alignment tool (Dobin et al., Bioinformatics 29:15-21 (2013)). The aligned reads were further quantified at the gene and transcript levels using RSEM (Li & Dewey, BMC Bioinformatics 12:323 (2011)). RNA-seqQC2 was used to evaluate quality metrics of the transcriptomic data (DeLuca et al., Bioinformatics 28:1530-2 (2012)).

HLA typing. HLA class I and class II molecular typing for melanoma patients were determined by PCR-rSSO (reverse sequence specific oligonucleotide probe), with ambiguities resolved by PCR-SSP (sequence specific primer) techniques (One Lambda Inc., BWH Tissue Typing Laboratory).

In vitro enrichment of antitumor T cells from peripheral blood (data not shown). Frozen PBMCs were thawed and then rested overnight in RPMI medium supplemented with L-glutamine, nonessential amino acids, HEPES, β-mercaptoethanol, sodium pyruvate, penicillin/streptomycin (Gibco, Thermofisher), and 10% AB-positive heat-inactivated human serum (Gemini Bioproduct, West Sacramento, CA, www.geminibio.com). Autologous melanoma cells were harvested from adherent cultures, irradiated (10.000 rad) and plated at least one day before the start of co-culture experiments, in 24-well cell culture plates at the density of 0.1-0.2×10⁶ cells/well. For in vitro expansion of tumor-specific T cells, 5×10⁶ PBMCs per well were added to the plates in the presence of IL-7 (5 ng/mL; Peprotech, Cranbury, NJ, www.peprotech.com). A minimum of 20×10⁶ PBMCs was needed to start the culture, and therefore only samples with adequate availability of viable cells were used for in vitro enrichment of antitumor T cells. On day 3, low-dose IL-2 (20 U/mL, Amgen, Thousand Oaks, CA, www.amgen.com) was added. Half-medium change and supplementation of cytokines were performed every 3 days, as described previously (Ott et al., Nature 547:217-21 (2017)). After 10 days, T cells were harvested, washed, and re-stimulated with irradiated autologous melanoma cells as previously described (Ott et al., Nature 547:217-21 (2017)). On day 20, T cell specificity was tested against non-irradiated autologous or third-party melanoma cells.

Single cell sorting of melanoma-reactive T cells (data not shown). After in vitro enrichment, 10×10⁶ cells were re-challenged with non-irradiated autologous melanoma cells (10:1 effector-target ratio) for 6 hours, in the presence of anti-human CD107a and CD107b antibodies (BV786, clones H4A3 and H4B4, BD Biosciences). Control effector cells were cultured in the absence of melanoma cells. Response to stimulation was evaluated by a cytokine secretion assay. Briefly, stimulated and control cells were first labeled with IL-2, TNFα, and IFNγ catch antibody (Miltenyi Biotec) for 5 minutes and then diluted in warm medium as per the manufacturer's protocol. After 45 minutes of incubation, cells were washed and labeled with FITC anti-human IFNγ, PE anti-human TNFα, APC anti-human IL-2 antibodies (Miltenyi Biotec), as well as with APC-Cy7 anti-human CD3 (clone UCHT1), PE-Cy7 anti-human CD8a (clone HIT8a), Pacific blue anti-human CD4 (clone OKT4) antibodies and Zombie Aqua die (all from Biolegend). After 30 minutes of incubation at 4° C., cells were washed, resuspended in medium, and sorted using a BD Aria cell sorter (BD Bioscience). The sorting gating strategy comprised the following sequential steps: i) exclusion of doublets through lymphocyte physical parameters, ii) gating on viable (Zombie-) CD3+CD8+CD107a/b+ events, and iii) gating on IL-2, TNFα, and IFNγ using the unstimulated control sample to define background signal. The sorting of melanoma reactive CD8+ T cells from PBMCs was carried out. After stimulation with autologous melanoma, degranulating CD107a/b+CD8+ T cells displaying secretion of at least one cytokine were single-cell sorted into 384-well plates. Positive thresholds were established using unstimulated controls. Sorting strategy for isolation of tumor cell populations for single-cell sequencing involved sorting of viable (Zombie-) CD45+CD3+ for Pt-A, Pt-C, and Pt-D tumor specimens, while viable (Zombie-) cells were sorted for Pt-C Rel specimen.

Viable CD3+CD8+ cells positive for >1 cytokine were single-cell sorted in 384 well plates (Eppendorf). Immediately after sorting, plates were centrifuged, frozen in dry ice and placed in −80° C. for storage until the time of analysis. For each sorted cell, all parameters were indexed, thus allowing post-sorting analysis of fluorescence intensities.

Intracellular staining and CD107a/b degranulation assay. For degranulation and intracellular cytokine detection, 0.25×10⁶ effector T cells (either from in vitro enriched antitumor T cells or from TCR-transduced T cell lines) were stimulated with 0.25×10⁵ adherent melanoma cells (effector:target ratio 10:1). For TCR-transduced cells, up to 4 TCR-transduced lines were labeled with 4 different dilutions of Cell Trace Violet dye (Life Technologies, Theimofisher) and pooled together before stimulation. Controls included mock stimulation in the absence of target cells (negative control) or in the presence of PMA (50 nanograms per milliliter (ng/mL), Sigma-Aldrich) and ionomycin (10 micrograms per milliliters (μg/mL), Sigma-Aldrich) (positive control). Effector and target cells were incubated at 37° C. in complete RPMI, in the presence of anti-human CD107a and CD107b antibodies (FITC, clones H4A3 and H4B4, Biolegend) and brefeldin A (10 μg/mL, Sigma-Aldrich) was added after 3 hours. After a total incubation time of 6 hours, cells were washed and stained at room temperature for 10 minutes with Zombie Aqua die (Biolegend). Antibodies specific for the following surface markers were then added: human CD3 (BV650, clone OKT3, Biolegend), human CD8 (BV785, clone RPA-T8, Biolegend) and murine TRBC (PE, clone H57-597, eBioscience, Thermofisher). After 20 minutes of incubation, cells were washed, fixed and permeabilized using fixation and permeabilization buffers (Biolegend), following the manufacturer's instructions. Cytokine intracellular staining was performed by incubating the cells for 30 minutes with the following antibodies: anti-human IFNγ (APC-Cy7, clone B27, Biolegend), TNFα. (PE-Cy7, clone Mab11, Biolegend) and IL-2 (APC, clone MQ1-17H12, eBioscience). Flow cytometric analysis was performed on an HTS-equipped BD Fortessa cytometer (BD Biosciences) and data were analyzed using Flowjo v10.3 software (BD Biosciences). Intracellular production of the 3 cytokines was measured on viable (Zombie-) CD3+CD8+CD107a/b+ degranulating T cells and expressed as percentage of total CD8+ T cells. Cytotoxicity of reconstructed TCRs was measured on pools of 4 TCR-transduced (mTRBC+) effector T cell lines that were labeled with 4 different dilutions of cell Trace Violet dye (Life Technologies).

Analysis of CD107a/b degranulation and concomitant cytokine production was carried out). The gating strategy consisted in identification of degranulating and cytokine producing cells (at least 1 cytokine) among CD8+ TCR-transduced (mTRBC+) lymphocytes. TCR-transduced effectors were labeled with different dilutions of Cell Trace (CT) Violet dye, allowing combination of up to 4 single effectors per pool. The analysis was then repeated for each effector population. The results (not shown) indicated the presence of cells that were positive for degranulation (CD107a/b+) and at least for one of the tested cytokines (IFNγ, TNFα, or IL-2).

RNA extraction for bulk TCR sequencing. Cryopreserved PBMCs were thawed and resuspended in RPMI medium (Gibco, ThermoFisher), supplemented with 10% fetal bovine serum and 1% penicillin-streptomycin (Gibco, ThermoFisher). CD3+ positive selection was performed using a Miltenyi CD3 beads, and total RNA was extracted using a QIAGEN RNeasy Mini kit.

Plate-based single cell TCR sequencing and bulk TCR sequencing analysis. Single-cell TCR sequencing of tumor-reactive T cells sorted in 384 well plates was performed by RNAse H-dependent targeted TCR amplification (rhTCRseq) of TCR transcripts using single-cell-amplified cDNA libraries as published previously (Li, S. et al., Nat Protoc 14, 2571-2594 (2019)). Beta TCR repertoire analysis in bulk RNA samples was performed using an adapted rhTCRseq protocol published previously (Li et al., Nat. Protoc. 14:2571-94 (2019)). Specifically, 10 ng bulk RNA was used in each RT reaction, and 6 to 8 replicates were done for each sample and excess RT primers were eliminated by exonuclease digestion, and then rhPCR was performed. After the sequencing library was made, it was sequenced using MiSeq 300 cycle Reagent Kit v2 on the Illumina sequencing system according to the manufacturer's protocol with 248 bp read 1, 48 bp read 2, 8 bp index 1, and 8 bp index 2. The sequencing data analysis was performed based on methods published previously (Li et al., Nat. Protoc. 14:2571-94 (2019)).

Peptide-HLA affinity and stability measurement (data not shown). Affinity and stability of HLA-peptide interactions were evaluated for those antigens that were able to trigger the activation of antitumor TCRs. For each discovered antigen specificity, HLA restriction for identified by measured TCR reactivity upon culture of monoallelic HLA cell lines (Abelin et al., Immunity 46:315-26 (2017); Sarkizova et al., Nat. Biotechnol. 38:199-209 (2020)) pulsed with the peptide of interest. When multiple HLA restrictions showed the ability to trigger TCR reactivity upon peptide binding, the HLA restriction capable of inducing maximal upregulation of CD137 expression on TCR-transduced T cells was selected. The analysis of HLA restriction was carried out by testing recognition against mono-allelic ILA lines (Abelin, Immunity. 2017 Feb. 21; 46(2):315-326) (data not shown), which indicated the specificity and HLA restriction of polyreactive tumor specific TCRs. Flow cytometry histograms depicting CD137 upregulation (x axis) measured on CD8+ T cells transduced with 2 polyreactive tumor-specific TCRs isolated from Pt-D TILs. Reactivity was measured following overnight co-culture of effector T cells with mono-allelic APC lines expressing single HLAs of Pt-D, pulsed with different peptides, including Ova peptide (negative control) or identified cognate antigens.

Analysis of the deconvolution of HLA restriction was carried out (data not shown), to determine the HLA restriction of tumor specific TCRs with identified cognate antigens. CD137 upregulation was measured on CD8+ T cells transduced with representative TCRs with identified antigen specificity. For each patient, a representative TCR specific for a different antigen specificity was tested. Reactivity of each TCR is tested against the corresponding cognate antigen, presented by APC cells stably transformed with single patient's HLAs, as available from previous studies (Sarkizova et al., Nat. Biotechnol. 38:199-209 (2020); Abelin et al., Immunity 46:315-326 (2017)). HLA restrictions that were able of triggering the highest TCR reactivity upon binding of cognate antigens were identified as cognate restrictions.

Peptide affinity and stability measurements (data not shown) were performed at Immunitrack (Copenhagen, Denmark) for peptide-HLA couples with available HLA alleles (7 of 9 MAA-HLA complexes and 11 of 14 NeoAg-HLA complexes). Affinity and stability assays were measured as previously described (Harndahl et al., J. Biomol. Screen. 14:173-180 (2009)). Acquired data allowed to calculate K_{d} of peptide-HLA interactions using GraphPad Prism 8 software.

Processing of tumor and blood specimens. After surgery, tumor tissue was carefully minced manually, suspended in a solution of collagenase D (200 U/mL) and DNAse I (20 U/mL) (Roche Life Sciences), transferred to a sealable plastic bag and incubated with regular agitation in a Seward Stomacher Lab Blender for 30-60 min. After digestion, any remaining clumps were removed and the single cell suspension was recovered, washed, and immediately frozen in aliquots and stored in vapor-phase liquid nitrogen until time of analysis. Patient peripheral blood mononuclear cells (PBMCs) were isolated by Ficoll/Hypaque (GE healthcare) density-gradient centrifugation and cryopreserved with 10% dimethylsulfoxide (Sigma-Aldrich) in fetal bovine serum (FBS, Gibco, Thermofisher). Cells from patients were stored in vapor-phase liquid nitrogen until time of analysis.

Cell sorting and CITEseq antibody labeling for single-cell sequencing. Tumor samples were thawed and then rested in RPMI containing 10% FBS and 1% penicillin/streptomycin for 4-6 hours. Subsequently, cells were filtered with a 100 μm cell-strain to remove debris, resuspended in fresh media at 10-20×10⁶ cells/mL, and labeled with Live/Dead Zombie Aqua (BioLegend) for 10 min at 4° C., following by staining with anti-human CD45 (PE-Cy7, 2D1, Biolegend) and anti-CD3 (APC-Cy7, UCHT1, Biolegend) for 20-30 min at 4° C. Cells were washed once with media and analyzed on a BD Aria cell sorter (BD Biosciences). For Pt-A, Pt-C and Pt-D, the following viable (Zombie Aqua −) populations were sorted: T cells (CD45+, CD3+), non-T immune cells (CD45+, CD3−) and non-immune cells enriched in tumor cells (CD45−) (see Sorting Strategies). For biopsies with low cell recovery, total viable cells were isolated using either flow-sorting (Pt-C Relapse) or a dead-cell removal kit (Miltenyi Biotec) (Pt-B; Table 5). After separation, cells were counted and resuspended 10×10⁶ cells/mL in PBS supplemented with 0.4% of ultrapure Bovine Serum Albumine (BSA, Invitrogen). Fc blocking was performed through incubation for 10 minutes at 4° C. with Human TruStain FcX™ (Biolegend). A mix of 69 TotalSeq™-C antibodies (Biolegend, Table 2-Table 4) was added; after 30-minutes of incubation at 4° C., cells were washed twice in PBS with BSA and submitted to single-cell sequencing.

TABLE 5
Metrics of single cell RNAseq, TCRseg, and TCR clonotype information

| | | Sample | | | | | |
|---|---|---|---|---|---|---|---|
| | | Pt-A | Pt-B | Pt-C | Pt-C rel | Pt-D | |
| | | Origin | | | | | |
| | | Axillary | | | | Axillary | |
| | | Lymph | | | | Lymph | |
| | | node | Skin | Lung | Skin | node | |
| | | Processing* | | | | | |
| | | FACS | Magnetic | FACS | | FACS | |
| | | Sorting on | selection | Sorting on | | Sorting on | |
| | | viable | with dead | viable | FACS | viable | |
| | | CD45+ | removal | CD45+ | Sorting: | CD45+ | |
| | | CD3+ | kit: viable | CD3+ | viable | CD3+ | |
| | | cells | cells | cells | cells | cells | Total |
| Single cell | # of replicates | 3 | 4 | 3 | 2 | 4 | 3* |
| RNA seq | # of CD3+ T cells | 19755 | 122 | 14330 | 192 | 30392 | 64791 |
| and | # of CITEseq | 10844 | 68 | 8818 | 14 | 10575 | 30319 |
| CITEseq | selected CD8+ | | | | | | |
| | TILs | | | | | | |
| | # of genes/CD8+ | 1589 | 461 | 1015 | 684 | 1024 | 1015* |
| | cell (median) | | | | | | |
| Single cell | cell # with | 8750 | 50 | 7353 | 12 | 8312 | 24477 |
| TCR seq | TCRαβ | | | | | | |
| | # of TCR | 2404 | 26 | 718 | 12** | 2280 | 5435** |
| | singletons | | | | | | |
| | # of TCR families † | 1030 | 7 | 247** | 0 | 520 | 1804 |
| | Total # of TCRαβ | 3434 | 33 | 965 | 12 | 2800 | 7239** |
| | clonotypes | | | | | | |
| # of | T_{Ex} primary | 289 | 3 | 88 | — | 117 | 497 |
| intratumoral | clusters | | | | | | |
| CD8+ TCR | | | | | | | |
| clonotype | T_{NExM} primary | 425 | 1 | 50 | — | 241 | 717 |
| families | clusters | | | | | | |
| with | | | | | | | |

*Processing sequenced population
**5 singletons TCRs from Pt-C-rel matched with 5 expanded TCRs from Pt-C biopsy
† greater than one cell per family

Specimens isolated from individual patients were sorted and processed as independent experiments, with experimental batches hence corresponding to the 4 analyzed patients. For each patient, at least one blood sample was processed, enriched for T cells and analyzed in parallel with the same isolation strategy, therefore serving as an internal quality control for all downstream analyses.

Single-cell transcriptome, TCR and surface epitope sequencing. Sample cell count and viability were assessed by trypan-blue dye exclusion (Sigma Aldrich), and cell density was adjusted to analyze −40,000 cells per sample. Up to 4 replicates were performed for CD45+CD3+ intratumoral populations (Table 5). Sample processing for single-cell gene expression (scRNA-seq) and TCR V(D)J clonotypes (scTCR-seq) was performed (Chromium Single Cell 5′ Library and Gel Bead Kit, 10× Genomics), following the manufacturer's recommendations. After Gel Bead-in-Emulsion reverse transcription (GEM-RT) reaction and clean-up, PCR amplification was performed to obtain cDNAs used for RNA-seq library generation. Subsequently, 5′ gene expression library construction, TCR V(D)J targeted enrichment library preparation (Chromium Single Cell V(D)J Enrichment Kit, Human T Cell), and cell surface protein library construction (Chromium Single Cell 5′ Feature Barcode Library Kit) were carried out according to the manufacturer's instructions. Quality controls for cDNA and sequencing libraries were performed using Bioanalyzer High Sensitivity DNA Kit (Agilent). All libraries were tagged with a sample barcode for multiplexed pooling with other libraries and sequenced on Illumina NovaSeq S4 platform. The sequencing parameters were: Read 1 of 150 bp, Read 2 of 150 bp, and Index 1 of 8 bp.

Processing of 10× single-cell data. Processing of scTCR data. TCR-seq data for each sample were processed using Cell Ranger software (version 3.1.0). TCRs were grouped in patient-specific TCR clonotype families based on TCRa-TCRI3 chain identity, allowing for a single amino acid substitution within the TCRa-TCRI3 CDR3. Cells with a single TCR chain were included and grouped with the matched clonotypes families. The resulting TCR clonotype families were ranked according to sample-specific size and incorporated into downstream analysis. This procedure was reiterated on all samples sequenced from the same patient and results were manually reviewed. The same strategy was also utilized to match TCR clonotypes from TILs with those isolated and sequenced from PBMCs upon in vitro co-culture with melanoma cells. Due to the low number of TCR clonotypes specific for Pt-C-rel specimen (n=7), Pt-C and Pt-C-rel TILs were analyzed together (referred as Pt-C within the text).

Processing and analysis of scRNAseq and CITEseq data. scRNA-seq data were processed with Cell Ranger software (version 3.1.0). scRNAseq count matrices and CITEseq antibody expression matrices were read into Seurat, version 3.2.0 (Stuart et al., Cell 177:1888-1902.e21 (2019)). For each batch of samples comprising all tumor or PBMCs single-cell data acquired for a single patient, a Seurat object was generated. Cells were filtered to retain those with ≤20% mitochondrial RNA content and with a number of unique molecular identifiers (UMIs) comprised between 250 and 10,000. Overall, scRNA-seq data comprised 1,006,058,131 transcripts in 288,238 cells that passed quality filters. scTCR data were integrated and cells with ≥3 TCRα chains, ≥3 TCRβ chains or 2 TCRα and 2 TCRβ chains were removed. scRNAseq data was normalized using Seurat NormalizeData function and CITEseq data using the center log-ratio (CLR) function. CITEseq signals were then expressed as relative to isotype controls signals of each single cell, by dividing each antibody signal by the average signal from 3 CITEseq isotype control antibodies used. For cells with an average isotype signal less than 1, all the CITEseq signals were increased of “1-mean isotype signal” value.

Each patient dataset was scaled and processed under principal components analysis using the ScaleData, FindVariableFeatures and RunPCA functions in Seurat. Serial custom filters were used to identify CD8+ T lymphocytes: first, UMAP areas with predominance of cells belonging to FACS sorted CD45+CD3+ populations (either processed from blood or tumor) and with high expression of the CD3E transcripts were selected. Second, possible contaminants belonging to B and myeloid lineages were removed by excluding cells characterized by either high expression of CD19 and ITGAM transcripts or positivity for CD19 or CD11b CITEseq antibodies. Finally, remaining events were grouped in CD8+ or CD4+ cells using the corresponding CITEseq antibodies, and CD8+CD4− lymphocytes were selected. Importantly, these steps were designed to maximize the ability to correctly detect CD8+ T cells by relying on the actual surface expression of the CD8a protein thus avoiding cell loss due to possible false-negatives at the RNA level. Cells classified as CD8+CD4− from tumor specimens of the 4 patients were combined using the RunHarmony function in Seurat with default parameters (Korsunsky et al., Nat. Methods 16:1289-1296 (2019)). Data were normalized, scaled, and PCAs computed as previously described (Korsunsky et al., Nat. Methods 16:1289-1296 (2019)). UMAP coordinates, neighbors, and clusters were calculated with the reduction parameter set to ‘harmony’. Cluster stability over objects with different resolutions was evaluated to select the appropriate level of resolution (0.6). Clusters composed of less than 200 cells were not characterized. Markers specific for each cluster were found using Seurat's FindAllMarkers function with min.pct set to 0.25 and logfc.threshold set to log(2) (Table 6). Comparison of TEs clusters (0,4,5,8,11) to the remaining single cells allowed the identification of a subset of genes upregulated or downregulated in exhausted cells enriched in antitumor specificities (see Table 5). Upregulated genes (adj p value<0.0001, log₂FC>1) constituted the core signature of tumor-specific cells.

TABLE 6
List of CITEseq Ab used of single cell sequencing

| Marker | Clone | Isotype | Barcode |
|---|---|---|---|
| B2m (*) | 2M2 | Mouse IgG1 | CAGCCCGATTAAGGT |
| | | | (SEQ ID NO: 1) |
| B7H4 | MIH43 | Mouse IgG1 | TGTATGTCTGCCTTG (SEQ |
| | | | ID NO: 2) |
| CD10 | HI10a | Mouse IgG1 | CAGCCATTCATTAGG (SEQ |
| | | | ID NO: 3) |
| CD117 (*) | 104D2 | Mouse IgG1 | AGACTAATAGCTGAC |
| | | | (SEQ ID NO: 4) |
| CD11a | TS2/4 | Mouse IgG1 | TATATCCTTGTGAGC (SEQ |
| | | | ID NO: 5) |
| CD11b | ICRF44 | Mouse IgG1 | GACAAGTGATCTGCA |
| | | | (SEQ ID NO: 6) |
| CD11c (*) | S-HCL-3 | Mouse IgG2b | TACGCCTATAACTTG (SEQ |
| | | | ID NO: 7) |
| IL7RA | A019D5 | Mouse IgG1 | GTGTGTTGTCCTATG (SEQ |
| | | | ID NO: 8) |
| CD134 (OX40) | Ber-ACT35 | Mouse IgG1 | AACCCACCGTTGTTA (SEQ |
| | (ACT35) | | ID NO: 9) |
| CD137 (41BB) | 4B4-1 | Mouse IgG1 | CAGTAAGTTCGGGAC |
| | | | (SEQ ID NO: 10) |
| CD138 (*) | DL-101 | Mouse IgG1 | GTATAGACCAAAGCC |
| | | | (SEQ ID NO: 11) |
| CD14 | M5E2 | Mouse IgG2a | TCTCAGACCTCCGTA (SEQ |
| | | | ID NO: 12) |
| CD15 | W6D3 | Mouse IgG1 | TCACCAGTACCTAGT (SEQ |
| | | | ID NO: 13) |
| CD152 (CTLA4) | BNI3 | Mouse IgG2a | ATGGTTCACGTAATC (SEQ |
| | | | ID NO: 14) |
| CD16 | 3G8 | Mouse IgG1 | AAGTTCACTCTTTGC (SEQ |
| | | | ID NO: 15) |
| CD163 (*) | GHI/61 | Mouse IgG1 | GCTTCTCCTTCCTTA (SEQ |
| | | | ID NO: 16) |
| CD18 | TS1/18 | Mouse IgG1 | TATTGGGACACTTCT (SEQ |
| | | | ID NO: 17) |
| CD183 (CXCR3) | G025H7 | Mouse IgG1 | GCGATGGTAGATTAT |
| | | | (SEQ ID NO: 18) |
| CD184 (CXCR4) | 12G5 | Mouse IgG2a | TCAGGTCCTTTCAAC (SEQ |
| | | | ID NO: 19) |
| CD19 | HIB19 | Mouse IgG1 | CTGGGCAATTACTCG (SEQ |
| | | | ID NO: 20) |
| CD194 (CCR4) | L291H4 | Mouse IgG1 | AGCTTACCTGCACGA |
| | | | (SEQ ID NO: 21) |
| CD197 (CCR7) | G043H7 | Mouse IgG2a | AGTTCAGTCAACCGA |
| | | | (SEQ ID NO: 22) |
| CD1c (*) | L161 | Mouse IgG1 | GAGCTACTTCACTCG (SEQ |
| | | | ID NO: 23) |
| CD1d (*) | 51.1 | Mouse IgG2b | TCGAGTCGCTTATCA (SEQ |
| | | | ID NO: 24) |
| CD20 | 2H7 | Mouse IgG2b | TTCTGGGTCCCTAGA (SEQ |
| | | | ID NO: 25) |
| CD223 | 11C3C65 | Mouse IgG1 | CATTTGTCTGCCGGT (SEQ |
| (LAG3) (*) | | | ID NO: 26) |
| CD226 (DNAM-1) | 11A8 | Mouse IgG1 | TCTCAGTGTTTGTGG (SEQ |
| | | | ID NO: 27) |
| CD244 (2B4) | C1.7 | Mouse IgG1 | TCGCTTGGATGGTAG (SEQ |
| | | | ID NO: 28) |
| CD25 | BC96 | Mouse IgG1 | TTTGTCCTGTACGCC (SEQ |
| | | | ID NO: 29) |
| CD27 | O323 | Mouse IgG1 | GCACTCCTGCATGTA (SEQ |
| | | | ID NO: 30) |
| CD274 (PDL1) | 29E.2A3 | Mouse IgG2b | GTTGTCCGACAATAC (SEQ |
| | | | ID NO: 31) |
| CD278 (ICOS) | C398.4A | Armenian Hamster | CGCGCACCCATTAAA |
| | | IgG | (SEQ ID NO: 32) |
| CD279 (PD1) | EH12.2H7 | Mouse IgG1 | ACAGCGCCGTATTTA (SEQ |
| | | | ID NO: 33) |
| CD28 | CD28.2 | Mouse IgG1 | TGAGAACGACCCTAA |
| | | | (SEQ ID NO: 34) |
| CD3 | UCHT1 | Mouse IgG1 | CTCATTGTAACTCCT (SEQ |
| | | | ID NO: 35) |
| CD31 (*) | WM59 | Mouse IgG1 | ACCTTTATGCCACGG (SEQ |
| | | | ID NO: 36) |
| CD314 (NKG2D) | 1D11 | Mouse IgG1 | CGTGTTTGTTCCTCA (SEQ |
| | | | ID NO: 37) |
| CD33 (*) | P67.6 | Mouse IgG1 | TAACTCAGGGCCTAT (SEQ |
| | | | ID NO: 38) |
| CD335 (NKp46) | 9E2 | Mouse IgG1 | ACAATTTGAACAGCG |
| | | | (SEQ ID NO: 39) |
| CD34 (*) | 581 | Mouse IgG1 | GCAGAAATCTCCCTT (SEQ |
| | | | ID NO: 40) |
| CD38 | HIT2 | Mouse IgG1 | TGTACCCGCTTGTGA (SEQ |
| | | | ID NO: 41) |
| CD39 | A1 | Mouse IgG1 | TTACCTGGTATCCGT (SEQ |
| | | | ID NO: 42) |
| CD4 | RPA-T4 | Mouse IgG1 | TGTTCCCGCTCAACT (SEQ |
| | | | ID NO: 43) |
| CD40 | 5C3 | Mouse IgG1 | CTCAGATGGAGTATG |
| | | | (SEQ ID NO: 44) |
| CD44 | BJ18 | Mouse IgG1 | AATCCTTCCGAATGT (SEQ |
| | | | ID NO: 45) |
| CD45 | HI30 | Mouse IgG1 | TGCAATTACCCGGAT (SEQ |
| | | | ID NO: 46) |
| CD45RA | HI100 | Mouse IgG2b | TCAATCCTTCCGCTT (SEQ |
| | | | ID NO: 47) |
| CD45RO | UCHL1 | Mouse IgG2a | CTCCGAATCATGTTG (SEQ |
| | | | ID NO: 48) |
| CD49f | GoH3 | Rat IgG2a | TTCCGAGGATGATCT (SEQ |
| | | | ID NO: 49) |
| CD5 | UCHT2 | Mouse IgG1 | CATTAACGGGATGCC |
| | | | (SEQ ID NO: 50) |
| CD56 (NCAM) | QA17A16 | Mouse IgG1 | TTCGCCGCATTGAGT (SEQ |
| | | | ID NO: 51) |
| CD57 (*) | QA17A04 | Mouse IgG1 | AACTCCCTATGGAGG |
| | | | (SEQ ID NO: 52) |
| CD62L | DREG-56 | Mouse IgG1 | GTCCCTGCAACTTGA (SEQ |
| | | | ID NO: 53) |
| CD69 | FN50 | Mouse IgG1 | GTCTCTTGGCTTAAA (SEQ |
| | | | ID NO: 54) |
| CD70 | 113-16 | Mouse IgG1 | CGCGAACATAAGAAG |
| | | | (SEQ ID NO: 55) |
| CD73 | AD2 | Mouse IgG1 | CAGTTCCTCAGTTCG (SEQ |
| | | | ID NO: 56) |
| CD80 | 2D10 | Mouse IgG1 | ACGAATCAATCTGTG |
| | | | (SEQ ID NO: 57) |
| CD86 | IT2.2 | Mouse IgG2b | GTCTTTGTCAGTGCA (SEQ |
| | | | ID NO: 58) |
| CD8a | RPA-T8 | Mouse IgG1 | GCTGCGCTTTCCATT (SEQ |
| | | | ID NO: 59) |
| CD95 | DX2 | Mouse IgG1 | CCAGCTCATTAGAGC |
| | | | (SEQ ID NO: 60) |
| HLADR | L243 | Mouse IgG2a | AATAGCGAGCAAGTA |
| | | | (SEQ ID NO: 61) |
| KLRG1 | 2F1/KLRG1 | Syrian hamster | GTAGTAGGCTAGACC |
| | | IgG | (SEQ ID NO: 62) |
| TCRab | IP26 | Mouse IgG1 | CGTAACGTAGAGCGA |
| | | | (SEQ ID NO: 63) |
| TCRgd* | B1 | Mouse IgG1 | CTTCCGATTCATTCA (SEQ |
| | | | ID NO: 64) |
| TIGIT | A15153G | Mouse IgG2a | TTGCTTACCGCCAGA (SEQ |
| | | | ID NO: 65) |
| Tim3* | F38-2E2 | Mouse IgG1 | TGTCCTACCCAACTT (SEQ |
| | | | ID NO: 66) |
| IgG1 isotype | MOPC-21 | Mouse IgG1 | CAGCCCGATTAAGGT |
| | | | (SEQ ID NO: 1) |
| IgG2a isotype | MOPC-173 | Mouse IgG2a | TGTATGTCTGCCTTG (SEQ |
| | | | ID NO: 2) |
| IgG2b isotype | MPC-11 | Mouse IgG2b | CAGCCATTCATTAGG (SEQ |
| | | | ID NO: 3) |

*Data not available for Pt-A samples

Phenotypic distribution of TCR clonotypes composed by >1 cell (defined as TCR clonotype families) was examined using the CD8+ clusters identified through Seurat clustering. To associate a cell state to each TCR clonotype family, a “primary cluster” was assigned by selecting the cluster with the largest representation of cells in the clone. In cases of a tie, in which the two largest representative clusters had equal counts, no primary cluster was assigned.

Cells expressing TCRs with in vitro identified antigenic specificities were compared to establish transcripts or surface proteins deregulated among T cells specific for different antigenic categories (viral epitopes, MAAs, NeoAgs). Comparisons were performed independently for each patient using the Seurat's FindAllMarkers function, and only significantly deregulated genes (adj p value<0.05, log₂FC>1 for scRNAseq data; log₂FC>0.4 for CITEseq data) in at least 2 out of 4 patients were selected. The same type of analysis was performed for each patient to compare T cells harboring TCRs with high (above the median) or low (below the median) avidity or normalized TCR-induced tumor-specific activation (as measured in vitro with CD137 assay, see below). No gene was found to be recurrently deregulated among TCR clonotype families with different avidity and antitumor activity.

To analyze the subpopulations of tumor-specific CD8+ cells, 7451 single cells expressing TCRs with in vitro confirmed tumor-specific TCRs (n=134) were subsetted, normalized and re-clustered with resolution 0.4 (which granted proper cluster stability). During this procedure, TCR related genes were removed to avoid clustering artifact produced by the dramatically reduced TCR diversity. Cluster specific genes were identified with Seurat's FindAllMarkers function and reported in Table 7-Table 11.

TABLE 7
Differentially expressed genes among the 12 clusters of CD8+
TILs identified by scRNA-seq (adjusted P value < 0.05)

| Cluster 0: T_{Ex} CD8 | | Cluster 2: T_{EM} 1 | | Cluster 2: T_{EM} 2 | |
|---|---|---|---|---|---|
| gene | avg_logFC | gene | avg_logFC | gene | avg_logFC |
| KRT86 | 2.4136621 | GYG1 | 0.8172213 | S1PR1 | 1.3065899 |
| ACP5 | 1.8418655 | GPR183 | 0.7001822 | GPR183 | 1.0640552 |
| CXCR6 | 1.6596442 | CXCL13 | −1.734487 | CCR7 | 0.9208029 |
| HMOX1 | 1.6491337 | HSPB1 | −1.209605 | ANXA1 | 0.8898539 |
| LAYN | 1.5559044 | GLUL | 0.8753817 | TCF7 | 0.8618741 |
| HAVCR2 | 1.4050654 | HMOX1 | −1.755758 | IL7R | 0.8234603 |
| PRF1 | 1.4028621 | HSP90AA1 | −0.73627 | MBP | 0.7900712 |
| SLC2A8 | 1.3736458 | PERP | 0.8096851 | VIM | 0.7780312 |
| CHST12 | 1.2472493 | GEM | −1.448285 | NKG7 | −0.846651 |
| GALNT2 | 1.2263637 | VCAM1 | −1.345849 | CTSW | −0.870258 |
| ENTPD1 | 1.0920758 | DNAJA1 | −0.802053 | DUSP4 | −0.998224 |
| LAG3 | 1.0601 | CD27 | −0.869287 | HSPH1 | −1.004014 |
| GZMB | 1.0555171 | HSPA1A | −1.19064 | HSPE1 | −1.01374 |
| PDCD1 | 1.0506139 | ID3 | −0.957854 | LSP1 | −1.018812 |
| CARD16 | 0.9697265 | NR4A2 | −1.023909 | HSPD1 | −1.048309 |
| CTLA4 | 0.9438091 | HSPA1B | −2.031975 | DNAJA1 | −1.075721 |
| SLA2 | 0.884999 | HSPD1 | −0.714002 | CD74 | −1.106657 |
| CD27 | 0.8013536 | JUNB | −0.854129 | HLA-DRB5 | −1.113838 |
| RALA | 0.7614218 | LAG3 | −0.745652 | HLA-DPB1 | −1.143947 |
| VCAM1 | 0.7595545 | HSPH1 | −0.721703 | HSP90AA1 | −1.169466 |
| SYNGR2 | 0.7555568 | RGS2 | −0.900195 | GZMB | −1.301819 |
| NKG7 | 0.7506139 | RGS1 | −0.848416 | LAG3 | −1.345224 |
| LSP1 | 0.7159047 | GATA3 | −0.790025 | CD27 | −1.366037 |
| CCL5 | 0.7121485 | PHLDA1 | −0.730273 | HLA-DPA1 | −1.402444 |
| LMNA | −0.727605 | FOSB | −1.022412 | HLA-DQA1 | −1.540706 |
| ANXA1 | −0.871862 | DUSP1 | −0.977568 | HLA-DRB1 | −1.569754 |
| GPR183 | −1.817057 | NFKBIA | −0.740723 | HSPB1 | −1.686551 |
| CCR7 | −2.027953 | DNAJB1 | −1.343857 | VCAM1 | −1.756093 |
| IL7R | −2.058816 | PDCD1 | −0.762573 | HLA-DRA | −1.808398 |
| TCF7 | −2.323058 | RHOB | −0.821483 | HSPA1A | −1.879661 |
| MTSS1 | 1.0790543 | ICOS | −0.70698 | CXCL13 | −2.429822 |
| PTMS | 0.7060496 | BHLHE40 | −0.804345 | GEM | −1.940271 |
| BATF | 0.7073517 | HLA-DRB1 | −0.693378 | S100A4 | −0.865874 |
| MCUB | −1.035826 | ZFAND2A | −0.890822 | RGS1 | −1.252445 |
| MBP | −1.005161 | CCL4 | −0.930144 | PTMS | −1.180215 |
| FOS | −1.266254 | FOS | −0.984264 | HMOX1 | −2.052834 |
| RAB27A | 0.7397483 | TUBB | −0.811373 | GZMA | −0.953218 |
| CD63 | 0.7331139 | | | MCUB | 0.7932615 |
| HOPX | 1.1368817 | | | HSPA1B | −2.492013 |
| TNFRSF18 | 0.879747 | | | BATF | −0.979571 |
| GADD45B | −1.170747 | | | IL32 | −0.735506 |
| PLPP1 | 1.069672 | | | HLA-DMA | −1.494637 |
| MCM5 | 0.8531565 | | | TNFRSF9 | −0.830153 |
| HMGA1 | −0.935381 | | | LGALS1 | −1.159753 |
| TNFSF10 | 0.8641462 | | | CD82 | −0.920667 |
| XCL1 | −1.276971 | | | HMGB2 | −1.015085 |
| PLK3 | −0.809413 | | | ID2 | −0.92326 |
| TAGAP | −0.732106 | | | CCL4 | −1.193417 |
| AHI1 | 0.7532266 | | | PSMB9 | −0.733615 |
| RARA | −0.88092 | | | NR4A2 | −1.128215 |
| FOSB | −0.931534 | | | GZMH | −0.851062 |
| CTSB | 0.709407 | | | RGS2 | −1.125671 |
| XCL2 | −1.239068 | | | BST2 | −0.894497 |
| PLSCR1 | 0.7820678 | | | DNAJB1 | −1.595408 |
| TUBB2A | −0.716931 | | | CACYBP | −0.861992 |
| CDC42EP3 | −0.722175 | | | CARD16 | −1.119713 |
| CARS | 0.8504097 | | | PDCD1 | −1.150405 |
| DUSP1 | −0.712521 | | | PRF1 | −0.945493 |
| DNAJB1 | −0.91726 | | | ID3 | −0.884443 |
| HSPA1B | −1.225033 | | | FYB1 | −0.804245 |
| | | | | ITGB2 | −0.79778 |
| | | | | ICOS | −1.033659 |
| | | | | CHST12 | −0.971611 |
| | | | | ANXA6 | −0.854806 |
| | | | | FABP5 | −0.765439 |
| | | | | JUNB | −0.846052 |
| | | | | TNFSF10 | −1.044788 |
| | | | | PHLDA1 | −0.847349 |
| | | | | LDLRAD4 | −0.808349 |
| | | | | EVL | −0.825475 |
| | | | | AHSA1 | −0.81836 |
| | | | | DOK2 | −0.787561 |
| | | | | BHLHE40 | −0.97308 |
| | | | | CRTAM | −1.08076 |
| | | | | TYMP | −0.88075 |
| | | | | TSPO | −0.732908 |
| | | | | ISG15 | −0.709982 |
| | | | | HLA-DQB1 | −0.770165 |
| | | | | SH3BGRL | −0.715615 |
| | | | | GALM | −0.861175 |
| | | | | PMAIP1 | −0.698804 |
| | | | | MTHFD2 | −0.702579 |
| | | | | XCL1 | −1.014759 |
| | | | | GCLM | 0.7136604 |
| | | | | ZFAND2A | −1.063685 |
| | | | | CHORDC1 | −0.759769 |
| | | | | MIR155HG | −0.784779 |
| | | | | CTSB | −0.775036 |
| | | | | FKBP4 | −0.723977 |
| | | | | FOSB | −0.81503 |
| | | | | GLA | −0.731156 |
| | | | | DUSP1 | −0.705953 |
| | | | | TUBB | −0.696002 |

TABLE 8
Differentially expressed genes among the 12 clusters of CD8+
TILs identified by scRNA-seq (adjusted P value < 0.05)

| Cluster 3: CD8 Effectors | | Cluster 4: T_{PE} CD8 | | Cluster 5: CD8 Mitotic | |
|---|---|---|---|---|---|
| gene | avg_logFC | p_val | p_val_adj | gene | avg_logFC |
| EGR1 | 2.6284941 | CAV1 | 1.7631972 | UBE2C | 4.1648936 |
| HSPA6 | 2.4620608 | GNG4 | 1.7379456 | PKMYT1 | 4.1591087 |
| FOS | 2.214825 | XCL1 | 1.5381438 | BIRC5 | 3.8259609 |
| HSPA1B | 2.1098065 | CRTAM | 1.3449958 | CDCA5 | 3.8182563 |
| GADD45B | 2.0423609 | CXCL13 | 1.2059375 | MKI67 | 3.5538006 |
| NR4A1 | 2.0091747 | GEM | 1.1671337 | HIST1H1B | 3.469622 |
| FOSB | 1.9022312 | XCL2 | 1.1259214 | ZWINT | 3.4250105 |
| ATF3 | 1.8416067 | ANXA1 | −1.516934 | KIFC1 | 3.3360113 |
| DNAJB1 | 1.8219649 | HLA-DRA | 0.9921856 | CDT1 | 3.3288778 |
| DUSP1 | 1.7837204 | BAG3 | 1.2782953 | TK1 | 3.2821617 |
| JUNB | 1.5563222 | HSPA1B | 0.9123036 | ASPM | 3.1207127 |
| CD69 | 1.4893487 | HLA-DQA1 | 1.0801252 | ASF1B | 3.1033768 |
| NR4A2 | 1.4728678 | HSPB1 | 0.9225696 | TYMS | 2.9847051 |
| NFKBIA | 1.4686878 | FABP5 | 0.9337088 | TOP2A | 2.9784604 |
| PPP1R15A | 1.3533858 | FTH1 | −0.720941 | CENPW | 2.973745 |
| KLF6 | 1.3337988 | SERPINH1 | 1.4499131 | CENPU | 2.9433021 |
| DNAJA1 | 1.1186912 | HLA-DPA1 | 0.873713 | CDCA8 | 2.9288522 |
| JUN | 1.0895146 | HLA-DRB1 | 0.9237493 | CDK1 | 2.9251914 |
| SRSF7 | 1.0262429 | HSPA1A | 1.0293133 | MAD2L1 | 2.6969811 |
| TSC22D3 | 0.9542911 | RGS2 | 0.8577402 | GGH | 2.6287199 |
| HSPA8 | 0.8158407 | CD74 | 0.7577011 | CKAP2L | 2.6154418 |
| GAPDH | −0.857877 | HSPD1 | 0.7846337 | CLSPN | 2.5780159 |
| SLC2A3 | 1.2747922 | HSPA6 | 0.9974754 | TUBB | 2.5287923 |
| ZFP36L1 | 1.0875748 | HSPE1 | 0.7307088 | TPX2 | 2.3367777 |
| S100A11 | −0.86922 | CD82 | 0.7061268 | SMC2 | 2.3204571 |
| IER2 | 1.3074524 | TOX | 0.9071727 | CKS1B | 2.3007942 |
| HSPA1A | 1.2962709 | HLA-DPB1 | 0.7672163 | STMN1 | 2.2802807 |
| EIF4A2 | 1.086535 | DNAJB1 | 0.9600001 | UBE2T | 2.1960825 |
| TGFB1 | −0.855682 | HLA-DMA | 0.7914759 | HIST1H1D | 2.1435424 |
| IFRD1 | 1.2250176 | GK | 0.8990892 | NRM | 2.1294973 |
| CCNL1 | 1.0988935 | ZFAND2A | 0.9334643 | KIF23 | 2.1023829 |
| BRD2 | 0.8029725 | NMB | 1.313455 | CENPM | 2.0438698 |
| HSP90AB1 | 0.6947002 | OASL | −0.801662 | CDKN3 | 2.0156414 |
| TUBB4B | 0.9332537 | DEDD2 | 0.8235358 | CENPN | 2.0142219 |
| PNRC1 | 0.7775079 | CMC1 | 0.902601 | LIG1 | 1.9557261 |
| APOBEC3G | −0.829357 | GPR183 | −1.373049 | DUT | 1.9271379 |
| HSPH1 | 0.895939 | ENC1 | 0.8418627 | MCM7 | 1.8947493 |
| RSRP1 | 1.2849181 | SELPLG | −0.910169 | NUDT1 | 1.8889323 |
| PKM | −0.81697 | GZMB | −0.833897 | FEN1 | 1.8759239 |
| SERTAD1 | 1.2350051 | GZMH | −0.82543 | DTYMK | 1.8560774 |
| S100A10 | −0.733554 | SLA2 | −0.758203 | CENPF | 1.8485738 |
| ANXA2 | −1.053671 | PDE4B | −0.735465 | TMEM106C | 1.8468593 |
| DEDD2 | 1.3145081 | CHST12 | −0.861954 | HMGN2 | 1.8092582 |
| TNFAIP3 | 0.7751152 | AKNA | −0.77073 | NUSAP1 | 1.8046398 |
| KLF10 | 1.635156 | ARL4C | −0.763513 | ATAD2 | 1.7851402 |
| ZFP36L2 | 0.8185774 | GLIPR1 | −0.862869 | PCNA | 1.774129 |
| KLF2 | 1.5158013 | UPP1 | −0.836333 | KIF22 | 1.7257036 |
| ISG20 | −1.010523 | FAM102A | −0.892557 | MCM3 | 1.6791161 |
| LSP1 | −0.740928 | IL7R | −0.767814 | PHF19 | 1.6275166 |
| ZFAND2A | 1.3120949 | PIK3R1 | −0.70737 | TUBA1B | 1.6187454 |
| ENO1 | −0.697996 | PRF1 | −0.814839 | HIST1H1E | 1.5691586 |
| H2AFX | 1.1812038 | FOXP1 | −0.82588 | TACC3 | 1.5647624 |
| CLK1 | 1.0941511 | ABLIM1 | −0.827471 | GSTM1 | 1.4852563 |
| EIF5 | 0.7631299 | GIMAP7 | −0.739657 | IDH2 | 1.3588451 |
| PPP1CA | −0.824686 | GYG1 | −0.75911 | HMGB2 | 1.3466364 |
| RSRC2 | 1.0841097 | | | CKS2 | 1.3097998 |
| PRF1 | −1.328131 | | | SMC4 | 1.2825869 |
| BTG2 | 1.2338494 | | | PTTG1 | 1.2536445 |
| RARRES3 | −0.805531 | | | CDKN2A | 1.2508293 |
| ATP5MF | −0.818489 | | | MT1E | 1.2337859 |
| MYLIP | 1.4049976 | | | EZH2 | 1.2222893 |
| SLA2 | −1.162304 | | | H2AFY | 1.2034567 |
| CSRNP1 | 0.9998543 | | | NUCB2 | 1.1870447 |
| MT2A | −0.751399 | | | NUCKS1 | 1.1809425 |
| ID2 | 1.0423139 | | | H2AFZ | 1.1755564 |
| FKBP1A | −0.761153 | | | DNMT1 | 1.169111 |
| CXCR3 | −0.886951 | | | TMPO | 1.1170964 |
| WDR1 | −0.768931 | | | MCM5 | 1.0691018 |
| PSMB9 | −0.699732 | | | HMGB1 | 1.0547274 |
| ZC3H12A | 1.2344524 | | | ANP32B | 1.0394864 |
| BAG3 | 1.2605443 | | | CALM3 | 1.0291234 |
| SNHG12 | 1.3104353 | | | HLA-DRA | 1.0038136 |
| ATP5MD | −0.790761 | | | ACTB | 0.9755329 |
| CHST12 | −1.325454 | | | PFN1 | 0.9467017 |
| COX5A | −0.761529 | | | PSMB9 | 0.9407674 |
| ISG15 | −0.898588 | | | H2AFV | 0.9242349 |
| OASL | −0.701272 | | | HLA-DMA | 0.9109371 |
| RABAC1 | −0.772264 | | | COX8A | 0.8625349 |
| ANXA5 | −0.785281 | | | CD74 | 0.7412291 |
| GZMB | −1.16534 | | | ACTG1 | 0.7072207 |
| DDIT4 | 1.1470154 | | | RPS27 | −0.833279 |
| ATP1B3 | −0.735765 | | | HLA-DPA1 | 0.8642941 |
| SNHG15 | 1.0890713 | | | CENPX | 1.268454 |
| NR4A3 | 1.1495251 | | | GMNN | 1.2816476 |
| RBX1 | −0.801164 | | | ANAPC11 | 0.9853228 |
| UBE2L6 | −0.792032 | | | DEK | 0.9439957 |
| ATP6V1F | −0.765639 | | | HLA-DRB1 | 0.8509425 |
| ZFAS1 | 0.9442759 | | | SKA2 | 1.3656226 |
| FUS | 0.7263168 | | | PSME2 | 0.8154572 |
| BRK1 | −0.710638 | | | ANP32E | 1.0614975 |
| DYNLRB1 | −0.769487 | | | LSM4 | 1.057246 |
| REL | 0.7876172 | | | HIST1H4C | 1.3733902 |
| CYB5R3 | −0.89389 | | | HMGN3 | 0.9610079 |
| TAGAP | 0.9378877 | | | SLC25A5 | 0.7144788 |
| APOBEC3C | −0.787465 | | | SMC1A | 1.1454093 |
| GGA2 | −0.85235 | | | CORO1A | 0.7041088 |
| MEAF6 | −0.727102 | | | RRM1 | 1.2581266 |
| ATF4 | 1.0071754 | | | MAD2L2 | 1.114623 |
| AP2M1 | −0.75088 | | | MT2A | 0.806628 |
| RASGEF1B | 1.2239604 | | | PRDX2 | 0.9400253 |
| PDCD5 | −0.949658 | | | LSM2 | 0.8899944 |
| PRDX5 | −0.753618 | | | PAFAH1B3 | 1.2194084 |
| DDIT3 | 1.2240309 | | | DNAJC9 | 0.9573988 |
| NEU1 | 0.8005274 | | | CXCL13 | 0.8136195 |
| SNHG8 | 1.0794125 | | | NAA38 | 1.079401 |
| NDUFS6 | −0.765437 | | | FABP5 | 0.750894 |
| TIGIT | −0.866976 | | | HINT2 | 1.0863307 |
| CD63 | −0.76324 | | | YEATS4 | 1.1518102 |
| ZNF331 | 0.7765036 | | | BANF1 | 0.8333021 |
| ARPC1B | −0.788726 | | | SIVA1 | 0.8524414 |
| AP2S1 | −0.819472 | | | PHPT1 | 0.8880963 |
| NUTF2 | −0.918644 | | | RANBP1 | 0.9497496 |
| CITED2 | 1.0891331 | | | SHMT2 | 1.0532812 |
| C4orf48 | −1.031954 | | | SNRPA | 0.8787086 |
| IFI6 | −0.971861 | | | RPA3 | 0.9114234 |
| SELPLG | −0.698664 | | | SNRNP25 | 1.2671602 |
| TXNIP | 1.4608888 | | | HMGB3 | 1.1936889 |
| SBDS | 0.8974556 | | | CDC25B | 1.1105866 |
| NDUFA12 | −0.738829 | | | SAE1 | 1.0914984 |
| LGALS3 | −1.107059 | | | TALDO1 | 0.8567458 |
| TOB1 | 0.9917836 | | | H2AFX | 0.7297075 |
| PIM2 | 1.0148126 | | | HSPB11 | 1.018457 |
| ZC3HAV1 | 0.7192845 | | | GSTP1 | 0.7598261 |
| YWHAH | −0.852122 | | | PTMS | 0.7292829 |
| SRSF3 | 0.9170539 | | | MRPL11 | 1.0416989 |
| TWF2 | −0.697411 | | | BLOC1S1 | 1.1020691 |
| UBE2E3 | −0.990035 | | | IFI27L2 | 0.7640351 |
| MX1 | −0.903005 | | | MYL6B | 1.0474121 |
| NEDD8 | −0.736296 | | | PYCARD | 0.9454261 |
| TMEM43 | −0.715854 | | | CARHSP1 | 0.7280042 |
| PLK3 | 0.9275239 | | | C12orf75 | 0.7692138 |
| CCNDBP1 | −0.697069 | | | PPIH | 0.9793698 |
| TNFSF10 | −1.146489 | | | DCTN3 | 0.8093296 |
| MGST3 | −0.787912 | | | MTHFD2 | 0.7071458 |
| TIPARP | 1.1163819 | | | ZFP36 | −0.868534 |
| TALDO1 | −0.750744 | | | SSRP1 | 0.9039223 |
| ZFAND5 | 0.7734945 | | | FDPS | 0.910687 |
| NFKBIZ | 1.1250844 | | | FIBP | 0.8381071 |
| CARHSP1 | −0.820825 | | | USP1 | 0.85974 |
| POLR2I | −0.702758 | | | ACAT2 | 0.9301694 |
| AP1M1 | −0.767649 | | | NDUFS8 | 0.7366133 |
| CCL4 | 1.4086325 | | | AKR7A2 | 0.9924679 |
| PER1 | 0.8913581 | | | UBE2S | 0.7544431 |
| ITGAE | −0.76091 | | | TSTD1 | 0.7023517 |
| CHORDC1 | 0.9603612 | | | MZT2A | 0.7935332 |
| CD58 | −0.746529 | | | CD70 | 0.8879923 |
| UBE2S | 0.9352033 | | | PRDX3 | 0.826205 |
| DDX3X | 0.7232301 | | | PNKD | 0.9225507 |
| CTSB | −0.803121 | | | LGALS1 | 0.8054274 |
| RHOH | 0.7209339 | | | MZT2B | 0.7755475 |
| STK17B | 0.6942898 | | | MT1F | 0.7553246 |
| EIF4A3 | 0.7552782 | | | DDX39A | 0.702235 |
| APLP2 | −0.76882 | | | ACOT7 | 0.9773843 |
| VPS35 | −0.750981 | | | MCRIP1 | 0.7667239 |
| PPP1R10 | 1.0567072 | | | HPRT1 | 0.7799835 |
| CCL4L2 | 1.362535 | | | CD38 | 0.6959274 |
| TYMP | −0.742651 | | | LMNB1 | 0.712403 |
| PTGER4 | 0.893969 | | | LSM3 | 0.7268035 |
| CKS2 | 0.8868014 | | | RPS29 | −0.813848 |
| CHMP1B | 1.0431482 | | | MIS18BP1 | 0.8056884 |
| TRA2B | 0.8368188 | | | IL7R | −1.564143 |
| KLRD1 | −0.746793 | | | WDR54 | 0.8089116 |
| PLEC | −0.824271 | | | VPS29 | 0.7053474 |
| ODC1 | 0.732672 | | | CKAP2 | 0.8298392 |
| CHD2 | 0.7200672 | | | PSIP1 | 0.7909241 |
| SAMD9 | −0.743079 | | | SMC3 | 0.7096266 |
| TUBB2A | 0.9235006 | | | CDK4 | 0.8208917 |
| SLC1A5 | 0.829616 | | | PFKL | 0.7943067 |
| AMD1 | 0.784211 | | | NCAPH2 | 0.8934149 |
| MRPL18 | 1.0309546 | | | YIF1B | 0.7715058 |
| HBP1 | 0.7458707 | | | MRPL37 | 0.7007583 |
| STMN1 | −0.732942 | | | POLD2 | 0.8806028 |
| GYG1 | −0.698846 | | | POP7 | 0.7304606 |
| TUBB | −0.792484 | | | LSM5 | 0.6963591 |
| SLC38A2 | 0.7673315 | | | RBBP8 | 0.8251378 |
| CD55 | 1.0676276 | | | NENF | 0.6989472 |
| TCP1 | 0.7241433 | | | GPAA1 | 0.7266429 |
| CCNH | 0.8651768 | | | H1FX | 0.7107028 |
| IFNG | 1.2926012 | | | LMNA | −0.699308 |
| DDX3Y | 0.7448862 | | | MCM6 | 0.6966701 |
| RGS2 | 0.8422315 | | | GPR183 | −1.653037 |
| ELL2 | 0.7424574 | | | ANXA1 | −1.024941 |
| CDKN1A | 0.8301992 | | | CDCA4 | 0.7142048 |
| YTHDC1 | 0.7316487 | | | GZMM | −1.03223 |
| CCDC59 | 0.7177924 | | | TCF7 | −1.295179 |
| RSBN1 | 0.7266217 | | | PBXIP1 | −0.808615 |
| PNP | 0.7404046 | | | MBP | −1.099391 |
| C1orf52 | 0.7608111 | | | GABARAPL1 | −0.782818 |
| CDC42EP3 | 0.7555168 | | | PRNP | −0.99913 |
| VSIR | 0.7118491 | | | AHNAK | −0.767394 |
| RHOB | 0.7335142 | | | MARCKSL1 | −0.903712 |
| | | | | ANXA2 | −0.711207 |
| | | | | TSPYL2 | −0.812617 |
| | | | | CD55 | −0.944367 |
| | | | | AKNA | −0.80597 |
| | | | | ABLIM1 | −0.904833 |
| | | | | KLRD1 | −0.931574 |
| | | | | PIK3R1 | −0.802447 |
| | | | | MAT2A | −0.776384 |
| | | | | PBX4 | −0.750409 |

TABLE 9
Differentially expressed genes among the 12 clusters of CD8+
TILs identified by scRNA-seq (adjusted P value < 0.05)

| Cluster 6: CD8 Apoptotic | | Cluster 7: NK-like | | Cluster 8: T_{TE} | |
|---|---|---|---|---|---|
| gene | avg_logFC | gene | avg_logFC | gene | avg_logFC |
| EEF1A1 | −1.001906 | KLRC3 | 1.9986002 | TRAV22 | 4.1649575 |
| MALAT1 | 1.498113 | GNLY | 2.1072776 | TRAV20 | 4.141344 |
| TMSB4X | −1.146229 | CD300A | 1.7769717 | TRBV4-1 | 3.734315 |
| RPLP1 | −0.893399 | FTH1 | 0.8691 | HLA-DRB1 | 1.2410385 |
| TPT1 | −1.012396 | PDE4A | 1.5816287 | CXCL13 | 1.4359608 |
| RPL41 | −0.86664 | HLA-DPA1 | −2.20895 | CD74 | 1.0032703 |
| RPS6 | −1.076357 | KLRD1 | 0.9179218 | HLA-DPA1 | 1.1331952 |
| RPS28 | −1.067722 | MATK | 1.3686826 | HLA-DRA | 1.2161239 |
| RPL15 | −0.949738 | CD74 | −1.291425 | HLA-DQA1 | 1.1711849 |
| PPIA | −1.185431 | LAG3 | −2.024968 | HLA-DPB1 | 1.0744034 |
| RPS14 | −0.904341 | HLA-DRB1 | −2.467126 | VCAM1 | 1.1576725 |
| RPS27 | −1.025444 | IL7R | 0.7182015 | CD27 | 1.0001704 |
| RPL9 | −1.038045 | PIK3R1 | 0.850706 | PON2 | 1.6963759 |
| RPL39 | −0.991323 | GPCPD1 | 1.0699313 | NKG7 | 0.7385088 |
| RPSA | −1.051883 | HLA-DRB5 | −1.708549 | LMNA | −1.324753 |
| RPL23A | −0.948983 | HLA-DRA | −2.476138 | DUSP4 | 0.6970691 |
| RPS23 | −0.864407 | XCL1 | 0.9219663 | LDHA | −0.935476 |
| RPS4X | −0.90535 | HLA-DPB1 | −1.539266 | TRBC2 | 0.8187905 |
| RPL10 | −0.705187 | TCF7 | 0.859965 | VIM | −1.072346 |
| RPS12 | −0.837361 | GZMA | −1.709272 | RPL37A | −0.767815 |
| RPL21 | −0.894296 | IFITM3 | 0.9461474 | S100A10 | −1.246135 |
| RPS15A | −0.782649 | CXCL13 | −4.867155 | RPS21 | −0.764479 |
| RPS16 | −0.970408 | PRKX | 0.9446129 | ENC1 | 1.293467 |
| RPLP2 | −0.842474 | CAST | 0.7564937 | CD8B | 0.7506916 |
| RPL35A | −0.834143 | NKG7 | −0.9132 | RPS27 | −0.718495 |
| RPS25 | −0.840113 | HLA-DQA1 | −2.486619 | RPS29 | −1.143416 |
| RPS27A | −0.733818 | LDLRAD4 | 0.9222027 | TGFB1 | −1.179097 |
| RPS3A | −0.786272 | XCL2 | 0.9229597 | CRTAM | 1.0421463 |
| MT-CO1 | 0.9431959 | CD2 | −0.90855 | FTH1 | −0.965293 |
| RPL18A | −0.760845 | CD8B | −1.019121 | TAGLN2 | −0.706119 |
| RPL37 | −0.89543 | DUSP4 | −0.830389 | RAB11FIP1 | 1.0723874 |
| RPS3 | −0.747554 | BATF | −1.396532 | ANXA1 | −1.722004 |
| RPL34 | −0.845181 | VCAM1 | −2.958728 | HLA-DMA | 1.0200564 |
| RPL32 | −0.773878 | HSPH1 | −1.174813 | ANXA2 | −1.633995 |
| RPL19 | −0.744368 | PLSCR1 | 0.7003036 | NR4A2 | 0.7471067 |
| RPS13 | −0.789723 | GZMK | −1.191753 | TRBC1 | −2.411627 |
| UBA52 | −0.9608 | JUN | −0.895875 | HSPB1 | 0.7186417 |
| MT-CO2 | 0.8438678 | CCL4 | −2.355765 | MS4A6A | 1.2831683 |
| RPS29 | −1.305561 | GZMH | −1.648996 | TNFRSF9 | 0.7445752 |
| RPS7 | −0.748179 | HSP90AA1 | −0.893612 | FYB1 | 0.8229333 |
| RPS9 | −0.77792 | BACH2 | 0.8700355 | SYNGR2 | 0.836385 |
| RPLP0 | −0.771712 | ID3 | 0.8269667 | TOB1 | 0.9527006 |
| RPL7A | −0.717485 | CD27 | −1.240426 | CHN1 | 1.068331 |
| RPL3 | −0.808226 | PTMS | −1.510357 | GABARAPL1 | −1.248137 |
| RPS18 | −0.73494 | LSP1 | −0.828269 | MBP | −2.36954 |
| RPL27 | −0.967778 | CMSS1 | 0.9533868 | RPL38 | −0.794547 |
| RPL8 | −0.704941 | ICOS | −1.968182 | GZMK | 0.7065114 |
| RPS5 | −0.759053 | SATB1 | 0.8309895 | BHLHE40 | 0.9612228 |
| RPL6 | −0.717506 | BCL2A1 | 0.8280144 | XCL1 | 0.8216036 |
| RPL26 | −0.903798 | LGALS1 | −1.238585 | RPS26 | −0.926279 |
| PFN1 | −0.996505 | HSPA1A | −1.525253 | MX1 | −2.018568 |
| TOMM7 | −1.046293 | SETD2 | 0.6991217 | GPR183 | −1.961277 |
| RPS24 | −0.70407 | PITPNC1 | 0.7148621 | MIR155HG | 0.9267396 |
| SH3BGRL3 | −0.830663 | PMAIP1 | −1.101039 | TOX | 1.0177974 |
| FTH1 | −0.718561 | DNAJB1 | −1.668352 | SIT1 | 1.2160716 |
| RPL24 | −0.726727 | HLA-DMA | −1.725525 | IL7R | −1.51928 |
| CFL1 | −0.846673 | HLA-DQB1 | −1.369745 | GATA3 | 0.7721385 |
| RPL27A | −1.059149 | PPP1R2 | −0.790098 | CD6 | −1.070812 |
| MT-CYB | 1.0088104 | H2AFJ | 0.7823999 | ISG20 | −1.021601 |
| RPS21 | −0.859513 | FYB1 | −1.017405 | OASL | −1.116579 |
| RPL37A | −0.773945 | SYNGR2 | −0.920952 | KLRD1 | −2.555898 |
| RPL36 | −0.733682 | CYTOR | −0.975268 | HIST1H4C | −1.147973 |
| BTF3 | −0.817644 | GZMB | −0.924113 | GZMM | −1.501225 |
| COX7C | −0.892722 | PPP1R15A | −0.722463 | TMX4 | 0.8599769 |
| RPL10A | −0.758222 | HSPB1 | −1.162508 | CD55 | −1.435768 |
| ATP5F1E | −0.858817 | HNRNPLL | −0.872731 | HERPUD1 | 0.6997207 |
| GAPDH | −0.695907 | HERPUD1 | −0.840554 | ANXA6 | 0.7522609 |
| ATP5MG | −0.819159 | MT1X | −1.016206 | RPL36A | −0.957788 |
| RPL38 | −1.01648 | SH3KBP1 | −0.930195 | GSTP1 | 0.7132762 |
| RACK1 | −0.751461 | HSPA1B | −2.287393 | CD70 | 1.0606998 |
| MIF | −0.783541 | CACYBP | −0.803285 | CRIP1 | −1.017498 |
| OST4 | −0.868184 | CD82 | −0.748567 | PMAIP1 | 0.7481091 |
| RPL22 | −0.800769 | LBH | −0.767572 | ISG15 | −1.304792 |
| HINT1 | −0.845741 | ALOX5AP | −1.173813 | CSNK1G3 | 1.0301081 |
| RPL4 | −0.819753 | NR4A2 | −0.893358 | MT-ND6 | −0.967973 |
| FTL | −0.809897 | JUNB | −0.958846 | ABLIM1 | −1.618469 |
| MT-ND4L | 1.0029727 | FKBP4 | −0.991974 | RGS2 | 0.6953525 |
| GABARAP | −0.881975 | DNAJA1 | −0.783603 | CYSTM1 | −0.923685 |
| COX7A2 | −0.967097 | JMJD6 | −0.732756 | MT1E | 0.7938442 |
| MYL12B | −0.777058 | CD3G | −0.80621 | RGS1 | 0.7065719 |
| RPS20 | −0.922236 | RGS2 | −0.933258 | FOXP1 | −1.363101 |
| ARHGDIB | −0.724151 | HMGB2 | −0.803329 | PBXIP1 | −0.754864 |
| EEF1B2 | −0.777486 | RGS1 | −0.872967 | AHNAK | −0.857794 |
| SOD1 | −0.769788 | WNK1 | −0.803379 | MCUB | −1.20692 |
| PSME1 | −0.832598 | CTSC | −0.788821 | SRRT | −0.949047 |
| RPL36AL | −0.698071 | NFKBIA | −0.778312 | PNPLA2 | −0.82357 |
| PRDX1 | −0.957052 | TCP1 | −0.765765 | DDX3Y | 0.7443051 |
| CALM2 | −0.747963 | RAB27A | −0.706396 | TCF7 | −1.433778 |
| COX8A | −0.907923 | PLK3 | −0.7734 | HLA-DQB1 | 0.7252342 |
| COX6C | −1.054724 | DUSP1 | −0.859848 | PIM1 | −1.149312 |
| EIF3F | −0.759302 | CARD16 | −0.77227 | EEF1G | −0.885221 |
| SLC25A5 | −0.740005 | PRF1 | −0.705467 | SQLE | 0.8910164 |
| PRDX6 | −1.109458 | FOSB | −0.747099 | HMGA1 | −1.281924 |
| UQCRB | −0.728551 | RHOB | −0.718014 | ARL4A | 0.8226307 |
| NDUFS5 | −0.741424 | ZC3H12A | −0.754218 | TSTD1 | 0.7997739 |
| RHOA | −0.715146 | | | MTSS1 | 0.895742 |
| ATP5MC2 | −0.776635 | | | IRF7 | −0.972512 |
| GNG5 | −0.762125 | | | ATP1B3 | −0.704205 |
| ARPC3 | −0.696588 | | | UPP1 | −1.127473 |
| SEC61B | −0.697368 | | | ENTPD1 | 0.7692546 |
| NKG7 | −0.762544 | | | DCXR | −0.704662 |
| COMMD6 | −0.895568 | | | MT1F | 0.8910065 |
| RARRES3 | −0.936859 | | | SELPLG | −0.707431 |
| S100A11 | −0.763918 | | | NAB1 | 0.8053468 |
| S100A10 | −0.796303 | | | ARL4C | −0.796534 |
| MT-CO3 | 0.8709231 | | | PHPT1 | 0.7894141 |
| ALDOA | −0.74419 | | | LGALS3 | −1.111399 |
| ATP6V0E1 | −0.718889 | | | IFITM3 | −0.903914 |
| RPL23 | −0.70147 | | | SLA2 | −0.695698 |
| ZFAS1 | −0.792619 | | | C4orf48 | −0.850649 |
| UQCRH | −1.017107 | | | SPN | 0.6941518 |
| UQCR10 | −0.846992 | | | AP1M1 | −0.72249 |
| COX7B | −0.843197 | | | BAX | 0.7434374 |
| MT-ND5 | 0.9325471 | | | KDM5B | 0.7615773 |
| ATP5MC3 | −0.769529 | | | N4BP2L2 | −0.716747 |
| FKBP1A | −0.976636 | | | ZNFX1 | −0.761609 |
| COX6B1 | −0.794063 | | | GIMAP7 | −0.720251 |
| PARK7 | −0.800527 | | | CDK6 | 0.7213275 |
| TXN | −0.856405 | | | NT5C3A | −0.705857 |
| ATP5MD | −0.884 | | | GYG1 | −0.755578 |
| DBI | −0.822645 | | | ATRAID | 0.7252325 |
| WDR83OS | −0.695237 | | | DDIT4 | −0.778731 |
| MT-ND2 | 1.1436824 | | | VOPP1 | 0.7008934 |
| MT-ND1 | 1.0172219 | | | AHR | 0.7172027 |
| SNRPG | −0.880237 | | | | |
| COX5A | −0.79362 | | | | |
| PSMB3 | −0.899117 | | | | |
| NDUFB2 | −0.842268 | | | | |
| LDHB | −0.772572 | | | | |
| MT-ATP6 | 1.0514006 | | | | |
| ATP5MF | −0.764081 | | | | |
| PEBP1 | −0.721986 | | | | |
| HMGN2 | −0.824501 | | | | |
| GSTP1 | −0.840787 | | | | |
| SPCS1 | −0.760045 | | | | |
| CRIP1 | −0.911837 | | | | |
| BSG | −0.773658 | | | | |
| MT-ND4 | 1.0183341 | | | | |
| RBX1 | −1.019124 | | | | |
| CD52 | −0.763831 | | | | |
| HIGD2A | −0.758912 | | | | |
| RPL36A | −0.783745 | | | | |
| PSME2 | −0.716242 | | | | |
| PPP1CA | −0.70895 | | | | |
| SUMO1 | −0.827648 | | | | |
| IFITM2 | −0.712511 | | | | |
| SEC61G | −0.997753 | | | | |
| C4orf3 | −0.973459 | | | | |
| UXT | −0.735207 | | | | |
| RPS4Y1 | −0.914357 | | | | |
| NDUFB4 | −0.976599 | | | | |
| EEF1G | −1.383125 | | | | |
| EIF3E | −0.793624 | | | | |
| CYSTM1 | −0.883332 | | | | |
| ANXA2 | −0.822798 | | | | |
| ATP5PB | −0.825776 | | | | |
| NDUFAB1 | −0.988636 | | | | |
| GHITM | −0.73832 | | | | |
| APRT | −0.869696 | | | | |
| ATP5F1C | −1.028595 | | | | |
| MT-ND3 | 1.0716418 | | | | |
| ATP5PF | −1.01724 | | | | |
| KRTCAP2 | −0.703594 | | | | |
| NME2 | −1.093267 | | | | |
| UQCR11 | −0.770996 | | | | |
| ATP6V1F | −0.790838 | | | | |
| TMEM258 | −0.879833 | | | | |
| TRAPPC1 | −1.0319 | | | | |
| TOMM22 | −0.849591 | | | | |
| SNRPF | −0.913438 | | | | |
| VAMP8 | −0.727005 | | | | |
| TOMM5 | −0.74395 | | | | |
| TSPO | −0.824596 | | | | |
| MDH2 | −0.709936 | | | | |
| MRPL51 | −0.953854 | | | | |
| GTF3C6 | −0.907606 | | | | |
| NDUFA2 | −0.714648 | | | | |
| NDUFA12 | −0.785809 | | | | |
| ARPC1B | −0.774326 | | | | |
| COPS9 | −0.881017 | | | | |
| GMFG | −0.810971 | | | | |
| SRI | −0.873174 | | | | |
| NUTF2 | −1.005329 | | | | |
| PDCD5 | −0.892389 | | | | |
| SEC11C | −0.774828 | | | | |
| SNRPC | −0.70111 | | | | |
| TALDO1 | −0.874562 | | | | |
| SRP9 | −0.704762 | | | | |
| PDCD6 | −0.706722 | | | | |
| NEDD8 | −0.886521 | | | | |
| DDT | −0.835636 | | | | |
| ASNA1 | −0.800898 | | | | |
| NDUFAF3 | −1.118265 | | | | |
| BANF1 | −0.791544 | | | | |
| ABRACL | −0.763073 | | | | |
| IFITM3 | −0.791236 | | | | |
| SMDT1 | −0.716544 | | | | |
| COX17 | −0.884637 | | | | |
| MGST3 | −0.799981 | | | | |
| ACP1 | −0.77785 | | | | |
| IFI6 | −0.759782 | | | | |
| UBE2E3 | −0.755455 | | | | |
| C4orf48 | −0.699895 | | | | |
| SH3BGRL | −0.722628 | | | | |
| COX7A2L | −0.912834 | | | | |
| MRPL14 | −0.824652 | | | | |
| TNFAIP3 | 0.9850269 | | | | |
| LGALS3 | −0.782995 | | | | |
| PYURF | −0.751288 | | | | |
| C14orf119 | −0.737208 | | | | |
| HMGA1 | −0.819327 | | | | |
| OCIAD2 | −0.771835 | | | | |
| DPH3 | −0.775845 | | | | |
| MT-ATP8 | 1.0811123 | | | | |
| CXCL13 | −1.062201 | | | | |
| NEAT1 | 1.8880947 | | | | |
| FUS | 1.1256287 | | | | |
| HSPA1B | 1.1343526 | | | | |
| EIF4A1 | 0.7408347 | | | | |
| JUN | 0.7904129 | | | | |
| RSRP1 | 1.2824085 | | | | |
| TSPYL2 | 1.0799198 | | | | |
| PPP1R15A | 0.7035984 | | | | |

TABLE 10
Differentially expressed genes among the 12 clusters of CD8+
TILs identified by scRNA-seq (adjusted P value < 0.05)

| Cluster 9: Treg-like | | Cluster 10: γδ like-T | | Cluster 11: T_{EX} | | Cluster 12: Naive | |
|---|---|---|---|---|---|---|---|
| gene | avg_logFC | gene | avg_logFC | gene | avg_logFC | gene | avg_logFC |
| CCR8 | 4.0105262 | TRDV2 | 5.4357506 | TRAV24 | 4.4536488 | LEF1 | 3.1018311 |
| CD4 | 3.5629338 | FEZ1 | 2.9644468 | TRBV5-5 | 3.9941724 | SELL | 2.4663875 |
| FOXP3 | 3.4898204 | TRGV9 | 2.7404902 | CCDC50 | 2.0158347 | MYC | 2.3632247 |
| TNFRSF4 | 3.1157804 | KLRB1 | 2.4914872 | HLA-DRB1 | 0.9351924 | RPL32 | 0.9007991 |
| TNFRSF18 | 2.2071503 | KLRC1 | 1.8492407 | VCAM1 | 1.1705088 | RPS3A | 0.8702556 |
| IL2RA | 2.9155581 | BCL2A1 | 1.6306606 | CTSW | 1.0049068 | RPS13 | 0.919175 |
| TMEM173 | 2.1966843 | RORA | 1.5442665 | NKG7 | 0.8420563 | CCL5 | −4.136921 |
| LTB | 1.96076 | VIM | 0.8289899 | CXCL13 | 0.9557477 | LTB | 1.8508183 |
| GK | 1.6054741 | CD8A | −1.3893 | HLA-DPA1 | 0.810545 | RPL13 | 0.7500003 |
| ATP1B1 | 1.8404132 | CD300A | 1.6090707 | DUSP4 | 0.7697006 | RPS8 | 0.9003568 |
| MAGEH1 | 2.0668577 | KLRD1 | 1.2305926 | GZMB | 0.9027038 | EEF1A1 | 0.7016184 |
| TBC1D4 | 1.6636887 | IL7R | 0.8202433 | HLA-DRA | 0.8294588 | RPS5 | 0.8648513 |
| BATF | 1.2066948 | CD8B | −2.06639 | HLA-DPB1 | 0.7019844 | RPL18A | 0.7639636 |
| LINC01943 | 1.5632077 | COTL1 | −1.229105 | LMNA | −1.38746 | RPS12 | 0.8145066 |
| IL32 | 1.1273892 | AQP3 | 1.4968366 | LSP1 | 0.7313476 | RPL11 | 0.7516635 |
| S100A4 | 1.1184135 | GNLY | 0.9845942 | CD27 | 0.8458902 | CCR7 | 1.5291043 |
| CTLA4 | 1.1244121 | PBX4 | 1.0173943 | ISG20 | −1.695194 | RPS23 | 0.7551688 |
| DNPH1 | 1.2161425 | TNFRSF25 | 1.3012023 | HLA-DQA1 | 0.8180888 | CST7 | −3.860904 |
| MAL | 1.8349833 | SATB1 | 1.1702973 | IL7R | −2.879724 | GIMAP7 | 1.6857822 |
| PGM2L1 | 1.593029 | MT2A | 1.1024972 | RPS21 | −0.701938 | GAPDH | −1.637692 |
| ANKRD10 | 1.3907537 | TNFSF14 | 1.228746 | S100A10 | −1.128662 | NKG7 | −4.60935 |
| CORO1B | 1.3326716 | CD27 | −2.093656 | LAG3 | 0.7367034 | RPS6 | 0.780294 |
| GADD45A | 1.4754916 | STAT4 | 0.8218771 | GATA3 | 0.9994215 | RPS14 | 0.694101 |
| CARD16 | 1.1048817 | PERP | 1.0235678 | VIM | −0.926682 | EEF1B2 | 0.984142 |
| MAST4 | 1.5399396 | ADAM8 | 0.9653245 | MTSS1 | 1.1498309 | RPL29 | 0.722209 |
| STAM | 1.3080029 | HLA-DPA1 | −1.663863 | MIR155HG | 1.0709502 | RPL22 | 0.9282519 |
| CD8A | −0.793241 | FASLG | 0.7561804 | TMX4 | 1.0257958 | RPL5 | 0.8539391 |
| RAB11FIP1 | 0.9221494 | HLA-DPB1 | −1.435871 | LDHA | −0.716104 | RPL34 | 0.7544123 |
| HTATIP2 | 1.4977771 | HSPH1 | −1.328333 | MBP | −3.080031 | RPL9 | 0.7660901 |
| ZFAND5 | 0.8945202 | GYG1 | 0.8421303 | MS4A6A | 1.1196853 | HLA-B | −0.764487 |
| CTSC | 0.9898437 | UPP1 | 0.7930035 | BHLHE40 | 0.8890715 | RPS18 | 0.8832921 |
| MIR4435- | 1.184614 | GLIPR1 | 0.715608 | FTH1 | −0.902085 | RPL3 | 0.7120236 |
| 2HG | | | | | | | |
| LAYN | 1.0507736 | GLUL | 0.884193 | ENTPD1 | 1.010371 | CD74 | −2.221084 |
| CCL5 | −0.82489 | CXCL13 | −2.604759 | RAB11FIP1 | 0.9501225 | DUSP2 | −2.620728 |
| SKAP1 | 1.0279734 | HLA-DRA | −1.922014 | GPR183 | −2.82725 | RPL10A | 0.7644458 |
| ICOS | 0.8794633 | HLA-DRB5 | −1.297799 | ANXA2 | −1.298555 | SRGN | −1.029185 |
| CD83 | 1.1393656 | RASSF5 | 0.7154809 | CD44 | −0.699247 | RPS4X | 0.7234911 |
| PIM2 | 0.8437716 | FYB1 | −1.35568 | TGFB1 | −0.881392 | DUSP4 | −3.403205 |
| CHST7 | 1.1632392 | HLA-DQA1 | −2.102944 | HAVCR2 | 0.8416995 | CD8A | −1.458601 |
| ARID5B | 0.776656 | VCAM1 | −3.011113 | PHLDA1 | 0.7273746 | COTL1 | −2.169776 |
| VIM | −0.801957 | MATK | 0.8740383 | CHD1 | 0.8356196 | H3F3B | −0.857136 |
| RORA | 1.0648666 | TIGIT | −1.819904 | RPS29 | −0.883652 | RPL4 | 0.7729882 |
| ACP5 | 0.8347176 | AUTS2 | 0.8947611 | CCR7 | −1.676109 | CREM | −1.967389 |
| PELI1 | 0.9958899 | HSPB1 | −1.575903 | GZMH | 0.7667659 | APOBEC3G | −2.829369 |
| GBP2 | 0.7338368 | IFNG | 1.3141078 | RGS2 | 0.7342196 | PASK | 1.8864812 |
| ANXA1 | −1.45956 | ITM2A | −0.864371 | RPL38 | −0.739589 | GIMAP4 | 1.5609036 |
| JMY | 1.0332791 | GEM | −2.576172 | CD70 | 1.1012014 | TUBA4A | −2.22683 |
| INPP5F | 0.9727375 | HSP90AA1 | −0.865944 | FAM3C | 1.2204347 | HLA-DPA1 | −3.094272 |
| FCMR | 0.9317552 | HLA-DRB1 | −1.680086 | CD55 | −1.625111 | GZMK | −3.875118 |
| CSNK1G3 | 0.8295237 | ECE1 | 0.8178385 | GZMA | 0.7349463 | CD55 | 1.1011774 |
| SEC11C | 0.7432302 | PPP1R2 | −0.932761 | SIRPG | 1.0436782 | HLA-DPB1 | −2.664975 |
| GRSF1 | 0.8692962 | RGS1 | −1.313094 | RPL36A | −1.064166 | CTSW | −2.153957 |
| TPP1 | 1.0494442 | LAG3 | −1.099098 | ITGB2 | 0.7389679 | NOP53 | 0.7979556 |
| ZNF292 | 0.8656622 | CD74 | −0.880815 | TCF7 | −2.834877 | HSP90AA1 | −1.697894 |
| FAS | 0.7657877 | ICOS | −1.772849 | MX1 | −1.950092 | ACTG1 | −1.203635 |
| OASL | −1.087432 | HMGB2 | −1.181928 | SQLE | 1.0242791 | CLIC1 | −1.409833 |
| SIRPG | 0.791985 | CRTAM | −2.023336 | DDX3Y | 0.8491767 | EMP3 | 0.9199997 |
| ATOX1 | 1.0092109 | DNAJB1 | −1.497444 | HIST1H4C | −0.971616 | LAG3 | −3.052268 |
| SELL | 0.8105598 | HLA-DMA | −1.802758 | GABARAPL1 | −0.935671 | PCSK1N | 1.4959689 |
| NMB | 0.7161968 | BATF | −1.037519 | GZMM | −1.432867 | HLA-DRB5 | −3.691422 |
| BACH1 | 0.7843654 | DUSP4 | −0.763571 | GALNT2 | 0.8197301 | FOXP1 | 1.0857696 |
| ZC3H12D | 0.9663012 | NR4A2 | −1.139392 | DOK2 | 0.7929413 | SH2D2A | −1.90348 |
| TGFB1 | −0.721313 | DNAJA1 | −0.832005 | MCUB | −1.56997 | HLA-DRB1 | −3.782845 |
| CDKN1A | 0.7290113 | HSPE1 | −0.834511 | HLA-DMA | 0.7036764 | OAZ1 | −0.71782 |
| APOBEC3G | −0.706492 | CACYBP | −0.887945 | CYSTM1 | −0.983482 | LDHA | −1.029557 |
| CD8B | −0.812281 | HSPA1A | −1.050586 | MT1E | 0.8692604 | MCL1 | −1.065167 |
| KLRD1 | −1.923841 | HERPUD1 | −0.896744 | TOX | 0.888913 | SRSF7 | −1.296452 |
| PRDM2 | 0.776895 | FABP5 | −0.831347 | IRF7 | −1.241066 | GZMA | −5.034984 |
| RAB9A | 0.7809211 | ARID4B | −0.752786 | RPS26 | −0.843665 | NDFIP1 | 0.8972782 |
| ZC3H7A | 0.7656268 | HSPD1 | −0.884172 | PIM1 | −1.370527 | LINC02446 | 1.3210265 |
| NDFIP2 | 0.6975322 | PSMB9 | −0.71288 | NAB1 | 0.8235167 | HSPH1 | −2.045475 |
| GLRX | 0.8107947 | LDLRAD4 | −0.944606 | TSTD1 | 0.8826885 | TCF7 | 1.0905082 |
| TCF7 | −1.770535 | RHBDD2 | −0.761215 | ICOS | 0.749391 | IL7R | 0.8935882 |
| WDR74 | 0.7107556 | HSPA1B | −2.003591 | PBXIP1 | −0.800521 | S1PR1 | 1.4093698 |
| CFAP20 | 0.7317195 | CALM3 | −0.830392 | TOB1 | 0.703826 | HLA-DRA | −3.093881 |
| ANXA2 | −0.926666 | GATA3 | −0.818299 | MT-ND6 | −0.824951 | FLT3LG | 1.4812326 |
| SRRT | −1.042361 | TNFRSF9 | −0.808518 | IDH2 | 0.7140276 | S100A4 | −2.196373 |
| CRIP1 | −0.808748 | RHOB | −0.852822 | CTLA4 | 0.7640172 | TNFRSF9 | −4.501738 |
| TRAT1 | −0.844491 | CARHSP1 | −0.884901 | ARL4A | 0.7712738 | LSP1 | −1.563162 |
| GGA2 | −1.003806 | EVL | −0.76711 | AHNAK | −0.820969 | HSPA1A | −3.320983 |
| ABLIM1 | −1.241958 | TCP1 | −0.733339 | NEDD9 | 0.7667021 | TNFRSF1B | −2.316395 |
| CD55 | −0.864346 | ITK | −0.904031 | PAG1 | 0.8226945 | SLC7A5 | −2.73668 |
| MBP | −0.954582 | AHSA1 | −0.817192 | UPP1 | −1.074763 | CXCR4 | −1.060418 |
| CYSTM1 | −0.733104 | CCND2 | −0.754277 | FAM129A | −0.813698 | ANXA5 | −2.169945 |
| PLIN2 | 0.727298 | SEPT9 | −0.764052 | TMEM43 | −0.921995 | CXCR3 | −2.209695 |
| MCUB | −0.800417 | | | EEF1G | −0.773201 | YPEL5 | −1.083112 |
| SLA2 | −0.71233 | | | SRRT | −0.806705 | TGFB1 | −1.268486 |
| GZMM | −0.824815 | | | DNPH1 | 0.8255552 | TNFAIP3 | −1.163783 |
| AKNA | −0.716782 | | | VAMP5 | 0.8504424 | PIM2 | 1.1093589 |
| GYG1 | −1.085671 | | | GNPTAB | 0.8415212 | RGS10 | 0.996937 |
| HIST1H4C | −0.782552 | | | HOPX | 0.7612492 | HSPA5 | −1.297112 |
| MX1 | −0.824955 | | | ISG15 | −1.147168 | GZMB | −5.634562 |
| STMN1 | −0.802449 | | | ABLIM1 | −1.16038 | SLA2 | −3.610318 |
| UBE2E3 | −0.712596 | | | AHR | 0.6941654 | DYNLL1 | −1.028731 |
| PRF1 | −0.849655 | | | CRIP1 | −0.835597 | NR4A2 | −2.163147 |
| GPR183 | −0.750799 | | | PNPLA2 | −0.715796 | PIM1 | 0.8475649 |

TABLE 11
Differentially expressed genes among the 12 clusters of CD8+
TILs identified by scRNA-seq (adjusted P value < 0.05)
Cluster 12: Naïve, continued

| gene | avg_logFC | gene | avg_logFC | gene | avg_logFC |
|---|---|---|---|---|---|
| RGS1 | −2.007444 | PLP2 | −0.726551 | MAP2K3 | −1.082561 |
| HNRNPLL | −2.259871 | SLC9A3R1 | −1.140133 | ANXA6 | −1.064213 |
| GZMH | −5.559501 | HLA-DMA | −2.481257 | CAPN2 | −1.26466 |
| LCP1 | −1.556294 | MSL3 | 0.8386496 | PPP1R18 | 0.8211664 |
| EIF3E | 0.7480257 | CHST12 | −1.759284 | ITGAE | −1.331598 |
| GGA2 | −2.239519 | NAA50 | −1.579606 | DNAJA1 | −0.911399 |
| APOBEC3C | −2.871525 | GNG2 | −0.787791 | ADSS | −1.018178 |
| HSP90AB1 | −0.842681 | CYTH1 | 0.7958409 | RAB5IF | −0.852059 |
| MAP1LC3B | −0.997556 | SYTL3 | −1.351878 | VPS37B | −1.185769 |
| LYSMD2 | 0.9326287 | ITGB2 | −1.653024 | ZYX | −0.983006 |
| RAC1 | −1.15406 | CEMIP2 | −1.833311 | GLUD1 | −0.701537 |
| HSPD1 | −1.423043 | ALOX5AP | −2.145966 | CARHSP1 | −1.301667 |
| ACTN4 | −1.808047 | EMD | −0.723956 | IL2RB | −1.202265 |
| ATP6VOC | −0.695837 | AKAP13 | −0.927575 | UBE2A | −0.69899 |
| CCL4 | −3.342685 | BRD2 | −0.725921 | NEAT1 | −0.914139 |
| JUN | −1.025995 | TSPYL2 | −1.058246 | IKZF1 | 0.8186215 |
| TMEM123 | 0.9223339 | HCST | −0.955287 | AKNA | −1.02888 |
| SQSTM1 | −0.772196 | FLNA | −0.945433 | UBE2E3 | −1.301276 |
| IFITM2 | −1.308406 | ITM2C | −1.471574 | TYMP | −1.389915 |
| HMGB2 | −1.978031 | LYST | −1.20753 | LDLRAD4 | −1.308125 |
| JPT1 | −1.326846 | STAT5A | −1.22917 | CCND3 | 0.7625174 |
| ABLIM1 | 0.7356189 | REL | −1.192297 | EML4 | −0.890032 |
| TIGIT | −2.094381 | DNAJC1 | −1.253594 | REEP5 | −0.814462 |
| CYTOR | −2.060321 | MT2A | −1.156412 | ICOS | −1.352758 |
| FAM129A | −1.597514 | IL21R | −1.11014 | CITED2 | −1.229021 |
| CXCL13 | −3.73057 | TSPO | −1.181183 | CD96 | −0.894116 |
| UCP2 | 0.9296114 | FNBP1 | −0.881543 | AKIRIN2 | −0.96075 |
| S100A6 | −0.89108 | SAMHD1 | 0.8639046 | MTHFD2 | −1.12238 |
| TENT5C | −2.349859 | CD58 | −1.565627 | RBPJ | −0.775384 |
| CD82 | −1.639023 | H2AFX | −1.461336 | HERPUD2 | −0.860416 |
| ARID5B | −1.737628 | SURF4 | −0.878596 | AHSA1 | −1.076308 |
| SELENOK | −0.902792 | SSH2 | 0.8570406 | ITGA4 | −0.901328 |
| S100A10 | −0.886804 | CFLAR | −0.890522 | RIC8A | −0.755246 |
| ZEB2 | −2.919207 | CTSC | −1.917763 | PDE4B | −0.886439 |
| SLA | −1.41938 | STAT1 | 0.7308651 | SH3GLB1 | −0.792837 |
| ITM2A | −1.13634 | ATP2B4 | −1.19855 | ITGB1 | −0.958536 |
| VCAM1 | −4.927951 | BHLHE40 | −2.088076 | HMGN2 | −0.766264 |
| DNAJB6 | −1.085269 | BATF | −0.996422 | TANK | −1.086946 |
| FCMR | 1.1462565 | YWHAH | −1.463963 | TPM4 | −1.040981 |
| ZFP36 | −0.765732 | CD8B | −0.726393 | IQGAP1 | −0.705167 |
| CD63 | −1.939733 | PIK3IP1 | 0.7355766 | NFATC2 | −0.884246 |
| HERPUD1 | −1.41317 | SRSF3 | −0.815615 | WAS | −0.982574 |
| EEF1G | 0.8243353 | TERF2IP | −0.833495 | CDKN1A | −1.073679 |
| NEU1 | −1.313181 | SEPT7 | −0.716834 | FOSB | −1.288121 |
| ID2 | −1.651057 | JUND | −0.887742 | SERTAD1 | −0.9228 |
| TESPA1 | 1.0536612 | GATA3 | −1.284797 | STX11 | −1.154706 |
| CD48 | 0.755309 | RHOB | −1.521768 | ZC3H12A | −1.259796 |
| USP3 | 0.9821247 | PTPN22 | −1.524718 | DDIT4 | −0.911404 |
| KLF6 | −0.778219 | SRRT | −1.079212 | BST2 | −0.905632 |
| HSPE1 | −1.287843 | GABARAPL1 | −0.844926 | IDI1 | −1.045088 |
| HLA-DQB1 | −2.287911 | RUNX3 | −0.971094 | APLP2 | −1.037887 |
| IL27RA | 0.8458598 | SLC16A3 | −1.56367 | CSRNP1 | −0.726792 |
| ANXA2 | −1.187192 | NOP58 | −0.758244 | GSPT1 | −0.819141 |
| LITAF | −0.844949 | PSMB9 | −0.835951 | AP3S1 | −0.982555 |
| LSM5 | 0.8522867 | GYG1 | −1.668351 | BIRC3 | −0.753348 |
| LGALS3 | −2.90612 | FABP5 | −1.065026 | RTF1 | −0.975509 |
| FYN | −1.128937 | NAMPT | −1.228629 | DHRS7 | −0.897773 |
| CDK2AP2 | −1.134411 | DENND4A | 0.8164918 | RANBP2 | −0.893724 |
| DOK2 | −1.946339 | CACYBP | −1.032436 | CNN2 | −0.79945 |
| IL32 | −0.999707 | ID3 | −1.895889 | MGAT1 | −0.922266 |
| WHRN | −2.093119 | HNRNPUL1 | −0.753547 | ARPP19 | −0.833464 |
| RARA | 0.7690077 | KPNA2 | −1.209748 | SLC2A3 | −0.746431 |
| PPP1R16B | −2.226482 | JMJD6 | −1.178913 | AC016831,7 | −0.701137 |
| KLRD1 | −2.302709 | DUSP1 | −1.199172 | CLK1 | −0.864406 |
| PHLDA1 | −1.574226 | DNAJB1 | −1.519161 | ZFAND5 | −0.9583 |
| WIPF1 | −0.952528 | GSTP1 | −0.777359 | CNPPD1 | −0.708058 |
| IL10RA | −1.255503 | CARD16 | −1.532169 | CHD1 | −0.754397 |
| HSPA1B | −3.008719 | OPTN | −0.839407 | TUBA1C | −0.717073 |
| TXN | −0.924672 | CCND2 | −1.252502 | CLDND1 | −1.021591 |
| FBL | 0.7342137 | TMX4 | −1.242403 | BTG3 | −0.880437 |
| HSPB1 | −1.719867 | PTP4A1 | −1.082787 | PTGER4 | −0.792996 |
| CRTAM | −2.596637 | PDIA6 | −1.199242 | CHORDC1 | −1.092194 |
| PIK3R1 | −1.668775 | SLBP | −1.037636 | RALGAPA1 | −0.876103 |
| RARRES3 | −0.992413 | SH3KBP1 | −1.212552 | ZFAND2A | −1.239999 |
| GEM | −3.020524 | CD27 | −1.158683 | PAF1 | −0.703059 |
| TMEM243 | 0.7804145 | CLEC2B | −0.728225 | PSMD13 | −0.694526 |
| KLF3 | 0.9862779 | EIF4A3 | −0.906533 | ARPC5L | −0.746693 |
| NR3C1 | −0.916019 | RAB27A | −1.040196 | GOLGB1 | −0.889028 |
| ATP1B3 | −1.059187 | EID1 | −0.883449 | MFSD10 | −0.872084 |

Comparisons with other datasets. The SingleR package was used to compute reference signatures from Sade-Feldman et al., Cell 176:1-20 (2019); Yost et al., Nat. Med. 25:1251-59 (2019); Oh et al., Cell 181:1612-25.e13 (2020)). First, count matrices were downloaded from the gene expression omnibus (GSE120575, GSE139555, and GSE149652, respectively). The scoter package was used to normalize expression values for SingleR, and 10% trimmed means for each gene across cells in clusters classified as CD8-related (Sade-Feldman et al. and Oh et al. datasets) or across cells with CD8A transcript expression (Yost et al dataset) were calculated. These data were used to train SingleR for classification of CD8+ TILs internal dataset upon normalization with the same seater function. Counts of cells based on the internal cluster assignment and external assignments performed by SingleR were computed and normalized as described in (Wu et al., Nature 579:274-8 (2020)), resulting in matrices documenting the similarities between internal and external T cell clusters.

Cluster distribution was analyzed on CD8+ TCRs reported in Sade-Feldman et al. (Sade-Feldman et al., Cell 176:1-20 (2019)). Due to the low number of TCRs available per patient, all CD8+ TCR clonotype families were considered, including TCR singletons.

A general primary cluster was assigned to each TCR clonotype: families with predominance of cells belonging to clusters 1, 2 and 3 in the Sade-Feldman dataset were classified as ‘Exhausted’, while families with preponderance of cells belonging to clusters 4 and 6 were classified as ‘Non-Exhausted’. Correspondence between cluster of the discovery and validation datasets was established unidirectionally by considering CD8+ populations described in Sade-Feldman et al. (Sade-Feldman et al., Cell 176:1-20 (2019)) having the highest correlations with clusters of the discovery cohort defined as T_{Ex} or T_{NExM}. Such information was used to serially trace the dynamics TCR classes within the peripheral blood, as assessed by bulk sequencing of TCRβ-chains.

Gene-signature enrichment analysis. Enrichment of gene-signatures was evaluated on 5 cluster of tumor-specific CD8+ T cells using Seurat's function AddModuleScore with default parameters (24 bins, ctrl=100). Internal signatures of CD8+ TILs consisted of top 100 genes upregulated in each cluster (adj p val <0.05, Table 4-Table 11). Gene-signatures of tumor-specific cells were composed by adding top 50 genes upregulated in each cluster of tumor specific cells (adj p_val<0.0001, log₂FC>0.4) to the core of tumor-specific genes (genes deregulated by all the tumor specific cells, as listed in Table 12). When cluster-specific genes overlapped with those already identified in the core of tumor-specific genes (Table 12), they were removed from such common core. Signatures of tumor specific cells reported in Table 12 were identified as follows: to analyze the subpopulations of tumor-specific CD8+ cells, 7,451 single cells expressing TCRs with in vitro confirmed tumor-specific TCRs (n=134) were normalized and reclustered with a resolution of 0.4 (which granted proper cluster stability). During this procedure, TCR-related genes were removed to avoid clustering artefact produced by the dramatically reduced TCR diversity. Cluster-specific genes were identified with the FindAllMarkers function.

TABLE 12
Differentially expressed genes in tumor-specific
CD8 TILS compared to virus-specific CD8 TILs

| gene | avg_logFC | p_val | p_val_adj | Signature |
|---|---|---|---|---|
| KRT86 | 2.787533396 | 0 | 0 | Tumor-specific |
| RDH10 | 2.251109741 | 0 | 0 | Tumor-specific |
| TYMS | 2.155442162 | 0 | 0 | Tumor-specific |
| HMOX1 | 2.126914736 | 0 | 0 | Tumor-specific |
| GNG4 | 2.044225166 | 0 | 0 | Tumor-specific |
| CXCL13 | 1.99960465 | 0 | 0 | Tumor-specific |
| AFAP1L2 | 1.804323147 | 0 | 0 | Tumor-specific |
| ACP5 | 1.78000315 | 0 | 0 | Tumor-specific |
| MYO1E | 1.756174868 | 1.85E−288 | 6.19E−284 | Tumor-specific |
| LAYN | 1.738202345 | 0 | 0 | Tumor-specific |
| TNS3 | 1.728386017 | 2.50E−274 | 8.40E−270 | Tumor-specific |
| TNFSF4 | 1.723108611 | 0 | 0 | Tumor-specific |
| AKAP5 | 1.704832641 | 0 | 0 | Tumor-specific |
| HAVCR2 | 1.661931261 | 0 | 0 | Tumor-specific |
| ENTPD1 | 1.657229907 | 0 | 0 | Tumor-specific |
| SLC2A8 | 1.592202895 | 0 | 0 | Tumor-specific |
| AC243829.4 | 1.579277373 | 0 | 0 | Tumor-specific |
| ZBED2 | 1.572500169 | 0 | 0 | Tumor-specific |
| MCM5 | 1.531211987 | 0 | 0 | Tumor-specific |
| CAV1 | 1.520917245 | 0 | 0 | Tumor-specific |
| GOLIM4 | 1.484157019 | 8.97E−213 | 3.01E−208 | Tumor-specific |
| TRAV21 | 1.481423493 | 1.63E−127 | 5.45E−123 | Tumor-specific |
| VCAM1 | 1.463531862 | 0 | 0 | Tumor-specific |
| PON2 | 1.44666638 | 9.58E−293 | 3.21E−288 | Tumor-specific |
| MTSS1 | 1.399193134 | 0 | 0 | Tumor-specific |
| CD38 | 1.343692172 | 0 | 0 | Tumor-specific |
| TRBV11-2 | 1.342660596 | 2.20E−131 | 7.38E−127 | Tumor-specific |
| MS4A6A | 1.340638727 | 0 | 0 | Tumor-specific |
| TOX2 | 1.325553691 | 3.46E−222 | 1.16E−217 | Tumor-specific |
| CSF1 | 1.307418821 | 4.17E−227 | 1.40E−222 | Tumor-specific |
| GALNT2 | 1.30705988 | 0 | 0 | Tumor-specific |
| FXYD2 | 1.306917186 | 2.90E−120 | 9.74E−116 | Tumor-specific |
| PLPP1 | 1.303412174 | 0 | 0 | Tumor-specific |
| LMCD1 | 1.279380309 | 7.91E−215 | 2.65E−210 | Tumor-specific |
| MYL6B | 1.272331736 | 7.02E−257 | 2.36E−252 | Tumor-specific |
| LAG3 | 1.258756326 | 0 | 0 | Tumor-specific |
| HLA-DRA | 1.25714647 | 0 | 0 | Tumor-specific |
| IGFLR1 | 1.255670648 | 0 | 0 | Tumor-specific |
| CCDC50 | 1.240690888 | 7.44E−182 | 2.49E−177 | Tumor-specific |
| CD27 | 1.233091896 | 0 | 0 | Tumor-specific |
| KIAA1324 | 1.229974839 | 5.09E−271 | 1.71E−266 | Tumor-specific |
| CDKN2A | 1.226923799 | 1.36E−242 | 4.56E−238 | Tumor-specific |
| CD70 | 1.220048716 | 0 | 0 | Tumor-specific |
| ABHD6 | 1.204736708 | 9.37E−201 | 3.14E−196 | Tumor-specific |
| CTLA4 | 1.183326931 | 0 | 0 | Tumor-specific |
| PDCD1 | 1.181586505 | 0 | 0 | Tumor-specific |
| GEM | 1.174205174 | 0 | 0 | Tumor-specific |
| NUSAP1 | 1.167439319 | 2.14E−273 | 7.17E−269 | Tumor-specific |
| TOX | 1.162922383 | 0 | 0 | Tumor-specific |
| CXCR6 | 1.159615457 | 8.85E−280 | 2.97E−275 | Tumor-specific |
| NMB | 1.154944308 | 2.28E−179 | 7.66E−175 | Tumor-specific |
| HOPX | 1.139465174 | 3.68E−246 | 1.23E−241 | Tumor-specific |
| CLIC3 | 1.13679439 | 1.07E−164 | 3.60E−160 | Tumor-specific |
| INPP5F | 1.134360649 | 2.04E−287 | 6.84E−283 | Tumor-specific |
| SNAP47 | 1.123479432 | 2.77E−250 | 9.28E−246 | Tumor-specific |
| TSHZ2 | 1.115501418 | 0 | 0 | Tumor-specific |
| HLA-DMA | 1.11390903 | 0 | 0 | Tumor-specific |
| SIT1 | 1.112726735 | 4.23E−256 | 1.42E−251 | Tumor-specific |
| HLA-DRB1 | 1.110296351 | 0 | 0 | Tumor-specific |
| TUBB | 1.106303696 | 1.54E−140 | 5.18E−136 | Tumor-specific |
| PYCARD | 1.086766852 | 1.66E−215 | 5.58E−211 | Tumor-specific |
| ADGRG1 | 1.083457585 | 7.55E−214 | 2.53E−209 | Tumor-specific |
| HLA-DQA1 | 1.082080048 | 0 | 0 | Tumor-specific |
| PRF1 | 1.078637206 | 0 | 0 | Tumor-specific |
| HLA-DPA1 | 1.075967448 | 0 | 0 | Tumor-specific |
| PTMS | 1.071661863 | 0 | 0 | Tumor-specific |
| CKS1B | 1.060237579 | 5.36E−142 | 1.80E−137 | Tumor-specific |
| HIPK2 | 1.049170081 | 8.07E−154 | 2.71E−149 | Tumor-specific |
| CHST12 | 1.037208651 | 0 | 0 | Tumor-specific |
| LSP1 | 1.036849405 | 0 | 0 | Tumor-specific |
| FAM3C | 1.034687412 | 7.76E−184 | 2.60E−179 | Tumor-specific |
| SLC1A4 | 1.023173656 | 3.95E−122 | 1.32E−117 | Tumor-specific |
| NUDT1 | 1.003708412 | 3.11E−176 | 1.04E−171 | Tumor-specific |
| DNPH1 | 1.000665255 | 0 | 0 | Tumor-specific |
| CARD16 | 0.997471171 | 0 | 0 | |
| MT1E | 0.988757378 | 5.61E−285 | 1.88E−280 | |
| GZMB | 0.987312438 | 0 | 0 | |
| CHN1 | 0.983060871 | 1.52E−225 | 5.11E−221 | |
| LGALS1 | 0.98223827 | 0 | 0 | |
| TRBV27 | 0.969798864 | 1.28E−109 | 4.30E−105 | |
| HSPB1 | 0.961817044 | 0 | 0 | |
| SIRPG | 0.950770389 | 5.13E−227 | 1.72E−222 | |
| INPP1 | 0.94941514 | 2.00E−144 | 6.72E−140 | |
| SEMA4A | 0.941956779 | 3.34E−181 | 1.12E−176 | |
| CENPM | 0.9370346 | 7.78E−145 | 2.61E−140 | |
| CD82 | 0.932357112 | 0 | 0 | |
| POLRIE | 0.928952211 | 9.10E−111 | 3.05E−106 | |
| IFI27L2 | 0.927583596 | 0 | 0 | |
| MPST | 0.922021111 | 3.93E−134 | 1.32E−129 | |
| HMGN3 | 0.921174909 | 9.59E−286 | 3.21E−281 | |
| CD74 | 0.920787748 | 0 | 0 | |
| UBE2F | 0.918172155 | 3.39E−95 | 1.14E−90 | |
| CASP1 | 0.917927283 | 1.77E−165 | 5.93E−161 | |
| RIN3 | 0.917160148 | 1.35E−126 | 4.51E−122 | |
| SYNGR2 | 0.916156023 | 0 | 0 | |
| TNFRSF18 | 0.915844834 | 6.60E−293 | 2.22E−288 | |
| STMN1 | 0.914275803 | 1.43E−122 | 4.80E−118 | |
| HINT2 | 0.912175661 | 4.66E−147 | 1.56E−142 | |
| NKG7 | 0.909800595 | 0 | 0 | |
| LINC01871 | 0.907029503 | 4.07E−122 | 1.36E−117 | |
| EZH2 | 0.905675719 | 8.43E−183 | 2.83E−178 | |
| HLA-DPB1 | 0.88903701 | 0 | 0 | |
| HMGB2 | 0.886359151 | 0 | 0 | |
| DUT | 0.879168785 | 1.25E−161 | 4.18E−157 | |
| TNFSF10 | 0.878363609 | 3.52E−266 | 1.18E−261 | |
| PHPT1 | 0.877377109 | 1.34E−287 | 4.51E−283 | |
| CTSW | 0.87285765 | 0 | 0 | |
| PPM1M | 0.871888734 | 5.86E−138 | 1.96E−133 | |
| CTSB | 0.86277139 | 8.72E−276 | 2.93E−271 | |
| TMEM106C | 0.857917665 | 1.91E−109 | 6.41E−105 | |
| CD151 | 0.85550404 | 6.51E−130 | 2.18E−125 | |
| MT1F | 0.854010301 | 1.52E−196 | 5.08E−192 | |
| PSMB9 | 0.853165514 | 0 | 0 | |
| SPATS2L | 0.848342658 | 1.81E−130 | 6.06E−126 | |
| SQLE | 0.847140329 | 4.06E−213 | 1.36E−208 | |
| HMGN2 | 0.84394579 | 0 | 0 | |
| BATF | 0.843922177 | 0 | 0 | |
| TNFRSF9 | 0.842152482 | 0 | 0 | |
| SERPINH1 | 0.841341462 | 1.55E−153 | 5.20E−149 | |
| PAM | 0.840073836 | 5.18E−210 | 1.74E−205 | |
| ZBTB32 | 0.839424584 | 3.33E−126 | 1.12E−121 | |
| LIMA1 | 0.837324591 | 1.58E−97 | 5.28E−93 | |
| BST2 | 0.832749216 | 0 | 0 | |
| WARS | 0.830670229 | 3.14E−104 | 1.05E−99 | |
| MCM7 | 0.830464987 | 1.60E−118 | 5.38E−114 | |
| AHI1 | 0.822095649 | 4.69E−302 | 1.57E−297 | |
| HLA-DRB5 | 0.821684857 | 0 | 0 | |
| TMPO | 0.82123987 | 1.78E−218 | 5.97E−214 | |
| RAB27A | 0.820436069 | 0 | 0 | |
| IDH2 | 0.819666087 | 1.83E−268 | 6.12E−264 | |
| SCCPDH | 0.81544616 | 5.54E−100 | 1.86E−95 | |
| SMC4 | 0.813446261 | 4.86E−244 | 1.63E−239 | |
| TSPO | 0.808024038 | 0 | 0 | |
| RAB11FIP1 | 0.806211133 | 0 | 0 | |
| SKA2 | 0.805617088 | 2.43E−94 | 8.16E−90 | |
| ANXA6 | 0.805353064 | 0 | 0 | |
| LINC01943 | 0.805034213 | 3.24E−124 | 1.09E−119 | |
| CENPX | 0.804314201 | 1.21E−135 | 4.07E−131 | |
| ATP6V0E2 | 0.80386301 | 2.52E−141 | 8.45E−137 | |
| ARL3 | 0.796933638 | 1.18E−125 | 3.95E−121 | |
| ID3 | 0.796614502 | 0 | 0 | |
| BCAS4 | 0.792518939 | 1.05E−107 | 3.52E−103 | |
| TMEM9 | 0.79192907 | 1.20E−91 | 4.01E−87 | |
| HAPLN3 | 0.787690607 | 3.17E−94 | 1.06E−89 | |
| MTHFD2 | 0.787315139 | 9.09E−304 | 3.05E−299 | |
| SPN | 0.776795453 | 9.09E−283 | 3.05E−278 | |
| DUSP5 | 0.774802648 | 7.83E−158 | 2.63E−153 | |
| NAB1 | 0.77257273 | 3.98E−200 | 1.34E−195 | |
| CARS | 0.764862773 | 2.05E−118 | 6.87E−114 | |
| FABP5 | 0.763817864 | 0 | 0 | |
| VAMP5 | 0.763355139 | 1.85E−157 | 6.19E−153 | |
| PTTG1 | 0.762284612 | 3.08E−220 | 1.03E−215 | |
| LSM2 | 0.753293303 | 1.19E−213 | 4.00E−209 | |
| ZBTB38 | 0.748366808 | 1.92E−220 | 6.43E−216 | |
| BLVRA | 0.74711535 | 1.13E−97 | 3.80E−93 | |
| PRDM1 | 0.7453314 | 5.93E−185 | 1.99E−180 | |
| CENPF | 0.73344933 | 1.89E−122 | 6.34E−118 | |
| RUNX2 | 0.731520755 | 6.11E−86 | 2.05E−81 | |
| CD63 | 0.730260108 | 1.12E−306 | 3.75E−302 | |
| MBD2 | 0.729783142 | 1.95E−79 | 6.53E−75 | |
| DUSP4 | 0.725281371 | 0 | 0 | |
| CAT | 0.720475056 | 2.21E−117 | 7.40E−113 | |
| RGS1 | 0.719156171 | 0 | 0 | |
| CARHSP1 | 0.718909807 | 9.58E−245 | 3.21E−240 | |
| NBL1 | 0.718323858 | 9.32E−126 | 3.13E−121 | |
| GALM | 0.717546123 | 4.18E−241 | 1.40E−236 | |
| ETFB | 0.717072014 | 4.57E−83 | 1.53E−78 | |
| TYMP | 0.711934594 | 6.58E−241 | 2.21E−236 | |
| TSTD1 | 0.711214013 | 3.86E−192 | 1.29E−187 | |
| NAA38 | 0.710098999 | 6.69E−121 | 2.24E−116 | |
| PRDX5 | 0.707416037 | 0 | 0 | |
| TNIP3 | 0.705855651 | 4.25E−91 | 1.42E−86 | |
| CALM3 | 0.702292021 | 0 | 0 | |
| ACSL1 | 0.701250573 | 2.42E−72 | 8.13E−68 | |
| TICAM1 | 0.697193545 | 1.07E−87 | 3.58E−83 | |
| CCND2 | 0.697005631 | 6.76E−277 | 2.27E−272 | |
| ACOT9 | 0.696614439 | 8.02E−107 | 2.69E−102 | |
| DUSP16 | 0.692199914 | 5.97E−117 | 2.00E−112 | |
| PAFAH1B3 | 0.692078916 | 2.87E−77 | 9.63E−73 | |
| FIBP | 0.691099001 | 5.39E−134 | 1.81E−129 | |
| MPG | 0.687885084 | 8.87E−135 | 2.97E−130 | |
| TRAF5 | 0.687802195 | 9.87E−108 | 3.31E−103 | |
| CCL3 | 0.687486075 | 8.62E−187 | 2.89E−182 | |
| PCNA | 0.684546458 | 3.09E−95 | 1.04E−90 | |
| SEC14L1 | 0.683979171 | 3.84E−234 | 1.29E−229 | |
| GMNN | 0.683854606 | 8.45E−86 | 2.83E−81 | |
| MSI2 | 0.682575503 | 8.12E−142 | 2.72E−137 | |
| SNX9 | 0.682462567 | 0 | 0 | |
| SHMT2 | 0.681746109 | 1.50E−110 | 5.05E−106 | |
| SFT2D1 | 0.68005122 | 1.72E−131 | 5.77E−127 | |
| GATA3 | 0.678637554 | 0 | 0 | |
| JOSD2 | 0.678274559 | 1.31E−87 | 4.39E−83 | |
| RALA | 0.670122939 | 0 | 0 | |
| CKS2 | 0.665762879 | 3.90E−184 | 1.31E−179 | |
| PLA2G16 | 0.663799837 | 4.32E−132 | 1.45E−127 | |
| HMGB3 | 0.653724154 | 5.50E−73 | 1.84E−68 | |
| YEATS4 | 0.652095425 | 2.43E−70 | 8.14E−66 | |
| HIST1H1E | 0.651088684 | 2.77E−100 | 9.29E−96 | |
| TMX4 | 0.650859913 | 4.27E−228 | 1.43E−223 | |
| SH3BGRL | 0.650453272 | 6.23E−231 | 2.09E−226 | |
| H2AFY | 0.650201723 | 4.13E−154 | 1.38E−149 | |
| GK | 0.648958047 | 3.94E−147 | 1.32E−142 | |
| JAKMIP1 | 0.648585172 | 1.29E−87 | 4.34E−83 | |
| TMEM14C | 0.648206642 | 1.91E−76 | 6.42E−72 | |
| CHPF | 0.647437657 | 1.62E−62 | 5.43E−58 | |
| FEN1 | 0.647077536 | 4.69E−55 | 1.57E−50 | |
| PLEKHF1 | 0.647037311 | 2.58E−62 | 8.66E−58 | |
| GZMA | 0.644387465 | 0 | 0 | |
| USP18 | 0.642639769 | 6.81E−64 | 2.28E−59 | |
| GFOD1 | 0.638512872 | 3.57E−121 | 1.20E−116 | |
| HSPD1 | 0.637634678 | 0 | 0 | |
| TMEM256 | 0.630027524 | 3.80E−104 | 1.27E−99 | |
| GBP4 | 0.629311677 | 6.57E−125 | 2.20E−120 | |
| PFKL | 0.625191626 | 4.90E−96 | 1.64E−91 | |
| SLC27A2 | 0.623493994 | 1.14E−67 | 3.83E−63 | |
| LY6E | 0.622558517 | 0 | 0 | |
| PNKD | 0.621409199 | 3.52E−83 | 1.18E−78 | |
| CORO1B | 0.62016619 | 3.67E−125 | 1.23E−120 | |
| CHST11 | 0.620066936 | 2.77E−106 | 9.30E−102 | |
| GRAMD1A | 0.619721838 | 1.03E−88 | 3.44E−84 | |
| ICOS | 0.618753975 | 1.05E−271 | 3.52E−267 | |
| AGPAT2 | 0.617513786 | 1.65E−88 | 5.54E−84 | |
| VOPP1 | 0.617265057 | 1.68E−127 | 5.62E−123 | |
| RGS2 | 0.615083653 | 0 | 0 | |
| EIF4EBP1 | 0.61392208 | 1.54E−116 | 5.15E−112 | |
| DCPS | 0.613230337 | 8.16E−55 | 2.74E−50 | |
| ATP5MC2 | 0.611534118 | 0 | 0 | |
| CUEDC2 | 0.61013866 | 1.16E−73 | 3.90E−69 | |
| SLC39A1 | 0.610039511 | 7.42E−73 | 2.49E−68 | |
| AKR7A2 | 0.609826581 | 8.63E−78 | 2.89E−73 | |
| CRTAM | 0.609559694 | 6.34E−104 | 2.13E−99 | |
| HSP90AA1 | 0.608264669 | 0 | 0 | |
| FKBP1A | 0.606539033 | 0 | 0 | |
| LSM4 | 0.602847318 | 1.84E−116 | 6.19E−112 | |
| ITM2A | 0.60191659 | 0 | 0 | |
| PLSCR1 | 0.601147295 | 1.90E−125 | 6.36E−121 | |
| PDE4DIP | 0.598989463 | 8.44E−114 | 2.83E−109 | |
| DNAJC4 | 0.598307254 | 6.66E−102 | 2.23E−97 | |
| PFN1 | 0.597999283 | 0 | 0 | |
| PSMB10 | 0.597714535 | 3.50E−178 | 1.17E−173 | |
| YIF1B | 0.596949239 | 4.62E−69 | 1.55E−64 | |
| ITGB7 | 0.596604791 | 1.65E−125 | 5.52E−121 | |
| PHTF1 | 0.593949506 | 1.98E−72 | 6.64E−68 | |
| PCED1B | 0.592425174 | 4.16E−93 | 1.40E−88 | |
| NDUFS8 | 0.590381964 | 1.74E−146 | 5.85E−142 | |
| BANF1 | 0.587339974 | 1.12E−170 | 3.75E−166 | |
| MIR155HG | 0.58657619 | 1.21E−184 | 4.04E−180 | |
| NUCB1 | 0.584803233 | 1.97E−149 | 6.62E−145 | |
| TRIM69 | 0.584344444 | 7.27E−73 | 2.44E−68 | |
| SNRNP25 | 0.584262372 | 1.54E−44 | 5.18E−40 | |
| BLOC1S1 | 0.583110721 | 3.58E−61 | 1.20E−56 | |
| BANP | 0.581966898 | 9.09E−118 | 3.05E−113 | |
| PSME2 | 0.581004034 | 5.37E−302 | 1.80E−297 | |
| ABI3 | 0.580217657 | 1.35E−91 | 4.51E−87 | |
| TFPT | 0.578375661 | 1.04E−58 | 3.48E−54 | |
| MCRIP1 | 0.576600482 | 4.51E−98 | 1.51E−93 | |
| HLA-DQB1 | 0.576176708 | 1.16E−152 | 3.88E−148 | |
| IFI6 | 0.57598701 | 2.18E−156 | 7.31E−152 | |
| ACAT2 | 0.572185642 | 5.04E−67 | 1.69E−62 | |
| TMED3 | 0.571555428 | 3.20E−62 | 1.07E−57 | |
| ITGAE | 0.571522006 | 1.51E−156 | 5.07E−152 | |
| ACTG1 | 0.571314257 | 0 | 0 | |
| PCBD1 | 0.571042437 | 9.50E−51 | 3.19E−46 | |
| DECR1 | 0.570511203 | 3.76E−77 | 1.26E−72 | |
| FKBP4 | 0.570267136 | 2.11E−172 | 7.08E−168 | |
| STIP1 | 0.569378344 | 2.20E−221 | 7.38E−217 | |
| N4BP2L1 | 0.567680081 | 6.36E−79 | 2.13E−74 | |
| LAGE3 | 0.567622438 | 2.36E−118 | 7.92E−114 | |
| CCDC117 | 0.565864365 | 1.96E−57 | 6.56E−53 | |
| PRDX3 | 0.564883857 | 2.03E−99 | 6.79E−95 | |
| COMT | 0.563683568 | 2.92E−70 | 9.79E−66 | |
| RHBDD2 | 0.56290247 | 3.90E−288 | 1.31E−283 | |
| COX8A | 0.562593466 | 0 | 0 | |
| YPEL2 | 0.562253925 | 2.63E−48 | 8.83E−44 | |
| HSPE1 | 0.559644133 | 0 | 0 | |
| EID1 | 0.559571923 | 1.06E−241 | 3.55E−237 | |
| REX1BD | 0.558173362 | 3.86E−133 | 1.29E−128 | |
| GNPTAB | 0.558107108 | 5.35E−70 | 1.80E−65 | |
| DGKZ | 0.557857658 | 5.21E−120 | 1.75E−115 | |
| IFI35 | 0.556067085 | 1.08E−100 | 3.63E−96 | |
| PXK | 0.554678977 | 4.07E−41 | 1.37E−36 | |
| OAS1 | 0.554651058 | 2.32E−80 | 7.80E−76 | |
| TANK | 0.553942713 | 4.73E−187 | 1.59E−182 | |
| CCDC6 | 0.553551696 | 5.50E−75 | 1.84E−70 | |
| RBPJ | 0.553488948 | 2.24E−204 | 7.51E−200 | |
| PDIA6 | 0.553140823 | 4.57E−156 | 1.53E−151 | |
| PAXX | 0.548752143 | 3.66E−178 | 1.23E−173 | |
| ZCRB1 | 0.548389632 | 4.69E−66 | 1.57E−61 | |
| CMPK2 | 0.547634635 | 2.91E−59 | 9.77E−55 | |
| PPM1G | 0.546299661 | 1.06E−263 | 3.57E−259 | |
| BHLHE40 | 0.545832977 | 1.31E−209 | 4.40E−205 | |
| CSNK1G3 | 0.545390853 | 2.58E−89 | 8.64E−85 | |
| ENC1 | 0.544825449 | 1.12E−105 | 3.75E−101 | |
| HIST1H1C | 0.543817102 | 1.23E−88 | 4.13E−84 | |
| ACTB | 0.543274484 | 0 | 0 | |
| COTL1 | 0.542061249 | 0 | 0 | |
| PHLDA1 | 0.540795645 | 1.33E−300 | 4.47E−296 | |
| NUCKS1 | 0.539923611 | 1.59E−105 | 5.32E−101 | |
| WAS | 0.538130126 | 2.48E−172 | 8.32E−168 | |
| NDFIP2 | 0.53796316 | 5.60E−68 | 1.88E−63 | |
| MYL12A | 0.537187587 | 1.31E−285 | 4.39E−281 | |
| OAS3 | 0.535665239 | 9.22E−88 | 3.09E−83 | |
| SLC25A46 | 0.534410642 | 2.90E−89 | 9.74E−85 | |
| NABP2 | 0.532642379 | 1.71E−45 | 5.74E−41 | |
| SLA2 | 0.532275149 | 4.57E−141 | 1.53E−136 | |
| IFITM2 | 0.530663825 | 1.47E−264 | 4.92E−260 | |
| NBDY | 0.529294741 | 8.10E−153 | 2.72E−148 | |
| LMNB1 | 0.529075272 | 2.74E−91 | 9.18E−87 | |
| ACOT7 | 0.528509761 | 6.83E−50 | 2.29E−45 | |
| CACYBP | 0.526589394 | 2.69E−266 | 9.03E−262 | |
| STMP1 | 0.526395286 | 1.58E−89 | 5.28E−85 | |
| AKR1A1 | 0.525993424 | 1.03E−64 | 3.44E−60 | |
| STAMBP | 0.525449792 | 1.72E−51 | 5.76E−47 | |
| C16orf87 | 0.524749411 | 6.04E−62 | 2.02E−57 | |
| PSME1 | 0.524368536 | 0 | 0 | |
| ATOX1 | 0.523822035 | 1.86E−52 | 6.24E−48 | |
| OTULIN | 0.523393001 | 3.96E−66 | 1.33E−61 | |
| CASP7 | 0.52103081 | 3.52E−54 | 1.18E−49 | |
| PPDPF | 0.520960573 | 0 | 0 | |
| CCL5 | 0.52092453 | 0 | 0 | |
| URM1 | 0.519671022 | 2.26E−64 | 7.56E−60 | |
| DOK2 | 0.518259067 | 2.04E−174 | 6.84E−170 | |
| C9orf16 | 0.518226227 | 1.04E−211 | 3.48E−207 | |
| ARF5 | 0.517516514 | 1.57E−214 | 5.28E−210 | |
| MAP4K1 | 0.517305616 | 8.85E−90 | 2.97E−85 | |
| PSTPIP1 | 0.516740812 | 1.48E−131 | 4.97E−127 | |
| RAC2 | 0.516396351 | 0 | 0 | |
| DRAP1 | 0.516245058 | 2.22E−263 | 7.45E−259 | |
| BAX | 0.515408376 | 1.04E−121 | 3.50E−117 | |
| NUCB2 | 0.515193814 | 2.04E−97 | 6.85E−93 | |
| DYNLRB1 | 0.515110371 | 3.92E−173 | 1.31E−168 | |
| PSMB8 | 0.514605084 | 8.76E−292 | 2.94E−287 | |
| NAP1L4 | 0.513631715 | 4.29E−125 | 1.44E−120 | |
| H1FX | 0.512148039 | 1.84E−75 | 6.18E−71 | |
| FASLG | 0.511772921 | 6.06E−99 | 2.03E−94 | |
| HIKESHI | 0.511755894 | 9.55E−64 | 3.20E−59 | |
| JAK3 | 0.510903099 | 5.42E−87 | 1.82E−82 | |
| PARK7 | 0.510194063 | 3.06E−284 | 1.03E−279 | |
| RARRES3 | 0.509735139 | 3.15E−278 | 1.06E−273 | |
| ELMO1 | 0.508324501 | 2.06E−107 | 6.92E−103 | |
| TRAFD1 | 0.50819736 | 2.97E−64 | 9.96E−60 | |
| GPAA1 | 0.507909965 | 8.15E−63 | 2.73E−58 | |
| ATP5MC1 | 0.507011546 | 2.01E−83 | 6.75E−79 | |
| EML2 | 0.506733955 | 3.91E−77 | 1.31E−72 | |
| ANXA5 | 0.504553818 | 2.27E−235 | 7.61E−231 | |
| FAM122C | 0.502543338 | 6.01E−37 | 2.02E−32 | |
| MAD2L2 | 0.50171398 | 8.27E−63 | 2.77E−58 | |
| MDH2 | 0.50136364 | 1.23E−160 | 4.13E−156 | |
| CCNB1IP1 | 0.50052214 | 4.90E−48 | 1.64E−43 | |
| PKM | 0.500352373 | 2.28E−279 | 7.66E−275 | |
| CAPZB | 0.500076178 | 1.29E−253 | 4.33E−249 | |
| EML4 | −0.501614907 | 1.32E−123 | 4.44E−119 | |
| RPL38 | −0.50453357 | 0 | 0 | |
| MGAT4A | −0.504674069 | 6.31E−86 | 2.12E−81 | |
| NR4A3 | −0.506264259 | 3.17E−32 | 1.06E−27 | |
| YIPF5 | −0.514148282 | 3.73E−115 | 1.25E−110 | |
| HTATIP2 | −0.514301925 | 2.83E−47 | 9.48E−43 | |
| NDUFAF4 | −0.516864766 | 2.19E−39 | 7.33E−35 | |
| CDKNIA | −0.5191157 | 1.68E−95 | 5.64E−91 | |
| SETD2 | −0.520107488 | 3.09E−93 | 1.04E−88 | |
| PNP | −0.520946747 | 1.82E−109 | 6.09E−105 | |
| RPS10 | −0.525169201 | 8.55E−80 | 2.87E−75 | |
| EPB41 | −0.52752689 | 4.38E−47 | 1.47E−42 | |
| OGDH | −0.527759713 | 7.58E−76 | 2.54E−71 | |
| AC020916.1 | −0.527800823 | 1.32E−14 | 4.42E−10 | |
| SYPL1 | −0.532663082 | 4.76E−103 | 1.60E−98 | |
| RNF145 | −0.534676512 | 4.38E−85 | 1.47E−80 | |
| SLC2A3 | −0.536765476 | 2.56E−54 | 8.58E−50 | |
| MFSD11 | −0.53914332 | 1.43E−43 | 4.80E−39 | |
| RPS27 | −0.539815448 | 0 | 0 | |
| S1PR4 | −0.542520144 | 4.11E−86 | 1.38E−81 | |
| TNIK | −0.542529569 | 8.81E−56 | 2.95E−51 | |
| ITGAV | −0.543772798 | 4.41E−45 | 1.48E−40 | |
| TRMT6 | −0.547994017 | 1.43E−62 | 4.79E−58 | |
| TUBB2A | −0.548112666 | 9.60E−65 | 3.22E−60 | |
| DENND4A | −0.551235848 | 3.54E−44 | 1.19E−39 | |
| ATP1A1 | −0.553398013 | 2.52E−188 | 8.46E−184 | |
| FASN | −0.55430111 | 2.70E−32 | 9.07E−28 | |
| RPS12 | −0.556858453 | 0 | 0 | |
| DDIT4 | −0.566284711 | 5.68E−83 | 1.91E−78 | |
| TXNIP | −0.575809312 | 1.57E−27 | 5.27E−23 | |
| PLK3 | −0.579873326 | 9.25E−131 | 3.10E−126 | |
| NFATC2 | −0.581057183 | 2.44E−146 | 8.17E−142 | |
| RPL27A | −0.58261166 | 0 | 0 | |
| RPL36A | −0.586820581 | 2.02E−232 | 6.79E−228 | |
| HIVEP2 | −0.588487998 | 9.81E−35 | 3.29E−30 | |
| CD28 | −0.590859181 | 3.16E−77 | 1.06E−72 | |
| TMEM123 | −0.593700127 | 7.86E−113 | 2.64E−108 | |
| PABPC1 | −0.59589576 | 0 | 0 | |
| CAMK1D | −0.596307178 | 1.63E−61 | 5.46E−57 | |
| CD44 | −0.596936561 | 0 | 0 | |
| PDE4B | −0.600328055 | 2.76E−198 | 9.25E−194 | |
| GABARAPL1 | −0.602604854 | 2.99E−277 | 1.00E−272 | |
| PMEPA1 | −0.610179399 | 3.73E−78 | 1.25E−73 | |
| FLOT1 | −0.612813409 | 7.93E−44 | 2.66E−39 | |
| LYAR | −0.617585073 | 2.41E−108 | 8.07E−104 | |
| PITPNC1 | −0.622001671 | 1.38E−98 | 4.64E−94 | |
| RASA3 | −0.622732395 | 2.90E−49 | 9.74E−45 | |
| YBX3 | −0.624826163 | 3.60E−126 | 1.21E−121 | |
| ANXA2 | −0.625993397 | 4.31E−266 | 1.44E−261 | |
| MPZL3 | −0.626246053 | 1.40E−77 | 4.69E−73 | |
| S100A10 | −0.628296838 | 0 | 0 | |
| STK38 | −0.629797419 | 5.70E−64 | 1.91E−59 | |
| PTGER4 | −0.633366244 | 1.12E−140 | 3.76E−136 | |
| UPP1 | −0.633823848 | 2.06E−177 | 6.91E−173 | |
| FTH1 | −0.63516818 | 0 | 0 | |
| CA5B | −0.635838084 | 3.90E−76 | 1.31E−71 | |
| PIM1 | −0.637291054 | 4.41E−164 | 1.48E−159 | |
| GLIPR1 | −0.63959124 | 1.03E−157 | 3.44E−153 | |
| FLT3LG | −0.640799762 | 2.57E−60 | 8.61E−56 | |
| FOXP1 | −0.641143893 | 2.04E−173 | 6.86E−169 | |
| TIPARP | −0.641510678 | 2.68E−62 | 8.98E−58 | |
| CLINT1 | −0.645304684 | 1.30E−59 | 4.37E−55 | |
| SSH2 | −0.651855277 | 2.70E−73 | 9.06E−69 | |
| TGFBR3 | −0.652175545 | 1.90E−76 | 6.36E−72 | |
| ABLIM1 | −0.656242176 | 1.30E−159 | 4.35E−155 | |
| RPS29 | −0.656620604 | 0 | 0 | |
| PBX4 | −0.65663684 | 1.21E−140 | 4.05E−136 | |
| RIPOR2 | −0.656711707 | 8.64E−65 | 2.90E−60 | |
| LINC02446 | −0.673116409 | 4.27E−51 | 1.43E−46 | |
| FAM102A | −0.678643395 | 2.44E−169 | 8.17E−165 | |
| KLF10 | −0.680889019 | 7.12E−45 | 2.39E−40 | |
| STOM | −0.6881398 | 6.12E−65 | 2.05E−60 | |
| CRIP2 | −0.690299497 | 2.35E−66 | 7.88E−62 | |
| GCLM | −0.694506209 | 1.19E−105 | 4.00E−101 | |
| AHNAK | −0.70010337 | 0 | 0 | |
| PRNP | −0.712306695 | 5.82E−288 | 1.95E−283 | |
| CDC42EP3 | −0.712605013 | 3.55E−140 | 1.19E−135 | |
| TES | −0.715637923 | 1.32E−171 | 4.41E−167 | |
| SLC25A4 | −0.720544326 | 4.16E−71 | 1.40E−66 | |
| MARCKSL1 | −0.722816742 | 9.78E−290 | 3.28E−285 | |
| BNIP3 | −0.727798278 | 4.32E−71 | 1.45E−66 | |
| SLAMF1 | −0.740232652 | 1.25E−46 | 4.20E−42 | |
| STAT4 | −0.744710681 | 1.04E−154 | 3.49E−150 | |
| GZMM | −0.752270824 | 2.59E−272 | 8.67E−268 | |
| GPR171 | −0.753178846 | 3.23E−60 | 1.08E−55 | |
| TLE4 | −0.756947357 | 2.20E−59 | 7.39E−55 | |
| BCL2A1 | −0.77822813 | 1.64E−89 | 5.49E−85 | |
| TRMO | −0.802453725 | 5.25E−55 | 1.76E−50 | |
| PIK3R1 | −0.816812088 | 7.27E−296 | 2.44E−291 | |
| CRYBG1 | −0.81968081 | 4.16E−168 | 1.40E−163 | |
| KCNA3 | −0.829840804 | 7.41E−76 | 2.48E−71 | |
| LMNA | −0.841663534 | 0 | 0 | |
| GPR132 | −0.856584822 | 1.46E−121 | 4.89E−117 | |
| KLF2 | −0.858651409 | 8.07E−131 | 2.71E−126 | |
| HMGA1 | −0.863267569 | 4.41E−249 | 1.48E−244 | |
| VIM | −0.86357162 | 0 | 0 | |
| SAMD3 | −0.863649844 | 1.42E−88 | 4.75E−84 | |
| CD55 | −0.918174016 | 0 | 0 | |
| ANKH | −0.929692764 | 1.20E−107 | 4.04E−103 | |
| LTB | −0.930676413 | 2.97E−196 | 9.98E−192 | |
| P2RY8 | −0.960748826 | 9.08E−199 | 3.04E−194 | |
| FOS | −0.966493749 | 5.91E−145 | 1.98E−140 | |
| AQP3 | −0.994829075 | 2.38E−128 | 7.97E−124 | |
| MCUB | −0.995906489 | 0 | 0 | |
| RARA | −1.017161878 | 3.17E−269 | 1.06E−264 | Virus-specific |
| GADD45B | −1.020159953 | 1.84E−241 | 6.16E−237 | Virus-specific |
| Clorf21 | −1.082633678 | 2.38E−141 | 7.99E−137 | Virus-specific |
| AOAH | −1.104377499 | 1.01E−99 | 3.38E−95 | Virus-specific |
| MATK | −1.147629494 | 1.11E−207 | 3.71E−203 | Virus-specific |
| SATB1 | −1.203581963 | 9.15E−221 | 3.07E−216 | Virus-specific |
| MBP | −1.236393024 | 0 | 0 | Virus-specific |
| ANTXR2 | −1.243363428 | 9.13E−145 | 3.06E−140 | Virus-specific |
| RORA | −1.256283197 | 1.44E−231 | 4.83E−227 | Virus-specific |
| CCR7 | −1.265116844 | 0 | 0 | Virus-specific |
| ANXA1 | −1.269591083 | 0 | 0 | Virus-specific |
| BACH2 | −1.318872683 | 1.59E−273 | 5.33E−269 | Virus-specific |
| GLUL | −1.327884647 | 1.06E−236 | 3.56E−232 | Virus-specific |
| TNFSF14 | −1.330285991 | 1.83E−215 | 6.13E−211 | Virus-specific |
| AUTS2 | −1.416932251 | 1.02E−228 | 3.41E−224 | Virus-specific |
| PERP | −1.437004665 | 0 | 0 | Virus-specific |
| EPHA4 | −1.537998697 | 8.01E−189 | 2.69E−184 | Virus-specific |
| TCF7 | −1.589936811 | 0 | 0 | Virus-specific |
| SELL | −1.622001601 | 5.50E−189 | 1.85E−184 | Virus-specific |
| MYC | −1.642719984 | 5.47E−159 | 1.83E−154 | Virus-specific |
| IL7R | −1.916913934 | 0 | 0 | Virus-specific |
| CD300A | −1.923637451 | 3.88E−283 | 1.30E−278 | Virus-specific |
| ITGA5 | −2.002996655 | 1.12E−237 | 3.74E−233 | Virus-specific |
| GPR183 | −2.026905124 | 0 | 0 | Virus-specific |
| KLF3 | −2.280854987 | 0 | 0 | Virus-specific |
| S1PR1 | −2.516754625 | 0 | 0 | Virus-specific |
| FOSB | −0.569878453 | 6.43E−09 | 0.000215507 | |

External gene-signatures were identified from published studies of human TILs or murine T cells. Specifically, genes signatures of clusters reported in single-cell dataset from Yost et al. (GSE139555) (Yost et al., Nat. Med. 25:1251-59 (2019)) were comprised the top 100 cluster-specific upregulated genes (adj p val <0.05) established using Seurat package. Gene signatures for human stem-cell like and terminally differentiated TILs (GSE140430) (Jansen et al., Nature 576:465-70 (2019)), murine memory precursor (MPEC) and short-lived effector cells (SLEC) (GSE8678) (Joshi et al., Immunity 27:281-95 (2007)), murine chronic infection-derived PD1+CXCRS+Tim3− and PD1+CXCRS-Tim3+ cells (GSE84105)(Im et al., Nature 537: 417-21 (2016)) were computed from analysis of published microarray experiments or bulk sequencing data. For each experimental group, top 100 upregulated genes with FDR<1% and log₂FC>1.5 were selected as signature.

Gene signatures from human CD39+CD69+ and CD39-CD69− TILs (Krishna et al., Science 370:1328-34 (2020)), human CD8+ TILs clusters (Sade-Feldman et al., Cell 176:1-20 (2019)), human exhausted melanoma TILs (Tirosh et al., Science 352:189-96 (2016)), murine progenitor exhausted and terminally exhausted T cells in B16 tumors or in chronic infections (Miller et al., Nat. Immunol. 20:326-36 (2019)), murine memory T cells and chronic infection-derived TCF+ T cells, TCF− T cells (Utzschneider et al., Immunity 45:415-27 (2016)), murine TCF1+ and TCF1− TILs (Siddiqui et al., Immunity 50:195-211.e10 (2019)), murine tissue resident memory T cells (TRM) and circulating memory T cells (Tcirc) (Milner et al., Nature 552:253-7 (2017)), murine TOX+ T cells (Scott et al., Nature 571:270-4 (2019)), murine T cells with TOX knock-out (Khan et al., Nature 571:211-8 (2019)) were obtained from deregulated genes listed elsewhere herein. When possible, top 100 upregulated genes with FDR<1% and log₂FC>1.5 were selected. Proliferation genes were removed from gene signatures derived from comparison of two T-cell populations to avoid over-scoring of tumor-specific proliferating cell (T Prol).

TCR reconstruction and expression in T cells for reactivity screening. In vitro TCR reconstruction and antigen specificity screening was performed for: i) TCRs from CD8+ TILs of discovery cohort, selected to be highly expanded within the intratumoral microenvironment or having a primary phenotype representative of all the cluster classified as T_{Ex} or T_{NExM}; ii) TCRs sequenced in melanoma specimens of validation cohort (Sade-Feldman et al., Cell 176:1-20 (2019)) and detected with high frequency in 7 patients with HLA-A02:01 restriction; iii) TCRs isolated from peripheral blood of patients of the discovery cohort after enrichment of antitumor T cell responses. Selection criteria also included the availability of reliable sequences of both TCRα and TCRβ chains; moreover, TCRs with single TCRα and TCRβ chains were preferred to TCRs with multiple chains; only for highly expanded TCRs with 2 TCRα or 2 TCRβ chains per cell, 2 different TCRs were studied. In such case the results of the most reactive TCR are reported.

The full-length TCRα and TCRβ chains, separated by a Furin SGSG P2A linker, were synthesized in the TCRB/TCRα orientation (Integrated DNA Technologies) and cloned into a lentiviral vector (LV) under the control of the pEF1a promoter using Gibson assembly (New England Biolabs Inc., Ipswich, MA, www.neb.com). Full-length TCRα V-J regions and TCRβ V-D-J regions were fused to optimized mouse TRA and TRB constant chains respectively, to allow preferential pairing of the introduced TCR chains, enhanced surface expression and functionality (Cohen et al., Cancer Res. 66:8878-86 (2006); Haga-Friedman et al., J. Immunol. 188:5538-46 (2012); Bialer et al., J, Immunol, 184:6232-41 (2010)). The cloning strategy was optimized to rapidly reconstruct up to 96 TCRs in parallel in 96-well plates with high efficiency. The assembled plasmids were transfected in 5-alpha competent E. coli bacteria (New England Biolabs), which were expanded in LB broth (ThermoFisher Scientific) supplemented with ampicillin (Sigma). Plasmids were purified using the 96 Miniprep Kit (Qiagen), resuspended in water and sequence-verified through standard sequencing (Eton).

T cells were enriched from PBMCs obtained by healthy subjects using the PanT cell selection kit (Miltenyi Biotech) and then activated with antiCD3/CD28 dynabeads (Gibco) in the presence of 5 ng/mL of IL-7 and IL-15 (Peprotech) and dispensed in 96 well plates. After 2 days, activated cells were transduced with a LV encoding the reconstructed TCRB-TCRA chains. Briefly, LV particles were generated by transient transfection of the lentiviral packaging Lenti-X 293T cells (Takahara) with the TCR-encoding and packaging plasmids (VSVg and PSPAX2)(Hu et al., Blood 132:1911-21 (2018)) using Transit LT-1 (Mirus). Parallel production of different LV encoding diverse TCRs was achieved by seeding packaging cells in 96 well plate format. LV supernatants were harvested each day for 3 consecutive days (day 1, 2 and 3 after transfection) and used on activated T cells on day 1, 2 and 3 after activation. To increase the transduction efficiency, spinoculation (2000 rpm, 2 hours, 37° C.) in the presence of 8 μg/mL of polybrene (ThermoFisher Scientific) was performed at day 2. Six days after activation, beads were removed using Dynal magnets and supernatant was replaced with complete medium supplemented with cytokines. Transduction efficiency was assessed quantifying by flow cytometry the percentage of T cells expressing the murine TCRB with the anti-mTCRB antibody (PE, clone H57-597, eBioscience). Transduced T cells were used 14 days post-transduction for TCR reactivity tests, as detailed below.

CD137 upregulation assay. TCR transduction signal resulting from antigen recognition was assessed measuring the upregulation of CD137 surface expression on effector T cells upon co-culture with target cells. To allow for simultaneous evaluation of up to 64 distinct TCRs, T cell lines expressing distinct reconstructed TCRs were pooled after labeling with a combination of cytoplasmatic dyes. Briefly, TCR-transduced T cell lines were washed, resuspended in PBS at 1×10⁶ cells/mL and labeled with a combination of 3 dyes (Cell Trace CFSE, Far Red or Violet Proliferation Kits, Life Technologies). Up to 4 dilutions per dye were created and then mixed, resulting in up to 64 color combinations. After incubation at 37° C. for 20 minutes, T cells were washed twice, resuspended in complete medium and divided in pools. Each pool contained as internal controls a population of mock-transduced lymphocytes and a population of T cells transduced with an irrelevant TCR. Additionally, for selected T cell pools, the TCR specific for the HLA-A*0201-restricted GILGFVFTL Flu peptide (Hu et al., Blood 132:1911-21 (2018)) was included as a positive control. Effector pools were plated in 96-well plates (0.25×10⁶ cells/well) with the following targets: i) patient-derived melanoma cell lines (0.25×10⁵ cells/well), either untreated or pre-treated with IFNγ (2000 U/mL, Peprotech); ii) patient PBMCs (0.25×10⁶ cells/well); iii) patient B cells (0.25×10⁶ cells/well), purified from PBMCs using anti-human CD19 microbeads (Miltenyi Biotec); iv) patient EBV-LCLs (0.25×10⁶ cells/well) alone or pulsed with peptides; v) medium, as negative control; vi) PHA (2 micrograms per milliliter (μg/mL), Sigma-Aldrich) or PMA (50 nanograms per milliliter (ng/mL), Sigma-Aldrich) and ionomycin (10 μg/mL, Sigma-Aldrich) as positive controls. Peptide-pulsing of target cells was performed by incubating EBV-LCLs in FBS-free medium at a density of 5×10⁶ cells/mL for 2 hours in the presence of individual peptides (10⁷ pg/mL, Genscript) or peptide pools (each at 10⁷ picograms per milliliter (pg/mL), JPT Peptide Technologies, Berlin, Germany, www.jpt.com) diluted in ultrapure DMSO (Sigma-Aldrich). Tested peptides comprised pools of: i) class I peptides (>70% purity) predicted from patient NeoAgs, as previously reported (Ott et al., Nature 547:217-21 (2017)); ii) overlapping 15mer peptides (>70% purity) spanning the entire length of 12 MAA-genes (MAGE-A1, MAGE-A3, MAGE-A4, MAGE-A9, MAGE-C, MAGE-D, MLANA, PMEL, TYR, DCT, PRAME, NYES0-1); iii) class I and II peptides (>70% purity) encoding immunogenic viral antigens (CEF pools, JPT Peptide Technologies). Tested peptides also included: individual crude peptides detected by mass spectrometry (MS) within HLA-class I binding immunopeptidomes of at least one patient-derived melanoma cell line, mapping to selected MAAs or NeoAgs and predicted to bind patient HLA alleles using NetMHCpan version 4.0; and individual crude peptides from MLANA protein, either predicted to bind class I HLAs of patients with high MLANA tumor expression (Pt-A, Pt-B and Pt-D) using NetMHCpan version 4.0 or reported to be highly immunogenic (Kawakami et al., J. Exp. Med. 180:347-52 (1994)) (Table 13-Table 15).

TABLE 13
Assay peptides covering predicted neoantigens included in vaccination treatment

| | | Sequence (mutant | | | Neo | | |
|---|---|---|---|---|---|---|---|
| | | amino acids in bold | SEQ ID | | Ag | | De- |
| Patient | Peptide ID | and boxed) | Nos: | NeoAg | Pool | Length | tected* |
| Pt-A | PtA-NeoAg pool#1-1 | 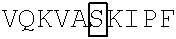 | SEQ ID NO: 67 | PHF21B p.P130S | 1 | 10 | N |
| Pt-A | PtA-NeoAg pool#1-2a | 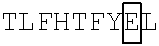 | SEQ ID NO: 68 | NLRC4 p.D368E | 1 | 9 | N |
| Pt-A | PtA-NeoAg pool#1-2b | 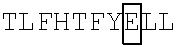 | SEQ ID NO: 69 | NLRC4 p.D368E | 1 | 10 | N |
| Pt-A | PtA-NeoAg pool#1-2c | 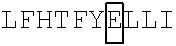 | SEQ ID NO: 70 | NLRC4 p.D368E | 1 | 10 | N |
| Pt-A | PtA-NeoAg pool#1-2d | 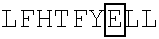 | SEQ ID NO: 71 | NLRC4 p.D368E | 1 | 9 | Y |
| Pt-A | PtA-NeoAg pool#1-2e | 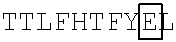 | SEQ ID NO: 72 | NLRC4 p.D368E | 1 | 10 | N |
| Pt-A | PtA-NeoAg pool#1-3a | 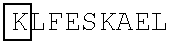 | SEQ ID NO: 73 | MECOM p.Q28K | 1 | 9 | N |
| Pt-A | PtA-NeoAg pool#1-3b | 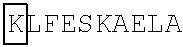 | SEQ ID NO: 74 | MECOM p.Q28K | 1 | 10 | N |
| Pt-A | PtA-NeoAg pool#1-4 | 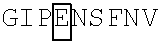 | SEQ ID NO: 75 | LUM p.G248E | 1 | 9 | N |
| Pt-A | PtA-NeoAg pool#2-1a | 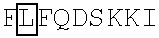 | SEQ ID NO: 76 | PRTG p.F1055L | 2 | 9 | N |
| Pt-A | PtA-NeoAg pool#2-1b | 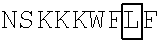 | SEQ ID NO: 77 | PRTG p.F1055L | 2 | 9 | Y |
| Pt-A | PtA-NeoAg pool#2-2a | 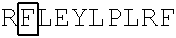 | SEQ ID NO: 78 | DCAKD p.S199F | 2 | 10 | N |
| Pt-A | PtA-NeoAg pool#2-2b | 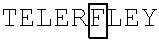 | SEQ ID NO: 79 | DCAKD p.S199F | 2 | 9 | N |
| Pt-A | PtA-NeoAg pool#2-2c | 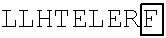 | SEQ ID NO: 80 | DCAKD p.S199F | 2 | 9 | Y |
| Pt-A | PtA-NeoAg pool#2-2d | 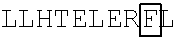 | SEQ ID NO: 81 | DCAKD p.S199F | 2 | 10 | N |
| Pt-A | PtA-NeoAg pool#2-3 | 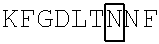 | SEQ ID NO: 82 | ADARB1 p.D340N | 2 | 9 | N |
| Pt-A | PtA-NeoAg pool#3-1a | 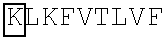 | SEQ ID NO: 83 | ACPP p.E34K | 3 | 9 | N |
| Pt-A | PtA-NeoAg pool#3-1b | 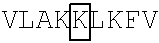 | SEQ ID NO: 84 | ACPP p.E34K | 3 | 9 | N |
| Pt-A | PtA-NeoAg pool#3-2 | 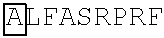 | SEQ ID NO: 85 | ARHGEF1 5 p.V651A | 3 | 9 | N |
| Pt-A | PtA-NeoAg pool#3-3a | 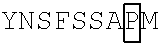 | SEQ ID NO: 86 | PRRC2C p.S2300P | 3 | 9 | N |
| Pt-A | PtA-NeoAg pool#3-3b | 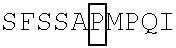 | SEQ ID NO: 87 | PRRC2C p.S2300P | 3 | 10 | N |
| Pt-A | PtA-NeoAg pool#3-4a | 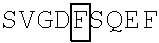 | SEQ ID NO: 88 | RUSC2 p.S569F | 3 | 9 | N |
| Pt-A | PtA-NeoAg pool#3-4b | 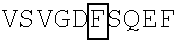 | SEQ ID NO: 89 | RUSC2 p.S569F | 3 | 10 | N |
| Pt-B | PtB-NeoAg pool#1-1 | 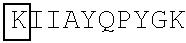 | SEQ ID NO: 90 | PRKCG p.E525K | 1 | 10 | N |
| Pt-B | PtB-NeoAg pool#1-2a | 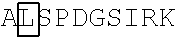 | SEQ ID NO: 91 | KCNC3 p.P715L | 1 | 10 | N |
| Pt-B | PtB-NeoAg pool#1-2b | 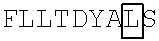 | SEQ ID NO: 92 | KCNC3 p.P715L | 1 | 9 | N |
| Pt-B | PtB-NeoAg pool#1-3a | 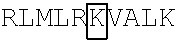 | SEQ ID NO: 93 | TLR3 p.R212K | 1 | 10 | N |
| Pt-B | PtB-NeoAg pool#1-3b | 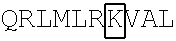 | SEQ ID NO: 94 | TLR3 p.R212K | 1 | 10 | N |
| Pt-B | PtB-NeoAg pool#1-3c | 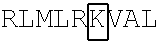 | SEQ ID NO: 95 | TLR3 p.R212K | 1 | 9 | N |
| Pt-B | PtB-NeoAg pool#1-3d | 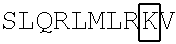 | SEQ ID NO: 96 | TLR3 p.R212K | 1 | 10 | N |
| Pt-B | PtB-NeoAg pool#1-4a | 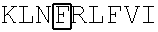 | SEQ ID NO: 97 | CRY1 p.S71F | 1 | 9 | N |
| Pt-B | PtB-NeoAg pool#1-4b | 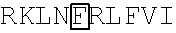 | SEQ ID NO: 98 | CRY1 p.S71F | 1 | 10 | N |
| Pt-B | PtB-NeoAg pool#1-4c | 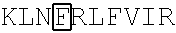 | SEQ ID NO: 99 | CRY1 p.S71F | 1 | 10 | N |
| Pt-B | PtB-NeoAg pool#2-1 | 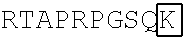 | SEQ ID NO: 100 | ENDOV p.E257K | 2 | 10 | N |
| Pt-B | PtB-NeoAg pool#2-2 | 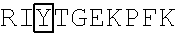 | SEQ ID NO: 101 | ZNF234 p.H282Y | 2 | 10 | N |
| Pt-B | PtB-NeoAg pool#2-3a | 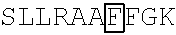 | SEQ ID NO: 102 | VPS16 p.S90F | 2 | 10 | N |
| Pt-B | PtB-NeoAg pool#2-3b | 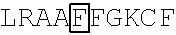 | SEQ ID NO: 103 | VPS16 p.S90F | 2 | 10 | N |
| Pt-B | PtB-NeoAg pool#2-4a | 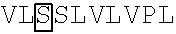 | SEQ ID NO: 104 | DNASE1L 1 p.P140S | 2 | 10 | N |
| Pt-B | PtB-NeoAg pool#2-4b | 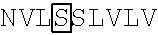 | SEQ ID NO: 105 | DNASE1L 1 p.P140S | 2 | 9 | N |
| Pt-B | PtB-NeoAg pool#2-4c | 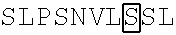 | SEQ ID NO: 106 | DNASE1L 1 p.P140S | 2 | 10 | N |
| Pt-B | PtB-NeoAg pool#2-5a | 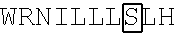 | SEQ ID NO: 107 | CARD16 p.P172S | 2 | 10 | N |
| Pt-B | PtB-NeoAg pool#2-5b | 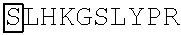 | SEQ ID NO: 108 | CARD16 p.P172S | 2 | 10 | N |
| Pt-B | PtB-NeoAg pool#3-1a | 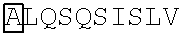 | SEQ ID NO: 109 | CCSER1 p.V195A | 3 | 10 | N |
| Pt-B | PtB-NeoAg pool#3-1b | 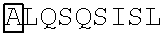 | SEQ ID NO: 110 | CCSER1 p.V195A | 3 | 9 | N |
| Pt-B | PtB-NeoAg pool#3-2 | 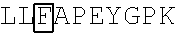 | SEQ ID NO: 111 | TBC1D14 p.S42F | 3 | 10 | N |
| Pt-B | PtB-NeoAg pool#3-3 | 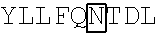 | SEQ ID NO: 112 | GTF3C2 p.D800N | 3 | 9 | N |
| Pt-B | PtB-NeoAg pool#3-4a | 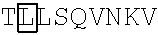 | SEQ ID NO: 113 | CIT p.P2056L | 3 | 9 | N |
| Pt-B | PtB-NeoAg pool#3-4b | 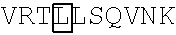 | SEQ ID NO: 114 | CIT p.P2056L | 3 | 10 | N |
| Pt-B | PtB-NeoAg pool#3-5a | 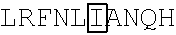 | SEQ ID NO: 115 | ADAMTS7 p.T961 | 3 | 10 | N |
| Pt-B | PtB-NeoAg pool#3-5b | 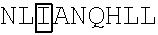 | SEQ ID NO: 116 | ADAMTS7 p.T961 | 3 | 9 | N |
| Pt-C | PtC-NeoAg pool#1-1 | 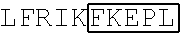 | SEQ ID NO: 117 | RALGAPB p.11403fs | 1 | 10 | N |
| Pt-C | PtC-NeoAg pool#1-2a | 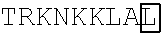 | SEQ ID NO: 118 | KMT2A p.P3239L | 1 | 9 | N |
| Pt-C | PtC-NeoAg pool#1-2b | 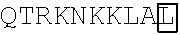 | SEQ ID NO: 119 | KMT2A p.P3239L | 1 | 10 | N |
| Pt-C | PtC-NeoAg pool#1-3a | 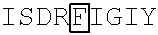 | SEQ ID NO: 120 | SMC4 p.L1262F | 1 | 9 | N |
| Pt-C | PtC-NeoAg pool#1-3b | 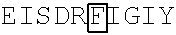 | SEQ ID NO: 121 | SMC4 p.L1262F | 1 | 10 | N |
| Pt-C | PtC-NeoAg pool#1-3c | 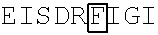 | SEQ ID NO: 122 | SMC4 p.L1262F | 1 | 9 | N |
| Pt-C | PtC-NeoAg pool#1-4a | 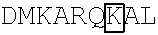 | SEQ ID NO: 123 | FAM50B p.E78K | 1 | 9 | N |
| Pt-C | PtC-NeoAg pool#1-4b | 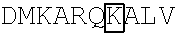 | SEQ ID NO: 124 | FAM50B p.E78K | 1 | 10 | N |
| Pt-C | PtC-NeoAg pool#1-5a | 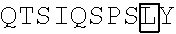 | SEQ ID NO: 125 | TP63 p.S364L | 1 | 10 | N |
| Pt-C | PtC-NeoAg pool#1-5b | 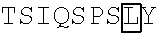 | SEQ ID NO: 126 | TP63 p.S364L | 1 | 9 | N |
| Pt-C | PtC-NeoAg pool#2-1 | 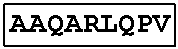 | SEQ ID NO: 127 | PISD p.R83fs | 2 | 9 | N |
| Pt-C | PtC-NeoAg pool#2-2 | 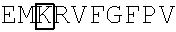 | SEQ ID NO: 128 | DNMT3A p.E642K | 2 | 10 | N |
| Pt-C | PtC-NeoAg pool#2-3 | 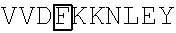 | SEQ ID NO: 129 | PDE1C p.L61F | 2 | 10 | N |
| Pt-C | PtC-NeoAg pool#2-4a | 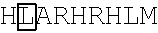 | SEQ ID NO: 130 | TEX2 p.P207L | 2 | 9 | N |
| Pt-C | PtC-NeoAg pool#2-4b | 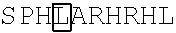 | SEQ ID NO: 131 | TEX2 p.P207L | 2 | 10 | N |
| Pt-C | PtC-NeoAg pool#2-5 | 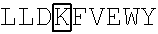 | SEQ ID NO: 132 | DCUN1D4 p.E281K | 2 | 9 | N |
| Pt-C | PtC-NeoAg pool#3-1a | 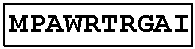 | SEQ ID NO: 133 | CADM4 p.V87fs | 3 | 10 | N |
| Pt-C | PtC-NeoAg pool#3-1b | 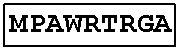 | SEQ ID NO: 134 | CADM4 p.V87fs | 3 | 9 | N |
| Pt-C | PtC-NeoAg pool#3-2 | 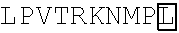 | SEQ ID NO: 135 | DTX4 p.P117L | 3 | 10 | N |
| Pt-C | PtC-NeoAg pool#3-3a | 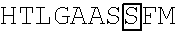 | SEQ ID NO: 136 | SETBP1 p.P1084S | 3 | 10 | N |
| Pt-C | PtC-NeoAg pool#3-3b | 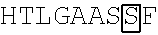 | SEQ ID NO: 137 | SETBP1 p.P1084S | 3 | 9 | N |
| Pt-C | PtC-NeoAg pool#3-4 | 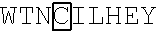 | SEQ ID NO: 138 | KRIT1 p.R600C | 3 | 9 | N |
| Pt-C | PtC-NeoAg pool#3-5 | 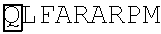 | SEQ ID NO: 139 | PTCD1 p.R8Q | 3 | 9 | N |
| Pt-C | PtC-NeoAg pool#4-1a | 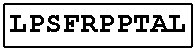 | SEQ ID NO: 140 | TNS1 p.G790fs | 4 | 10 | N |
| Pt-C | PtC-NeoAg pool#4-1b | 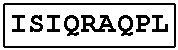 | SEQ ID NO: 141 | TNS1 p.G790fs | 4 | 9 | N |
| Pt-C | PtC-NeoAg pool#4-2 | 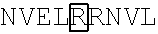 | SEQ ID NO: 142 | NCOA6 p.P1371R | 4 | 9 | N |
| Pt-C | PtC-NeoAg pool#4-3a | 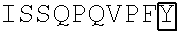 | SEQ ID NO: 143 | DNMBP p.H421Y | 4 | 10 | N |
| Pt-C | PtC-NeoAg pool#4-3b | 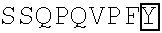 | SEQ ID NO: 144 | DNMBP p.H421Y | 4 | 9 | N |
| Pt-C | PtC-NeoAg pool#4-4 | 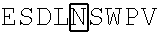 | SEQ ID NO: 145 | TBX10 p.D256N | 4 | 9 | N |
| Pt-C | PtC-NeoAg pool#4-5a | 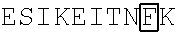 | SEQ ID NO: 146 | EEA1 p.L1161F | 4 | 10 | N |
| Pt-C | PtC-NeoAg pool#4-5b | 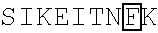 | SEQ ID NO: 147 | EEA1 p.L1161F | 4 | 9 | N |
| Pt-C | PtC-NeoAg pool#4-5c | 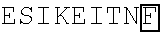 | SEQ ID NO: 148 | EEA1 p.L1161F | 4 | 9 | N |
| Pt-D | PtD-NeoAg pool#1-1a | 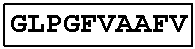 | SEQ ID NO: 149 | CRELD2 fs | 1 | 10 | N |
| Pt-D | PtD-NeoAg pool#1-1b | 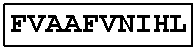 | SEQ ID NO: 150 | CRELD2 fs | 1 | 10 | N |
| Pt-D | PtD-NeoAg pool#1-1c | 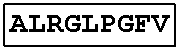 | SEQ ID NO: 151 | CRELD2 fs | 1 | 9 | N |
| Pt-D | PtD-NeoAg pool#1-2 | 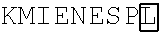 | SEQ ID NO: 152 | SETBP1 p.S477L | 1 | 9 | N |
| Pt-D | PtD-NeoAg pool#1-3 | 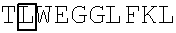 | SEQ ID NO: 153 | UBE21 p.P52L | 1 | 10 | N |
| Pt-D | PtD-NeoAg pool#1-4a | 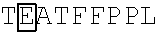 | SEQ ID NO: 154 | BAZ2B p.G126E | 1 | 9 | N |
| Pt-D | PtD-NeoAg pool#1-4b | 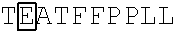 | SEQ ID NO: 155 | BAZ2B p.G126E | 1 | 10 | N |
| Pt-D | PtD-NeoAg pool#2-1a | 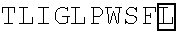 | SEQ ID NO: 156 | GALC p.P154L | 2 | 10 | N |
| Pt-D | PtD-NeoAg pool#2-1b | 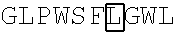 | SEQ ID NO: 157 | GALC p.P154L | 2 | 10 | N |
| Pt-D | PtD-NeoAg pool#2-2a | 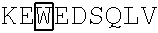 | SEQ ID NO: 158 | DDX60 p.C1567W | 2 | 9 | N |
| Pt-D | PtD-NeoAg pool#2-2b | 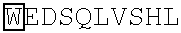 | SEQ ID NO: 159 | DDX60 p.C1567W | 2 | 10 | N |
| Pt-D | PtD-NeoAg pool#2-2c | 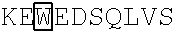 | SEQ ID NO: 160 | DDX60 p.C1567W | 2 | 10 | N |
| Pt-D | PtD-NeoAg pool#2-3 | 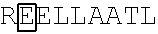 | SEQ ID NO: 161 | KPTN p.G39E | 2 | 9 | N |
| Pt-D | PtD-NeoAg pool#3-1a | 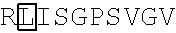 | SEQ ID NO: 162 | NUP35 p.S53L | 3 | 10 | N |
| Pt-D | PtD-NeoAg pool#3-1b | 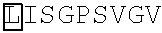 | SEQ ID NO: 163 | NUP35 p. S53L | 3 | 9 | N |
| Pt-D | PtD-NeoAg pool#3-2 | 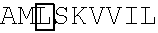 | SEQ ID NO: 164 | CIT p.P1749L | 3 | 9 | N |
| Pt-D | PtD-NeoAg pool#3-3a | 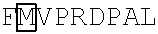 | SEQ ID NO: 165 | USP32 p.L1312M | 3 | 9 | N |
| Pt-D | PtD-NeoAg pool#3-3b | 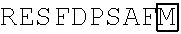 | SEQ ID NO: 166 | USP32 p.L1312M | 3 | 10 | N |
| Pt-D | PtD-NeoAg pool#3-4 | 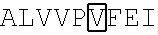 | SEQ ID NO: 167 | B3GNT1 p.A259V | 3 | 9 | N |
| Pt-A^{†} | PtA-MS-NeoAg#1 | 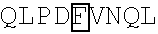 | SEQ ID NO: 168 | TMEM214 p.S605F | — | 9 | Y |
| Pt-C^{†} | PtC-MS-NeoAg#1 | 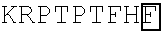 | SEQ ID NO: 169 | MACF1 p.S7278F | — | 9 | Y |
| Pt-C^{†} | PtC-MS-NeoAg#2 | 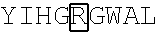 | SEQ ID NO: 170 | NCEH1 p.G115R | — | 8 | Y |
| Pt-D^{†} | PtD-MS-NeoAg#1 | 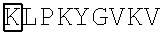 | SEQ ID NO: 171 | RPL5 p.E82K | — | 9 | Y |

*Detected by MS in HLA class I immunopeptidome of melanoma cell lines
^{†}an additional Neoantigen detected in Melanoma HLA class I immunopeptidome

TABLE 14
Peptide pools comprising pool of 15mers with 11 aa overlap

| MAA Gene | # of Peptides |
|---|---|
| MAGEA1 | 75 |
| MAGEA3 | 76 |
| MAGEA4 | 77 |
| MAGEA9 | 76 |
| MAGEC1 | 283 |
| MAGED4 | 183 |
| MLANA | 27 |
| PMEL | 163 |
| TYR | 117 |
| DCT | 127 |
| PRAME | 125 |
| NYESO-1 | 43 |

TABLE 15
Individual Peptides

| Patient | Peptide ID | MAA Gene | Sequence | SEQ ID NOS | Length | Detected* |
|---|---|---|---|---|---|---|
| Pt-A | PtA-MAGE#1 | MAGEA1 | KVLEYVIKV | SEQ ID NO: | 9 | Y |
| | | | | 186 | | |
| Pt-A | PtA-MAGE#2 | MAGEA1 | SAYGEPRKL | SEQ ID NO: | 9 | Y |
| | | | | 187 | | |
| Pt-A | PtA-MAGE#3 | MAGEA2 | SVFAHPRKL | SEQ ID NO: | 9 | Y |
| | | | | 188 | | |
| Pt-A | PtA-MAGE#4 | MAGEA4 | GVYDGREHTV | SEQ ID NO: | 10 | Y |
| | | | | 189 | | |
| Pt-A | PtA-MAGE#5 | MAGEA6 | KIWEELSVLEV | SEQ ID NO: | 11 | Y |
| | | | | 190 | | |
| Pt-A | PtA-MAGE#6 | MAGED1 | MLRDIIREY | SEQ ID NO: | 9 | Y |
| | | | | 191 | | |
| Pt-A | PtA-MAGE#7 | MAGED1 | EYTDVYPEI | SEQ ID NO: | 9 | Y |
| | | | | 192 | | |
| Pt-A | PtA-MAGE#8 | MAGED1 | AANKSEMAF | SEQ ID NO: | 9 | Y |
| | | | | 193 | | |
| Pt-A | PtA-MAGE#9 | MAGED2 | SLFGDVKKL | SEQ ID NO: | 9 | Y |
| | | | | 194 | | |
| Pt-A | PtA-MAGE#10 | MAGED2 | YSLEKVFGI | SEQ ID NO: | 9 | Y |
| | | | | 195 | | |
| Pt-A | PtA-MAGE#11 | MAGED2 | SMMQTLLTV | SEQ ID NO: | 9 | Y |
| | | | | 196 | | |
| Pt-A | PtA-MAGE#12 | MAGED2 | NADPQAVTM | SEQ ID NO: | 9 | Y |
| | | | | 197 | | |
| Pt-A | PtA-MAGE#13 | MAGEF1 | VQPSKYHFL | SEQ ID NO: | 9 | Y |
| | | | | 198 | | |
| Pt-A | PtA-MAGE#14 | MAGED1 | FVLEKKFGI | SEQ ID NO: | 9 | Y |
| | | | | 199 | | |
| Pt-A | PtA-MAGE#15 | MAGEC2 | SIKKKVLEF | SEQ ID NO: | 9 | Y |
| | | | | 200 | | |
| Pt-A | PtA-MAGE#16 | MAGEA5 | KVADLIHFL | SEQ ID NO: | 9 | Y |
| | | | | 201 | | |
| Pt-A | PtA-MAGE#17 | MAGEA9B | KVAELVHFL | SEQ ID NO: | 9 | Y |
| | | | | 202 | | |
| Pt-A | PtA-MAGE#18 | MAGEC2 | FVYGEPREL | SEQ ID NO: | 9 | Y |
| | | | | 203 | | |
| Pt-A | PtA-MAGE#19 | MAGEC2 | GVYAGREHFV | SEQ ID NO: | 10 | Y |
| | | | | 204 | | |
| Pt-A | PtA-MAGE#20 | MAGED1 | KEIDKEEHL | SEQ ID NO: | 9 | Y |
| | | | | 205 | | |
| Pt-A | PtA-MAGE#21 | MAGED1 | LEKKFGIQL | SEQ ID NO: | 9 | Y |
| | | | | 206 | | |
| Pt-A | PtA-MAGE#22 | MAGED2 | LEKVFGIQL | SEQ ID NO: | 9 | Y |
| | | | | 207 | | |
| Pt-A | PtA-MLANA#1 | MLANA | AEEAAGIGI | SEQ ID NO: | 9 | N |
| | | | | 208 | | (predicted) |
| Pt-A | PtA-MLANA#2 | MLANA | AEQSPPPY | SEQ ID NO: | 8 | N |
| | | | | 209 | | (predicted) |
| Pt-A | PtA-MLANA#3 | MLANA | ALMDKSLHV | SEQ ID NO: | 9 | Y |
| | | | | 210 | | |
| Pt-A | PtA-MLANA#4 | MLANA | EDAHFIYGY | SEQ ID NO: | 9 | N |
| | | | | 211 | | (predicted) |
| Pt-A | PtA-MLANA#5 | MLANA | GILTVILGV | SEQ ID NO: | 9 | N |
| | | | | 212 | | (predicted) |
| Pt-A | PtA-MLANA#6 | MLANA | NAPPAYEKL | SEQ ID NO: | 9 | N |
| | | | | 213 | | (predicted) |
| Pt-A | PtA-MLANA#7 | MLANA | RALMDKSLHV | SEQ ID NO: | 10 | N |
| | | | | 214 | | (predicted) |
| Pt-A | PtA-MLANA#8 | MLANA | REDAHFIYGY | SEQ ID NO: | 10 | N |
| | | | | 215 | | (predicted) |
| Pt-A | PtA-MLANA#9 | MLANA | RRNGYRALM | SEQ ID NO: | 9 | N |
| | | | | 216 | | (predicted) |
| Pt-A | PtA-MLANA#10 | MLANA | RRNGYRALMDK | SEQ ID NO: | 11 | N |
| | | | | 217 | | (predicted) |
| Pt-A | PtA-MLANA#11 | MLANA | RRRNGYRALM | SEQ ID NO: | 10 | N |
| | | | | 218 | | (predicted) |
| Pt-A | PtA-MLANA#12 | MLANA | TRRCPQEGF | SEQ ID NO: | 9 | N |
| | | | | 219 | | (predicted) |
| Pt-A | PtA-MLANA#13 | MLANA | VVPNAPPAY | SEQ ID NO: | 9 | N |
| | | | | 220 | | (predicted) |
| Pt-A | PtA-MLANA#14 | MLANA | YRALMDKSLHV | SEQ ID NO: | 11 | N |
| | | | | 221 | | (predicted) |
| Pt-A | PtA-MLANA#15 | MLANA | AAGIGILTV | SEQ ID NO: | 9 | N (reported |
| | | | | 222 | | to be |
| | | | | | | immunogenic) |
| Pt-A | PtA-PMEL#1 | PMEL | KTWGQYWQV | SEQ ID NO: | 9 | Y |
| | | | | 223 | | |
| Pt-A | PtA-PMEL#2 | PMEL | AMLGTHTMEV | SEQ ID NO: | 10 | Y |
| | | | | 224 | | |
| Pt-A | PtA-PMEL#3 | PMEL | ALDGGNKHFL | SEQ ID NO: | 10 | Y |
| | | | | 225 | | |
| Pt-A | PtA-PMEL#4 | PMEL | SLADTNSLAVV | SEQ ID NO: | 11 | Y |
| | | | | 226 | | |
| Pt-A | PtA-PMEL#5 | PMEL | ITDQVPFSV | SEQ ID NO: | 9 | Y |
| | | | | 227 | | |
| Pt-A | PtA-PMEL#6 | PMEL | RYGSFSVTL | SEQ ID NO: | 9 | Y |
| | | | | 228 | | |
| Pt-A | PtA-PMEL#7 | PMEL | LYPEWTEAQRL | SEQ ID NO: | 11 | Y |
| | | | | 229 | | |
| Pt-A | PtA-PMEL#8 | PMEL | GQVPLIVGI | SEQ ID NO: | 9 | Y |
| | | | | 230 | | |
| Pt-A | PtA-PMEL#9 | PMEL | HQILKGGSGTY | SEQ ID NO: | 11 | Y |
| | | | | 231 | | |
| Pt-A | PtA-PMEL#10 | PMEL | HSSSHWLRLP | SEQ ID NO: | 10 | Y |
| | | | | 232 | | |
| Pt-A | PtA-PMEL#11 | PMEL | ILKGGSGTY | SEQ ID NO: | 9 | Y |
| | | | | 233 | | |
| Pt-A | PtA-PMEL#12 | PMEL | LIMPGQEAGLGQ | SEQ ID NO: | 15 | Y |
| | | | VPL | 234 | | |
| Pt-A | PtA-PMEL#13 | PMEL | TEAQRLDCW | SEQ ID NO: | 9 | Y |
| | | | | 235 | | |
| Pt-A | PtA-PMEL#14 | PMEL | KQDFSVPQL | SEQ ID NO: | 9 | Y |
| | | | | 236 | | |
| Pt-A | PtA-PMEL#15 | PMEL | LIYRRRLMK | SEQ ID NO: | 9 | Y |
| | | | | 237 | | |
| Pt-A | PtA-PMEL#16 | PMEL | SCPIGENSPL | SEQ ID NO: | 10 | Y |
| | | | | 238 | | |
| Pt-A | PtA-TYR#1 | TYR | FLPWHRLFL | SEQ ID NO: | 9 | Y |
| | | | | 239 | | |
| Pt-A | PtA-TYR#2 | TYR | LLMEKEDYHSL | SEQ ID NO: | 11 | Y |
| | | | | 240 | | |
| Pt-A | PtA-TYR#3 | TYR | MLLAVLYCL | SEQ ID NO: | 9 | Y |
| | | | | 241 | | |
| Pt-A | PtA-TYR#4 | TYR | SYLEQASRI | SEQ ID NO: | 9 | Y |
| | | | | 242 | | |
| Pt-A | PŁA-TYR#5 | TYR | EEYNSHQSL | SEQ ID NO: | 9 | Y |
| | | | | 243 | | |
| Pt-A | PtA-TYR#6 | TYR | AMVGAVLTA | SEQ ID NO: | 9 | Y |
| | | | | 244 | | |
| Pt-A | PtA-DCT#1 | DCT | SLDDYNHLV | SEQ ID NO: | 9 | Y |
| | | | | 245 | | |
| Pt-A | PtA-PRAME#1 | PRAME | SIQSRYISM | SEQ ID NO: | 9 | Y |
| | | | | 246 | | |
| Pt-A | PtA-PRAME#2 | PRAME | FLRGRLDQL | SEQ ID NO: | 9 | Y |
| | | | | 247 | | |
| Pt-A | PtA-PRAME#3 | PRAME | SLLQHLIGL | SEQ ID NO: | 9 | Y |
| | | | | 248 | | |
| Pt-A | PtA-PRAME#4 | PRAME | GLSNLTHVL | SEQ ID NO: | 9 | Y |
| | | | | 249 | | |
| Pt-A | PtA-PRAME#5 | PRAME | SQFLSLQCL | SEQ ID NO: | 9 | Y |
| | | | | 250 | | |
| Pt-A | PtA-PRAME#6 | PRAME | PYLGQMINL | SEQ ID NO: | 9 | Y |
| | | | | 251 | | |
| Pt-A | PtA-PRAME#7 | PRAME | FLKEGACDEL | SEQ ID NO: | 10 | Y |
| | | | | 252 | | |
| Pt-A | PtA-PRAME#8 | PRAME | LYVDSLFFL | SEQ ID NO: | 9 | Y |
| | | | | 253 | | |
| Pt-A | PtA-PRAME#9 | PRAME | RLDQLLRHV | SEQ ID NO: | 9 | Y |
| | | | | 254 | | |
| Pt-A | PtA- | PRAME | SQSPSVSQL | SEQ ID NO: | 9 | Y |
| | PRAME#10 | | | 255 | | |
| Pt-A | PtA- | PRAME | VLYPVPLESY | SEQ ID NO: | 10 | Y |
| | PRAME#11 | | | 256 | | |
| Pt-A | PtA- | PRAME | HARLRELL | SEQ ID NO: | 8 | Y |
| | PRAME#12 | | | 257 | | |
| Pt-A | PtA- | PRAME | LAYLHARL | SEQ ID NO: | 8 | Y |
| | PRAME#13 | | | 258 | | |
| Pt-A | PtA- | PRAME | YLHARLREL | SEQ ID NO: | 9 | Y |
| | PRAME#14 | | | 259 | | |
| Pt-B | PtB-MLANA#1 | MLANA | AEEAAGIGI | SEQ ID NO: | 9 | N |
| | | | | 260 | | (predicted) |
| Pt-B | PtB-MLANA#2 | MLANA | ALMDKSLHV | SEQ ID NO: | 9 | Y |
| | | | | 261 | | |
| Pt-B | PtB-MLANA#3 | MLANA | GILTVILGV | SEQ ID NO: | 9 | N |
| | | | | 262 | | (predicted) |
| Pt-B | PtB-MLANA#4 | MLANA | NAPPAYEKL | SEQ ID NO: | | N |
| | | | | 263 | 9 | (predicted) |
| Pt-B | PtB-MLANA#5 | MLANA | RALMDKSLHV | SEQ ID NO: | 10 | N |
| | | | | 264 | | (predicted) |
| Pt-B | PtB-MLANA#6 | MLANA | REDAHFIYGY | SEQ ID NO: | 10 | N |
| | | | | 265 | | (predicted) |
| Pt-B | PtB-MLANA#7 | MLANA | RRNGYRALM | SEQ ID NO: | 9 | N |
| | | | | 266 | | (predicted) |
| Pt-B | PtB-MLANA#8 | MLANA | RRNGYRALMDK | SEQ ID NO: | 11 | N |
| | | | | 267 | | (predicted) |
| Pt-B | PtB-MLANA#9 | MLANA | RRRNGYRALM | SEQ ID NO: | 10 | N |
| | | | | 268 | | (predicted) |
| Pt-B | PtB-MLANA#10 | MLANA | TRRCPQEGF | SEQ ID NO: | 9 | N |
| | | | | 269 | | (predicted) |
| Pt-B | PtB-MLANA#11 | MLANA | YRALMDKSLHV | SEQ ID NO: | 11 | N |
| | | | | 270 | | (predicted) |
| Pt-B | PtB-MLANA#12 | MLANA | AAGIGILTV | SEQ ID NO: | 9 | N (reported |
| | | | | 271 | | to be |
| | | | | | | immunogenic) |
| Pt-B | PtB-PMEL#1 | PMEL | KTWGQYWQV | SEQ ID NO: | 9 | Y |
| | | | | 272 | | |
| Pt-B | PtB-PMEL#2 | PMEL | AMLGTHTMEV | SEQ ID NO: | 10 | Y |
| | | | | 273 | | |
| Pt-B | PtB-PMEL#3 | PMEL | ALDGGNKHFL | SEQ ID NO: | 10 | Y |
| | | | | 274 | | |
| Pt-B | PtB-PMEL#4 | PMEL | SLADTNSLAVV | SEQ ID NO: | 11 | Y |
| | | | | 275 | | |
| Pt-B | PtB-PMEL#5 | PMEL | ALNFPGSQK | SEQ ID NO: | 9 | Y |
| | | | | 276 | | |
| Pt-B | PtB-PMEL#6 | PMEL | GTATLRLVK | SEQ ID NO: | 9 | Y |
| | | | | 277 | | |
| Pt-B | PtB-PMEL#7 | PMEL | LDGGNKHFL | SEQ ID NO: | 9 | Y |
| | | | | 278 | | |
| Pt-B | PtB-PMEL#8 | PMEL | LVLKRCLLH | SEQ ID NO: | 9 | Y |
| | | | | 279 | | |
| Pt-B | PtB-PMEL#9 | PMEL | MDLVLKRCL | SEQ ID NO: | 9 | Y |
| | | | | 280 | | |
| Pt-B | PtB-PMEL#10 | PMEL | LRTKAWNR | SEQ ID NO: | 8 | Y |
| | | | | 281 | | |
| Pt-B | PtB-PMEL#11 | PMEL | ITDQVPFSV | SEQ ID NO: | 9 | Y |
| | | | | 282 | | |
| Pt-B | PtB-PMEL#12 | PMEL | RYGSFSVTL | SEQ ID NO: | 9 | Y |
| | | | | 283 | | |
| Pt-B | PtB-PMEL#13 | PMEL | LYPEWTEAQRL | SEQ ID NO: | 11 | Y |
| | | | | 284 | | |
| Pt-B | PtB-PMEL#14 | PMEL | GQVPLIVGI | SEQ ID NO: | 9 | Y |
| | | | | 285 | | |
| Pt-B | PtB-PMEL#15 | PMEL | HQILKGGSGTY | SEQ ID NO: | 11 | Y |
| | | | | 286 | | |
| Pt-B | PtB-PMEL#16 | PMEL | HSSSHWLRLP | SEQ ID NO: | 10 | Y |
| | | | | 287 | | |
| Pt-B | PtB-PMEL#17 | PMEL | ILKGGSGTY | SEQ ID NO: | 9 | Y |
| | | | | 288 | | |
| Pt-B | PtB-PMEL#18 | PMEL | LIMPGQEAGLGQ | SEQ ID NO: | 15 | Y |
| | | | VPL | 289 | | |
| Pt-B | PtB-PMEL#19 | PMEL | TEAQRLDCW | SEQ ID NO: | 9 | Y |
| | | | | 290 | | |
| Pt-B | PtB-PMEL#20 | PMEL | KQDFSVPQL | SEQ ID NO: | 9 | Y |
| | | | | 291 | | |
| Pt-B | PtB-PMEL#21 | PMEL | LIYRRRLMK | SEQ ID NO: | 9 | Y |
| | | | | 292 | | |
| Pt-B | PtB-PMEL#22 | PMEL | SCPIGENSPL | SEQ ID NO: | 10 | Y |
| | | | | 293 | | |
| Pt-B | PtB-TYR#1 | TYR | NIFDLSAPEKD | SEQ ID NO: | 15 | Y |
| | | | KFFA | 294 | | |
| Pt-B | PtB-TYR#2 | TYR | FLPWHRLFL | SEQ ID NO: | 9 | Y |
| | | | | 295 | | |
| Pt-B | PtB-TYR#3 | TYR | LLMEKEDYHSL | SEQ ID NO: | 11 | Y |
| | | | | 296 | | |
| Pt-B | PtB-TYR#4 | TYR | KDLGYDYSY | SEQ ID NO: | 9 | Y |
| | | | | 297 | | |
| Pt-B | PtB-TYR#5 | TYR | MLLAVLYCL | SEQ ID NO: | 9 | Y |
| | | | | 298 | | |
| Pt-B | PtB-TYR#6 | TYR | SYLEQASRI | SEQ ID NO: | 9 | Y |
| | | | | 299 | | |
| Pt-B | PtB-TYR#7 | TYR | EEYNSHQSL | SEQ ID NO: | 9 | Y |
| | | | | 300 | | |
| Pt-B | PtB-TYR#8 | TYR | AMVGAVLTA | SEQ ID NO: | 9 | Y |
| | | | | 301 | | |
| Pt-B | PtB-DCT#1 | DCT | SLDDYNHLV | SEQ ID NO: | 9 | Y |
| | | | | 302 | | |
| Pt-B | PtB-DCT#2 | DCT | GTYEGLLRR | SEQ ID NO: | 9 | Y |
| | | | | 303 | | |
| Pt-B | PtB-PRAME#1 | PRAME | SIQSRYISM | SEQ ID NO: | 9 | Y |
| | | | | 304 | | |
| Pt-B | PtB-PRAME#2 | PRAME | FLRGRLDQL | SEQ ID NO: | 9 | Y |
| | | | | 305 | | |
| Pt-B | PtB-PRAME#3 | PRAME | DQLLRHVM | SEQ ID NO: | 8 | Y |
| | | | | 306 | | |
| Pt-B | PtB-PRAME#4 | PRAME | SLLQHLIGL | SEQ ID NO: | 9 | Y |
| | | | | 307 | | |
| Pt-B | PtB-PRAME#5 | PRAME | GLSNLTHVL | SEQ ID NO: | 9 | Y |
| | | | | 308 | | |
| Pt-B | PtB-PRAME#6 | PRAME | SQFLSLQCL | SEQ ID NO: | 9 | Y |
| | | | | 309 | | |
| Pt-B | PtB-PRAME#7 | PRAME | PYLGQMINL | SEQ ID NO: | 9 | Y |
| | | | | 310 | | |
| Pt-B | PtB-PRAME#8 | PRAME | TSPRRLVEL | SEQ ID NO: | 9 | Y |
| | | | | 311 | | |
| Pt-B | PtB-PRAME#9 | PRAME | FLKEGACDEL | SEQ ID NO: | 10 | Y |
| | | | | 312 | | |
| Pt-B | PtB- | PRAME | LYVDSLFFL | SEQ ID NO: | 9 | Y |
| | PRAME#10 | | | 313 | | |
| Pt-B | PtB- | PRAME | RLDQLLRHV | SEQ ID NO: | 9 | Y |
| | PRAME#11 | | | 314 | | |
| Pt-B | PtB- | PRAME | SQSPSVSQL | SEQ ID NO: | 9 | Y |
| | PRAME#12 | | | 315 | | |
| Pt-B | PtB- | PRAME | VLYPVPLESY | SEQ ID NO: | 10 | Y |
| | PRAME#13 | | | 316 | | |
| Pt-B | PtB- | PRAME | HARLRELL | SEQ ID NO: | 8 | Y |
| | PRAME#14 | | | 317 | | |
| Pt-B | PtB- | PRAME | LAYLHARL | SEQ ID NO: | 8 | Y |
| | PRAME#15 | | | 318 | | |
| Pt-B | PtB- | PRAME | YLHARLREL | SEQ ID NO: | 9 | Y |
| | PRAME#16 | | | 319 | | |
| Pt-C | PtC-MAGE#1 | MAGED1 | DVYPEIIER | SEQ ID NO: | 9 | Y |
| | | | | 320 | | |
| Pt-C | PtC-MAGE#2 | MAGEC2 | NAVGVYAGR | SEQ ID NO: | 9 | Y |
| | | | | 321 | | |
| Pt-C | PtC-MAGE#3 | MAGED1 | EAVLWEALR | SEQ ID NO: | 9 | Y |
| | | | | 322 | | |
| Pt-C | PtC-MAGE#4 | MAGED2 | RPGIHHSL | SEQ ID NO: | 8 | Y |
| | | | | 323 | | |
| Pt-C | PtC-MAGE#5 | MAGEC2 | ESIKKKVL | SEQ ID NO: | 8 | Y |
| | | | | 324 | | |
| Pt-C | PtC-MAGE#6 | MAGED1 | FVLEKKFGI | SEQ ID NO: | 9 | Y |
| | | | | 325 | | |
| Pt-C | PtC-MAGE#7 | MAGEC2 | SIKKKVLEF | SEQ ID NO: | 9 | Y |
| | | | | 326 | | |
| Pt-C | PtC-MAGE#8 | MAGEA1 | FPSLREAAL | SEQ ID NO: | 9 | Y |
| | | | | 327 | | |
| Pt-C | PtC-MAGE#9 | MAGED1 | EALRKMGL | SEQ ID NO: | 8 | Y |
| | | | | 328 | | |
| Pt-C | PtC-MAGE#10 | MAGEA2 | QVMPKTGL | SEQ ID NO: | 8 | Y |
| | | | | 329 | | |
| Pt-C | PtC-MAGE#11 | MAGED4 | DANRPSTAF | SEQ ID NO: | 9 | Y |
| | | | | 330 | | |
| Pt-C | PtC-MAGE#12 | MAGED2 | EIDKNDHLY | SEQ ID NO: | 9 | Y |
| | | | | 331 | | |
| Pt-C | PtC-MAGE#13 | MAGEA12 | EPFTKAEM | SEQ ID NO: | 8 | Y |
| | | | | 332 | | |
| Pt-C | PtC-MAGE#14 | MAGEC2 | KYKDYFPVIL | SEQ ID NO: | 10 | Y |
| | | | | 333 | | |
| Pt-C | PtC-MAGE#15 | MAGED2 | SRGPIAFWA | SEQ ID NO: | 9 | Y |
| | | | | 334 | | |
| Pt-C | PtC-MAGE#16 | MAGEA1 | TTINFTRQR | SEQ ID NO: | 9 | Y |
| | | | | 335 | | |
| Pt-C | PtC-PRAME#1 | PRAME | SIQSRYISM | SEQ ID NO: | 9 | Y |
| | | | | 336 | | |
| Pt-C | PtC-PRAME#2 | PRAME | FLRGRLDQL | SEQ ID NO: | 9 | Y |
| | | | | 337 | | |
| Pt-C | PtC-PRAME#3 | PRAME | DQLLRHVM | SEQ ID NO: | 8 | Y |
| | | | | 338 | | |
| Pt-C | PtC-PRAME#4 | PRAME | SLLQHLIGL | SEQ ID NO: | 9 | Y |
| | | | | 339 | | |
| Pt-C | PtC-PRAME#5 | PRAME | GLSNLTHVL | SEQ ID NO: | 9 | Y |
| | | | | 340 | | |
| Pt-C | PtC-PRAME#6 | PRAME | SQFLSLQCL | SEQ ID NO: | 9 | Y |
| | | | | 341 | | |
| Pt-C | PtC-PRAME#7 | PRAME | GQHLHLETF | SEQ ID NO: | 9 | Y |
| | | | | 342 | | |
| Pt-C | PtC-PRAME#8 | PRAME | PYLGQMINL | SEQ ID NO: | 9 | Y |
| | | | | 343 | | |
| Pt-C | PtC-PRAME#9 | PRAME | TSPRRLVEL | SEQ ID NO: | 9 | Y |
| | | | | 344 | | |
| Pt-C | PtC- | PRAME | FLKEGACDEL | SEQ ID NO: | 10 | Y |
| | PRAME#10 | | | 345 | | |
| Pt-C | PtC- | PRAME | LYVDSLFFL | SEQ ID NO: | 9 | Y |
| | PRAME#11 | | | 346 | | |
| Pt-C | PtC- | PRAME | RLDQLLRHV | SEQ ID NO: | 9 | Y |
| | PRAME#12 | | | 347 | | |
| Pt-C | PtC- | PRAME | SQSPSVSQL | SEQ ID NO: | 9 | Y |
| | PRAME#13 | | | 348 | | |
| Pt-C | PtC- | PRAME | VLYPVPLESY | SEQ ID NO: | 10 | Y |
| | PRAME#14 | | | 349 | | |
| Pt-C | PtC- | PRAME | HARLRELL | SEQ ID NO: | 8 | Y |
| | PRAME#15 | | | 350 | | |
| Pt-C | PtC- | PRAME | LAYLHARL | SEQ ID NO: | 8 | Y |
| | PRAME#16 | | | 351 | | |
| Pt-C | PtC- | PRAME | YLHARLREL | SEQ ID NO: | 9 | Y |
| | PRAME#17 | | | 352 | | |
| Pt-D | PtD-MAGE#1 | MAGEA1 | KVLEYVIKV | SEQ ID NO: | 9 | Y |
| | | | | 353 | | |
| Pt-D | PtD-MAGE#2 | MAGEA1 | ALREEEEGV | SEQ ID NO: | 9 | Y |
| | | | | 354 | | |
| Pt-D | PtD-MAGE#3 | MAGEA11 | ALREEGEGV | SEQ ID NO: | 9 | Y |
| | | | | 355 | | |
| Pt-D | PID-MAGE#4 | MAGEA2 | SVFAHPRKL | SEQ ID NO: | 9 | Y |
| | | | | 356 | | |
| Pt-D | PtD-MAGE#5 | MAGEA4 | GVYDGREHTV | SEQ ID NO: | 10 | Y |
| | | | | 357 | | |
| Pt-D | PtD-MAGE#6 | MAGEA6 | KIWEELSVLEV | SEQ ID NO: | 11 | Y |
| | | | | 358 | | |
| Pt-D | PtD-MAGE#7 | MAGED1 | MLRDIIREY | SEQ ID NO: | 9 | Y |
| | | | | 359 | | |
| Pt-D | PtD-MAGE#8 | MAGED1 | EYTDVYPEI | SEQ ID NO: | 9 | Y |
| | | | | 360 | | |
| Pt-D | PtD-MAGE#9 | MAGED2 | SLFGDVKKL | SEQ ID NO: | 9 | Y |
| | | | | 361 | | |
| Pt-D | PtD-MAGE#10 | MAGED2 | YSLEKVFGI | SEQ ID NO: | 9 | Y |
| | | | | 362 | | |
| Pt-D | PtD-MAGE#11 | MAGED2 | SMMQTLLTV | SEQ ID NO: | 9 | Y |
| | | | | 363 | | |
| Pt-D | PtD-MAGE#12 | MAGED2 | NADPQAVTM | SEQ ID NO: | 9 | Y |
| | | | | 364 | | |
| Pt-D | PtD-MAGE#13 | MAGEF1 | VQPSKYHFL | SEQ ID NO: | 9 | Y |
| | | | | 365 | | |
| Pt-D | PtD-MAGE#14 | MAGED1 | FVLEKKFGI | SEQ ID NO: | 9 | Y |
| | | | | 366 | | |
| Pt-D | PtD-MAGE#15 | MAGEC2 | SIKKKVLEF | SEQ ID NO: | 9 | Y |
| | | | | 367 | | |
| Pt-D | PtD-MAGE#16 | MAGEA5 | KVADLIHFL | SEQ ID NO: | 9 | Y |
| | | | | 368 | | |
| Pt-D | PtD-MAGE#17 | MAGEA9B | KVAELVHFL | SEQ ID NO: | 9 | Y |
| | | | | 369 | | |
| Pt-D | PtD-MAGE#18 | MAGEC2 | FVYGEPREL | SEQ ID NO: | 9 | Y |
| | | | | 370 | | |
| Pt-D | PtD-MAGE#19 | MAGEC2 | GVYAGREHFV | SEQ ID NO: | 10 | Y |
| | | | | 371 | | |
| Pt-D | PtD-MAGE#20 | MAGED1 | KEIDKEEHL | SEQ ID NO: | 9 | Y |
| | | | | 372 | | |
| Pt-D | PtD-MAGE#21 | MAGED1 | LEKKFGIQL | SEQ ID NO: | 9 | Y |
| | | | | 373 | | |
| Pt-D | PtD-MAGE#22 | MAGED2 | LEKVFGIQL | SEQ ID NO: | 9 | Y |
| | | | | 374 | | |
| Pt-D | PtD-MLANA#1 | MLANA | AEEAAGIGI | SEQ ID NO: | 9 | N |
| | | | | 375 | | (predicted) |
| Pt-D | PtD-MLANA#2 | MLANA | ALMDKSLHV | SEQ ID NO: | 9 | Y |
| | | | | 376 | | |
| Pt-D | PtD-MLANA#3 | MLANA | GILTVILGV | SEQ ID NO: | 9 | N |
| | | | | 377 | | (predicted) |
| Pt-D | PtD-MLANA#4 | MLANA | RALMDKSLHV | SEQ' ID NO: | 10 | N |
| | | | | 378 | | (predicted) |
| Pt-D | PtD-MLANA#5 | MLANA | REDAHFIYGY | SEQ ID NO: | 10 | N |
| | | | | 379 | | (predicted) |
| Pt-D | PtD-MLANA#6 | MLANA | RRNGYRALM | SEQ ID NO: | 9 | N |
| | | | | 380 | | (predicted) |
| Pt-D | PtD-MLANA#7 | MLANA | RRRNGYRALM | SEQ ID NO: | 10 | N |
| | | | | 381 | | (predicted) |
| Pt-D | PtD-MLANA#8 | MLANA | TRRCPQEGF | SEQ ID NO: | 9 | N |
| | | | | 382 | | (predicted) |
| Pt-D | PtD-MLANA#9 | MLANA | VVPNAPPAY | SEQ ID NO: | 9 | N |
| | | | | 383 | | (predicted) |
| Pt-D | PtD- | MLANA | YRALMDKSLHV | SEQ ID NO: | 11 | N |
| | MLANA#10 | | | 384 | | (predicted) |
| Pt-D | PtD- | MLANA | AAGIGILTV | SEQ ID NO: | 9 | N (reported |
| | MLANA#11 | | | 385 | | to be |
| | | | | | | immunogenic) |
| Pt-D | PD-PMEL#1 | PMEL | KTWGQYWQV | SEQ ID NO: | 9 | Y |
| | | | | 386 | | |
| Pt-D | PtD-PMEL#2 | PMEL | AMLGTHTMEV | SEQ ID NO: | 10 | Y |
| | | | | 387 | | |
| Pt-D | PtD-PMEL#3 | PMEL | ALDGGNKHFL | SEQ ID NO: | 10 | Y |
| | | | | 388 | | |
| Pt-D | PtD-PMEL#4 | PMEL | SLADTNSLAVV | SEQ ID NO: | 11 | Y |
| | | | | 389 | | |
| Pt-D | PtD-PMEL#5 | PMEL | LDGGNKHFL | SEQ ID NO: | 9 | Y |
| | | | | 390 | | |
| Pt-D | PtD-PMEL#6 | PMEL | MDLVLKRCL | SEQ ID NO: | 9 | Y |
| | | | | 391 | | |
| Pt-D | PtD-PMEL#7 | PMEL | ITDQVPFSV | SEQ ID NO: | 9 | Y |
| | | | | 392 | | |
| Pt-D | PtD-PMEL#8 | PMEL | RYGSFSVTL | SEQ ID NO: | 9 | Y |
| | | | | 393 | | |
| Pt-D | PtD-PMEL#9 | PMEL | LYPEWTEAQRL | SEQ ID NO: | 11 | Y |
| | | | | 394 | | |
| Pt-D | PID-TYR#1 | TYR | NIFDLSAPEKD | SEQ ID NO: | 15 | Y |
| | | | KFFA | 395 | | |
| Pt-D | PtD-TYR#2 | TYR | FLPWHRLFL | SEQ ID NO: | 9 | Y |
| | | | | 396 | | |
| Pt-D | PtD-TYR#3 | TYR | LLMEKEDYHSL | SEQ ID NO: | 11 | Y |
| | | | | 397 | | |
| Pt-D | PtD-TYR#4 | TYR | MLLAVLYCL | SEQ ID NO: | 9 | Y |
| | | | | 398 | | |
| Pt-D | PtD-TYR#5 | TYR | SYLEQASRI | SEQ ID NO: | 9 | Y |
| | | | | 399 | | |
| Pt-D | PtD-TYR#6 | TYR | EEYNSHQSL | SEQ ID NO: | 9 | Y |
| | | | | 400 | | |
| Pt-D | PtD-DCT#1 | DCT | SLDDYNHLV | SEQ ID NO: | 9 | Y |
| | | | | 401 | | |
| Pt-D | PtD-PRAME#1 | PRAME | SIQSRYISM | SEQ ID NO: | 9 | Y |
| | | | | 402 | | |
| Pt-D | PtD-PRAME#2 | PRAME | FLRGRLDQL | SEQ ID NO: | 9 | Y |
| | | | | 403 | | |
| Pt-D | PtD-PRAME#3 | PRAME | SLLQHLIGL | SEQ ID NO: | 9 | Y |
| | | | | 404 | | |
| Pt-D | PtD-PRAME#4 | PRAME | GLSNLTHVL | SEQ ID NO: | 9 | Y |
| | | | | 405 | | |
| Pt-D | PtD-PRAME#5 | PRAME | SQFLSLQCL | SEQ ID NO: | 9 | Y |
| | | | | 406 | | |
| Pt-D | PtD-PRAME#6 | PRAME | GQHLHLETF | SEQ ID NO: | 9 | Y |
| | | | | 407 | | |
| Pt-D | PtD-PRAME#7 | PRAME | TSPRRLVEL | SEQ ID NO: | 9 | Y |
| | | | | 408 | | |
| Pt-D | PtD-PRAME#8 | PRAME | FLKEGACDEL | SEQ ID NO: | 10 | Y |
| | | | | 409 | | |
| Pt-D | PtD-PRAME#9 | PRAME | LYVDSLFFL | SEQ ID NO: | 9 | Y |
| | | | | 410 | | |
| Pt-D | PtD- | PRAME | RLDQLLRHV | SEQ ID NO: | 9 | Y |
| | PRAME#10 | | | 411 | | |
| Pt-D | PtD- | PRAME | SQSPSVSQL | SEQ ID NO: | 9 | Y |
| | PRAME#11 | | | 412 | | |
| Pt-D | PtD- | PRAME | VLYPVPLESY | SEQ ID NO: | 10 | Y |
| | PRAME#12 | | | 413 | | |
| Pt-D | PtD- | PRAME | HARLRELL | SEQ ID NO: | 8 | Y |
| | PRAME#13 | | | 414 | | |
| Pt-D | PtD- | PRAME | LAYLHARL | SEQ ID NO: | 8 | Y |
| | PRAME#14 | | | 415 | | |
| Pt-D | PtD- | PRAME | YLHARLREL | SEQ ID NO: | 9 | Y |
| | PRAME#15 | | | 416 | | |

*Detected by MS in HLA class I immunopeptidome of melanoma cell lines

The analysis of CD137 upregulation upon in vitro stimulation allows the identification of T cell reactive against tumor cells or tumor antigens. TCR-transduced effectors were labeled with different combinations of 3 dyes (Cell Trace (CT) CFSE, Far Red or Violet), with up to 4 dilutions per dye, allowing identification of single effectors. The analysis was repeated for each effector population. CD137 upregulation was measured on transduced (mTRBC+) CD8+ cells upon overnight incubation with different target cells. The same strategy was adapted to test patient PBMC upon in vitro enrichment of anti-melanoma specificities (data not shown), with the additional gating on viable (Zombie Aqua −) CD3+ cells prior identification of CD8+ cells (as reported for the sorting of melanoma reactive CD8+ T cells).

Following overnight co-incubation of effector and target cells, TCR reactivity was assessed by flow cytometric detection of CD137 upregulation on CD8+ transduced T cells, using the following antibodies: anti-human CD8a (BV785, clone RPA-T8, Biolegend), anti-mouse TRBC (PE-Cy7, clone H57-597, eBioscience) and anti-human CD137 (PE, clone 4B4-1, Biolegend). To test in vitro enriched antimelanoma T cells from patients' PBMCs, anti-human CD3 (APC-Cy-7, clone UCHT1, Biolegend) and Zombie Aqua viability die (Biolegend) were included in the staining procedure. Data were acquired on a high throughput sampler (HTS)-equipped Fortessa cytometer (BD Biosciences) and analyzed using Flowjo v10.3 software (BD Biosciences). For each tested condition, background signal measured on CD8+ T cell transduced with an irrelevant TCR was subtracted. Based on CD137 upregulation upon challenge with the different targets, each TCR was classified as: i) tumor-specific (conventional or inflammation responsive, based on the response detected against melanoma cell lines without or with IFNγ pretreatment, respectively); ii) non-tumor-reactive; and iii) tumor/control reactive. A TCR was considered tumor-reactive if the level of background-subtracted CD137 upon coculture with melanoma cells was at least 5% with 2 standard deviations higher than that of the unstimulated control (mean value from 3 replicates per condition). Activation-dependent TCR downregulation was manually evaluated to further corroborate ongoing TCR signal transduction.

In peptide deconvolution analyses, peptide recognition was calculated by subtracting the background detected with DMSO-pulsed EBV-LCLs from the CD137 upregulation level measured from the peptide-pulsed EBV-LCLs. When TCRs specific for individual peptides were identified, reactivity was validated and titrated using EBV-LCL cells pulsed with increasing doses of pure peptides (from 10⁰-10⁸ pg/mL). For NeoAg-specific TCRs, titration was performed for both mutated and wildtype antigens. To define the recognition affinity for each TCR-peptide pair, results of titration curves were normalized, and EC50 values were calculated using GraphPad Prism 8 software. Finally, HLA restriction of tumor-specific TCRs with identified specificity was determined by measuring CD137 upregulation upon stimulation with available monoallelic HLA lines (Sarkizova et al., Nat. Biotechnol. 38:199-209 (2020); Abelin et al., Immunity 46:315-26 (2017)) (expressing single patients' HLAs) pulsed with peptide of interest.

Statistical analysis. The following statistical tests were used in this study, as indicated throughout the text: 1) Spearman's correlation coefficients and associated two-sided P values were computed using R to test the null hypothesis that the correlation coefficient is zero; (2) two tailed Fisher's exact test were performed with R to calculate significance of deviation of a distribution from the null hypothesis of no differential distribution (FIG. 2); (3) Welch t tests were performed using the GraphPad Prism 8 software to obtain the two-sided P value of the null hypothesis that the two groups have equal means; (4) Wilcoxon rank sum test was performed with R for data with high variance to test whether mean ranks differ; (5) Ratio-paired parametric t-tests were performed using the GraphPad Prism 8 software, to obtain the two-sided P value of the null hypothesis that the paired values of two groups have ratio equal to 1; (6) Linear regressions were performed on LOG-transformed values of different parameters using GraphPad Prism 8 software, which provided R² values and two-sided P value of the null hypothesis that the regression coefficient is zero; and (7) Normalized Shannon Index was calculated on patient-specific TCRs or on all available TCR clonotypes as follows: k: number of TCRs clonotypes, n=total count of cells, f: frequency.

No statistical methods were used to predetermine sample size. The experiments were not randomized. The investigators were not blinded to allocation during experiments and outcome assessment.

Code availability. Code used for data analysis included the Broad Institute Picard Pipeline (WES/RNA-seq), GATK4 v4.0, Mutect2 v2.7.0 (sSNV and indel identification), NetMHCpan 4.0 (neoantigen binding prediction), ContEst (contamination estimation), ABSOLUTE v1.1 (purity/ploidy estimation), STAR v2.6.1c (sequencing alignment), RSEM v1.3.1 (gene expression quantification), Seurat v3.2.0 (single-cell sequencing analysis), Harmony v1.0 (single-cell data normalization), SingleR v3.22, Scanpy v1.5.1, Python v3.7.4 (for comparison with other single cell datasets) that are each publicly available. Computer code used to generate the analyses is available at github.com/kstromhaug/oliveira-stromhaug-melanoma-tcrs-phenotypes.

Data availability statement. Single-cell RNA, TCR and CITEseq sequencing are available through dbGaP portal (study Id 26121, accession number phs001451.v3.p1).

### Example 2: Distinct Tumor-Infiltrating CD8+ TCR Clonotype Families Segregate as Having Either Exhausted or Non-Exhausted Cell States

Identifying tumor-infiltrating T cells and their tumor specificity is a major obstacle to the reliable identification of usable TIL and the identification of tumor-reactive TCRs. The focus of this study was five tumor specimens collected from skin, axillary lymph node or lung from 4 patients (Pt-A, Pt-B, Pt-C, and Pt-D) with stage III or IV melanoma that were previously reported. See, Ott et al., Nature 547:217-21 (2017); Hu et al., Blood 132:1911-21 (2018). The tumor biopsies were harvested from Pt-A, Pt-B, Pt-C, and Pt-D at time of surgery and were analyzed with single-cell sequencing and TCR specificity. Peripheral blood samples were collected before and after immunotherapy and were used for isolation of tumor-reactive T cells at serial time-points (TP). To characterize the phenotype and clonality of the CD8+ TILs (FIG. 1A), high-throughput single-cell transcriptome (scRNAseq), TCR sequencing (scTCR-seq) coupled with detection of surface proteins (i.e., CITEseq (Stoeckius et al., Nat. Methods 14:865-8 (2017)), Table 1) was used for more definitive identification of CD4/CD8 T cell subpopulations and conventional T cell differentiation states; of which a schematic is illustrated in FIG. 1A, which shows sample collection, processing, and single-cell sequencing analysis of melanoma and peripheral blood samples. The dataset of transcriptomes from 30,319 single CD8+ T cells derived predominantly from the 3 of 5 biopsies with modest or high T cell infiltration, see Table 2-Table 4. Flow cytometry plots indicated the proportion of T lymphocytes (defined as CD45+CD3+) infiltrating 5 tumor biopsies subjected to single-cell sequencing (data not shown). Tissue origin for each tumor sample is indicated in Table XX. CD4+ and CD8+ TILs were identified using density plots showing CITEseq antibody signals for CD4+ and CD8+ antibodies. normalized signals were calculated as CD4 and CD8 CITEseq signals relative to isotype controls for all sequenced cells that were identified as T cells after flow sorting and computational filtering.

CD8+ TILs clustered into 13 subsets (FIG. 1B left, Table 2-Table 4), classified based on RNA and surface protein expression of a panel of T cell-related genes and by cross-labelling with reference gene signatures from external single-cell datasets of human TILs (Sade-Feldman et al., Cell 176:1-20 (2019); Yost et al., Nat. Med. 25:1251-59 (2019); Oh et al., Cell 181:1612-25.e13 (2020)), as illustrated in FIG. 4A-4C. Uniform manifold approximation and projection (UMAP) of scRNA-seq data from CD8+ melanoma-infiltrating T cells defined by CD8-CITEseq (Cellular Indexing of Transcriptomes and Epitopes by Sequencing) antibody positivity (left) is shown in FIG. 1B. Clusters are labeled with inferred cell states and metaclusters. The same UMAP (right), show T cells marked based on intra-patient TCR clone frequency defined through scTCR-seq. The top 100 TCR clonotype families from four patients the cluster distribution of the top 100 CD8+ dominant TCR clonotype families from tumor biopsies of 4 patients included in the discovery cohort is shown in FIG. 1C. Colors (black, White, gray) denote primary cell states, as delineated in FIG. 1B. Spearman correlation of normalized cluster distribution of dominant TCR clonotype families composed by >5 cells was demonstrated (data not shown).

Heatmaps depicting the mean cluster expression of a panel of T-cell related genes, measured by scRNAseq (left panel) and the mean surface expression of the corresponding proteins measured through CITEseq (right panel) is shown in FIG. 4A. Clusters (columns) are labelled using the annotation provided in FIG. 1B; markers (rows) are grouped based on their biological function. Grey—unevaluable markers (CD45 isoforms for scRNASeq) or which were not assessed (for CITESeq). CITESeq CD3 surface expression was poorly detected because of the presence of competing CD3 sorting antibody. Violin plots quantifying relative transcriptional expression of genes (columns) with high differential expression among CD8+ TIL clusters (rows) is shown in FIG. 4B. UMAPs depicting the single-cell expression of representative T cell markers among CD8+ TILs either through detection of surface protein expression with CITEseq (Ab), or through scRNAseq (RNA) is shown in FIG. 4C. The characterization of the CD8+ TIL clusters was validated using independent reference gene-signatures (Sade-Feldman et al., Cell 176:1-20 (2019); Yost et al., Nat. Med. 25:1251-59 (2019); Oh et al., Cell/81:1612-25.e13 (2020)), by cross-labelling oT cell clusters defined in the present study (as in FIG. 1B) versus published reference gene-signatures.

Rare CD45RA+CD62L+CCR7+IL7Rα+ naïve T cells (T_{N}, Cluster 12, FIG. 4A) could be distinguished from remaining clusters of differentiated CD45RO+CD95+ cells. These included effector memory (T_{EM}) and memory (T_{M}) CD8 T cells (Clusters 1 and 2, respectively) expressing memory markers (IL7R, TCF7), albeit with differential transcription of effector cytokines (GZMA, GZMB, GZMH, PRF1). Cluster 3 matched reported activated CD8+ cells (Ta ct), marked by the high expression of the transcription factor NR4A1 and heat shock proteins. A large proportion of CD8+ TILs displayed high levels of inhibitory and cytotoxic markers: Cluster®, together with 2 Pt-C-specific clusters (Clusters 8 and 11), exhibited high association with published terminally exhausted (T_{TE}) TILs, and shared robust expression of inhibitory molecules (PDCM, TIGIT, HAVCR2, LAG3), regulators of tissue residency (ITGAE, ZNF683) and cytotoxicity (PRF-1, IFNG, FASLG). Size and patient distribution of the 13 clusters was identified from CD8+ TIL scRNAseq and represented for each patient (data not shown); The analyzed CD8+ dataset is predominantly composed by cells from 3 patients (Pt-A, Pt-C and Pt-D). Two clusters were found to be patient-specific (clusters 8 and 11). Right: UMAPs depicting cluster distribution of patient-specific CD8+ TILs. Cluster 4 was marked by the highest expression of the transcription factor TOX and differed from T_{TE} based on higher expression of memory-associated transcripts (TCF7, CCR7, IL7R), consistent with previously identified progenitor exhausted T cells (T_{PE}). See, Miller et al., Nat. Immunol. 20:326-36 (2019). Five additional minor clusters were identified: proliferating cells (Cluster 5, Tₚᵣₒₗ), apoptotic cells (Cluster 6, T_{Ap}), NK-like CD8+ cells (Cluster 7), contaminant T reg-like cells with low CD4 expression and low surface binding of the CD8-CITEseq antibody (Cluster 9), and γδ-like T cells (Cluster 10).

The relationship between phenotype and TCR clonality of the CD8+ TILs was evaluated: scTCR-seq allowed detection of TCR α- or β-chains in 24,477 cells that were subsequently grouped into 7,239 distinct clonotypes by matching V, J, and CDR3 regions (Table 2-Table 4). Of the 1804 TCR clonotype families (defined as clonotypes with >1 cell), highly expanded T-cell clones were distributed most predominantly in cells with exhausted phenotypes (FIG. 1B-right). Clonotypes families were divided based on their size and their number and overall frequencies were analyzed (data not shown). Intra-cluster TCR diversity was maximal among T_{N} cells, and progressively decreased with transition from memory to exhausted phenotypes, as determined among CD8+ T cells in each cluster using normalized Shannon index (data not shown). Most of TCR clonotypes were confined to a defined area of the UMAP (data not shown). Strikingly, the cluster distributions of cells harboring the same TCRs fell in one of two distinct patterns, wherein the predominant phenotype per clonotype was either ‘non-Exhausted Memory’ (T_{NExM}, clusters 1, 2, 7 and 10) or ‘Exhausted’ (T_{Ex}, clusters 0, 4, 5, 8 and 11) (FIG. 1C). The acquisition of an exhausted phenotype encompassed the T_{TE}, T_{PE} and Tₚᵣₒₗ CD8+ T lymphocytes, thus linking together these diverse differentiation stages of exhausted cells, with Pt-C having higher numbers of exhausted progenitor cells within expanded TCR clonotypes (FIG. 1C). In contrast, the less differentiated T_{M} and T_{EM} cells segregated together, but were negatively correlated with T cells bearing exhaustion phenotypes. Thus, for most clonotype families, CD8+ T cells were either distributed among clusters with exhausted phenotypes or among non-exhausted ones. Such a pattern of distribution allowed the assignment of a “primary cluster” to each expanded TCR clonotype family as an approximate phenotype.

### Example 3: TCR Clonotypes with Exhausted Phenotypes are Enriched in Melanoma-Reactive Specificities

The detection of these two distinct phenotypic patterns, each delineated based on TCR identity, led to the hypothesis that this separation was driven by the recognition of different classes of antigens, resulting in different potential for antitumor reactivity. The ability of the most highly represented TCRs whose primary clusters were either T_{Ex} or T_{NExM} to recognize autologous melanoma cells was thus tested. A representative set of dominant TCR clonotypes (123 T_{Ex}, 49 T_{NExM}) were cloned and expressed in T cells from healthy individuals, as illustrated in FIG. 2A, which shows the workflow for in vitro TCR reconstruction and specificity screening. Multiple TCRs are cloned, expressed in healthy donor T cells (top panel, FIG. 2A). Pools of effectors with 3 dies (CFSE, cell-trace Violet, cell-trace Far Red) expressing individual TCRs are tested for reactivity against patient-derived melanoma cells, controls or Epstein-Barr virus lymphoblastoid cell lines (EBV-LCLs) (middle and bottom panels, FIG. 2A), that could be pulsed with peptides from neoantigens (NeoAgs), melanoma associated antigens (MAAs) or viral antigens selected from mass spectrometry (MS) detection of human leukocyte antigen (HLA)-class I tumor immune peptidome, from computational prediction or from commonly availability peptide pools. The reactivity of dominant TCRs originating from cells in exhausted (T_{Ex}, top) or non-exhausted memory (T_{NExM}, bottom) clusters infiltrating 4 melanoma specimens is shown in FIG. 2B. CD137 upregulation was measured on TCR-transduced (mTRBC+) CD8+ T cells cultured alone (no target) or in the presence of autologous melanoma cells (Mel, with or without IFNγ pre-treatment) or controls (PBMCs, B cells and EBV-LCLs). Background detected on CD8+ T cells transduced with an irrelevant TCR was subtracted. For each TCR clonotype tested (rows), the primary cluster and frequency detected among patient CD8+ TILs are scored in the tracks left of the heat map, while classification of TCR reactivities are scored on the right track. UT: level of reactivity of untransduced CD8+ lymphocytes.

Multicolor labeling (CFSE, cell-trace Violet, cell-trace Far Red) of effector cell lines transduced with individual TCRs enabled parallel screening of their antigenic specificities using standard multiparametric flow cytometry (see EXAMPLE 1: Materials and Methods). The transduction of TCR signal, detected as upregulation of the activation molecule CD137 (Wolff et al., Blood 110:201-10 (2007)), was measured upon co-culture of effector cell pools against patient-derived melanoma cell lines, each confirmed by genomic and transcriptomic characterization to recapitulate the features of the parental tumor, and against non-tumor controls (autologous peripheral blood mononuclear cells (PBMCs), B cells and EBV-immortalized lymphoblastoid cell lines (EBV-LCLs)).

The purity of tumor cultures, originated from patient biopsies, was assessed by flow cytometry (data not shown) by staining cells with isotype controls or surface markers (identifying melanoma (using melanoma chondroitin sulfate proteoglycan, MCSP) or fibroblast lineages (fibroblast antigen). Consistent with previous reports (Campoli et al., Crit. Rev. Immunol. 24:267-96 (2004)), MCSP was expressed in 3 of 4 tumor cultures, with each lacking substantive fibroblast contamination. The flow cytometric assessment of HLA class I surface expression on established melanoma cell lines was carried out. Surface expression was measured with a pan-HLA class I antibody or with an HLA-A:02-specific antibody (bottom in basal culture conditions or upon exposure to IFNγ for 72 hours, compared to isotype control (data not shown)). The identification of mutation in patient-derived melanoma cell lines vs. corresponding parental tumors allowed to demonstrate that melanoma cell lines recapitulated the genomic profiles of parental tumors (data not shown). For all patients, mutation calling from whole-exome sequencing (WES) of tumor biopsies and cell lines was performed through comparison with autologous PBMCs serving as germline controls. For each cell line-parental tumor pairs, the numbers and frequencies of shared or sample-specific mutations was analyzed. For each mutation, variant allele frequencies (VAF) detected in the parental tumors and derived cell lines was reported (data not shown). For both, tumor purity inferred from single-cell data (parental tumors) or detected by flow cytometry (cell lines) is indicated. The high concordance between the genomic mutations detected in paired specimens demonstrates that the melanoma cell lines are reflective of the corresponding parental tumors. Similarity between the transcriptional profile of parental tumors and corresponding cell lines was identified through analysis of expression of HLA class I genes and melanoma-related genes, measured through bulk RNA-seq. The same data were generated for non-tumor fibroblasts, as negative controls. HLA class I immunopeptidome of patient-derived melanoma cell lines cultured with or without IFNγ was determined using mass spectrometry (MS) after immunoprecipitation of peptide-HLA class I complexes.

In total, 102 of 123 (83%) T_{Ex} TCRs analyzed across 4 patients were confirmed to be tumor-specific (see, for example, FIG. 2B). For Pt-A, 13 of 53 (25%) TCRs tested displayed tumor reactivity only following IFNγ-induced upregulation of tumor antigen presentation and HLA surface expression. For the clonotypes from T_{NExM} clusters, only 5 of 49 TCRs (10%) exhibited tumor recognition (FIG. 2B), while 11 (22%) non-tumor reactive TCRs recognized EBV-LCLs, supporting their likely specificity for viral antigens. TCRs cloned from T_{Ex} clusters, and not from T_{NExM} clusters, conferred both activation and cytotoxic potential to transduced lymphocytes (FIG. 5).

TCR reactivity was classified based on CD137 upregulation of TCR transduced T cell lines upon challenge with patient-derived melanoma cells (Mel, with or without IFNγ pre-treatment) or controls (PBMCs, B cells and EBV-LCLs). A TCR was defined as tumor-specific if it recognized only the autologous melanoma cell line but did not upregulate CD137 when challenged with autologous controls. Flow cytometry plots (not shown) depicting CD137 upregulation measured on CD8+ T cells transduced with TCRs isolated from Pt-A and cultured with melanoma or control targets represented examples and thresholds for the classification of tumor or non-tumor reactive TCRs. Background reactivity was estimated by measuring CD137 upregulation on CD8+ T cells transduced with an irrelevant TCR. Cytotoxic potential provided by TCRs with exhausted or non-exhausted primary clusters isolated from all 4 studied patients was analyzed, to investigate the ability of each TCR to transduce signals resulting in production of cytotoxic cytokines. Degranulation (CD107a/b+) and concomitant production of cytokines (IFNγ, TNF and IL-2) were assessed through intracellular staining, gating on TCR-transduced (mTRBC+) CD8+ T cells cultured alone or in the presence of autologous melanoma. Each dot represents the result for a single TCR isolated from CD8+ TILs, reported based on its primary phenotypic cluster (as defined in FIG. 1B). For each analyzed TCR, background cytotoxicity from CD8+ T cells transduced with an irrelevant TCR was subtracted. Open dots (FIG. 5) depict the basal level of activation of untransduced cells. Overall, these data indicate that antitumor cytotoxicity mainly resides among TCR clonotypes with exhausted primary clusters.

Additionally, 5 T_{NExM} TCRs demonstrated non-specific recognition of both tumor and control cells. Overall, T_{Ex} TCR clonotypes were enriched in antitumor specificities, while T_{NExM} TCR clonotypes were enriched in anti-EBV specificities (p<0.0001, Fisher's exact test, FIG. 2C). Moreover, TCR sequences with known antiviral specificities mined from a TCR database (Chen et al., Nucleic Acids Res 49:D468-D474 (2021)) could be matched only to 4 TCR clonotypes with T_{NExM} primary cluster (FIG. 2D). The proportion of TCRs classified as tumor-specific (left) or EBV-specific (right) among TCR clonotypes reconstructed from T_{Ex} or T_{NExM} clusters is shown in FIG. 2C. P values are calculated using Fisher's exact test on total distribution of tested TCRs. The number of TCRs from T_{Ex} or T_{NExM} clusters that perfectly matched with known TCR sequences from TCRdb (Chen et al., Nucleic. Acids. Res. 49:D468-D474 (2021) are shown in FIG. 2D. The reactivity and phenotypic distribution of TCRs isolated from peripheral blood, traced within the tumor microenvironment, was determined (data not shown). FIG. 2E is a UMAP of scRNA-seq data from CD8+ TILs bearing any of 134 TCRs with in vitro verified antitumor specificity showing the cell states of tumor-specific (TS) CD8+ TILs. The cluster distribution was of 134 tumor-specific TCR clonotypes, grouped based on their primary cluster.

In a complementary evaluation of blood-derived T cells, TCRs were isolated from cells with confirmed melanoma reactivity, in order to discover their phenotype through mapping of those TCRs to the expression states delineated from TIL analysis (data not shown). Circulating CD8+ T cells were FACS-sorted on the basis of degranulation and concomitant cytokine release following in vitro stimulation of PBMC (collected before or after immune treatments) with autologous melanoma cell lines (FIG. 1A-right). Across 4 patients, plate-based scTCRseq of 1737 circulating CD8+ T cells resulted in identification of 491 TCRα/TCRβ chain pairs (EXAMPLE 1: Materials and Methods), and 414 TCRs were reconstructed and screened in vitro against autologous melanoma and controls. Tumor specificity was established for 216 (52%) of blood-derived TCRs (data not shown), while 61 (15%) showed non-specific reactivity and 137 (33%) were not reactive against tumor cells. Sixty-seven blood-derived TCRs (51 tumor-specific, 16 non-tumor-reactive) could be tracked back to CD8+ TILs by the matching of TCRα/TCRβ chain pair information across these two tissue compartments. Again, it was observed that TCRs with tumor specificity preferentially exhibited a T_{Ex} phenotype, while the majority of non-tumor reactive TCRs were traced to the T_{NExM} clusters (p<0.0001, Fisher's exact test, FIG. 2C-bottom).

PBMCs collected at serial timepoints (TP1: before immunotherapy, TP2-TP3: 16-52 weeks after immunotherapy) were cultured with autologous melanoma cell lines to enrich for antitumor TCRs (data not shown). After two rounds of stimulation, the reactivity of effector CD8+ T cells was assessed by measuring: degranulation and cytokine production; or CD137 upregulation upon re-challenge with melanoma. The specificity of the response was supported by the low recognition of HLA-mismatched unrelated melanoma. Negative controls (culture in the absence of target cells) and positive controls (polyclonal stimulators, PHA or PMA-ionomycin) were used. FACS sorting strategy for the isolation of tumor-reactive T cells was carried out. CD8+ effectors with active degranulation and concomitant cytokine production were identified using cytokine secretion assays (see EXAMPLE 1: Materials and Methods) upon stimulation without any target or in the presence of autologous melanoma. CD107a/b+ cells secreting at least one of the measured cytokines (IFNγ, TNF and IL-2) were single-cell sorted and sequenced. TCR clonotypes were identified upon single-cell sorting and scTCRseq of melanoma-reactive CD8+ T cells from the 4 studied patients.

TCRs isolated and sequenced from anti-melanoma cultures were reconstructed, expressed in CD8+ T cells and screened against melanoma (pdMel-CL, with or without IFNγ pre-treatment) or controls (PBMCs, B cells and EBV-LCLs) (data not shown). TCRs were classified to identify: tumor-specific TCRs; non-tumor reactive TCRs; and tumor/control reactive TCRs. Reactivity was calculated by subtracting the background of lymphocytes transduced with an irrelevant TCR from CD137 expression of CD8+ cells transduced with the reconstructed TCR. The classification of TCR reactivity for all reconstructed TCRs can be summarized as follows: Tumor-specific (reactive only towards tumor cells); Non-tumor reactive (no reactivity detected against tumor cells); tumor/control reactive (reactive against tumor and non-tumor samples). Degranulation (CD107a/b+) and concomitant production of cytokines (IFNγ, TNF and IL-2) were measured through intracellular flow cytometry on TCR transduced (mTRBC+) CD8+ T cells cultured alone or in the presence of autologous pdMel-CLs. Each measure was performed a single TCR isolated from CD8+ TILs (upon subtraction of background activation measured on CD8+ lymphocytes transduced with an irrelevant TCR) and reported in comparison the basal level of cytotoxicity of untransduced cells. Intratumoral cluster distribution of cells bearing tumor-specific or non-tumor reactive TCRs were isolated from blood and traced within the tumor microenvironment.

To validate the results in an independent cohort, 94 clonally expanded TCRs sequenced from CD8+ TILs of 7 patients with metastatic melanoma previously characterized by scRNAseq (Sade-Feldman et al., Cell 176:1-20 (2019)) (Table 5) were reconstructed. Antiviral specificity was established for 7 TCRs, either by testing TCR-transduced T cells against autologous EBV-LCLs (n=5, FIG. 6A) or by matching TCR sequences with a database of known TCR specificities (Chen et al., Nucleic Acids Res. 49:D468-D474 (2021)) (n=2). Activation upon stimulation with EBV-LCLs pulsed with peptide pools covering 12 known melanoma-associated antigens (MAAs) was used as a proxy of antitumor specificity (FIG. 6B). In total, 22 MAA-specific TCRs and 7 virus-specific clonotypes (FIG. 6C) that were expressed by CD8+ T cells with distinct transcriptomic profiles were identified: the former mapped preferentially to previously described memory clusters, while the latter almost exclusively to exhausted subsets (p<0.0001, Fisher's exact test, FIG. 6D-6E). Direct comparison of virus- and MAA-specific cells highlighted transcriptional upregulation of exhaustion genes (PDCD1, HAVCR2, CTLA4; FIG. 6F). Thus, T cells with capacity for antitumor recognition clearly reside predominantly within the exhausted compartment rather than within the less differentiated T_{NExM} compartment, and the acquisition of these T_{Ex} profiles within the tumor microenvironment is an antigen-driven process.

The unambiguous determination of the antitumor reactivity of 134 in vitro reconstructed TCRs from the discovery cohort, prompted a deeper investigation into the cellular phenotypes of those true tumor-specific (TS) CD8+ T cells. First, the average phenotype of TS-TCR clonotypes were analyzed: as expected, they could be readily distinguished from virus-specific T cells based on the deregulation of 98 RNA transcripts (FIG. 6G-6H, Table 5), which included known transcription factors (TCF7/TOX and genes (IL7R, CCR7/PDCD1, HAVCR2, ENTPD1) associated with the regulation of memory/exhaustion cell states. Six surface proteins were highly expressed on TS-TCR clonotypes (CD27, CD38, CD39, CD69, ICOS, PD1), the highest of which were PD1 and CD39, which were previously associated to antitumor responses (Scheper et al., Nat. Med. 25:89-94 (2019); Simoni et al., Nature 557:575-579 (2018); Gros et al., J. Clin. Invest. 124:2246-2259 (2014); Duhen et al., Nat. Commun. 9:1-13 (2018)). These upregulated transcripts or surface proteins may be used in methods described herein as a memory marker, to efficiently select exhausted T cells.

Antigen specificity screening of 94 TCRs sequenced from clonally expanded CD8+ T cells isolated from tumor biopsies of 7 patients with metastatic melanoma from Sade-Feldman et al. (Sade-Feldman et al., Cell 176:1-20 (2019)) is shown in FIG. 6A-6C. After TCR reconstruction and expression in T cells, reactivity was measured as CD137 upregulation on TCR-transduced (mTRBC+) CD8+ cells upon culture with autologous EBV-LCLs pulsed with peptide pools covering immunogenic viral epitopes (CEF) as shown in FIG. 6A. Unstimulated cells were analyzed as negative control. Results are reported after subtraction of background CD137 expression on T cells transduced with an irrelevant TCR. Five TCRs (black dots) recognized unpulsed EBV-LCLs thereby documenting specificity for EBV epitopes. TCR antitumor reactivity is shown in FIG. 6B, evaluated upon culture with autologous EBV-LCLs pulsed with peptide pools of 12 known MAAs. Background detected upon culture with DMSO-pulsed EBV-LCLs was subtracted. Additional positive and negative controls were an irrelevant peptide (Ova) and polyclonal stimulators (PHA or PMA/ionomycin), respectively. Dots above 10% threshold denote MAA-reactive TCRs. Patient distribution of TCR specificities is summarized in FIG. 6C where either discovered from reconstruction and screening of 94 TCRs or present within a database of human TCRs with known specificities (TCRdb) (Chen, S.-Y., et al., Nucleic Acids Res 49, D468-D474 (2021)). FIG. 6D-6F show single-cell phenotype of TILs with antiviral or anti-MAA TCRs identified in the validation cohort from Sade-Feldman et al. (Sade-Feldman et al., Cell 176:1-20 (2019)). FIG. 6D shows the t-SNE plot of CD8+ TILs highlighting the spatial distribution of cells harboring TCRs with identified antigen specificity. Pie charts shown in FIG. 6E summarize the assignment of single cells harboring antiviral (top) or anti-MAA (bottom) TCRs to one of the previously reported 6 clusters (Sade-Feldman et al., Cell 176:1-20 (2019)). FIG. 6F shows RNA transcripts differentially expressed between antiviral and anti-MAA cells (log₂FC>1.5, adj. p value<0.05). The heatmap reports Z scores, calculated from average gene expression of each TCR clonotype family (columns) Antigen classes are reported on top the heatmap. FIG. 6G-6H show the analysis of deregulated genes in exhausted clusters (T_{Ex}), enriched in tumor-reactive T cells, from the discovery cohort. Average gene expression, reported as Z scores, for each TCR clonotype family (columns) validated in vitro as tumor-specific (right, 134 TCRs) or defined as virus-specific (left, 17 TCRs) is shown in FIG. 6G. The heatmap reports 98 RNA transcripts (adj. P<0.0001, log₂FC>1) and 6 surface proteins (bottom rows, adj.P<0.0001, log₂FC>0.4) detected through scRNAseq and CITEseq respectively. FIG. 6H shows plots depicting expression of representative RNA-transcripts (top) or surface proteins (bottom) in each TCR clonotype family with antiviral (black) or antitumor (grey) specificity. Dots depict the average gene-expression in each TCR clonotype, with size proportional to the frequency of the TCR clonotype within patient-specific CD8+ TILs.

A heatmap depicting the top 20 overexpressed genes in each TS-cluster showing the cell states of tumor-specific (TS) CD8+ TILs was obtained (data not shown). Heatmaps depicting expression of a panel of T cell related transcripts detected through scRNAseq or surface proteins detected through CITEseq were obtained (data not shown). Z scores document the trends in expression among subpopulations of tumor-specific CD8+ cells (columns). Enrichment in expression of gene signatures among identified clusters of tumor-specific (TS) CD8+ cells (columns) was seen. Single cells with tumor-specific TCRs were divided in clusters as reported in FIG. 2E, and scored for the expression of gene signatures defined from analysis of CD8 TILs of the discovery cohort (left), reported in external datasets of sequenced human CD8+ TILs (middle), or defined from published murine studies (right) (see EXAMPLE 1: Materials and Methods and Table 2-Table 4). Average enrichment score was calculated for each cluster and reported as Z score.

Second, the fine differences among TS-CD8+ T cells were captured by re-clustering the 7451 single cells that comprised the 134 TS-TCR clonotype families. Then, 5 TS-clusters (FIG. 2E, Table 7-Table 11) were identified, which were scored based on enrichment of gene-signatures annotated from internal or external published data (Table 2-Table 4) and based on the RNA and surface protein expression characteristics of a set of T cell-related genes. Thus identified were: i) TS-T_{TE} cells, which resembled human (Sade-Feldman et al., Cell 176:1-20 (2019); Yost et al., Nat. Med. 25:1251-59 (2019)) and murine (Miller et al., Nat. Immunol. 20:326-336 (2019); Utzschneider et al., Immunity 45:415-27 (2016); Im et al., Nature 537:417-21 (2016); Siddiqui et al., Immunity 50:195-211.e10 (2019)) T_{TE}, were enriched in PRF1 and GZMB transcripts and displayed high expression of exhaustion proteins (PD1, Tim-3, LAG3, CD39); ii) TS-TAc t cells, corresponding to tissue resident memory cells in a state of acute activation (Yost et al., Nat. Med. 25:1251-59 (2019); Milner et al., Nature 552:253-7 (2017)), given their high expression of IFNG and heat shock protein-transcripts; iii) TS-T_{PE} cells, characterized by TCF7 and CCR7 positivity, high levels of activation molecules (HLA-DR, CD137), lower expression of inhibitory proteins, but absent cytotoxic potential, consistent with previously described T_{PE} (Miller et al., Nat. Immunol. 20:326-336 (2019); Utzschneider et al., Immunity 45:415-27 (2016); Im et al., Nature 537:417-21 (2016); Siddiqui et al., Immunity 50:195-211.e10 (2019)); iv) TS-Tₚᵣₒₗ cells, highly exhausted, but in active proliferation; v) TS-TE M cells, which resembled human and murine memory T cells with stem-like properties (Yost et al., Nat. Med. 25:1251-59 (2019); Miller et al., Nat. Immunol. 20:326-336 (2019); Joshi et al., Immunity 27:281-95 (2007); Jansen et al., Nature 576:465-70 (2019); Krishna et al., Science 370:1328-34 (2020)) because of the highest expression of memory markers (TCF7, IL7R), low level of exhaustion and concomitant expression of effector cytokines. When such transcriptional profiles were analyzed in relation to TCR clonality, the TS-TCR clonotypes were skewed towards a TS-T_{TE} or a TS-T_{Act} phenotype (66.4% and 11.9% of total TCRs respectively, even as the cellular members of each TCR clonotype family could acquire any of the TS-phenotypes (FIG. 2F). Only a minor portion of TS cells or TS-TCR clonotypes acquired TS-T P E or TS-T_{EM} states. Thus, CD8+ T cells bearing antitumor TCRs could acquire any of 5 distinct phenotypic states, but their activation and differentiation within the tumor microenvironment led to the preferential accumulation as dysfunctional cells rather than as effectors capable of perpetuating functional immunologic memory.

### Example 4: Antigen Specificities and Avidities of Tumor-Reactive TCRs

How TCR specificity and reactivity against MAAs (Andersen et al., Cancer Res. 72:1642-50 (2012); Murata et al., Elife 9:1-22 (2020)) and tumor neoantigens (NeoAgs) (Ott et al., Nature 547:217-21 (2017); Kalaora et al., Cancer Discov. 8:1366-75 (2018)) are linked to intratumoral cell state has not been well-characterized. To address this challenge in the discovery cohort, the reactivity of 561 TCRs from CD8+ TILs or PBMCs were tested, of which 299 TCRs were found to be tumor-specific. Reactivity of TCRs against cognate antigens was determined based on co-culture with autologous EBV-LCLs pulsed with hundreds of peptides corresponding to: i) personal NeoAgs, defined by prediction pipelines (Table 13) or detected as displayed on autologous tumor cells in the context of HLA class I by mass spectrometry; ii) public MAAs, tested as 12 commercially available pools of overlapping peptides spanning their entire length, or as individual peptides detected from the immunopeptidomes (Table 14-Table 15); or iii) a collection of common viral antigens (see EXAMPLE 1: Materials and Methods).

In total, the antigenic specificity (‘de-orphanize’) for 180 of 561 TCRs (166 of 299 (56%) tumor-specific, 14 of 261 (5%) non-tumor specific) was defined. The 166 tumor-specific TCRs recognized 14 personal NeoAgs and 5 public MAAs, as illustrated in FIG. 7A-7C. Antitumor TCRs isolated from HLA-A*02:01+ patients (Pt-A, Pt-B and Pt-D) were tested for the ability to cross-recognize allogeneic HLA-A*02:01+ melanoma cells. Melanoma reactivity was measured as CD137 upregulation on TCR-transduced (mTRBC+) CD8+ cells upon culture with autologous or allogeneic HLA-A*02:01-matched melanomas Tumor specificity was ruled out through parallel detection of CD137 upregulation upon challenge with matched non-tumor controls (PBMCs).

Antigen specificity screening of 299 antitumor TCRs is shown in FIG. 7A-7B. Upregulation of CD137 was assessed by flow cytometry on CD8+ T cells transduced with previously identified tumor-specific TCRs upon culture with autologous EBV-LCLs. Background, assessed using DMSO-pulsed target cells, was subtracted from each condition. Antigen recognition tested with pools of peptides corresponding to predicted immunogenic NeoAgs (see Table 13), known MAAs (see Table 14-Table 15) or immunogenic viral epitopes is shown in FIG. 7A. Reactivity was also assessed against an irrelevant peptide (Ova) or in the presence of polyclonal stimulators (PHA or PMA/ionomycin) as negative and positive controls, respectively. The black dots show the activation levels of a control Flu-specific HLA-A*02:01-restricted TCR. The dark dots above the 10% threshold show confirmed antigen-reactive TCRs, with the highest reactivity against a particular antigens, as per the legend, compared to the other tested antigens; white dots indicate TCRs reactive against an antigen which was not the highest of the panel of antigens tested, and hence considered a cross-reactive response; grey dots—negative responses. Analysis of the deconvolution of antigen specificity of TCRs reactive to NeoAg-peptide pools was carried out (data not shown), which indicated the deconvolution of the antigen specificity of TCRs reactive to NeoAg-peptide pools. After detection of TCR-reactivity in the presence of specific NeoAg-peptide pools (FIG. 7A), the identified NeoAg-reactive TCRs were tested for CD137 upregulation upon culture with autologous EBV-LCLs pulsed with individual NeoAg peptides comprising the pool. Background reactivity measured in the presence of DMSO-pulsed target cells was subtracted from reported data. The data (not shown) indicated a response to deconvoluted cognate antigens.

Antigen specificity tested using NeoAg or MAA-peptides detected by HLA-class I mass spectrometry (MS) immunopeptidome of melanoma cell lines (see Table 13-Table 15) with the addition of the MLANA protein (not retrieved by MS but known as highly immunogenic. See, Kawakami et al., J. Exp. Med. 180:347-52 (1994)) is shown in FIG. 7B. The dots above the 10% threshold indicate confirmed antigen-reactive TCRs, with the highest reactivity against a particular antigens, compared to the other tested antigens; the open dots denote TCRs reactive against an antigen which was not the highest of the panel of antigens tested, and hence considered a cross-reactive response; Distribution of antigen specificities of antitumor TCRs per patient successfully de-orphanized after screening is shown FIG. 7C. Each single slide, colored with different gray scales, denote the distinct peptides recognized by individual antitumor TCRs. Note that TCRs classified as specific for antigenic pools (n=11) represent CD8-restricted specificities showing reactivity against peptide pools (FIG. 7A), but not towards single peptides (FIG. 7B), likely due to the absence of the specific cognate antigen within the tested panels of epitopes in FIG. 7B.

In rare cases (n=3, Pt-D), the TCR reactivity against multiple targets (MAA-NeoAg or NeoAg-NeoAg) was documented, presented within the same HLA context. To link antigen specificity with the TIL-defined phenotypes, attention was focused on the 72 MAA- or NeoAg-specific TCRs either detected only in TILs or shared between TILs and blood; these constituted 4.7 to 43.9% of CD8+ TILs per patient (FIG. 3A). Antitumor MAA- and NeoAg-specific TCRs similarly displayed an exhausted phenotype, as demonstrated by a predominant distribution among T_{Ex} TILs clusters; in contrast, cells bearing “bystander” non-tumor reactive TCRs with antiviral specificity distinctly exhibited a T_{NExM} profile (FIG. 3B). Direct comparison of cells bearing the de-orphanized TCRs resulted in no differences between the transcriptional profiles of MAA and NeoAg-specific clonotypes, while both categories of tumor-specific TCRs shared the downregulation of memory markers (IL7R, CCR7, SELL, TCF7) and upregulation of exhaustion genes (PDCD1, HAVCR2, LAG3, CTLA4, ENTPD1, TOX) compared to cells bearing viral specificities (FIG. 8). Again, co-expression of the PD1 inhibitory molecule and the CD39 ectonucleosidase allowed the highest and most consistent distinction of MAA and NeoAg-specific clonotypes from virus-reactive cells. The strength of TCR tumor reactivity, measured in vitro through the CD137 upregulation assay, was not associated with a differential gene expression profile. Thus, the recognition of tumor antigens but not the class of tumor antigens per se appeared to determine the intratumoral phenotype of these CD8+ T cells.

The overall number of evaluated TCRs (pie chart), classified based on their tumor specificity and compartment of detection (blood or tumor) was analyzed (data not shown) to investigate the distribution of tested TCR clonotype families relative to the overall number of CD8+ TILs, based on their reactivity (tumor specific or non-tumor reactive). A summary of the de-orphanized antigen specificity of intratumoral TCRs with confirmed antitumor reactivity, showing percentage of CD8+ TCRs with a detected antigen specificity for particular MAAs or NeoAgs is shown in FIG. 3A. Each individual cognate antigen (MAAs or NeoAgs) is uniquely indicated with individual slices. The UMAPs of the phenotypic distribution of T cells bearing antitumor TCRs specific for MAAs or NeoAgs or TCRs specific for viral peptides is shown in FIG. 3B. The parameters affecting the avidity of antitumor TCRs were investigated and included: the RNA expression of TCR-targeted genes detected in the autologous melanoma cell line; the peptide-HLA complex affinity, and the peptide-HLA complex stability, as determined experimentally through biochemical assays (data not shown). Peptide-HLA affinity and stability could be measured for 7 of 9 MAA-antigens and 11 of 14 NeoAg targets. The effect of the position of the mutated residue within NeoAg peptides on TCR avidities as well as on peptide-HLA affinities and stabilities was investigated and determined (data not shown).

A heatmap showing genes differentially expressed between CD8+ TILs with identified MAA, NeoAg-specific or virus-specific TCRs is shown in FIG. 8. Comparisons were performed independently for each patient, and only significantly deregulated genes (adj. p<0.05, log₂FC>1 for scRNAseq data; log₂FC>0.4 for CITE-seq data) in at least 2 out of 4 patients were selected. No deregulated gene was found upon comparison of single-cells with MAA or NeoAg-TCRs; 60 RNA transcripts and 2 surface proteins resulted from comparison of MAA and/or NeoAg cells vs viral cells. Heatmap intensities depict Z scores of average gene expression within a TCR clonotype (columns). Top tracks: annotations of antigen specificity, normalized antitumor TCR reactivity, TCR avidity and patient of origin. To define the avidity of antitumour TCRs, TCR-dependent CD137 upregulation was measured on TCR-transduced (mTRBC⁺) CD8⁺ cells upon culture with patient-derived EBV-LCLs pulsed with increasing concentrations of the cognate antigen (MAAs in the top panel; NeoAgs in bottom panels) as shown in FIG. 9. Reactivity to DMSO-pulsed targets (0) and autologous melanoma (pdMel-CLs) are reported on the left; for NeoAg-specific TCRs, the dashed lines report reactivity against wild-type peptides. The EC₅₀ values were calculated from titration curves, with high EC₅₀ values corresponding to low TCR avidities (data not shown). Means with s.d. are reported, with TCR numbers corresponding to that reported in the legend of FIG. 9. Most of the NeoAg-specific TCRs display higher avidities than MAA-specific TCRs.

The expression levels of MAA or NeoAg transcripts (from bulk RNA-seq data) from which the analyzed epitopes are generated, were determined, as a measure of cognate peptide abundance in tumor cells, as analyzed from four patient-derived cell lines. The assessment of the affinity and stability of peptide-HLA complexes were determined experimentally, which indicated the strength and durability of interactions between cognate antigens and corresponding HLAs. The interactions between reported MAA or NeoAg peptides and their HLA restriction (assessed in vitro as described in Oliveira et al., Nature (2021)) were measured as previously described (Harndahl et al., J. Biomol. Screen 14:173-180 (2009)). High values corresponded to low affinity or to stable interactions.

### Example 5: Blood Dynamics of Intratumoral TCR Clonotypes Correlate with Outcome

Little is known about the dynamics of intratumural T cell clones in the periphery. To explore this, bulk TCR-sequencing of T cells from peripheral blood samples collected serially from patients over a period of 30-50 months was performed, using TCRβ-chain sequences of those CD8+ clonotypes with intratumoral T_{Ex} or T_{NExM} primary clusters as natural barcodes (data not shown). Then, Pt-A, Pt-C and Pt-D, from whom many intratumoral TCR clonotypes per T_{Ex} or T_{NExM} compartment were identified, became the focus (Table 2-Table 4).

Both T_{Ex} and T_{NExM} cells (marked by distinct TCRβ-chains) were detectable in peripheral blood, but their relative proportions and dynamics were quite different. A greater proportion of T_{NExM}-clonotypes were detected, which resulted in far more stably abundant circulating T_{NExM} TCRs than those with T_{Ex} phenotypes (p<0.0001, Fisher's exact test). Since the cells bearing T_{NExM} clonotypes were enriched in virus-reactive specificities, their relatively high circulating frequencies reflect their expected role in host immunosurveillance. The data thus support the idea that many tissue-resident T_{NExM} likely represent cells that are infiltrating tumors not due to active antigen recognition of melanoma antigens, but rather from blood perfusion or recognition of non-tumor antigens. Second, the T_{Ex}-TCR clonotype families were relatively rare among circulating T cells, consistent with the predominant residence of these high tumor-specific cells within the tumor microenvironment, where stimulation by tumor antigens could lead to acquisition of the observed dysfunction. Thus, intratumoral exhaustion state of TCR clonotypes was negatively associated with their levels of persistence in peripheral circulation. In line with these findings, the 166 antitumor TCRs with MAA or NeoAg specificity were rarely detectable in peripheral blood (median per time-point: 16 TCRs in 4 patients, 9.6%). Likewise, the majority (median per timepoint of 15 of 18 (83%) across 4 patients) of traced TCRs with antiviral specificity were present in the circulation at high frequency, consistent with their memory non-exhausted phenotype (data not shown). A similar behavior was noted for the very rare antitumor T_{NExM} TCRs (data not shown).

Finally, the relationship between levels of circulating T_{NExM} and T_{Ex} CD8+ T cells and clinical outcome was explored: the analysis was extended to an independent cohort of 14 patients with metastatic melanoma treated with immune checkpoint blockade, as previously reported (Sade-Feldman et al., Cell 176:1-20 (2019)) (Table 5). Reanalysis of this scRNAseq-TIL dataset identified clusters resembling TEA (corresponding to the published clusters 1, 2 and 3) and T_{NExM} (published clusters 4 and 6) (see EXAMPLE 1: Materials and Methods). Bulk sequencing of TCRβ-chains of T cells isolated from blood specimens from the same patients was performed to measure the frequencies of circulating T cell clonotypes corresponding to different TIL phenotypes. Consistent with the initial analysis, intratumoral T_{NExM} TCR clonotypes were stable and predominant among circulating T cells in most of the analyzed patients. Conversely, circulating T_{Ex} CD8+ T cells were quite rare but persisted at levels that correlated with the long-term outcomes: strikingly, the majority of patients who eventually succumbed to disease displayed higher levels of circulating TE A-related TCRs, both before and after immune checkpoint blockade. Compared to T_{NExM}, TEA CD8+ T cells were more abundant in patients who experienced progression, including patients who eventually died after immunotherapy, compared to responder patients. These peripheral blood dynamics mirrored the different proportions of exhausted T cells within the intratumoral microenvironment, highlighting how the frequency of circulating TCR clonotypes with a tumor-exhausted phenotype can potentially distinguish between patients with beneficial or poor response to immune checkpoint blockade.

The peripheral blood dynamics of T cells bearing TCRs detected in CD8+ TILs with primary exhausted or non-exhausted memory clusters were evaluated. For each category, levels of circulating TCR clonotypes with in vitro verified antitumor reactivity were determined. TCRs were quantified through bulk sequencing of TCRβ-chains of sorted CD3+ T cells from serial peripheral blood sampling of the 3 patients with available deep-resolution TIL sequencing results. Numbers—median number of TCRs detected longitudinally out of the total number of TCRs within each category. Behaviour of T cell dynamics was evaluated based on the clinical history and time of sample collection of each patient. The circulating levels of T cells harboring TCRs detected among intratumoral CD8+ T cell families classified as non-exhausted or exhausted, as determined from single-cell analysis of TCR Sade-Feldman et al., Cell 176:1-20 (2019)). Samples were collected from 14 melanoma patients (Sade-Feldman et al., Cell 176:1-20 (2019)) who experienced long-term remission (blue, n=7) or poor clinical outcome (orange, n=7) after immunotherapy treatment. Patients with good clinical outcome were further divided into those who did (n=4) or did not experience (n=3) disease progression following treatment. Single dots show values for patient with a single time-point available. The ratio of exhausted vs. non-exhausted TCR families for the validation cohort was calculated and compared among patients with or without long-term disease remission. The median ratio of TCR clonotypes cells with a T_{Ex} vs T_{NExM} intratumoral phenotype was calculated from peripheral blood using population frequencies measured through bulk TCR sequencing or on tumor specimens using the number of CD8 TCR families detected in published single-cell sequencing data (Sade-Feldman et al., Cell 176:1-20 (2019)) P values for significant comparisons (among responders and non responders) were calculated by Welch's t-test.

Peripheral blood dynamics of T cells containing TCRs with in vitro defined antigen specificity were evaluated. TCRs were quantified through bulk sequencing of TCRB-chains of sorted CD3+ T cells from serial peripheral blood sampling of the 4 melanoma patients within the discovery cohort. The median number of TCRs detected longitudinally out of the total number of TCRs within each category was evaluated. CD8+ TCR clonotypes identified in CD8+ TILs were traced within serial peripheral blood samples harvested from an independent cohort of melanoma patients (n=14) treated with immune checkpoint blockade therapies and with available scRNASeq data generated from TILs (Sade-Feldman et al., Cell 176:1-20 (2019)). TCRs were classified as exhausted (red) or non-exhausted (blue) based on their phenotypic primary cluster assessed by scRNAseq. Quantification of circulating TCR clonotypes was performed through bulk sequencing of TCRβ chains on circulating CD3+ cells and reported as percentage of total TCR sequences detected. Patient clinical outcomes were grouped as: survivors who did not experienced post-therapy disease recurrence (n=4); survivors who experienced disease progression after immunotherapy (n=3); and deceased patients (n=7). Analysis was conducted at different timepoint, taking into consideration the clinical history of patients and timeline of sample collection.

By analyzing the single-cell profile of truly tumor-reactive TCR clonotype, the transcriptional heterogeneity of tumor-specific CD8+ T cells was established, characterized by the acquisition of 5 distinct cell states, namely tumor specific terminally exhausted T cells (T_{TE}), activated T cells (T_{Act}), proliferating T cells (Tₚᵣₒₗ), progenitor exhausted T cells (T_{PE}), and effector memory T cells (T_{EM}). The antitumor specificity of the individual TCRs appeared to affect the relative proportion of each phenotype per clonotype family, since the transcriptional profiles for the majority of cells were dramatically skewed towards a highly exhausted T cell state (Scheper et al., Nat. Med. 25:89-94 (2019); Simoni et al., Nature 557:575-579 (2018); Gros et al., J. Clin. Invest. 124:2246-2259 (2014); Duhen et al., Nat. Commun. 9:1-13 (2018)) devoid of memory properties, were only moderately represented within a progenitor exhausted compartment, and only rarely within the CD39− PD1− memory compartment (Jansen et al., Nature 576:465-70 (2019); Krishna et al., Science 370:1328-34 (2020)). The CD39− PD1− memory compartment is further described as being CD69− and TIM3−, highly expressing CD27, CD28, and CD44, and CD45RA+. It is to be understood that markers described as negative herein includes low levels of relative expression as well as cells completely lacking (i.e., negative) a marker. For most TCR clonotype families, non-exhausted tumor-specific memory cells were quite rare, requiring the sequencing of hundreds of cells to detect even a single TCR clonotype family with this phenotype.

Second, the ability to directly identify the cognate antigens of TCRs with confirmed tumor antigen specificity establishes key relationships between tumor recognition and TCR properties. Strikingly, MAA- and NeoAg-specific TCRs drive the acquisition of remarkably similar intratumoral phenotypes, thus demonstrating that the tumor-specificity is associated with a dysfunctional cell state regardless of the type of tumor antigen recognized. Although the MAA- and NeoAg-specific T cells converged on a similar level of exhaustion, this was triggered by stimulation of TCRs with different properties. It was found that MAA-specific TCRs exhibited low avidity—not unanticipated since high avidity TCRs recognizing MAAs would be expected to undergo thymic deletion to avoid potential autoimmune recognition of MAA-expressing healthy tissues. On the other hand, MAA-specific TCRs could display high tumor recognition since their cognate antigens were abundantly available (due to high tumor expression). The majority of NeoAg-specific TCRs, by contrast, were of dramatically higher avidity that was generated by the high affinity and increased stability of mutated peptide-HLA interactions, and that was exerted towards cognate antigen expressed at relatively lower levels. In total, these observations point to the impact of central tolerance on the generation of tumor antigen-specific T cell immunity.

Third, evidence of circulating T cells that were clonally related to tumor-infiltrating exhausted tumor-specific T cells was discovered, and that their levels were correlated with disease persistence. Thus, it was concluded that patients with progressive disease have relatively abundant levels of T cells circulating with high tumor specificity and yet poor functional phenotype. In this scenario, chronic tumor co-stimulation, reflecting an incomplete response to immunotherapy, could result in an increased fraction of tumor-specific T cells locked in a poorly reversible exhausted functional state (Philip et al., Nature 545:452-6 (2017); Schietinger et al., Immunity 45:389-401 (2016)).

Data herein underscore the importance of generating new non-exhausted T cells in order to achieve a productive antitumor response. Indeed, a growing body of studies have suggested that effective antitumor responses mediated by immunotherapy arise from new specificities generated outside of the tumor and hence not subject to active exhaustion (Yost et al., Nat. Med. 25:1251-59 (2019); Wu et al., Nature 579:274-8 (2020)). Other possibilities include the notions that effective therapy might revive intratumoral T_{PE} precursor cells endowed with regenerative potential or might expand those rare T cells with both a non-exhausted memory phenotype and antitumor specificity. To this point, it is noted that Pt-C, who achieved complete response after immune checkpoint blockade, was characterized by the presence within the tumor microenvironment of antitumor TCR clonotypes having a T_{PE} primary cluster, and relatively few tumor-specific TCR clonotypes with T_{NExM} phenotypes (FIG. 2B). In line with this observation, a recent study revealed that melanoma patients with higher frequencies of intratumoral T_{PE} cells experienced a longer duration of response to checkpoint-blockade therapy (Miller et al., Nat. Immunol. 20:326-336 (2019)). Moreover, even if quite rare, less-exhausted tumor-specific cells can be expanded from TILs upon ex vivo activation, to acquire a reinvigorated memory phenotype (recently described as CD39− CD69−) that associated with response to therapy and long-term persistence (Krishna et al., Science 370:1328-34 (2020)).

Finally, since the data point to the potent antitumor recognition potential of CD39+PD1+ T_{Ex} cells, the present disclosure contemplates arming T cells having a desirable memory stem cell-like phenotype with TCRs of the discovered specificities to achieve effective and personalized tumor cytotoxicity may be achieved upon adoptive transfer of such gene-modified T cells (Leon et al., Semin. Immunol. 49:1-11 (2020)). This study provides an understanding of the interrelatedness of TCR specificity and phenotype, and the disentanglement of these two features, which enable creation of effective anti-cancer cellular therapies.

### Example 6—T Cells Infiltrating Clear Cell Renal Cell Carcinoma are Highly Exhausted

Investigation of whether the findings in melanoma could be extended to other solid tumors, such as clear cell renal cell carcinoma (ccRCC), was carried out. Despite the typical high levels of T cell infiltration, ccRCC appears to lack benefit from the accumulation of potentially cytotoxic cells within the tumor microenvironment (TME) since immune cell infiltration in ccRCC has not been equated with improved response to immunotherapy. Without being bound by theory, this might be due to the presence of T cells in a state of exhaustion and dysfunction that can only be partially reinvigorated by immunotherapies. To unravel the T cell phenotypes within ccRCC lesions, tumor infiltrating lymphocytes (TILs) isolated from 11 tumor biopsies collected before therapy were profiled through paired 5′ single cell transcriptome (scRNA-seq) and T-cell receptor sequencing (scTCR-seq) (FIG. 10A). Availability of tissues isolated at surgery from kidney region with absent tumor invasion (normal kidney) allowed to determine the T cell states enriched within the TME. Five patients were further selected for the analyses of TCR specificity, which enabled assessment of which are the T cell clonotypes with high antitumor reactivity (FIG. 10A).

After filtering T cells for expression of CD8 transcripts (see EXAMPLE 1: Materials and Methods), 40,421 CD8+ cytotoxic TILs that could be assigned to 10 transcriptionally-defined clusters were obtained (FIG. 10B). These clusters were classified based on RNA expression of T cell-related genes and by cross-labeling with reference gene-signatures from external single-cell datasets of human TILs. The composite expression of genes associated with T cell memory or exhaustion, were used to devise scores related to these cell states that were then applied to characterize the identified cell clusters (FIG. 10C-left). Phenotypic similarities between the identified T cell clusters allowed to define 3 major metaclusters (FIG. 10B-left): subsets as terminal exhausted (T_{TE}) or proliferating (TProl) TILs were characterized by the highest expression of exhaustion markers (PDCD1, TIGIT, LAGS, HAVCR2, CTLA4, TOX) and therefore could be annotated within the compartment of exhausted TILs (T_{Ex}). Conversely, subsets 4 and 6 were highly enriched in gene-signatures of memory T cells in the absence of expression of exhaustion genes, and therefore define a metacluster of non-exhausted memory TILs (T_{NExM}). While being characterized by modest exhaustion, clusters 1 and 3 showed the general lower expression of RNA transcripts characteristic of cells undergoing apoptosis (data not shown), and therefore were grouped as apoptotic TILs (T_{Ap}). ScTCR-seq revealed that highly expanded clonotype families were distributed predominantly in cells with exhausted phenotypes (FIG. 10B-right). This suggested that recognition of tumor antigens within the TME could drive the expansion of T cell clones together with the acquisition of an exhausted phenotype. In support of this, T_{Ex} clusters showed strong transcriptional similarities to the reported profiles of experimentally confirmed tumor-reactive TILs in melanoma, while T_{NExM} clusters were enriched in signatures of T cells with reported in vitro verified specificity for viral antigens (FIG. 10C-right). Importantly, TILs with exhausted features expanded mainly within the TME, and not within adjacent kidney tissue without tumor-cell infiltration (normal kidney, FIG. 10D, p.0,0025). These data show that in RCC tumor specimens, T cells with an exhausted phenotype can be identified through the expression of PDCD1, HAVCR2, CTLA4, ENTPD1, LAGS, and TOX markers. Exhausted T cells are the reservoirs of T cell clones with TCRs that are expanded within the tumor microenvironment. The identification of exhausted and expanded clonotypes is at the basis of the proposed method. These findings align with emerging evidence that even in ccRCC, interactions with tumor antigens shape the phenotype of TILs towards an exhaustion program.

### Example 7—TCR Clonotypes Anti-ccRCC Potential are Highly Exhausted

To verify that the antitumor potential of TILs could drive the acquisition of an exhausted state within the TME of ccRCC lesions, the TCRs expressed by T_{Ex}-TILs were investigated. The ability of the most highly represented TCRs with T_{Ex} phenotype (n=207) to recognize autologous tumor cell cultures was tested in 5 ccRCC patients (Pt-A-E) (FIG. 11A). Upon cloning and lentiviral transduction in T cells from healthy individuals, effector cells expressing individual TCRs were multicolour-labelled to enable parallel screening of antigenic specificities using multiparametric flow cytometry, as previously described (Oliveira et al., Nature (2021)). Transduction of the TCR signal was measured as CD137 upregulation upon co-culture of effector pools against short term cultures of autologous tumor cells and against non-tumor controls (autologous peripheral blood mononuclear cells, B cells and Epstein-Barr virus-immortalized lymphoblastoid cell lines (EBV-LCLs)). In parallel, the reactivity of 104 TCR clonotypes expanded among T_{NexM}-T cells infiltrating tumor or normal biopsies was monitored. Un-transduced (UT) T cells were analyzed as negative controls. In total, 14% (range 7-54%) of tested TCRs showed the specific recognition of autologous tumors (FIG. 11B). Strikingly, most of the detected antitumor reactivity was concentrated within T_{Ex}-TCRs (FIG. 11A). Conversely, only 3 T_{NExM}-TCRs exhibited specific tumor recognition in the absence of reactivity towards non-tumor controls (FIG. 11A). Of note, a relevant portion of T_{NExM}-TCRs recognized EBV-LCLs at high levels, supporting their specificity for viral antigens. Overall, T_{Ex}-TCRs clonotypes were highly enriched in anti-tumour specificities (p<0.0001) (FIG. 11C). These data document that T cell clones with high antitumor potential have a preferential exhausted phenotype; therefore, T cell receptors with reactivity against ccRCC tumor cells can be isolated from the exhausted compartment of T cells infiltrating tumor lesions, thus supporting the proposed method for isolating antitumor TCRs from expanded and exhausted intratumoral T cells. This is doable also in ccRCC, thus providing the application of the proposed methods to different carcinomas. These experiments further demonstrate that non-exhausted T cells can be modified with the exogenous nucleic acid comprising a sequence encoding a TCR expressed on an exhausted T cells, thus generating T cells with antitumor potential that can recognize tumors. These observations document that the proposed strategy for the isolation of antitumor TCR and modification of T cells is able to generate T cells with antitumor activity in vitro.

### Example 8—Specificity and Phenotype of TCR Clonotypes in ccRCC

To better investigate the phenotype od T cell clonotypes in relation with the specificity of their TCRs, the putative tumor antigens recognized by antitumor TILs were screened. The specificity of ccRCC TCRs isolated from TILs was determined based on co-culture with autologous EBV-LCLs pulsed with hundreds of peptides corresponding to: (i) personal neoantigens (NeoAgs) defined from whole exome sequencing of primary tumors, (ii) public tumor associated antigens (TAAs) inferred as overexpressed in tumor cells through RNA-seq of primary tumor or detected within respective human leukocyte antigen (HLA) class I immunopeptidomes of primary tumors; or (iii) common viral antigens, available as peptide pools. Only 2 tested TAAs (ANGPTL4 and IGFBP3) were able to trigger TCR reactivity. For one TAA (ANGPTL4) we were able to define the minimal cognate antigen which was able to elicit the reactivity of 2 TCRs (FIG. 12A-top). Only 1 out of 289 NeoAgs tested across 5 patients was able to trigger the reactivity of 11 CD8+ intratumoral TCRs (FIG. 12A-middle); however, when detected, NeoAg-specific TCRs exhibited the highest level of antitumor activity and avidity. Finally, a group of TCRs with no reactivity against autologous tumor was able to recognize immunogenic viral antigens (FIG. 12A-bottom). This prompted us to investigate the phenotype on antigen-specific TCR clonotypes (FIG. 12B): low avidity TAA-specific T cell clones or high avidity NeoAg-specific TILs were highly expanded and were predominantly distributed across the T_{Ex} compartment (FIG. 12B); conversely, virus-specific TCRs identified in our screening or matching known viral specificity reported in public databases exhibited a non-exhausted phenotype and were mainly distributed across the memory cell states. These bystander T cells did not exhibit direct tumor recognition (FIG. 12A-bottom) and were characterized by high expression of markers of characteristic of productive T cell responses (FIG. 12C, TCF7, IL7R, SELL), which are able to control and eradicate the cognate antigens. Conversely, TAA and NeoAg-specific T cell responses shred similar expression of exhaustion markers (FIG. 12C). These data document that similarly to melanoma, in ccRCC antitumor TILs with specificity for tumor antigens (such as tumor associated antigens or neoantigens) can be isolated from TILs with high expression of exhaustion gene-signatures (PDCD1, ENTPD1, CXCL13, TOX LAG3). Evidence is further provided that redirecting natural T cells with TCR specific for tumor antigens is able to generate T cells with in vitro reactivity against antitumor cells.

All patent publications and non-patent publications are indicative of the level of skill of those skilled in the art to which this disclosure pertains. All these publications, as well as sequences, are herein incorporated by reference to the same extent as if each individual publication were specifically and individually indicated as being incorporated by reference. Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A method of identifying T cell receptor (TCR) sequences expressed in exhausted T cells from a subject with a cancer, comprising:
collecting T cells from a tumor biopsy from the subject;
assigning the T cells into a plurality of clonotype families on the basis of TCR sequences determined by single cell T cell receptor sequencing (scTCRseq);
identifying an expanded clonotype family from among the plurality of clonotype families, wherein the T cells within the identified expanded clonotype family expresses one or more exhaustion markers comprising a) one or more of PDCD1, HAVCR2, CTLA4, ENTPD1, LAG3, and TOXRNA transcripts determined using high-throughput single cell transcriptome sequencing (scRNA seq), and/or b) one or more of PD1, Tim-3, CTLA4, CD39, and LAG3 surface proteins; and
sequencing a TCR sequence from a T cell in the expanded TCR clonotype family.

2. The method of claim 1, wherein the T cells are CD8+ T cells.

3. The method of claim 1, wherein the one or more exhaustion markers are determined using cellular indexing of transcriptomes and epitopes by sequencing (CITEseq).

4. The method of claim 1, wherein the one or more exhaustion markers comprise PD1 and CD39 proteins.

5. The method of claim 1, wherein the one or more exhaustion markers comprise PDCD1 and ENTPD1 RNA transcripts.

6. The method of claim 1, further comprising generating a cDNA encoding said TCR sequence.

7. A method of treating cancer in a subject, the method comprising:
administering to a subject in need thereof non-exhausted T cells modified with an exogenous nucleic acid comprising a sequence encoding a TCR expressed in an exhausted T cell isolated from the subject or from a subject who has a malignant tumor;
wherein the exhausted T cell expresses one or more exhaustion markers comprising a) one or more of PDCD1, HAVCR2, CTLA4, ENTPD1, LAG3, and TOXRNA transcripts, and/or b) one or more of PD1, Tim-3, CTLA4, CD39, and LAG3 surface proteins.

8. The method of claim 7, wherein the exhausted T cell is a CD8+ T cell.

9. The method of claim 7, wherein the exhausted T cells contain PD1 and CD39 surface proteins.

10. The method of claim 7, wherein the exhausted T cells co-express PDCD1 and ENTPD1 gene transcripts.

11. The method of claim 7, wherein the exhausted T cells comprise two or more groups of T cells, wherein the TCR of each group is different.

12. The method of claim 7, wherein the non-exhausted T cells are autologous non-exhausted T cells.

13. The method of claim 7, wherein the non-exhausted T cells are obtained from the peripheral blood of the subject.

14. The method of claim 7, wherein the non-exhausted T cells are memory T cells.

15. The method of claim 7, wherein the subject has a carcinoma.

16. The method of claim 15, wherein the subject has lung cancer.

17. The method of claim 15, wherein the subject has breast cancer.

18. The method of claim 15, wherein the subject has gastrointestinal cancer.

19. The method of claim 15, wherein the subject has colorectal cancer.

20. The method of claim 7, wherein the subject has melanoma.

21. The method of any one of claim 7, wherein the subject has lymphoma.

22. The method of any one of claim 7, wherein the subject has a sarcoma.

23. The method of claim 7, wherein the subject has renal cell carcinoma.

24. A non-exhausted T cell, modified with:
an exogenous nucleic acid comprising a sequence encoding a TCR expressed in an exhausted T cell in a subject with a cancer, wherein the exhausted T cell expresses one or more exhaustion markers comprising a) one or more of PDCD1, HAVCR2, CTLA4, ENTPD1, LAG3, and TOX RNA transcripts and/or b) one or more of PD1, Tim-3, CTLA4, CD39, and LAG3 surface proteins.

25. The non-exhausted T cell of claim 24, wherein the exhausted T cell contains one or more exhaustion markers comprising PDCD1 and ENTPD1 RNA transcripts.

26. The non-exhausted T cell of claim 24, wherein the exhausted T cell contains PD1 and CD39 surface proteins.

27. The non-exhausted T cell of claim 24, which is an autologous non-exhausted T cell.

28. The non-exhausted T cell of claim 24, which is an allogeneic non-exhausted T cell.

29. The non-exhausted T cell of claim 24, wherein the exhausted T cell is a CD8+ T cell.

30. The non-exhausted T cell of claim 29, which is a memory T cell.

31. The non-exhausted T cell of claim 24, wherein the exhausted T cell is a CD4+ helper T cell.


SEQUENCE LISTING (416 sequences)

SEQ ID NO: 1 - DNA, 15 bp, synthetic construct
  source 1..15: mol_type = other DNA
cagcccgattaaggt

SEQ ID NO: 2 - DNA, 15 bp, synthetic construct
  source 1..15: mol_type = other DNA
tgtatgtctgccttg

SEQ ID NO: 3 - DNA, 15 bp, synthetic construct
  source 1..15: mol_type = other DNA
cagccattcattagg

SEQ ID NO: 4 - DNA, 15 bp, synthetic construct
  source 1..15: mol_type = other DNA
agactaatagctgac

SEQ ID NO: 5 - DNA, 15 bp, synthetic construct
  source 1..15: mol_type = other DNA
tatatccttgtgagc

SEQ ID NO: 6 - DNA, 15 bp, synthetic construct
  source 1..15: mol_type = other DNA
gacaagtgatctgca

SEQ ID NO: 7 - DNA, 15 bp, synthetic construct
  source 1..15: mol_type = other DNA
tacgcctataacttg

SEQ ID NO: 8 - DNA, 15 bp, synthetic construct
  source 1..15: mol_type = other DNA
gtgtgttgtcctatg

SEQ ID NO: 9 - DNA, 15 bp, synthetic construct
  source 1..15: mol_type = other DNA
aacccaccgttgtta

SEQ ID NO: 10 - DNA, 15 bp, synthetic construct
  source 1..15: mol_type = other DNA
cagtaagttcgggac

SEQ ID NO: 11 - DNA, 15 bp, synthetic construct
  source 1..15: mol_type = other DNA
gtatagaccaaagcc

SEQ ID NO: 12 - DNA, 15 bp, synthetic construct
  source 1..15: mol_type = other DNA
tctcagacctccgta

SEQ ID NO: 13 - DNA, 15 bp, synthetic construct
  source 1..15: mol_type = other DNA
tcaccagtacctagt

SEQ ID NO: 14 - DNA, 15 bp, synthetic construct
  source 1..15: mol_type = other DNA
atggttcacgtaatc

SEQ ID NO: 15 - DNA, 15 bp, synthetic construct
  source 1..15: mol_type = other DNA
aagttcactctttgc

SEQ ID NO: 16 - DNA, 15 bp, synthetic construct
  source 1..15: mol_type = other DNA
gcttctccttcctta

SEQ ID NO: 17 - DNA, 15 bp, synthetic construct
  source 1..15: mol_type = other DNA
tattgggacacttct

SEQ ID NO: 18 - DNA, 15 bp, synthetic construct
  source 1..15: mol_type = other DNA
gcgatggtagattat

SEQ ID NO: 19 - DNA, 15 bp, synthetic construct
  source 1..15: mol_type = other DNA
tcaggtcctttcaac

SEQ ID NO: 20 - DNA, 15 bp, synthetic construct
  source 1..15: mol_type = other DNA
ctgggcaattactcg

SEQ ID NO: 21 - DNA, 15 bp, synthetic construct
  source 1..15: mol_type = other DNA
agcttacctgcacga

SEQ ID NO: 22 - DNA, 15 bp, synthetic construct
  source 1..15: mol_type = other DNA
agttcagtcaaccga

SEQ ID NO: 23 - DNA, 15 bp, synthetic construct
  source 1..15: mol_type = other DNA
gagctacttcactcg

SEQ ID NO: 24 - DNA, 15 bp, synthetic construct
  source 1..15: mol_type = other DNA
tcgagtcgcttatca

SEQ ID NO: 25 - DNA, 15 bp, synthetic construct
  source 1..15: mol_type = other DNA
ttctgggtccctaga

SEQ ID NO: 26 - DNA, 15 bp, synthetic construct
  source 1..15: mol_type = other DNA
catttgtctgccggt

SEQ ID NO: 27 - DNA, 15 bp, synthetic construct
  source 1..15: mol_type = other DNA
tctcagtgtttgtgg

SEQ ID NO: 28 - DNA, 15 bp, synthetic construct
  source 1..15: mol_type = other DNA
tcgcttggatggtag

SEQ ID NO: 29 - DNA, 15 bp, synthetic construct
  source 1..15: mol_type = other DNA
tttgtcctgtacgcc

SEQ ID NO: 30 - DNA, 15 bp, synthetic construct
  source 1..15: mol_type = other DNA
gcactcctgcatgta

SEQ ID NO: 31 - DNA, 15 bp, synthetic construct
  source 1..15: mol_type = other DNA
gttgtccgacaatac

SEQ ID NO: 32 - DNA, 15 bp, synthetic construct
  source 1..15: mol_type = other DNA
cgcgcacccattaaa

SEQ ID NO: 33 - DNA, 15 bp, synthetic construct
  source 1..15: mol_type = other DNA
acagcgccgtattta

SEQ ID NO: 34 - DNA, 15 bp, synthetic construct
  source 1..15: mol_type = other DNA
tgagaacgaccctaa

SEQ ID NO: 35 - DNA, 15 bp, synthetic construct
  source 1..15: mol_type = other DNA
ctcattgtaactcct

SEQ ID NO: 36 - DNA, 15 bp, synthetic construct
  source 1..15: mol_type = other DNA
acctttatgccacgg

SEQ ID NO: 37 - DNA, 15 bp, synthetic construct
  source 1..15: mol_type = other DNA
cgtgtttgttcctca

SEQ ID NO: 38 - DNA, 15 bp, synthetic construct
  source 1..15: mol_type = other DNA
taactcagggcctat

SEQ ID NO: 39 - DNA, 15 bp, synthetic construct
  source 1..15: mol_type = other DNA
acaatttgaacagcg

SEQ ID NO: 40 - DNA, 15 bp, synthetic construct
  source 1..15: mol_type = other DNA
gcagaaatctccctt

SEQ ID NO: 41 - DNA, 15 bp, synthetic construct
  source 1..15: mol_type = other DNA
tgtacccgcttgtga

SEQ ID NO: 42 - DNA, 15 bp, synthetic construct
  source 1..15: mol_type = other DNA
ttacctggtatccgt

SEQ ID NO: 43 - DNA, 15 bp, synthetic construct
  source 1..15: mol_type = other DNA
tgttcccgctcaact

SEQ ID NO: 44 - DNA, 15 bp, synthetic construct
  source 1..15: mol_type = other DNA
ctcagatggagtatg

SEQ ID NO: 45 - DNA, 15 bp, synthetic construct
  source 1..15: mol_type = other DNA
aatccttccgaatgt

SEQ ID NO: 46 - DNA, 15 bp, synthetic construct
  source 1..15: mol_type = other DNA
tgcaattacccggat

SEQ ID NO: 47 - DNA, 15 bp, synthetic construct
  source 1..15: mol_type = other DNA
tcaatccttccgctt

SEQ ID NO: 48 - DNA, 15 bp, synthetic construct
  source 1..15: mol_type = other DNA
ctccgaatcatgttg

SEQ ID NO: 49 - DNA, 15 bp, synthetic construct
  source 1..15: mol_type = other DNA
ttccgaggatgatct

SEQ ID NO: 50 - DNA, 15 bp, synthetic construct
  source 1..15: mol_type = other DNA
cattaacgggatgcc

SEQ ID NO: 51 - DNA, 15 bp, synthetic construct
  source 1..15: mol_type = other DNA
ttcgccgcattgagt

SEQ ID NO: 52 - DNA, 15 bp, synthetic construct
  source 1..15: mol_type = other DNA
aactccctatggagg

SEQ ID NO: 53 - DNA, 15 bp, synthetic construct
  source 1..15: mol_type = other DNA
gtccctgcaacttga

SEQ ID NO: 54 - DNA, 15 bp, synthetic construct
  source 1..15: mol_type = other DNA
gtctcttggcttaaa

SEQ ID NO: 55 - DNA, 15 bp, synthetic construct
  source 1..15: mol_type = other DNA
cgcgaacataagaag

SEQ ID NO: 56 - DNA, 15 bp, synthetic construct
  source 1..15: mol_type = other DNA
cagttcctcagttcg

SEQ ID NO: 57 - DNA, 15 bp, synthetic construct
  source 1..15: mol_type = other DNA
acgaatcaatctgtg

SEQ ID NO: 58 - DNA, 15 bp, synthetic construct
  source 1..15: mol_type = other DNA
gtctttgtcagtgca

SEQ ID NO: 59 - DNA, 15 bp, synthetic construct
  source 1..15: mol_type = other DNA
gctgcgctttccatt

SEQ ID NO: 60 - DNA, 15 bp, synthetic construct
  source 1..15: mol_type = other DNA
ccagctcattagagc

SEQ ID NO: 61 - DNA, 15 bp, synthetic construct
  source 1..15: mol_type = other DNA
aatagcgagcaagta

SEQ ID NO: 62 - DNA, 15 bp, synthetic construct
  source 1..15: mol_type = other DNA
gtagtaggctagacc

SEQ ID NO: 63 - DNA, 15 bp, synthetic construct
  source 1..15: mol_type = other DNA
cgtaacgtagagcga

SEQ ID NO: 64 - DNA, 15 bp, synthetic construct
  source 1..15: mol_type = other DNA
cttccgattcattca

SEQ ID NO: 65 - DNA, 15 bp, synthetic construct
  source 1..15: mol_type = other DNA
ttgcttaccgccaga

SEQ ID NO: 66 - DNA, 15 bp, synthetic construct
  source 1..15: mol_type = other DNA
tgtcctacccaactt

SEQ ID NO: 67 - protein, 10 aa, synthetic construct
VQKVASKIPF

SEQ ID NO: 68 - protein, 9 aa, synthetic construct
TLFHTFYEL

SEQ ID NO: 69 - protein, 10 aa, synthetic construct
TLFHTFYELL

SEQ ID NO: 70 - protein, 10 aa, synthetic construct
LFHTFYELLI

SEQ ID NO: 71 - protein, 9 aa, synthetic construct
LFHTFYELL

SEQ ID NO: 72 - protein, 10 aa, synthetic construct
TTLFHTFYEL

SEQ ID NO: 73 - protein, 9 aa, synthetic construct
KLFESKAEL

SEQ ID NO: 74 - protein, 10 aa, synthetic construct
KLFESKAELA

SEQ ID NO: 75 - protein, 9 aa, synthetic construct
GIPENSFNV

SEQ ID NO: 76 - protein, 9 aa, synthetic construct
FLFQDSKKI

SEQ ID NO: 77 - protein, 9 aa, synthetic construct
NSKKKWFLF

SEQ ID NO: 78 - protein, 10 aa, synthetic construct
RFLEYLPLRF

SEQ ID NO: 79 - protein, 9 aa, synthetic construct
TELERFLEY

SEQ ID NO: 80 - protein, 9 aa, synthetic construct
LLHTELERF

SEQ ID NO: 81 - protein, 10 aa, synthetic construct
LLHTELERFL

SEQ ID NO: 82 - protein, 9 aa, synthetic construct
KFGDLTNNF

SEQ ID NO: 83 - protein, 9 aa, synthetic construct
KLKFVTLVF

SEQ ID NO: 84 - protein, 9 aa, synthetic construct
VLAKKLKFV

SEQ ID NO: 85 - protein, 9 aa, synthetic construct
ALFASRPRF

SEQ ID NO: 86 - protein, 9 aa, synthetic construct
YNSFSSAPM

SEQ ID NO: 87 - protein, 10 aa, synthetic construct
SFSSAPMPQI

SEQ ID NO: 88 - protein, 9 aa, synthetic construct
SVGDFSQEF

SEQ ID NO: 89 - protein, 10 aa, synthetic construct
VSVGDFSQEF

SEQ ID NO: 90 - protein, 10 aa, synthetic construct
KIIAYQPYGK

SEQ ID NO: 91 - protein, 10 aa, synthetic construct
ALSPDGSIRK

SEQ ID NO: 92 - protein, 9 aa, synthetic construct
FLLTDYALS

SEQ ID NO: 93 - protein, 10 aa, synthetic construct
RLMLRKVALK

SEQ ID NO: 94 - protein, 10 aa, synthetic construct
QRLMLRKVAL

SEQ ID NO: 95 - protein, 9 aa, synthetic construct
RLMLRKVAL

SEQ ID NO: 96 - protein, 10 aa, synthetic construct
SLQRLMLRKV

SEQ ID NO: 97 - protein, 9 aa, synthetic construct
KLNFRLFVI

SEQ ID NO: 98 - protein, 10 aa, synthetic construct
RKLNFRLFVI

SEQ ID NO: 99 - protein, 10 aa, synthetic construct
KLNFRLFVIR

SEQ ID NO: 100 - protein, 10 aa, synthetic construct
RTAPRPGSQK

SEQ ID NO: 101 - protein, 10 aa, synthetic construct
RIYTGEKPFK

SEQ ID NO: 102 - protein, 10 aa, synthetic construct
SLLRAAFFGK

SEQ ID NO: 103 - protein, 10 aa, synthetic construct
LRAAFFGKCF

SEQ ID NO: 104 - protein, 10 aa, synthetic construct
VLSSLVLVPL

SEQ ID NO: 105 - protein, 9 aa, synthetic construct
NVLSSLVLV

SEQ ID NO: 106 - protein, 10 aa, synthetic construct
SLPSNVLSSL

SEQ ID NO: 107 - protein, 10 aa, synthetic construct
WRNILLLSLH

SEQ ID NO: 108 - protein, 10 aa, synthetic construct
SLHKGSLYPR

SEQ ID NO: 109 - protein, 10 aa, synthetic construct
ALQSQSISLV

SEQ ID NO: 110 - protein, 9 aa, synthetic construct
ALQSQSISL

SEQ ID NO: 111 - protein, 10 aa, synthetic construct
LLFAPEYGPK

SEQ ID NO: 112 - protein, 9 aa, synthetic construct
YLLFQNTDL

SEQ ID NO: 113 - protein, 9 aa, synthetic construct
TLLSQVNKV

SEQ ID NO: 114 - protein, 10 aa, synthetic construct
VRTLLSQVNK

SEQ ID NO: 115 - protein, 10 aa, synthetic construct
LRFNLIANQH

SEQ ID NO: 116 - protein, 9 aa, synthetic construct
NLIANQHLL

SEQ ID NO: 117 - protein, 10 aa, synthetic construct
LFRIKFKEPL

SEQ ID NO: 118 - protein, 9 aa, synthetic construct
TRKNKKLAL

SEQ ID NO: 119 - protein, 10 aa, synthetic construct
QTRKNKKLAL

SEQ ID NO: 120 - protein, 9 aa, synthetic construct
ISDRFIGIY

SEQ ID NO: 121 - protein, 10 aa, synthetic construct
EISDRFIGIY

SEQ ID NO: 122 - protein, 9 aa, synthetic construct
EISDRFIGI

SEQ ID NO: 123 - protein, 9 aa, synthetic construct
DMKARQKAL

SEQ ID NO: 124 - protein, 10 aa, synthetic construct
DMKARQKALV

SEQ ID NO: 125 - protein, 10 aa, synthetic construct
QTSIQSPSLY

SEQ ID NO: 126 - protein, 9 aa, synthetic construct
TSIQSPSLY

SEQ ID NO: 127 - protein, 9 aa, synthetic construct
AAQARLQPV

SEQ ID NO: 128 - protein, 10 aa, synthetic construct
EMKRVFGFPV

SEQ ID NO: 129 - protein, 10 aa, synthetic construct
VVDFKKNLEY

SEQ ID NO: 130 - protein, 9 aa, synthetic construct
HLARHRHLM

SEQ ID NO: 131 - protein, 10 aa, synthetic construct
SPHLARHRHL

SEQ ID NO: 132 - protein, 9 aa, synthetic construct
LLDKFVEWY

SEQ ID NO: 133 - protein, 10 aa, synthetic construct
MPAWRTRGAI

SEQ ID NO: 134 - protein, 9 aa, synthetic construct
MPAWRTRGA

SEQ ID NO: 135 - protein, 10 aa, synthetic construct
LPVTRKNMPL

SEQ ID NO: 136 - protein, 10 aa, synthetic construct
HTLGAASSFM

SEQ ID NO: 137 - protein, 9 aa, synthetic construct
HTLGAASSF

SEQ ID NO: 138 - protein, 9 aa, synthetic construct
WTNCILHEY

SEQ ID NO: 139 - protein, 9 aa, synthetic construct
QLFARARPM

SEQ ID NO: 140 - protein, 10 aa, synthetic construct
LPSFRPPTAL

SEQ ID NO: 141 - protein, 9 aa, synthetic construct
ISIQRAQPL

SEQ ID NO: 142 - protein, 9 aa, synthetic construct
NVELRRNVL

SEQ ID NO: 143 - protein, 10 aa, synthetic construct
ISSQPQVPFY

SEQ ID NO: 144 - protein, 9 aa, synthetic construct
SSQPQVPFY

SEQ ID NO: 145 - protein, 9 aa, synthetic construct
ESDLNSWPV

SEQ ID NO: 146 - protein, 10 aa, synthetic construct
ESIKEITNFK

SEQ ID NO: 147 - protein, 9 aa, synthetic construct
SIKEITNFK

SEQ ID NO: 148 - protein, 9 aa, synthetic construct
ESIKEITNF

SEQ ID NO: 149 - protein, 10 aa, synthetic construct
GLPGFVAAFV

SEQ ID NO: 150 - protein, 10 aa, synthetic construct
FVAAFVNIHL

SEQ ID NO: 151 - protein, 9 aa, synthetic construct
ALRGLPGFV

SEQ ID NO: 152 - protein, 9 aa, synthetic construct
KMIENESPL

SEQ ID NO: 153 - protein, 10 aa, synthetic construct
TLWEGGLFKL

SEQ ID NO: 154 - protein, 9 aa, synthetic construct
TEATFFPPL

SEQ ID NO: 155 - protein, 10 aa, synthetic construct
TEATFFPPLL

SEQ ID NO: 156 - protein, 10 aa, synthetic construct
TLIGLPWSFL

SEQ ID NO: 157 - protein, 10 aa, synthetic construct
GLPWSFLGWL

SEQ ID NO: 158 - protein, 9 aa, synthetic construct
KEWEDSQLV

SEQ ID NO: 159 - protein, 10 aa, synthetic construct
WEDSQLVSHL

SEQ ID NO: 160 - protein, 10 aa, synthetic construct
KEWEDSQLVS

SEQ ID NO: 161 - protein, 9 aa, synthetic construct
REELLAATL

SEQ ID NO: 162 - protein, 10 aa, synthetic construct
RLISGPSVGV

SEQ ID NO: 163 - protein, 9 aa, synthetic construct
LISGPSVGV

SEQ ID NO: 164 - protein, 9 aa, synthetic construct
AMLSKVVIL

SEQ ID NO: 165 - protein, 9 aa, synthetic construct
FMVPRDPAL

SEQ ID NO: 166 - protein, 10 aa, synthetic construct
RESFDPSAFM

SEQ ID NO: 167 - protein, 9 aa, synthetic construct
ALVVPVFEI

SEQ ID NO: 168 - protein, 9 aa, synthetic construct
QLPDFVNQL

SEQ ID NO: 169 - protein, 9 aa, synthetic construct
KRPTPTFHF

SEQ ID NO: 170 - protein, 9 aa, synthetic construct
YIHGRGWAL

SEQ ID NO: 171 - protein, 9 aa, synthetic construct
KLPKYGVKV

SEQ ID NO: 172-185 - skipped (14 entries)

SEQ ID NO: 186 - protein, 9 aa, Homo sapiens
KVLEYVIKV

SEQ ID NO: 187 - protein, 9 aa, Homo sapiens
SAYGEPRKL

SEQ ID NO: 188 - protein, 9 aa, Homo sapiens
SVFAHPRKL

SEQ ID NO: 189 - protein, 10 aa, Homo sapiens
GVYDGREHTV

SEQ ID NO: 190 - protein, 11 aa, Homo sapiens
KIWEELSVLEV

SEQ ID NO: 191 - protein, 9 aa, Homo sapiens
MLRDIIREY

SEQ ID NO: 192 - protein, 9 aa, Homo sapiens
EYTDVYPEI

SEQ ID NO: 193 - protein, 9 aa, Homo sapiens
AANKSEMAF

SEQ ID NO: 194 - protein, 9 aa, Homo sapiens
SLFGDVKKL

SEQ ID NO: 195 - protein, 9 aa, Homo sapiens
YSLEKVFGI

SEQ ID NO: 196 - protein, 9 aa, Homo sapiens
SMMQTLLTV

SEQ ID NO: 197 - protein, 9 aa, Homo sapiens
NADPQAVTM

SEQ ID NO: 198 - protein, 9 aa, Homo sapiens
VQPSKYHFL

SEQ ID NO: 199 - protein, 9 aa, Homo sapiens
FVLEKKFGI

SEQ ID NO: 200 - protein, 9 aa, Homo sapiens
SIKKKVLEF

SEQ ID NO: 201 - protein, 9 aa, Homo sapiens
KVADLIHFL

SEQ ID NO: 202 - protein, 9 aa, Homo sapiens
KVAELVHFL

SEQ ID NO: 203 - protein, 9 aa, Homo sapiens
FVYGEPREL

SEQ ID NO: 204 - protein, 10 aa, Homo sapiens
GVYAGREHFV

SEQ ID NO: 205 - protein, 9 aa, Homo sapiens
KEIDKEEHL

SEQ ID NO: 206 - protein, 9 aa, Homo sapiens
LEKKFGIQL

SEQ ID NO: 207 - protein, 9 aa, Homo sapiens
LEKVFGIQL

SEQ ID NO: 208 - protein, 9 aa, Homo sapiens
AEEAAGIGI

SEQ ID NO: 209 - protein, 8 aa, Homo sapiens
AEQSPPPY

SEQ ID NO: 210 - protein, 9 aa, Homo sapiens
ALMDKSLHV

SEQ ID NO: 211 - protein, 9 aa, Homo sapiens
EDAHFIYGY

SEQ ID NO: 212 - protein, 9 aa, Homo sapiens
GILTVILGV

SEQ ID NO: 213 - protein, 9 aa, Homo sapiens
NAPPAYEKL

SEQ ID NO: 214 - protein, 10 aa, Homo sapiens
RALMDKSLHV

SEQ ID NO: 215 - protein, 10 aa, Homo sapiens
REDAHFIYGY

SEQ ID NO: 216 - protein, 9 aa, Homo sapiens
RRNGYRALM

SEQ ID NO: 217 - protein, 11 aa, Homo sapiens
RRNGYRALMDK

SEQ ID NO: 218 - protein, 10 aa, Homo sapiens
RRRNGYRALM

SEQ ID NO: 219 - protein, 9 aa, Homo sapiens
TRRCPQEGF

SEQ ID NO: 220 - protein, 9 aa, Homo sapiens
VVPNAPPAY

SEQ ID NO: 221 - protein, 11 aa, Homo sapiens
YRALMDKSLHV

SEQ ID NO: 222 - protein, 9 aa, Homo sapiens
AAGIGILTV

SEQ ID NO: 223 - protein, 9 aa, Homo sapiens
KTWGQYWQV

SEQ ID NO: 224 - protein, 10 aa, Homo sapiens
AMLGTHTMEV

SEQ ID NO: 225 - protein, 10 aa, Homo sapiens
ALDGGNKHFL

SEQ ID NO: 226 - protein, 11 aa, Homo sapiens
SLADTNSLAVV

SEQ ID NO: 227 - protein, 9 aa, Homo sapiens
ITDQVPFSV

SEQ ID NO: 228 - protein, 9 aa, Homo sapiens
RYGSFSVTL

SEQ ID NO: 229 - protein, 11 aa, Homo sapiens
LYPEWTEAQRL

SEQ ID NO: 230 - protein, 9 aa, Homo sapiens
GQVPLIVGI

SEQ ID NO: 231 - protein, 11 aa, Homo sapiens
HQILKGGSGTY

SEQ ID NO: 232 - protein, 10 aa, Homo sapiens
HSSSHWLRLP

SEQ ID NO: 233 - protein, 9 aa, Homo sapiens
ILKGGSGTY

SEQ ID NO: 234 - protein, 15 aa, Homo sapiens
LIMPGQEAGLGQVPL

SEQ ID NO: 235 - protein, 9 aa, Homo sapiens
TEAQRLDCW

SEQ ID NO: 236 - protein, 9 aa, Homo sapiens
KQDFSVPQL

SEQ ID NO: 237 - protein, 9 aa, Homo sapiens
LIYRRRLMK

SEQ ID NO: 238 - protein, 10 aa, Homo sapiens
SCPIGENSPL

SEQ ID NO: 239 - protein, 9 aa, Homo sapiens
FLPWHRLFL

SEQ ID NO: 240 - protein, 11 aa, Homo sapiens
LLMEKEDYHSL

SEQ ID NO: 241 - protein, 9 aa, Homo sapiens
MLLAVLYCL

SEQ ID NO: 242 - protein, 9 aa, Homo sapiens
SYLEQASRI

SEQ ID NO: 243 - protein, 9 aa, Homo sapiens
EEYNSHQSL

SEQ ID NO: 244 - protein, 9 aa, Homo sapiens
AMVGAVLTA

SEQ ID NO: 245 - protein, 9 aa, Homo sapiens
SLDDYNHLV

SEQ ID NO: 246 - protein, 9 aa, Homo sapiens
SIQSRYISM

SEQ ID NO: 247 - protein, 9 aa, Homo sapiens
FLRGRLDQL

SEQ ID NO: 248 - protein, 9 aa, Homo sapiens
SLLQHLIGL

SEQ ID NO: 249 - protein, 9 aa, Homo sapiens
GLSNLTHVL

SEQ ID NO: 250 - protein, 9 aa, Homo sapiens
SQFLSLQCL

SEQ ID NO: 251 - protein, 9 aa, Homo sapiens
SQFLSLQCL

SEQ ID NO: 252 - protein, 10 aa, Homo sapiens
FLKEGACDEL

SEQ ID NO: 253 - protein, 9 aa, Homo sapiens
LYVDSLFFL

SEQ ID NO: 254 - protein, 9 aa, Homo sapiens
RLDQLLRHV

SEQ ID NO: 255 - protein, 9 aa, Homo sapiens
SQSPSVSQL

SEQ ID NO: 256 - protein, 10 aa, Homo sapiens
VLYPVPLESY

SEQ ID NO: 257 - protein, 8 aa, Homo sapiens
HARLRELL

SEQ ID NO: 258 - protein, 8 aa, Homo sapiens
LAYLHARL

SEQ ID NO: 259 - protein, 9 aa, Homo sapiens
YLHARLREL

SEQ ID NO: 260 - protein, 9 aa, Homo sapiens
AEEAAGIGI

SEQ ID NO: 261 - protein, 9 aa, Homo sapiens
ALMDKSLHV

SEQ ID NO: 262 - protein, 9 aa, Homo sapiens
GILTVILGV

SEQ ID NO: 263 - protein, 9 aa, Homo sapiens
NAPPAYEKL

SEQ ID NO: 264 - protein, 10 aa, Homo sapiens
RALMDKSLHV

SEQ ID NO: 265 - protein, 10 aa, Homo sapiens
REDAHFIYGY

SEQ ID NO: 266 - protein, 9 aa, Homo sapiens
RRNGYRALM

SEQ ID NO: 267 - protein, 11 aa, Homo sapiens
RRNGYRALMDK

SEQ ID NO: 268 - protein, 10 aa, Homo sapiens
RRRNGYRALM

SEQ ID NO: 269 - protein, 9 aa, Homo sapiens
TRRCPQEGF

SEQ ID NO: 270 - protein, 11 aa, Homo sapiens
YRALMDKSLHV

SEQ ID NO: 271 - protein, 9 aa, Homo sapiens
AAGIGILTV

SEQ ID NO: 272 - protein, 9 aa, Homo sapiens
KTWGQYWQV

SEQ ID NO: 273 - protein, 10 aa, Homo sapiens
AMLGTHTMEV

SEQ ID NO: 274 - protein, 10 aa, Homo sapiens
ALDGGNKHFL

SEQ ID NO: 275 - protein, 11 aa, Homo sapiens
SLADTNSLAVV

SEQ ID NO: 276 - protein, 9 aa, Homo sapiens
ALNFPGSQK

SEQ ID NO: 277 - protein, 9 aa, Homo sapiens
GTATLRLVK

SEQ ID NO: 278 - protein, 9 aa, Homo sapiens
LDGGNKHFL

SEQ ID NO: 279 - protein, 9 aa, Homo sapiens
LVLKRCLLH

SEQ ID NO: 280 - protein, 9 aa, Homo sapiens
MDLVLKRCL

SEQ ID NO: 281 - protein, 8 aa, Homo sapiens
LRTKAWNR

SEQ ID NO: 282 - protein, 9 aa, Homo sapiens
ITDQVPFSV

SEQ ID NO: 283 - protein, 9 aa, Homo sapiens
RYGSFSVTL

SEQ ID NO: 284 - protein, 11 aa, Homo sapiens
LYPEWTEAQRL

SEQ ID NO: 285 - protein, 9 aa, Homo sapiens
GQVPLIVGI

SEQ ID NO: 286 - protein, 11 aa, Homo sapiens
HQILKGGSGTY

SEQ ID NO: 287 - protein, 10 aa, Homo sapiens
HSSSHWLRLP

SEQ ID NO: 288 - protein, 9 aa, Homo sapiens
ILKGGSGTY

SEQ ID NO: 289 - protein, 15 aa, Homo sapiens
LIMPGQEAGLGQVPL

SEQ ID NO: 290 - protein, 9 aa, Homo sapiens
TEAQRLDCW

SEQ ID NO: 291 - protein, 9 aa, Homo sapiens
KQDFSVPQL

SEQ ID NO: 292 - protein, 9 aa, Homo sapiens
LIYRRRLMK

SEQ ID NO: 293 - protein, 10 aa, Homo sapiens
SCPIGENSPL

SEQ ID NO: 294 - protein, 15 aa, Homo sapiens
NIFDLSAPEKDKFFA

SEQ ID NO: 295 - protein, 9 aa, Homo sapiens
FLPWHRLFL

SEQ ID NO: 296 - protein, 11 aa, Homo sapiens
LLMEKEDYHSL

SEQ ID NO: 297 - protein, 9 aa, Homo sapiens
KDLGYDYSY

SEQ ID NO: 298 - protein, 9 aa, Homo sapiens
MLLAVLYCL

SEQ ID NO: 299 - protein, 9 aa, Homo sapiens
SYLEQASRI

SEQ ID NO: 300 - protein, 9 aa, Homo sapiens
EEYNSHQSL

SEQ ID NO: 301 - protein, 9 aa, Homo sapiens
AMVGAVLTA

SEQ ID NO: 302 - protein, 9 aa, Homo sapiens
SLDDYNHLV

SEQ ID NO: 303 - protein, 9 aa, Homo sapiens
GTYEGLLRR

SEQ ID NO: 304 - protein, 9 aa, Homo sapiens
SIQSRYISM

SEQ ID NO: 305 - protein, 9 aa, Homo sapiens
FLRGRLDQL

SEQ ID NO: 306 - protein, 8 aa, Homo sapiens
DQLLRHVM

SEQ ID NO: 307 - protein, 9 aa, Homo sapiens
SLLQHLIGL

SEQ ID NO: 308 - protein, 9 aa, Homo sapiens
GLSNLTHVL

SEQ ID NO: 309 - protein, 9 aa, Homo sapiens
SQFLSLQCL

SEQ ID NO: 310 - protein, 9 aa, Homo sapiens
PYLGQMINL

SEQ ID NO: 311 - protein, 9 aa, Homo sapiens
TSPRRLVEL

SEQ ID NO: 312 - protein, 10 aa, Homo sapiens
FLKEGACDEL

SEQ ID NO: 313 - protein, 9 aa, Homo sapiens
LYVDSLFFL

SEQ ID NO: 314 - protein, 9 aa, Homo sapiens
RLDQLLRHV

SEQ ID NO: 315 - protein, 9 aa, Homo sapiens
SQSPSVSQL

SEQ ID NO: 316 - protein, 10 aa, Homo sapiens
VLYPVPLESY

SEQ ID NO: 317 - protein, 8 aa, Homo sapiens
HARLRELL

SEQ ID NO: 318 - protein, 8 aa, Homo sapiens
LAYLHARL

SEQ ID NO: 319 - protein, 9 aa, Homo sapiens
YLHARLREL

SEQ ID NO: 320 - protein, 9 aa, Homo sapiens
DVYPEIIER

SEQ ID NO: 321 - protein, 9 aa, Homo sapiens
NAVGVYAGR

SEQ ID NO: 322 - protein, 9 aa, Homo sapiens
EAVLWEALR

SEQ ID NO: 323 - protein, 8 aa, Homo sapiens
RPGIHHSL

SEQ ID NO: 324 - protein, 8 aa, Homo sapiens
ESIKKKVL

SEQ ID NO: 325 - protein, 9 aa, Homo sapiens
FVLEKKFGI

SEQ ID NO: 326 - protein, 9 aa, Homo sapiens
SIKKKVLEF

SEQ ID NO: 327 - protein, 9 aa, Homo sapiens
FPSLREAAL

SEQ ID NO: 328 - protein, 8 aa, Homo sapiens
EALRKMGL

SEQ ID NO: 329 - protein, 8 aa, Homo sapiens
QVMPKTGL

SEQ ID NO: 330 - protein, 9 aa, Homo sapiens
DANRPSTAF

SEQ ID NO: 331 - protein, 9 aa, Homo sapiens
EIDKNDHLY

SEQ ID NO: 332 - protein, 8 aa, Homo sapiens
EPFTKAEM

SEQ ID NO: 333 - protein, 10 aa, Homo sapiens
KYKDYFPVIL

SEQ ID NO: 334 - protein, 9 aa, Homo sapiens
SRGPIAFWA

SEQ ID NO: 335 - protein, 9 aa, Homo sapiens
TTINFTRQR

SEQ ID NO: 336 - protein, 9 aa, Homo sapiens
SIQSRYISM

SEQ ID NO: 337 - protein, 9 aa, Homo sapiens
FLRGRLDQL

SEQ ID NO: 338 - protein, 8 aa, Homo sapiens
DQLLRHVM

SEQ ID NO: 339 - protein, 9 aa, Homo sapiens
SLLQHLIGL

SEQ ID NO: 340 - protein, 9 aa, Homo sapiens
GLSNLTHVL

SEQ ID NO: 341 - protein, 9 aa, Homo sapiens
SQFLSLQCL

SEQ ID NO: 342 - protein, 9 aa, Homo sapiens
GQHLHLETF

SEQ ID NO: 343 - protein, 9 aa, Homo sapiens
PYLGQMINL

SEQ ID NO: 344 - protein, 9 aa, Homo sapiens
TSPRRLVEL

SEQ ID NO: 345 - protein, 10 aa, Homo sapiens
FLKEGACDEL

SEQ ID NO: 346 - protein, 9 aa, Homo sapiens
LYVDSLFFL

SEQ ID NO: 347 - protein, 9 aa, Homo sapiens
RLDQLLRHV

SEQ ID NO: 348 - protein, 9 aa, Homo sapiens
SQSPSVSQL

SEQ ID NO: 349 - protein, 10 aa, Homo sapiens
VLYPVPLESY

SEQ ID NO: 350 - protein, 8 aa, Homo sapiens
HARLRELL

SEQ ID NO: 351 - protein, 8 aa, Homo sapiens
LAYLHARL

SEQ ID NO: 352 - protein, 9 aa, Homo sapiens
YLHARLREL

SEQ ID NO: 353 - protein, 9 aa, Homo sapiens
KVLEYVIKV

SEQ ID NO: 354 - protein, 9 aa, Homo sapiens
ALREEEEGV

SEQ ID NO: 355 - protein, 9 aa, Homo sapiens
ALREEGEGV

SEQ ID NO: 356 - protein, 9 aa, Homo sapiens
SVFAHPRKL

SEQ ID NO: 357 - protein, 10 aa, Homo sapiens
GVYDGREHTV

SEQ ID NO: 358 - protein, 11 aa, Homo sapiens
KIWEELSVLEV

SEQ ID NO: 359 - protein, 9 aa, Homo sapiens
MLRDIIREY

SEQ ID NO: 360 - protein, 9 aa, Homo sapiens
EYTDVYPEI

SEQ ID NO: 361 - protein, 9 aa, Homo sapiens
SLFGDVKKL

SEQ ID NO: 362 - protein, 9 aa, Homo sapiens
YSLEKVFGI

SEQ ID NO: 363 - protein, 9 aa, Homo sapiens
SMMQTLLTV

SEQ ID NO: 364 - protein, 9 aa, Homo sapiens
NADPQAVTM

SEQ ID NO: 365 - protein, 9 aa, Homo sapiens
VQPSKYHFL

SEQ ID NO: 366 - protein, 9 aa, Homo sapiens
FVLEKKFGI

SEQ ID NO: 367 - protein, 9 aa, Homo sapiens
SIKKKVLEF

SEQ ID NO: 368 - protein, 9 aa, Homo sapiens
KVADLIHFL

SEQ ID NO: 369 - protein, 9 aa, Homo sapiens
KVAELVHFL

SEQ ID NO: 370 - protein, 9 aa, Homo sapiens
FVYGEPREL

SEQ ID NO: 371 - protein, 10 aa, Homo sapiens
GVYAGREHFV

SEQ ID NO: 372 - protein, 9 aa, Homo sapiens
KEIDKEEHL

SEQ ID NO: 373 - protein, 9 aa, Homo sapiens
LEKKFGIQL

SEQ ID NO: 374 - protein, 9 aa, Homo sapiens
LEKVFGIQL

SEQ ID NO: 375 - protein, 9 aa, Homo sapiens
AEEAAGIGI

SEQ ID NO: 376 - protein, 9 aa, Homo sapiens
ALMDKSLHV

SEQ ID NO: 377 - protein, 9 aa, Homo sapiens
GILTVILGV

SEQ ID NO: 378 - protein, 10 aa, Homo sapiens
RALMDKSLHV

SEQ ID NO: 379 - protein, 10 aa, Homo sapiens
REDAHFIYGY

SEQ ID NO: 380 - protein, 9 aa, Homo sapiens
RRNGYRALM

SEQ ID NO: 381 - protein, 10 aa, Homo sapiens
RRRNGYRALM

SEQ ID NO: 382 - protein, 9 aa, Homo sapiens
TRRCPQEGF

SEQ ID NO: 383 - protein, 9 aa, Homo sapiens
VVPNAPPAY

SEQ ID NO: 384 - protein, 11 aa, Homo sapiens
YRALMDKSLHV

SEQ ID NO: 385 - protein, 9 aa, Homo sapiens
AAGIGILTV

SEQ ID NO: 386 - protein, 9 aa, Homo sapiens
KTWGQYWQV

SEQ ID NO: 387 - protein, 10 aa, Homo sapiens
AMLGTHTMEV

SEQ ID NO: 388 - protein, 10 aa, Homo sapiens
ALDGGNKHFL

SEQ ID NO: 389 - protein, 11 aa, Homo sapiens
SLADTNSLAVV

SEQ ID NO: 390 - protein, 9 aa, Homo sapiens
LDGGNKHFL

SEQ ID NO: 391 - protein, 9 aa, Homo sapiens
MDLVLKRCL

SEQ ID NO: 392 - protein, 9 aa, Homo sapiens
ITDQVPFSV

SEQ ID NO: 393 - protein, 9 aa, Homo sapiens
RYGSFSVTL

SEQ ID NO: 394 - protein, 11 aa, Homo sapiens
LYPEWTEAQRL

SEQ ID NO: 395 - protein, 15 aa, Homo sapiens
NIFDLSAPEKDKFFA

SEQ ID NO: 396 - protein, 9 aa, Homo sapiens
FLPWHRLFL

SEQ ID NO: 397 - protein, 11 aa, Homo sapiens
LLMEKEDYHSL

SEQ ID NO: 398 - protein, 9 aa, Homo sapiens
MLLAVLYCL

SEQ ID NO: 399 - protein, 9 aa, Homo sapiens
SYLEQASRI

SEQ ID NO: 400 - protein, 9 aa, Homo sapiens
EEYNSHQSL

SEQ ID NO: 401 - protein, 9 aa, Homo sapiens
SLDDYNHLV

SEQ ID NO: 402 - protein, 9 aa, Homo sapiens
SIQSRYISM

SEQ ID NO: 403 - protein, 9 aa, Homo sapiens
FLRGRLDQL

SEQ ID NO: 404 - protein, 9 aa, Homo sapiens
SLLQHLIGL

SEQ ID NO: 405 - protein, 9 aa, Homo sapiens
GLSNLTHVL

SEQ ID NO: 406 - protein, 9 aa, Homo sapiens
SQFLSLQCL

SEQ ID NO: 407 - protein, 9 aa, Homo sapiens
GQHLHLETF

SEQ ID NO: 408 - protein, 9 aa, Homo sapiens
TSPRRLVEL

SEQ ID NO: 409 - protein, 10 aa, Homo sapiens
FLKEGACDEL

SEQ ID NO: 410 - protein, 9 aa, Homo sapiens
LYVDSLFFL

SEQ ID NO: 411 - protein, 9 aa, Homo sapiens
RLDQLLRHV

SEQ ID NO: 412 - protein, 9 aa, Homo sapiens
SQSPSVSQL

SEQ ID NO: 413 - protein, 10 aa, Homo sapiens
VLYPVPLESY

SEQ ID NO: 414 - protein, 8 aa, Homo sapiens
HARLRELL

SEQ ID NO: 415 - protein, 8 aa, Homo sapiens
LAYLHARL

SEQ ID NO: 416 - protein, 9 aa, Homo sapiens
YLHARLREL